(19) United States
(12) Patent Application Publication
(10) Pub. No.: US 2023/0332145 A1
(43) Pub. Date: Oct. 19, 2023

(54) BIFUNCTIONAL MOLECULES AND METHODS OF USING THEREOF

(71) Applicant: Flagship Pioneering, Inc., Cambridge, MA (US)

(72) Inventors: Nathan Wilson STEBBINS, Cambridge, MA (US); Benjamin Andrew PORTNEY, Somerville, MA (US); Eric Bruno VALEUR, Newton, MA (US); Chih-Chi Yuan, Chestnut Hill, MA (US); Mitchell Guttman, Los Angeles, CA (US)

(73) Assignee: Flagship Pioneering, Inc., Cambridge, MA (US)

(21) Appl. No.: 17/914,307

(22) PCT Filed: Mar. 24, 2021
(86) PCT No.: PCT/US2021/024008

§ 371 Date: Sep. 23, 2022

Related U.S. Application Data
(60) Provisional application No. 62/994,246, filed on Mar. 24, 2020.

(51) Int. Cl. C12N 15/113; C12N 15/115; A61K 47/54

(52) U.S. Cl. CPC C12N 15/113; A61K 47/549; C12N 15/115; C12N 2310/11; C12N 2310/16; C12N 2310/3231; C12N 2310/3519

(57) ABSTRACT

The present disclosure relates generally to compositions of synthetic bifunctional molecules comprising a first domain that specifically binds to a target ribonucleic acid sequence and a second domain that specifically binds to a target protein, and uses thereof.

### BACKGROUND

At any given time, the amount of a particular protein in a cell reflects the balance between that protein’s synthetic and degradative biochemical pathways. On the synthetic side of this balance, protein production starts at transcription and continues with translation. Thus, control of these processes plays a critical role in determining what proteins are present in a cell and in what amounts. In addition, the way in which a cell processes its RNA transcripts and newly made proteins also greatly influences protein levels. The amounts and types of mRNA molecules in a cell reflect the function of that cell. In fact, thousands of transcripts are produced every second in every cell. Given this statistic, it is not surprising that the primary control point for gene expression is usually at the very beginning of the protein production process — the initiation of transcription. RNA transcription makes an efficient control point because many proteins can be made from a single mRNA molecule. Indeed, diseases or their symptoms can be prevented, ameliorated, or treated by selectively increasing the transcription or the RNA level of a relevant gene.

An binding specificity between binding partners may provide tools to effectively deliver molecules to a specific target, for example, to selectively increase the transcription or the RNA level of a gene.

### SUMMARY

In some aspects, a synthetic bifunctional molecule as described herein comprises: a first domain comprising a first small molecule or an antisense oligonucleotide (ASO), wherein the first domain specifically binds to a target ribonucleic acid (RNA) sequence; and a second domain comprising a second small molecule or an aptamer, wherein the second domain specifically binds to a target endogenous protein; and wherein the first domain is conjugated to the second domain. In some embodiments, the target endogenous protein is an intracellular protein. In some embodiments, the first domain is conjugated to the second domain by a linker molecule. In some embodiments, the linker molecule is a chemical linker. In some embodiments, the first domain is an ASO. In some embodiments, the ASO comprises one or more locked nucleic acids (LNA), one or more modified nucleobases, or a combination thereof. In some embodiments, the ASO may include any useful modification, such as to the sugar, the nucleobase, or the intemucleoside linkage (e.g., to a linking phosphate / to a phosphodiester linkage / to the phosphodiester backbone). In some embodiments, the ASO comprises at least two locked nucleic acids. In some embodiments, the ASO comprises at least three locked nucleotides. In some embodiments, the ASO comprises at least four locked nucleotides. In some embodiments, the ASO comprises at least five locked nucleotides. In some embodiments, the ASO comprises at least six locked nucleotides In some embodiments, the ASO comprises one to seven locked nucleotides. In some embodiments, the ASO comprises a 5′ locked terminal nucleotide, a 3′ locked terminal nucleotide, or a 5′ and a 3′ locked terminal nucleotides. In some embodiments, the ASO comprises a locked nucleotide at an internal position in the ASO. In some embodiments, the ASO comprises a sequence comprising 30% to 60% GC content. In some embodiments, the ASO comprises a length from 8 to 30 nucleotides. In some embodiments, the ASO comprises a length from 12 to 25 nucleotides. In some embodiments, the ASO comprises a length from 14 to 24 nucleotides. In some embodiments, the ASO comprises a length from 16 to 20 nucleotides. In some embodiments, the ASO is selected from the group consisting of those listed in Tables 1A and 1B. In some embodiments, the first domain is a first small molecule. In some embodiments, the first small molecule is selected from the group consisting of those listed in Table 2. In some embodiments, the second domain is a second small molecule. In some embodiments, the second small molecule is selected from those listed in Table 3. In some embodiments, the second small molecule is an organic compound having a molecular weight of 900 daltons or less. In some embodiments, the second small molecule is an organic compound having a molecular weight of 600 daltons or less. In some embodiments, the second small molecule is JQ1. In some embodiments, the second small molecule is iBET762. In some embodiments, the second small molecule is ibrutinib. In some embodiments, the second domain is an aptamer. In some embodiments, the aptamer is selected from those listed in Table 3. In some embodiments, the linker is conjugated at a 5′ end or a 3′ end of the ASO. In some embodiments, the linker is conjugated at an internal position on the ASO. In some embodiments, the synthetic bifunctional molecule further comprising a third domain conjugated to the first domain, the linker, the second domain, or any combination thereof. In some embodiments, the third domain comprises a third small molecule. In some embodiments, the third domain enhances uptake of the synthetic bifunctional molecule by a cell. In some embodiments, the synthetic bifunctional molecule further comprises one or more second domains. In some embodiments, each of the one or more second domains specifically binds to a single target endogenous protein. In some embodiments, the target ribonucleic acid sequence is a nuclear RNA or a cytoplasmic RNA. In some embodiments, the nuclear RNA or the cytoplasmic RNA is a long noncoding RNA (lncRNA), pre-mRNA, mRNA, microRNA, enhancer RNA, transcribed RNA, nascent RNA, chromosome-enriched RNA, ribosomal RNA, membrane enriched RNA, or mitochondrial RNA. In some embodiments, the target ribonucleic acid is an intron. In some embodiments, the target ribonucleic acid is an exon. In some embodiments, the target ribonucleic acid is an untranslated region. In some embodiments, the target ribonucleic acid is a region translated into proteins.

In some aspects, a synthetic bifunctional molecule as described herein comprises: a first domain comprising a first small molecule or an antisense oligonucleotide (ASO), wherein the first domain specifically binds to a target ribonucleic acid (RNA) sequence; a plurality of second domains, each comprising a second small molecule or an aptamer, wherein each of the plurality of second domains specifically binds to a target endogenous protein; and a linker that conjugates the first domain to the plurality of second domains. In some embodiments, each of the plurality of second domains comprises the second small molecule. In some embodiments, the synthetic bifunctional molecule comprises 2, 3, 4, or 5 second domains. In some embodiments, the plurality of second domains comprises the same domain. In some embodiments, the plurality of second domains comprises different domains. In some embodiments, the plurality of second domains binds to a same target endogenous protein. In some embodiments, the plurality of second domains binds to different target endogenous proteins. In some embodiments, the synthetic bifunctional molecule further comprises a third domain conjugated to the first domain, the linker, the plurality of second domains, or any combination thereof. In some embodiments, the third domain comprises a third small molecule. In some embodiments, the third domain enhances uptake of the synthetic bifunctional molecule by a cell. In some embodiments, the target endogenous protein is an intracellular protein. In some embodiments, the target endogenous protein is an enzyme or a regulatory protein. In some embodiments, the second domain binds to an active site or an allosteric site on the target endogenous protein. In some embodiments, binding of the second domain to the target endogenous protein is noncovalent or covalent. In some embodiments, binding of the second domain to the target endogenous protein is covalent and reversible or covalent and irreversible. In some embodiments, the target endogenous protein increases transcription of a gene selected from those listed in Table 4 or Table 5. In some embodiments, a ribonucleic acid comprising the target nucleic acid sequence increases transcription of a gene selected from those listed in Table 4 or Table 5. In some embodiments, transcription of the gene is upregulated or increased. In some embodiments, the gene is associated with a disease from those listed in Table 5. In some embodiments, the gene is associated with a disease or disorder. In some embodiments, the disease is any disorder caused by an organism. In some embodiments, the organism is a prion, a bacteria, a virus, a fungus, or a parasite. In some embodiments, the disease or disorder is a cancer, a metabolic disease, an inflammatory disease, an autoimmune disease, a cardiovascular disease, an infectious disease, a genetic disease, or a neurological disease. In some embodiments, the disease is a cancer and wherein the target gene is an oncogene. In some embodiments, the disease is a haploinsufficiency disease or a loss of function disease.

In some aspects, a method of increasing transcription or an RNA level of a gene in a cell comprises: administering to a cell a synthetic bifunctional molecule comprising: a first domain comprising a first small molecule or an antisense oligonucleotide (ASO), wherein the first domain specifically binds to a target ribonucleic acid sequence; a second domain comprising a second small molecule or an aptamer, wherein the second domain specifically binds to a target endogenous protein; and a linker that conjugates the first domain to the second domain; wherein the target endogenous protein increases transcription or an RNA level of a gene in the cell. In some embodiments, the method increases transcription of the gene. In some embodiments, the method increases the RNA level of the gene. In some embodiments, the cell is a human cell. In some embodiments, the human cell is infected with a virus. In some embodiments, the human cell is a cancer cell. In some embodiments, the cell is a bacterial cell. In some embodiments, the linker is a chemical linker. In some embodiments, the first domain is an ASO. In some embodiments, the ASO comprises one or more locked nucleic acids (LNA), one or more modified nucleobases, or a combination thereof. In some embodiments, the ASO may include any useful modification, such as to the sugar, the nucleobase, or the internucleoside linkage (e.g., to a linking phosphate / to a phosphodiester linkage / to the phosphodiester backbone). In some embodiments, the ASO comprises at least two locked nucleotides. In some embodiments, the ASO comprises at least three locked nucleotides. In some embodiments, the ASO comprises at least four locked nucleotides. In some embodiments, the ASO comprises at least five locked nucleotides. In some embodiments, the ASO comprises at least six locked nucleotides. In some embodiments, the ASO comprises one to seven locked nucleotides. In some embodiments, the ASO comprises a 5′ locked terminal nucleotide, a 3′ locked terminal nucleotide, or a 5′ and a 3′ locked terminal nucleotides. In some embodiments, the ASO comprises a locked nucleotide at an internal position in the ASO. In some embodiments, the ASO comprises a sequence comprising 30% to 60% GC content. In some embodiments, the ASO comprises a length from 8 to 30 nucleotides. In some embodiments, the ASO comprises a length from 12 to 25 nucleotides. In some embodiments, the ASO comprises a length from 14 to 24 nucleotides. In some embodiments, the ASO comprises a length from 16 to 20 nucleotides. In some embodiments, the first domain is a first small molecule. In some embodiments, the first small molecule is selected from the group consisting of Table 2. In some embodiments, the second domain is a second small molecule. In some embodiments, the second small molecule binds to a protein (e.g, an intracellular protein). In some embodiments, the second small molecule is selected from Table 3. In some embodiments, the second small molecule is an organic compound having a molecular weight of 900 daltons or less. In some embodiments, the second small molecule is JQ1. In some embodiments, the second small molecule is iBET762. In some embodiments, the second small molecule is ibrutinib. In some embodiments, the second domain is an aptamer. In some embodiments, the aptamer is selected from Table 3. In some embodiments, the linker is conjugated at a 5′ end or a 3′ end of the ASO. In some embodiments, the linker is conjugated at an internal position on the ASO. In some embodiments, the synthetic bifunctional molecule further comprises a third domain conjugated to the first domain, the linker, the second domain, or a combination thereof. In some embodiments, the third domain comprises a third small molecule. In some embodiments, the third domain enhances uptake of the synthetic bifunctional molecule by the cell. In some embodiments, the synthetic bifunctional molecule further comprises one or more second domains. In some embodiments, each of the one or more second domains specifically binds to a single target endogenous protein. In some embodiments, the target ribonucleic acid sequence is a nuclear RNA or a cytoplasmic RNA. In some embodiments, the nuclear RNA or the cytoplasmic RNA is a long noncoding RNA (lncRNA), pre-mRNA, mRNA, microRNA, enhancer RNA, transcribed RNA, nascent RNA, chromosome-enriched RNA, ribosomal RNA, membrane enriched RNA, or mitochondrial RNA. In some embodiments, the target sequence is an intron or an exon. In some embodiments, the target sequence is translated or untranslated region on an mRNA or pre-mRNA.

In some aspects, a method of increasing transcription or an RNA level of a gene in a cell comprises: administering to a cell a synthetic bifunctional molecule comprising: a first domain comprising a first small molecule or an antisense oligonucleotide (ASO), wherein the first domain specifically binds to a target ribonucleic acid (RNA) sequence; a plurality of second domains, each comprising a second small molecule or an aptamer, wherein each of the plurality of second domains specifically bind to a target endogenous protein; and a linker that conjugates the first domain to the plurality of second domains; wherein the target endogenous protein increases transcription of a gene in the cell. In some embodiments, the method increases transcription of the gene. In some embodiments, the method increases the RNA level of the gene. In some embodiments, each of the plurality of second domains comprises the second small molecule. In some embodiments, the plurality of second domains is 2, 3, 4, or 5 second domains. In some embodiments, each of the plurality of second domains comprises the same domain. In some embodiments, each of the plurality of second domains comprises different domains. In some embodiments, each of the plurality of second domains binds to a same target endogenous protein. In some embodiments, each of the plurality of second domains binds to different target endogenous proteins. In some embodiments, the synthetic bifunctional molecule further comprises a third domain conjugated to the first domain, the linker, the second domain, or a combination thereof. In some embodiments, the third domain comprises a third small molecule. In some embodiments, the third domain enhances uptake of the synthetic bifunctional molecule by the cell. In some embodiments, the target endogenous protein is an intracellular protein. In some embodiments, the target endogenous protein is an enzyme or a regulatory protein. In some embodiments, each of the plurality of second domains specifically bind to an active site or an allosteric site on the target endogenous protein. In some embodiments, binding of each of the plurality of second domains to the target endogenous protein is noncovalent or covalent. In some embodiments, binding of each of the plurality of second domains to the target endogenous protein is covalent and reversible or covalent and irreversible. In some embodiments, the gene is selected from Table 4 or Table 5. In some embodiments, transcription of the gene is upregulated or increased. In some embodiments, the gene is associated with a disease from Table 5. In some embodiments, the gene is associated with a disease or disorder. In some embodiments, the disease is any disorder caused by an organism. In some embodiments, the organism is a prion, a bacteria, a virus, a fungus, or a parasite. In some embodiments, the disease or disorder is a cancer, a metabolic disease, an inflammatory disease, an autoimmune disease, a cardiovascular disease, an infectious disease, a genetic disease, or a neurological disease.

### BRIEF DESCRIPTION OF THE DRAWINGS

The following detailed description of the embodiments of the present disclosure will be better understood when read in conjunction with the appended drawings. For the purpose of illustrating the present disclosure, there are shown in the drawings embodiments, which are presently exemplified. It should be understood, however, that the present disclosure is not limited to the precise arrangement and instrumentalities of the embodiments shown in the drawings.

FIG. 1 is an image showing that the conjugate of Ibrutinib and an ASO, an exemplary embodiment of the bifunctional molecules as provided herein, forms a tertiary complex with Bruton’s Tyrosine Kinase (BTK) via Ibrutinib and the Cy5-labeled IVT RNA via the ASO, respectively.

FIG. 2 shows PVT1 ASO1-JQ1 induced MYC expression. PVT1 ASO1-JQ1 were transfected to HEK293T cells at 400, 200, 100, and 50 nM by RNAiMax. Cells were harvested 24 hours after transfection for qPCR analysis. Free JQ1, free PVT1 ASO, and Scramble ASO-JQ1 (Scr-JQ1) were tested as negative controls.

FIGS. 3A and 3B depicts negative controls for PVT1 ASO1-JQ1 showing specificity of the molecule. Two Scramble ASOs and eight non-PVT1 targeting (NPT) ASOs were conjugated to JQ1 and transfected to HEK293T cells at 100 nM by RNAiMax. Cells were harvested 24 hours after transfection for qPCR analysis. Free JQ1 and free PVT1 ASO1 were tested as additional negative controls (FIG. 3A). PVT1 ASO1 binder and free JQ1 were transfected together to HEK293T cells at 100 nM by RNAiMax. Cells were harvested 24 hours after transfection for qPCR analysis. Free JQ1, PVT1 ASO1 binder, and PVT1 ASO1 degrader were tested as additional negative controls (FIG. 3B).

FIG. 4 shows that PVT1 ASO1-(-)JQ1 is inactive in inducing MYC expression. PVT1 ASO1-(-)JQ1 was transfected to HEK293T cells at 100 nM by RNAiMax. Cells were harvested 24 hours after transfection for qPCR analysis. Free JQ1, free PVT1 ASO, and Scramble ASO-JQ1 (ScrB-JQ1) were tested as negative controls.

FIG. 5 depicts dose titration of PVT1 ASO1-JQ1. PVT1 ASO1-JQ1 and controls were transfected to HEK293T cells at indicated doses by RNAiMax. Cells were harvested 24 hours after transfection for qPCR analysis.

FIG. 6 is an image showing that swapping nucleotides in the center of PVT1 ASO1 sequences inactivate the PVT1 ASO1-JQ1 molecule. 2 to 5 nucleotides within PVT1 ASO1 sequence were swapped (gray blocks within black bars on left side of figure). PVT1 ASO1-JQ1 molecules were transfected at 100 nM to HEK293T cells by RNAiMax (right side of figure). Cells were harvested 24 hours after transfection for qPCR analysis. Results showed that swapping the first two nucleotides at the 5′end or the first 4 nucleotides at the 3′end had less impact on the activities of the molecules (e.g., PVT1-Scr1, PVT1-Scr4 and PVT1-Scr8); while swapping nucleotides in the center of the ASO sequence had a pronounced impact on the activities.

FIGS. 7 and 8 depict that PVT1 ASO1-JQ1 treatment increases MYC gene transcript (FIG. 7) and also MYC protein (FIG. 8) in cells.

FIG. 9 depicts two different linkers between ASO and small molecule showing similar activities. V1 PVT1 ASO1-JQ1 And V2 PVT1 ASO1-JQ1 were transfected to HEK293T cells at 400, 200, 100, 50, 25, 12.5, 6.25, 3.125 nM by RNAiMax. Cells were harvested 24 hours after transfection for qPCR analysis. Free JQ1, PVT1 ASO1, Scramble ASO-JQ1 (ScrB-JQ1), and V2 PVT1 ASO1-JQ1 were included as negative controls.

FIG. 10 depicts PVT1 ASO1-iBET762 induced MYC expression. PVT1 ASO1-iBET762 were transfected to HEK293T cells at 400, 200, 100, and 50 nM by RNAiMax. Cells were harvested 24 hours after transfection for qPCR analysis. Free iBET762, free PVT1 ASO, and Scramble ASO-iBET762 (Scr-iBET762) were tested as negative controls.

FIGS. 11A and 11B depicts additional PVT1 ASO-JQ1 molecules inducing MYC expression. Genomic localization of PVT1 ASO1 to ASO20 (FIG. 11A). PVT1 ASO1-JQ1 to PVT1 ASO20-JQ1 were transfected to HEK293T cells at 400, 133, 44, and 15 nM by RNAiMax (FIG. 11B). Cells were harvested 24 hours after transfection for qPCR analysis.

FIG. 12 depicts additional PVT1 ASO-iBET762 molecules inducing MYC expression. PVT1 ASO1-iBET762 to PVT1 ASO20-iBET762 were transfected to HEK293T cells at 400, 133, 44, and 15 nM by RNAiMax. Cells were harvested 24 hours after transfection for qPCR analysis.

FIGS. 13A-13B depicts defining an active pocket supporting the increase of MYC expression. PVT1 ASO1-JQ1, PVT1 ASO30-JQ1 to PVT1 ASO33-JQ1 were transfected to HEK293T cells at 400, 133, 44, and 15 nM by RNAiMax. Cells were harvested 24 hours after transfection for qPCR analysis. Results showed that PVT1 ASO30-JQ1 to PVT1 ASO33-JQ1 did not increase MYC expression (FIG. 13A). Genomic localization of PVT1 ASO1 to ASO20, and ASO29 to ASO33. The identified active pocket (Active pocket 1) is indicated (FIG. 13B).

FIGS. 14A-14C depict PVT1 ASO-JQ1 molecules inducing MYC expression. Genomic localization of PVT1 ASO21 to ASO29 was shown (FIG. 14A). Control PVT1 ASO1-JQ1, and PVT1 ASO21-JQ1 to PVT1 ASO29-JQ1 were transfected to HEK293T cells at 400, 133, 44, and 15 nM by RNAiMax. Cells were harvested 24 hours after transfection for qPCR analysis (FIG. 14B). Genomic localization of PVT1 ASO24 and ASO25. The identified active pocket (active pocket 2) is indicated in FIG. 14C.

FIG. 15 depicts MYC ASO-JQ1 molecules inducing MYC expression. MYC ASO1-JQ1 to PVT1 ASO6-JQ1 and control PVT1 ASO1-JQ1 were transfected to HEK293T cells at 400, 133, 44, and 15 nM by RNAiMax. Cells were harvested 24 hours after transfection for qPCR analysis.

FIG. 16 depicts MYC ASO-iBET762 molecules inducing MYC expression. MYC ASO1-iBET762 to PVT1 ASO6-iBET762 and control PVT1 ASO1-iBET762 were transfected to HEK293T cells at 400, 133, 44, and 15 nM by RNAiMax. Cells were harvested 24 hours after transfection for qPCR analysis.

FIG. 17 depicts SCN1A ASO1-JQ1 molecules inducing SCN1A expression. Each of JQ1, SCN1A-ASO1, Scr-JQ1, and SCN1A ASO1-JQ1 (“SCN1A-JQ1”) was transfected to SK-N-AS cells at 100, 50, 25, 12.5, 6.25, and 3.125 nM by RNAiMax. Cells were harvested 48 hours after transfection for qPCR analysis.

FIG. 18 depicts SCN1A ASO1-iBET762 molecules inducing SCN1A expression. Each of iBET762, SCN1A-ASO1, Scr- iBET762, and SCN1A ASO1-iBET762 (“SCN1A-iBET762”) was transfected to SK-N-AS cells at 100, 50, 25, 12.5, 6.25, and 3.125 nM by RNAiMax. Cells were harvested 48 hours after transfection for qPCR analysis.

FIG. 19 depicts qRT-PCR showing the RNA levels of HSP70, MALAT1, and ACTB, after RNA immunoprecipitation (RIP) of BTK protein in cells that were transfected with BTK and ibrutinib-conjugated ASOs targeting HSP70 and MALAT1.

FIG. 20 depicts that SYNGAP1 ASO2-JQ1 increased SYNGAP1 expression. SYNGAP1 ASO1-JQ1 to SYNGAP1 ASO4-JQ1 were transfected to HEK293T cells at 200, and 67 nM by RNAiMax. Cells were harvested 48 hours after transfection for qPCR analysis.

### DETAILED DESCRIPTION

The present disclosure generally relates to bifunctional molecules. Generally, the bifunctional molecules are designed and synthesized to bind to two or more unique targets. A first target can be a nucleic acid sequence, for example a RNA. A second target can be a protein, peptide, or other effector molecule. The bifunctional molecules described herein comprise a first domain that specifically binds to a target nucleic acid sequence (e.g., a target RNA sequence) and a second domain that specifically binds to a target protein. Bifunctional molecule compositions, preparations of compositions thereof and uses thereof are also described.

The present disclosure is described with respect to particular embodiments and with reference to certain figures but thepresent disclosure is not limited thereto but only by the claims. Terms as set forth hereinafter are generally to be understood in their common sense unless indicated otherwise.

The synthetic bifunctional molecules comprising a first domain that specifically binds to a target RNA sequence and a second domain that specifically binds to a target endogenous protein, compositions comprising such bifunctional molecules, methods of using such bifunctional molecules, etc. as described herein are based in part on the examples which illustrate how the bifunctional molecules comprising different components, for example, unique sequences, different lengths, and modified nucleotides (e.g., locked nucleotides), be used to achieve different technical effects (e.g., increasing a RNA level or transcription in a cell). It is on the basis of inter alia these examples that the description hereinafter contemplates various variations of the specific findings and combinations considered in the examples.

### Bifunctional Molecule

In one aspect, the present disclosure relates to a bifunctional molecule comprising a first domain that binds to a target nucleic acid sequence (e.g., an RNA sequence) and a second domain that binds to a target protein. The bifunctional molecules described herein are designed and synthesized so that a first domain is conjugated to a second domain.

### First Domain

The bifunctional molecule as described herein comprise a first domain that specifically binds to a target nucleic acid sequence (e.g., an RNA sequence). In some embodiments, the first domain comprises a small molecule or an antisense oligonucleotide (ASO).

### Antisense Oligonucleotide (ASO)

In some embodiments, the first domain of the bifunctional molecule as described herein, which specifically binds to a target RNA sequence, is an ASO.

Routine methods can be used to design a nucleic acid that binds to the target sequence with sufficient specificity. As used herein, the terms “nucleotide,” “oligonucleotide,” and “nucleic acid” are used interchangeably. In some embodiments, the methods include using bioinformatics methods known in the art to identify regions of secondary structure. As used herein, the term “secondary structure” refers to the basepairing interactions within a single nucleic acid polymer or between two polymers. For example, the secondary structures of RNA include, but are not limited to, a double-stranded segment, bulge, internal loop, stem-loop structure (hairpin), two-stem junction (coaxial stack), pseudoknot, g-quadruplex, quasi-helical structure, and kissing hairpins. For example, “gene walk” methods can be used to optimize the activity of the nucleic acid; for example, a series of oligonucleotides of 10-30 nucleotides spanning the length of a target RNA or a gene can be prepared, followed by testing for activity. Optionally, gaps, e.g., of 5-10 nucleotides or more, can be left between the target sequences to reduce the number of oligonucleotides synthesized and tested.

Once one or more target regions, segments or sites have been identified, e.g., within a sequence of interest, nucleotide sequences are chosen that are sufficiently complementary to the target, i.e., that hybridize sufficiently well and with sufficient specificity (i.e., do not substantially bind to other non-target RNAs), to give the desired effect, e.g., binding to the RNA.

As described herein, hybridization means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. Complementary, as used herein, refers to the capacity for precise pairing between two nucleotides. For example, if a nucleotide at a certain position of an oligonucleotide is capable of hydrogen bonding with a nucleotide at the same position of a RNA molecule, then the ASO and the RNA are considered to be complementary to each other at that position. The ASO and the RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the ASO and the RNA target. For example, if a base at one position of the ASO is capable of hydrogen bonding with a base at the corresponding position of a RNA, then the bases are considered to be complementary to each other at that position. 100% complementarity is not required.

It is understood in the art that a complementary nucleic acid sequence need not be 100% complementary to that of its target nucleic acid to be specifically hybridisable. A complementary nucleic acid sequence for purposes of the present methods is specifically hybridisable when binding of the sequence to the target RNA molecule or the target gene elicit the desired effects as described herein, and there is a sufficient degree of complementarity to avoid non-specific binding of the sequence to non-target RNA sequences under conditions in which specific binding is desired, e.g., under physiological conditions in the case of in vivo assays or therapeutic treatment, and in the case of in vitro assays, under conditions in which the assays are performed under suitable conditions of stringency.

In general, the ASO useful in the methods described herein have at least 80% sequence complementarity to a target region within the target nucleic acid, e.g., 90%, 95%, or 100% sequence complementarity to the target region within an RNA. For example, an antisense compound in which 18 of 20 nucleobases of the antisense oligonucleotide are complementary, and would therefore specifically hybridize, to a target region would represent 90 percent complementarity. Percent complementarity of an ASO with a region of a target nucleic acid can be determined routinely using basic local alignment search tools (BLAST programs) (Altschul et al, J. Mol. Biol., 1990, 215, 403-410; Zhang and Madden, Genome Res., 1997, 7, 649-656). The ASO that hybridizes to an RNA can be identified through routine experimentation. In general, the ASO must retain specificity for their target, i.e., must not directly bind to other than the intended target.

In certain embodiments, the ASO described herein comprises modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, one nucleoside comprising a modified nucleobase is in the central region of a modified oligonucleotide. In certain such embodiments, the sugar moiety of said nucleoside is a 2′-β-D-deoxyribosyl moiety. In certain such embodiments, the modified nucleobase is selected from: 5-methyl cytosine, 2-thiopyrimidine, 2-thiothymine, 6-methyladenine, inosine, pseudouracil, or 5-propynepyrimidine.

In certain embodiments, the ASO described herein comprises modified and/or unmodified intemucleoside linkages arranged along the oligonucleotide or region thereof in a defined pattern or motif. In certain embodiments, each intemucleoside linkage is a phosphodiester intemucleoside linkage (P=O). In certain embodiments, each intemucleoside linkage of a modified oligonucleotide is a phosphorothioate intemucleoside linkage (P=S). In certain embodiments, each intemucleoside linkage of a modified oligonucleotide is independently selected from a phosphorothioate intemucleoside linkage and phosphodiester intemucleoside linkage. In certain embodiments, each phosphorothioate intemucleoside linkage is independently selected from a stereorandom phosphorothioate, a (Sp) phosphorothioate, and a (Rp) phosphorothioate. In certain embodiments, the intemucleoside linkages within the central region of a modified oligonucleotide are all modified. In certain such embodiments, some or all of the intemucleoside linkages in the 5′-region and 3′-region are unmodified phosphate linkages. In certain embodiments, the terminal intemucleoside linkages are modified. In certain embodiments, the intemucleoside linkage motif comprises at least one phosphodiester intemucleoside linkage in at least one of the 5′-region and the 3′-region, wherein the at least one phosphodiester linkage is not a terminal intemucleoside linkage, and the remaining intemucleoside linkages are phosphorothioate intemucleoside linkages. In certain such embodiments, all of the phosphorothioate linkages are stereorandom. In certain embodiments, all of the phosphorothioate linkages in the 5′-region and 3′-region are (Sp) phosphorothioates, and the central region comprises at least one Sp, Sp, Rp motif. In certain embodiments, populations of modified oligonucleotides are enriched for modified oligonucleotides comprising such intemucleoside linkage motifs.

In certain embodiments, the ASO comprises a region having an alternating intemucleoside linkage motif. In certain embodiments, oligonucleotides comprise a region of uniformly modified intemucleoside linkages. In certain such embodiments, the intemucleoside linkages are phosphorothioate intemucleoside linkages. In certain embodiments, all of the intemucleoside linkages of the oligonucleotide are phosphorothioate intemucleoside linkages. In certain embodiments, each intemucleoside linkage of the oligonucleotide is selected from phosphodiester or phosphate and phosphorothioate. In certain embodiments, each intemucleoside linkage of the oligonucleotide is selected from phosphodiester or phosphate and phosphorothioate and at least one intemucleoside linkage is phosphorothioate.

In certain embodiments, ASO comprises at least 6 phosphorothioate intemucleoside linkages. In certain embodiments, the oligonucleotide comprises at least 8 phosphorothioate intemucleoside linkages. In certain embodiments, the oligonucleotide comprises at least 10 phosphorothioate intemucleoside linkages. In certain embodiments, the oligonucleotide comprises at least one block of at least 6 consecutive phosphorothioate intemucleoside linkages. In certain embodiments, the oligonucleotide comprises at least one block of at least 8 consecutive phosphorothioate intemucleoside linkages. In certain embodiments, the oligonucleotide comprises at least one block of at least 10 consecutive phosphorothioate intemucleoside linkages. In certain embodiments, the oligonucleotide comprises at least block of at least one 12 consecutive phosphorothioate intemucleoside linkages. In certain such embodiments, at least one such block is located at the 3′ end of the oligonucleotide. In certain such embodiments, at least one such block is located within 3 nucleosides of the 3′ end of the oligonucleotide.

In certain embodiments, the ASO comprises one or more methylphosphonate linkages. In certain embodiments, modified oligonucleotides comprise a linkage motif comprising all phosphorothioate linkages except for one or two methylphosphonate linkages. In certain embodiments, one methylphosphonate linkage is in the central region of an oligonucleotide.

In certain embodiments, it is desirable to arrange the number of phosphorothioate intemucleoside linkages and phosphodiester intemucleoside linkages to maintain nuclease resistance. In certain embodiments, it is desirable to arrange the number and position of phosphorothioate intemucleoside linkages and the number and position of phosphodiester intemucleoside linkages to maintain nuclease resistance. In certain embodiments, the number of phosphorothioate intemucleoside linkages may be decreased and the number of phosphodiester intemucleoside linkages may be increased. In certain embodiments, the number of phosphorothioate intemucleoside linkages may be decreased and the number of phosphodiester intemucleoside linkages may be increased while still maintaining nuclease resistance. In certain embodiments it is desirable to decrease the number of phosphorothioate intemucleoside linkages while retaining nuclease resistance. In certain embodiments it is desirable to increase the number of phosphodiester intemucleoside linkages while retaining nuclease resistance.

The ASOs described herein can be short or long. The ASOs may be from 8 to 200 nucleotides in length, in some instances between 10 and 100, in some instances between 12 and 50.

In some embodiments, the ASO comprises the length of from 8 to 30 nucleotides. In some embodiments, the ASO comprises the length of from 9 to 30 nucleotides. In some embodiments, the ASO comprises the length of from 10 to 30 nucleotides. In some embodiments, the ASO comprises the length of from 11 to 30 nucleotides. In some embodiments, the ASO comprises the length of from 12 to 30 nucleotides. In some embodiments, the ASO comprises the length of from 13 to 30 nucleotides. In some embodiments, the ASO comprises the length of from 14 to 30 nucleotides. In some embodiments, the ASO comprises the length of from 15 to 30 nucleotides. In some embodiments, the ASO comprises the length of from 16 to 30 nucleotides. In some embodiments, the ASO comprises the length of from 17 to 30 nucleotides. In some embodiments, the ASO comprises the length of from 18 to 30 nucleotides. In some embodiments, the ASO comprises the length of from 19 to 30 nucleotides. In some embodiments, the ASO comprises the length of from 20 to 30 nucleotides.

In some embodiments, the ASO comprises the length of from 8 to 29 nucleotides. In some embodiments, the ASO comprises the length of from 9 to 29 nucleotides. In some embodiments, the ASO comprises the length of from 10 to 28 nucleotides. In some embodiments, the ASO comprises the length of from 11 to 28 nucleotides. In some embodiments, the ASO comprises the length of from 12 to 28 nucleotides. In some embodiments, the ASO comprises the length of from 13 to 28 nucleotides. In some embodiments, the ASO comprises the length of from 14 to 28 nucleotides. In some embodiments, the ASO comprises the length of from 15 to 28 nucleotides. In some embodiments, the ASO comprises the length of from 16 to 28 nucleotides. In some embodiments, the ASO comprises the length of from 17 to 28 nucleotides. In some embodiments, the ASO comprises the length of from 18 to 28 nucleotides. In some embodiments, the ASO comprises the length of from 19 to 28 nucleotides. In some embodiments, the ASO comprises the length of from 20 to 28 nucleotides.

In some embodiments, the ASO comprises the length of from 8 to 27 nucleotides. In some embodiments, the ASO comprises the length of from 9 to 27 nucleotides. In some embodiments, the ASO comprises the length of from 10 to 26 nucleotides. In some embodiments, the ASO comprises the length of from 10 to 25 nucleotides. In some embodiments, the ASO comprises the length of from 10 to 24 nucleotides. In some embodiments, the ASO comprises the length of from 11 to 24 nucleotides. In some embodiments, the ASO comprises the length of from 12 to 24 nucleotides. In some embodiments, the ASO comprises the length of from 13 to 24 nucleotides. In some embodiments, the ASO comprises the length of from 14 to 24 nucleotides. In some embodiments, the ASO comprises the length of from 15 to 24 nucleotides. In some embodiments, the ASO comprises the length of from 16 to 24 nucleotides. In some embodiments, the ASO comprises the length of from 17 to 28 nucleotides. In some embodiments, the ASO comprises the length of from 18 to 24 nucleotides. In some embodiments, the ASO comprises the length of from 19 to 24 nucleotides. In some embodiments, the ASO comprises the length of from 20 to 24 nucleotides.

In some embodiments, the ASO comprises the length of from 10 to 27 nucleotides. In some embodiments, the ASO comprises the length of from 11 to 26 nucleotides. In some embodiments, the ASO comprises the length of from 12 to 25 nucleotides. In some embodiments, the ASO comprises the length of from 12 to 24 nucleotides. In some embodiments, the ASO comprises the length of from 12 to 23 nucleotides. In some embodiments, the ASO comprises the length of from 12 to 22 nucleotides. In some embodiments, the ASO comprises the length of from 12 to 21 nucleotides. In some embodiments, the ASO comprises the length of from 12 to 20 nucleotides.

In some embodiments, the ASO comprises the length of from 16 to 27 nucleotides. In some embodiments, the ASO comprises the length of from 16 to 26 nucleotides. In some embodiments, the ASO comprises the length of from 16 to 25 nucleotides. In some embodiments, the ASO comprises the length of from 16 to 24 nucleotides. In some embodiments, the ASO comprises the length of from 16 to 23 nucleotides. In some embodiments, the ASO comprises the length of from 16 to 22 nucleotides. In some embodiments, the ASO comprises the length of from 16 to 21 nucleotides. In some embodiments, the ASO comprises the length of from 16 to 20 nucleotides.

In some embodiments, the ASO comprises the length of 8, 9, 10, 11, 12, 13, 14, 15, 16, 17, 18, 19, 20, 21, 22, 23, 24, 25, 26, 27, 28, 29 or more nucleotides, and 30, 29, 28, 27, 26, 25, 24, 23, 22, 21, 20, 19, 18, 17, 16, 15, 14, 13, 12, 11, 10, 9 or fewer nucleotides.

As used herein, the term “GC content” or “guanine-cytosine content” refers to the percentage of nitrogenous bases in a DNA or RNA molecule that are either guanine (G) or cytosine (C). This measure indicates the proportion of G and C bases out of an implied four total bases, also including adenine and thymine in DNA and adenine and uracil in RNA. In some embodiments, the ASO comprises a sequence comprising from 30% to 60% GC content. In some embodiments, the ASO comprises a sequence comprising from 35% to 60% GC content. In some embodiments, the ASO comprises a sequence comprising from 40% to 60% GC content. In some embodiments, the ASO comprises a sequence comprising from 45% to 60% GC content. In some embodiments, the ASO comprises a sequence comprising from 50% to 60% GC content. In some embodiments, the ASO comprises a sequence comprising from 30% to 55% GC content. In some embodiments, the ASO comprises a sequence comprising from 30% to 50% GC content. In some embodiments, the ASO comprises a sequence comprising from 30% to 45% GC content. In some embodiments, the ASO comprises a sequence comprising from 30% to 40% GC content. In some embodiments, the ASO comprises a sequence comprising 30, 31, 32, 33, 34, 35, 36, 37, 38, 39, 40, 41, 42, 43, 44, 45, 46, 47, 48, 49, 50, 51, 52, 53, 54, 55, 56, 57, 58, 59% or more and 60, 59, 58, 57, 56, 55, 54, 53, 52, 51, 50, 49, 48, 47, 46, 45, 44, 43, 42, 41, 40, 39, 38, 37, 36, 35, 34, 33, 32, 31% or less GC content.

In some embodiments, the nucleotide comprises at least one or more of: a length of from 10 to 30 nucleotides; a sequence comprising from 30% to 60% GC content; and at least one locked nucleotide. In some embodiments, the nucleotide comprises at least two or more of: a length of from 10 to 30 nucleotides; a sequence comprising from 30% to 60% GC content; and at least one locked nucleotide. In some embodiments, the nucleotide comprises a length of from 10 to 30 nucleotides; a sequence comprising from 30% to 60% GC content; and at least one locked nucleotide.

The ASO can be any contiguous stretch of nucleic acids. In some embodiments, the ASO can be any contiguous stretch of deoxyribonucleic acid (DNA), RNA, non-natural, artificial nucleic acid, modified nucleic acid or any combination thereof. The ASO can be a linear nucleotide. In some embodiments, the ASO is an oligonucleotide. In some embodiments, the ASO is a single stranded polynucleotide. In some embodiments, the polynucleotide is pseudo-double stranded (e.g., a portion of the single stranded polynucleotide self-hybridizes).

In some embodiments, the ASO is an unmodified nucleotide. In some embodiments, the ASO is a modified nucleotide. As used herein, the term “modified nucleotide” refers to a nucleotide with at least one modification to the sugar, the nucleobase, or the internucleoside linkage.

In some embodiments, the ASOs described herein is single stranded, chemically modified and synthetically produced. In some embodiments, the ASOs described herein may be modified to include high affinity RNA binders (e.g., locked nucleic acids (LNAs)) as well as chemical modifications. In some embodiments, the ASO comprises one or more residues that are modified to increase nuclease resistance, and/or to increase the affinity of the ASO for the target sequence. In some embodiments, the ASO comprises a nucleotide analogue. In some embodiments, the ASO may be expressed inside a target cell, such as a neuronal cell, from a nucleic acid sequence, such as delivered by a viral (e.g. lentiviral, AAV, or adenoviral) or non-viral vector.

In some embodiments, the ASO as described herein can comprise one or more substitutions, insertions and/or additions, deletions, and covalent modifications with respect to reference sequences.

In some embodiments, the ASO as described herein includes one or more post-transcriptional modifications (e.g., capping, cleavage, polyadenylation, splicing, poly-A sequence, methylation, acylation, phosphorylation, methylation of lysine and arginine residues, acetylation, and nitrosylation of thiol groups and tyrosine residues, etc). The one or more post-transcriptional modifications can be any post-transcriptional modification, such as any of the more than one hundred different nucleoside modifications that have been identified in RNA (Rozenski, J, Crain, P, and McCloskey, J. (1999). The RNA Modification Database: 1999 update. Nucl Acids Res 27: 196-197).

In some embodiments, the ASO as described herein may include any useful modification, such as to the sugar, the nucleobase, or the internucleoside linkage (e.g., to a linking phosphate / to a phosphodiester linkage / to the phosphodiester backbone). In some embodiments, the ASO as described herein may include a modified nucleobase, a modified nucleoside, or a combination thereof.

In some embodiments, modified nucleobases are selected from: 5-substituted pyrimidines, 6-azapyrimidines, alkyl or alkynyl substituted pyrimidines, alkyl substituted purines, and N-2, N-6 and 0-6 substituted purines. In some embodiments, modified nucleobases are selected from: 2-aminopropyladenine, 5 -hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (-C=C-CH3) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as l,3-diazaphenoxazine-2-one, l,3-diazaphenothiazine-2-one and 9-(2-aminoethoxy)-l,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone.

In some further embodiments, the ASO as described herein comprises at least one nucleoside selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the ASO as described herein comprises at least one nucleoside selected from the group consisting of 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the ASO as described herein comprises at least one nucleoside selected from the group consisting of 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In some embodiments, the nucleotides as described herein comprises at least one nucleoside selected from the group consisting of inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

Further nucleobases include those disclosed in Merigan et ah, U.S. 3,687,808, those disclosed in The Concise Encyclopedia Of Polymer Science And Engineering, Kroschwitz, J.I., Ed., John Wiley & Sons, 1990, 858-859; Englisch et al., Angewandte Chemie, International Edition, 1991, 30, 613; Sanghvi, Y.S., Chapter 15, Antisense Research and Applications, Crooke, S.T. and Lebleu, B., Eds., CRC Press, 1993, 273-288; and those disclosed in Chapters 6 and 15, Antisense Drug Technology, Crooke S.T., Ed., CRC Press, 2008, 163-166 and 442-443.

In some embodiments, modified nucleosides comprise double-headed nucleosides having two nucleobases. Such compounds are described in detail in Sorinas et al, J. Org. Chem, 2014 79: 8020-8030.

In some embodiments, the ASO comprises or consists of a modified oligonucleotide complementary to an target nucleic acid comprising one or more modified nucleobases. In some embodiments, the modified nucleobase is 5-methylcytosine. In some embodiments, each cytosine is a 5-methylcytosine.

In some embodiments, one or more atoms of a pyrimidine nucleobase in the ASO may be replaced or substituted with optionally substituted amino, optionally substituted thiol, optionally substituted alkyl (e.g., methyl or ethyl), or halo (e.g., chloro or fluoro). In some embodiments, modifications (e.g., one or more modifications) are present in each of the sugar and the internucleoside linkage. Modifications may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof. Additional modifications are described herein.

In some embodiments, the ASO as described herein includes at least one N(6)methyladenosine (m6A) modification. In some embodiments, the N(6)methyladenosine (m6A) modification can reduce immunogeneicity of the nucleotide as described herein.

In some embodiments, the modification may include a chemical or cellular induced modification. For example, some nonlimiting examples of intracellular RNA modifications are described by Lewis and Pan in “RNA modifications and structures cooperate to guide RNA-protein interactions” from Nat Reviews Mol Cell Biol, 2017, 18:202-210.In some embodiments, chemical modifications to the nucleotide as described herein may enhance immune evasion. The ASO as described herein may be synthesized and/or modified by methods well established in the art, such as those described in “Current protocols in nucleic acid chemistry,” Beaucage, S.L. et al. (Eds.), John Wiley & Sons, Inc., New York, NY, USA, which is hereby incorporated herein by reference. Modifications include, for example, end modifications, e.g., 5′ end modifications (phosphorylation (mono-, di- and tri-), conjugation, inverted linkages, etc.), 3′ end modifications (conjugation, DNA nucleotides, inverted linkages, etc.), base modifications (e.g., replacement with stabilizing bases, destabilizing bases, or bases that base pair with an expanded repertoire of partners), removal of bases (abasic nucleotides), or conjugated bases. The modified nucleotide bases may also include 5-methylcytidine and pseudouridine. In some embodiments, base modifications may modulate expression, immune response, stability, subcellular localization, to name a few functional effects, of the nucleotide as described herein. In some embodiments, the modification includes a bi-orthogonal nucleotides, e.g., an unnatural base. See for example, Kimoto et al, Chem Commun (Camb), 2017, 53:12309, DOI: 10.1039/c7cc06661a, which is hereby incorporated by reference.

In some embodiments, the ASO descibred herein may comprise one or more of (A) modified nucleosides and (B) Modified Internucleoside Linkages.

### (A) Modified Nucleosides

Modified nucleosides comprise a modified sugar moiety, a modified nucleobase, or both a modified sugar moiety and a modified nucleobase.

### 1. Certain Modified Sugar Moieties

In certain embodiments, sugar moieties are non-bicyclic, modified furanosyl sugar moieties. In certain embodiments, modified sugar moieties are bicyclic or tricyclic furanosyl sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified furanosyl sugar moieties comprising one or more acyclic substituent, including but not limited to substituents at the 2′, 3′, 4′, and/or 5′ positions. In certain embodiments, the furanosyl sugar moiety is a ribosyl sugar moiety. In certain embodiments, the furanosyl sugar moiety is a β-D-ribofuranosyl sugar moiety. In certain embodiments one or more acyclic substituent of non-bicyclic modified sugar moieties is branched. Examples of 2′ -substituent groups suitable for non-bicyclic modified sugar moieties include but are not limited to: 2′-F, 2′—OCH₃ (“2′—OMe” or “2′—O—methyl”), and 2′—O(CH₂)₂OCH₃ (“2′-MOE”). In certain embodiments, 2′-substituent groups are selected from among: halo, allyl, amino, azido, SH, CN, OCN, CF₃, OCF₃, O-C₁-C₁₀ alkoxy, O-C₁-C₁₀ substituted alkoxy, C₁-C₁₀ alkyl, C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. 6,531,584; Cook et al., U.S. 5,859,221; and Cook et al., U.S. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 3′-substituent groups include 3′-methyl (see Frier, et al., The ups and downs of nucleic acid duplex stability: structure-stability studies on chemically-modified DNA:RNA duplexes. Nucleic Acids Res., 25, 4429-4443, 1997.) Examples of 4′-substituent groups suitable for non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-allyl, 5′-ethyl, 5′-vinyl, and 5′-methoxy. In certain embodiments, non-bicyclic modified sugars comprise more than one non-bridging sugar substituent, for example, 2′-F -5 ‘-methyl sugar moieties and the modified sugar moieties and modified nucleosides described in Migawa et al., WO 2008/101157 and Rajeev et al., US2013/0203836. 2′,4′-difluoro modified sugar moieties have been described in Martinez-Montero, et al., Rigid 2′, 4′-difluororibonucleosides: synthesis, conformational analysis, and incorporation into nascent RNA by HCV polymerase. J. Org. Chem., 2014, 79:5627-5635. Modified sugar moieties comprising a 2′-modification (OMe or F) and a 4′-modification (OMe or F) have also been described in Malek-Adamian, et al., J. Org. Chem, 2018, 83: 9839-9849.

In certain embodiments, a 2′-substituted nucleoside or non-bicyclic 2′-modified nucleoside comprises a sugar moiety comprising a non-bridging 2′ -substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted nucleoside or non-bicyclic 2′-modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═0)—N(H)CH₃ (“NMA”).

In certain embodiments, a 2′-substituted nucleoside or non-bicyclic 2′-modified nucleoside comprises a sugar moiety comprising a non-bridging 2′-substituent group selected from: F, OCH₃, and OCH₂CH₂OCH₃.

In certain embodiments, the 4′ O of 2′-deoxyribose can be substituted with a S to generate 4′-thio DNA (see Takahashi, et al., Nucleic Acids Research 2009, 37: 1353-1362). This modification can be combined with other modifications detailed herein. In certain such embodiments, the sugar moiety is further modified at the 2′ position. In certain embodiments the sugar moiety comprises a 2′-fluoro. A thymidine with this sugar moiety has been described in Watts, et al., J. Org. Chem. 2006, 71(3): 921-925 (4′-S-fluoro5-methylarauridine or FAMU).

Certain modifed sugar moieties comprise a bridging sugar substituent that forms a second ring resulting in a bicyclic sugar moiety. In certain such embodiments, the bicyclic sugar moiety comprises a bridge between the 4′ and the 2′ furanose ring atoms. In certain such embodiments, the furanose ring is a ribose ring. Examples of sugar moieties comprising such 4′ to 2′ bridging sugar substituents include but are not limited to bicyclic sugars comprising: 4′-CH₂—2′, 4′—(CH₂)₂—2′, 4′—(CH₂)₃—2′, 4′—CH₂—O—2′ (“LNA”), 4′—CH₂—S—2′, 4′—(CH₂)₂—O—2′ (“ENA”), 4′—CH(CH₃)—0—2′ (referred to as “constrained ethyl” or “cEt” when in the S configuration), 4′—CH₂—O—CH₂—2′, 4′—CH₂—N(R)—2′, 4′—CH(CH₂OCH₃)—O—2′ (“constrained MOE” or “cMOE”) and analogs thereof (see, e.g., Seth et al., U.S. 7,399,845, Bhat et al., U.S. 7,569,686, Swayze et al., U.S. 7,741,457, and Swayze et al., U.S. 8,022,193), 4′—C(CH₃)(CH₃)—O—2′ and analogs thereof (see, e.g., Seth et al., U.S. 8,278,283), 4′—CH₂—N(OCH₃)—2′ and analogs thereof (see, e.g., Prakash et al., U.S. 8,278,425), 4′—CH₂—O—N(CH₃)—2′ (see, e.g., Allerson et al., U.S. 7,696,345 and Allerson et al., U.S. 8,124,745), 4′—CH₂—C(H)(CH₃)—2′ (see, e.g., Zhou, et al, J. Org. Chem., 2009, 74, 118-134), 4′—CH₂—C(═CH₂)—2′ and analogs thereof (see e.g., Seth et al., U.S. 8,278,426), 4′-C(RₐR_{b})—N(R)—O—2′, 4′—C(RₐR_{b})—O—N(R)—2′, 4′—CH₂—O—N(R)—2′, and 4′—CH₂—N(R)—O—2′, wherein each R, Rₐ, and R_{b}, is. independently, H, a protecting group, or C₁-C₁₂ alkyl (see, e.g. Imanishi et al., U.S. 7,427,672), 4′—C(═O)—N(CH₃)₂—2′, 4′—C(═0)—N(R)₂—2′, 4′—C(═S)—N(R)₂—2′ and analogs thereof (see, e.g., Obika et al., WO2011052436A1, Yusuke, W02017018360A1).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—. —C(Rₐ)═N—, —C(═NRₐ)—, —C(═O)—, —C(═S)—, —O—, —Si(Rₐ)₂—, —S(═O)ₓ—, and —N(Rₐ)—; wherein: x is 0, 1, or 2; n is 1, 2, 3, or 4; each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJi, NJ₁J₂. SJ₁, N₃, COOJ₁, acyl (C(═O)—H), substituted acyl, CN, sulfonyl (S(═O)₂—J₁), or sulfoxyl (S(═O)—J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al, Nucleic Acids Research, 1997, 25(22), 4429-4443, Albaek et al, J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2017, 129, 8362-8379; Elayadi et al.,; Christiansen, et al., J. Am. Chem. Soc. 1998, 120, 5458-5463; Wengel et al., U.S. 7,053,207; Imanishi et al., U.S. 6,268,490; Imanishi et al. U.S. 6,770,748; Imanishi et al., U.S. RE44,779; Wengel et al., U.S. 6,794,499; Wengel et al., U.S. 6,670,461; Wengel et al., U.S. 7,034,133; Wengel et al., U.S. 8,080,644; Wengel et al, U.S. 8,034,909; Wengel et al., U.S. 8,153,365; Wengel et al., U.S. 7,572,582; and Ramasamy et al., U.S. 6,525,191; Torsten et al., WO 2004/106356; Wengel et al., WO 1999/014226; Seth et al., WO 2007/134181; Seth et al., U.S. 7,547,684; Seth et al., U.S. 7,666,854; Seth et al., U.S. 8,088,746; Seth et al., U.S. 7,750, 131; Seth et al., U.S. 8,030,467; Seth et al., U.S. 8,268,980; Seth et al., U.S. 8,546,556; Seth et al., U.S. 8,530,640; Migawa et al., U.S. 9,012,421; Seth et al., U.S. 8,501,805; and U.S. Pat. Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an UNA nucleoside (described herein) may be in the α-U configuration or in the β-D configuration as follows:

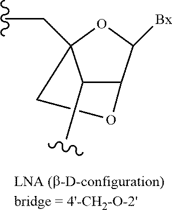

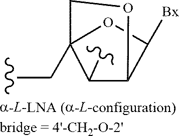

α-U-methyleneoxy (4′—CH₂—O—2′) or α-U-UNA bicyclic nucleosides have been incorporated into antisense oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., FNA) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

Nucleosides comprising modified furanosyl sugar moieties and modified furanosyl sugar moieties may be referred to by the position(s) of the substitution(s) on the sugar moiety of the nucleoside. The term “modified” following a position of the furanosyl ring, such as“2′-modified”, indicates that the sugar moiety comprises the indicated modification at the 2′ position and may comprise additional modifications and/or substituents. A 4′-2′ bridged sugar moiety is 2′-modified and 4′-modified, or, alternatively,“2′, 4′-modified”. The term “substituted” following a position of the furanosyl ring, such as “2′ -substituted” or “2′-4′-substituted”, indicates that is the only position(s) having a substituent other than those found in unmodified sugar moieties in oligonucleotides. Accordingly, the following sugar moieties are represented by the following formulas.

In the context of a nucleoside and/or an oligonucleotide, a non-bicyclic, modified furanosyl sugar moiety is represented by formula I:

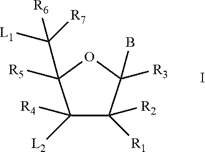

wherein B is a nucleobase; and L₁ and L₂ are each, independently, an internucleoside linkage, a terminal group, a conjugate group, or a hydroxyl group. Among the R groups, at least one of R₃₋₇ is not H and/or at least one of R₁ and R₂ is not H or OH. In a 2′-modified furanosyl sugar moiety, at least one of R₁ and R₂ is not H or OH and each of R₃₋₇ is independently selected from H or a substituent other than H. In a 4′-modified furanosyl sugar moiety, R₅ is not H and each of R₁-₄, ₆, ₇ are independently selected from H and a substituent other than H; and so on for each position of the furanosyl ring. The stereochemistry is not defined unless otherwise noted.

In the context of a nucleoside and/or an oligonucleotide, a non-bicyclic, modified, substituted fuamosyl sugar moiety is represented by formula I, wherein B is a nucleobase; and Li and L₂ are each, independently, an internucleoside linkage, a terminal group, a conjugate group, or a hydroxyl group. Among the R groups, either one (and no more than one) of R₃₋₇ is a substituent other than H or one of R₁ or R₂ is a substituent other than H or OH. The stereochemistry is not defined unless otherwise noted. Examples of non-bicyclic, modified, substituted furanosyl sugar moieties include 2′-substituted ribosyl, 4′-substituted ribosyl, and 5′-substituted ribosyl sugar moieties, as well as substituted 2′-deoxyfuranosyl sugar moieties, such as 4′-substituted 2′-deoxyribosyl and 5′-substituted 2′-deoxyribosyl sugar moieties.

In the context of a nucleoside and/or an oligonucleotide, a 2′-substituted ribosyl sugar moiety is represented by formula II:

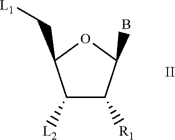

wherein B is a nucleobase; and L₁ and L₂ are each, independently, an internucleoside linkage, a terminal group, a conjugate group, or a hydroxyl group. R₁ is a substituent other than H or OH. The stereochemistry is defined as shown.

In the context of a nucleoside and/or an oligonucleotide, a 4′-substituted ribosyl sugar moiety is represented by formula III:

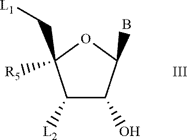

wherein B is a nucleobase; and L₁ and L₂ are each, independently, an internucleoside linkage, a terminal group, a conjugate group, or a hydroxyl group. R₅ is a substituent other than H. The stereochemistry is defined as shown.

In the context of a nucleoside and/or an oligonucleotide, a 5′-substituted ribosyl sugar moiety is represented by formula IV:

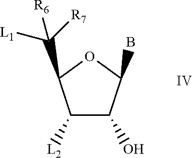

wherein B is a nucleobase; and L₁ and L₂ are each, independently, an internucleoside linkage, a terminal group, a conjugate group, or a hydroxyl group. R₆ or R₇ is a substituent other than H. The stereochemistry is defined as shown.

In the context of a nucleoside and/or an oligonucleotide, a 2′-deoxyfuranosyl sugar moiety is represented by formula V:

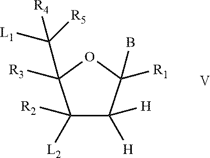

wherein B is a nucleobase; and L₁ and L₂ are each, independently, an internucleoside linkage, a terminal group, a conjugate group, or a hydroxyl group. Each of R₁₋₅ are independently selected from H and a non-H substituent. If all of R₁₋₅ are each H, the sugar moiety is an unsubstituted 2′-deoxyfuranosyl sugar moiety The stereochemistry is not defined unless otherwise noted.

In the context of a nucleoside and/or an oligonucleotide, a 4′-substituted 2′-deoxyribosyl sugar moiety is represented by formula VI:

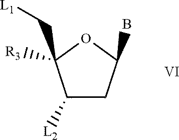

wherein B is a nucleobase; and L₁ and L₂ are each, independently, an internucleoside linkage, a terminal group, a conjugate group, or a hydroxyl group. R₃ is a substituent other than H. The stereochemistry is defined as shown.

In the context of a nucleoside and/or an oligonucleotide, a 5′-substituted 2′-deoxyribosyl sugar moiety is represented by formula VII:

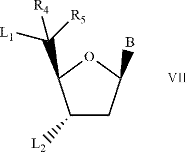

wherein B is a nucleobase; and L₁ and L₂ are each, independently, an internucleoside linkage, a terminal group, a conjugate group, or a hydroxyl group. R₄ or R₅ is a substituent other than H. The stereochemistry is defined as shown.

Unsubstituted 2′-deoxyfuranosyl sugar moieties may be unmodified (β-D-2′-deoxyribosyl) or modified. Examples of modified, unsubstituted 2′-deoxyfuranosyl sugar moieties include β-E-2′-deoxyribosyl, α-L-2′-deoxyribosyl, α-D-2′-deoxyribosyl, and β-D-xylosyl sugar moieties. For example, in the context of a nucleoside and/or an oligonucleotide, a β-L-2′-deoxyribosyl sugar moiety is represented by formula VIII:

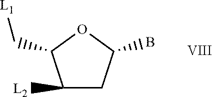

wherein B is a nucleobase; and L₁ and L₂ are each, independently, an internucleoside linkage, a terminal group, a conjugate group, or a hydroxyl group. The stereochemistry is defined as shown. Synthesis of α-L-ribosyl nucleotides and β-D-xylosyl nucleotides has been described by Gaubert, et al., Tetehedron 2006, 62: 2278-2294. Additional isomers of DNA and RNA nucleosides are described by Vester, et al., “Chemically modified oligonucleotides with efficient RNase H response,” Bioorg. Med. Chem. Letters, 2008, 18: 2296-2300.

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. 7,875,733 and Bhat et al., U.S. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), altritol nucleic acid (“ANA”), mannitol nucleic acid (“MNA”) (see. e.g., Leumann, CJ. Bioorg. &Med. Chem. 2002, 10, 841-854), fluoro HNA (“F-HNA”, see e.g. Swayze et al., U.S. 8,088,904; Swayze et al., U.S. 8,440,803; Swayze et al., U.S. 8,796,437; and Swayze et al., U.S. 9,005,906; F-HNA can also be referred to as a F-THP or 3′-fluoro tetrahydropyran), F-CeNA, and 3′-ara-HNA, having the formulas below, where L₁ and L₂ are each, independently, an internucleoside linkage linking the modified THP nucleoside to the remainder of an oligonucleotide or one of L₁ and L₂ is an internucleoside linkage linking the modified THP nucleoside to the remainder of an oligonucleotide and the other of L₁ and L₂ is H, a hydroxyl protecting group, a linked conjugate group, or a 5′ or 3 ‘-terminal group.

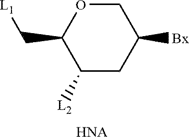

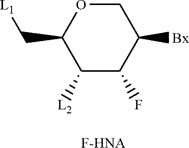

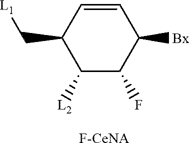

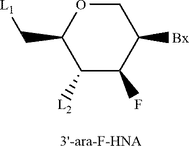

Additional sugar surrogates comprise THP compounds having the formula:

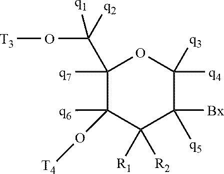

wherein, independently, for each of said modified THP nucleoside, Bx is a nucleobase moiety; T₃ and T₄ are each, independently, an internucleoside linkage linking the modified THP nucleoside to the remainder of an oligonucleotide or one of T₃ and T₄ is an internucleoside linkage linking the modified THP nucleoside to the remainder of an oligonucleotide and the other of T₃ and T₄ is H, a hydroxyl protecting group, a linked conjugate group, or a 5′ or 3′-terminal group; q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having no heteroatoms. For example, nucleosides comprising bicyclo [3.1.0]-hexane have been described (see, e.g., Marquez, et al., J. Med. Chem. 1996, 39:3739-3749).

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. 5,698,685; Summerton et al., U.S. 5,166,315; Summerton et al., U.S. 5,185,444; and Summerton et al., U.S. 5,034,506). As used here, the term “morpholino” means a sugar surrogate comprising the following structure:

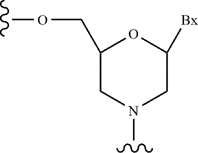

In certain embodiments, morpholinos may be modified, for example by adding or altering various substituent groups from the above morpholino structure. Such sugar surrogates are referred to herein as “modifed morpholinos.” In certain embodiments, morpholino residues replace a full nucleotide, including the internucleoside linkage, and have the structures shown below, wherein Bx is a heterocyclic base moiety.

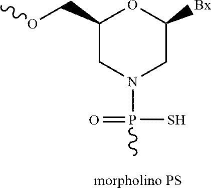

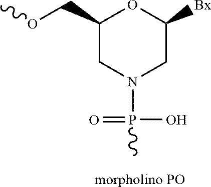

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), glycol nucleic acid (“GNA”, see Schlegel, et al., J. Am. Chem. Soc. 2017, 139:8537-8546) and nucleosides and oligonucleotides described in Manoharan et al., WO2011/133876.

Many other bicyclic and tricyclic sugar and sugar surrogate ring systems are known in the art that can be used in modified nucleosides. Certain such ring systems are described in Hanessian, et al., J. Org. Chem., 2013, 78: 9051-9063 and include bcDNA and tcDNA. Modifications to bcDNA and tcDNA, such as 6′-fluoro, have also been described (Dogovic and Ueumann, J. Org. Chem., 2014, 79: 1271-1279).

In certain embodiments, modified nucleosides are DNA mimics. “DNA mimic” means a nucleoside other than a DNA nucleoside wherein the nucleobase is directly linked to a carbon atom of a ring bound to a second carbon atom within the ring, wherein the second carbon atom comprises a bond to at least one hydrogen atom, wherein the nucleobase and at least one hydrogen atom are trans to one another relative to the bond between the two carbon atoms.

In certain embodiments, a DNA mimic comprises a structure represented by the formula below:

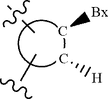

wherein Bx represents a heterocyclic base moiety.

In certain embodiments, a DNA mimic comprises a structure represented by one of the formulas below:

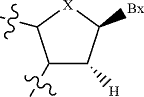

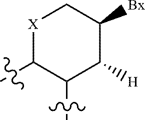

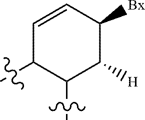

wherein X is O or S and Bx represents a heterocyclic base moiety.

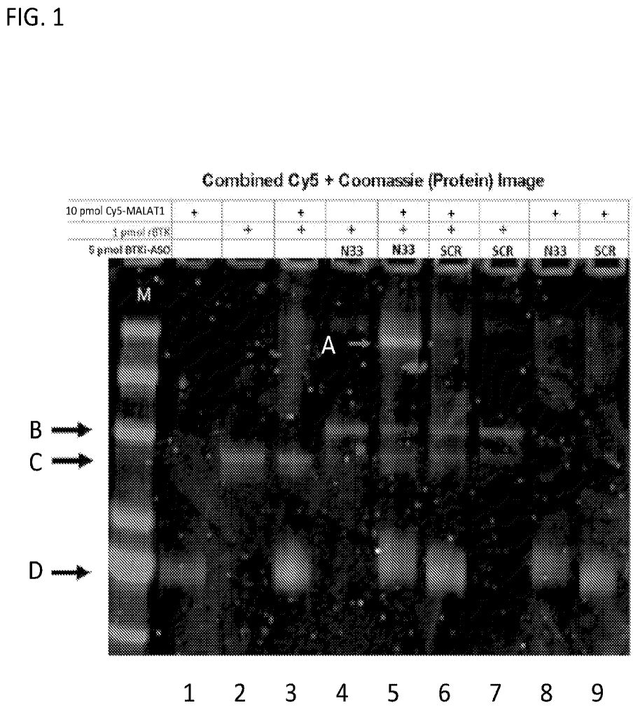

In certain embodiments, a DNA mimic is a sugar surrogate. In certain embodiments, a DNA mimic is a cycohexenyl or hexitol nucleic acid. In certain embodiments, a DNA mimic is described in FIG. 1 of Vester, et al., “Chemically modified oligonucleotides with efficient RNase H response,” Bioorg. Med. Chem. Letters, 2008, 18: 2296-2300, incorporated by reference herein. In certain embodiments, a DNA mimic nucleoside has a formula selected from:

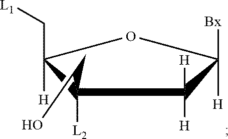

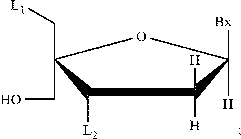

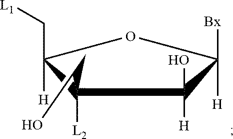

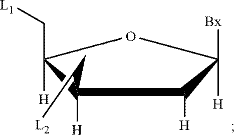

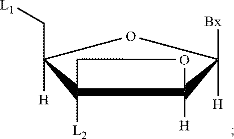

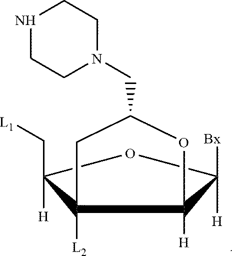

wherein Bx is a heterocyclic base moiety, and L₁ and L₂ are each, independently, an internucleoside linkage linking the modified THP nucleoside to the remainder of an oligonucleotide or one of L₁ and L₂ is an internucleoside linkage linking the modified nucleoside to the remainder of an oligonucleotide and the other of L₁ and L₂ is H, a hydroxyl protecting group, a linked conjugate group, or a 5′ or 3′-terminal group. In certain embodiments, a DNA mimic is α,β-constrained nucleic acid (CAN), 2′,4′-carbocyclic-LNA, or 2′, 4′-carbocyclic-ENA. In certain embodiments, a DNA mimic has a sugar moiety selected from among: 4′-C- hydroxymethyl-2′-deoxyribosyl, 3′-C-hydroxymethyl-2′-deoxyribosyl, 3′-C-hydroxymethyl- arabinosyl, 3′-C-2′-O-arabinosyl, 3′-C-methylene-extended-xyolosyl, 3′-C-2′-O-piperazino- arabinosyl. In certain embodiments, a DNA mimic has a sugar moiety selected from among: 2′- methylribosyl, 2′-S-methylribosyl, 2′-aminoribosyl, 2′-NH(CH₂)-ribosyl, 2′-NH(CH₂)₂-ribosyl, 2′-CH2-F-ribosyl, 2′-CHF2-ribosyl, 2′-CF3-ribosyl, 2′=CF2 ribosyl, 2′-ethylribosyl, 2′- alkenylribosyl, 2′-alkynylribosyl, 2′-O-4′-C-methyleneribosyl, 2′-cyanoarabinosyl, 2′- chloroarabinosyl, 2′-fluoroarabinosyl, 2′-bromoarabinosyl, 2′-azidoarabinosyl, 2′- methoxyarabinosyl, and 2′-arabinosyl. In certain embodiments, a DNA mimic has a sugar moiety selected from 4′-methyl-modified deoxyfuranosyl, 4′-F-deoxyfuranosyl, 4′-OMe- deoxyfuranosyl. In certain embodiments, a DNA mimic has a sugar moiety selected from among: 5′-methyl-2′-β-D-deoxyribosyl, 5′-ethyl-2′-β-D-deoxyribosyl, 5′-allyl-2′-β-D-deoxyribosyl, 2 - fluoro-β-D-arabinofuranosyl. In certain embodiments, DNA mimics are listed on page 32-33 of PCT/US00/267929 as B-form nucleotides, incorporated by reference herein in its entirety.

### 2. Modified Nucleobases

In certain embodiments, modified nucleobases are selected from: 5-substituted pyrimidines, 6-azapyrimidines, alkyl or alkynyl substituted pyrimidines, alkyl substituted purines, and N-2, N-6 and O-6 substituted purines. In certain embodiments, modified nucleobases are selected from: 2-aminopropyladenine, 5 -hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C═C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as l,3-diazaphenoxazine-2-one, l,3-diazaphenothiazine-2-one and 9-(2-aminoethoxy)-l,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. 3,687,808, those disclosed in The Concise Encyclopedia Of Polymer Science And Engineering, Kroschwitz, J.I., Ed., John Wiley & Sons, 1990, 858-859; Englisch et al., Angewandte Chemie, International Edition, 1991, 30, 613; Sanghvi, Y.S., Chapter 15, Antisense Research and Applications, Crooke, S.T. and Lebleu, B., Eds., CRC Press, 1993, 273-288; and those disclosed in Chapters 6 and 15, Antisense Drug Technology, Crooke S.T., Ed., CRC Press, 2008, 163-166 and 442-443. In certain embodiments, modified nucleosides comprise double-headed nucleosides having two nucleobases. Such compounds are described in detail in Sorinas et al., J. Org. Chem, 2014 79: 8020-8030.

Publications that teach the preparation of certain of the above noted modified nucleobases as well as other modified nucleobases include without limitation, Manoharan et al., US2003/0158403; Manoharan et al., US2003/0175906; Dinh et al., U.S. 4,845,205; Spielvogel et al., U.S. 5,130,302; Rogers et al., U.S. 5,134,066; Bischofberger et al., U.S. 5,175,273; Urdea et al., U.S. 5,367,066; Benner et al., U.S. 5,432,272; Matteucci et al., U.S. 5,434,257; Gmeiner et al., U.S. 5,457,187; Cook et al., U.S. 5,459,255; Froehler et al., U.S. 5,484,908; Matteucci et al., U.S. 5,502,177; Hawkins et al., U.S. 5,525,711; Haralambidis et al., U.S. 5,552,540; Cook et al., U.S. 5,587,469; Froehler et al., U.S. 5,594,121; Switzer et al., U.S. 5,596,091; Cook et al., U.S. 5,614,617; Froehler et al., U.S. 5,645,985; Cook et al., U.S. 5,681,941; Cook et al., U.S. 5,811,534; Cook et al., U.S. 5,750,692; Cook et al., U.S. 5,948,903; Cook et al., U.S. 5,587,470; Cook et al., U.S. 5,457,191; Matteucci et al., U.S. 5,763,588; Froehler et al., U.S. 5,830,653; Cook et al., U.S. 5,808,027; Cook et al., 6,166,199; and Matteucci et al., U.S. 6,005,096.

In certain embodiments, compounds comprise or consist of a modified oligonucleotide complementary to an target nucleic acid comprising one or more modified nucleobases. In certain embodiments, the modified nucleobase is 5-methylcytosine. In certain embodiments, each cytosine is a 5-methylcytosine.

### (B) Modified Internucleoside Linkages

In certain embodiments, compounds described herein having one or more modified internucleoside linkages are selected over compounds having only phosphodiester intemucleoside linkages because of desirable properties such as, for example, enhanced cellular uptake, enhanced affinity for target nucleic acids, and increased stability in the presence of nucleases.

In certain embodiments, compounds comprise or consist of a modified oligonucleotide complementary to a target nucleic acid comprising one or more modified internucleoside linkages. In certain embodiments, the modified internucleoside linkages are phosphorothioate linkages. In certain embodiments, each internucleoside linkage of an antisense compound is a phosphorothioate internucleoside linkage.

In certain embodiments, nucleosides of modified oligonucleotides may be linked together using any internucleoside linkage. The two main classes of internucleoside linkages are defined by the presence or absence of a phosphoms atom. Representative phosphorus-containing internucleoside linkages include unmodified phosphodiester internucleoside linkages, modified phosphotriesters such as THP phosphotriester and isopropyl phosphotriester, phosphonates such as methylphosphonate, isopropyl phosphonate, isobutyl phosphonate, and phosphonoacetate, phosphoramidates, phosphorothioate, and phosphorodithioate (“HS—P═S″). Representative non-phosphorus containing intemucleoside linkages include but are not limited to methylenemethylimino (—CH₂—N(CH₃)—O—CH₂—), thiodiester, thionocarbamate (—O—C(═O)(NH)—S—); siloxane (—O—SiH₂—O—); formacetal, thioacetamido (TANA), alt-thioformacetal, glycine amide, and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. Methods of preparation of phosphorous-containing and non-phosphorous-containing internucleoside linkages are well known to those skilled in the art.

Representative internucleoside linkages having a chiral center include but are not limited to alkylphosphonates and phosphorothioate s. Modified oligonucleotides comprising intemucleoside linkages having a chiral center can be prepared as populations of modified oligonucleotides comprising stereorandom intemucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate intemucleoside linkages are stereorandom. Such modified oligonucleotides can be generated using synthetic methods that result in random selection of the stereochemical configuration of each phosphorothioate linkage. All phosphorothioate linkages described herein are stereorandom unless otherwise specified. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate intemucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such chirally enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration. In certain embodiments, modified oligonucleotides comprising (Rp) and/or (Sp) phosphorothioates comprise one or more of the following formulas, respectively, wherein “B” indicates a nucleobase:

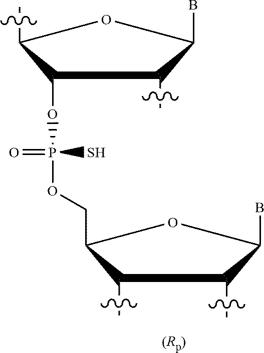

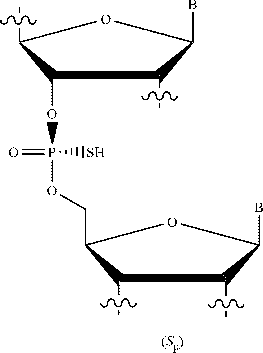

Unless otherwise indicated, chiral internucleoside linkages of modified oligonucleotides described herein can be stereorandom or in a particular stereochemical configuration.

Neutral internucleoside linkages include, without limitation, phosphotriesters, phosphonates, MMI (3′—CH₂—N(CH₃)—O—5′), amide-3 (3′—CH₂—C(═O)—N(H)—5′), amide-4 (3′—CH₂—N(H)—C(═O)—5′), formacetal (3′—O—CH₂—O—5′), methoxypropyl, and thioformacetal (3′—S—CH₂—O—5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y.S. Sanghvi and P.D. Cook, Eds., ACS Symposium Series 580; Chapters 3 and 4, 40-65). Further neutral internucleoside linkages include nonionic linkages comprising mixed N, O, S and CH2 component parts.

In certain embodiments, nucleic acids can be linked 2′ to 5′ rather than the standard 3′ to 5′ linkage. Such a linkage is illustrated herein:

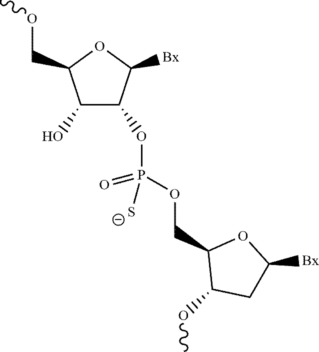

In the context of a nucleoside and/or an oligonucleotide, a non-bicyclic, 2′-linked modified furanosyl sugar moiety is represented by formula IX:

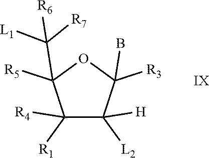

wherein B is a nucleobase; L₁ is an internucleoside linkage, a terminal group, a conjugate group, or a hydroxyl group and L₂ is an internucleoside linkage. The stereochemistry is not defined unless otherwise noted.

In certain embodiments, nucleosides can be linked by vinicinal 2′, 3′-phosphodiester bonds. In certain such embodiments, the nucleosides are threofuranosyl nucleosides (TNA; see Bala, et al., J Org. Chem. 2017, 82:5910-5916). A TNA linkage is shown herein:

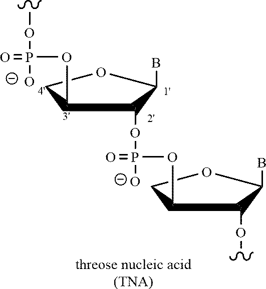

Additional modified linkages include α,β-D-CNA type linkages and related conformationally-constrained linkages, shown below. Synthesis of such molecules has been described previously (see Dupouy, et al., Angew. Chem. Int. Ed. Engl., 2014, 45: 3623-3627; Borsting, et al. Tetahedron, 2004, 60: 10955-10966; Ostergaard, et al.,ACS Chem. Biol. 2014, 9: 1975-1979; Dupouy, et al., Eur. J. Org. Chem., 2008, 1285-1294; Martinez, et al., PLoS One, 2011, 6:e25510; Dupouy, et al., Eur. J Org. Chem., 2007, 5256-5264; Boissonnet, et al., New J. Chem., 2011, 35: 1528-1533).

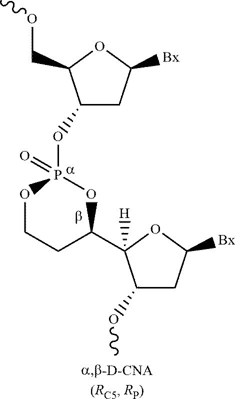

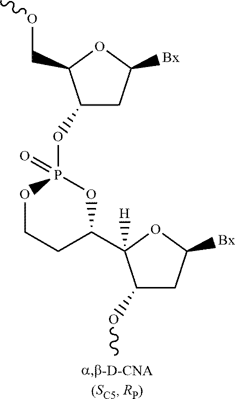

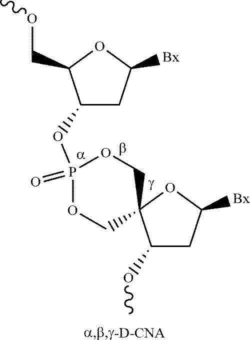

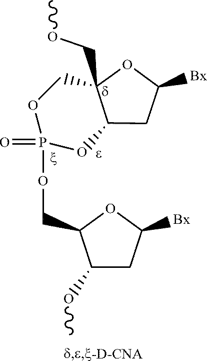

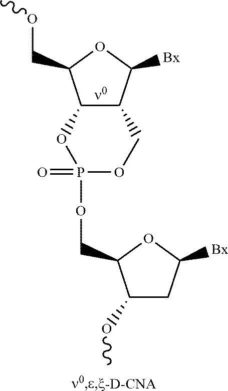

In some embodiments, the ASOs described herein is at least partially complementary to a target ribonucleotide. In some embodiments, the ASOs are complementary nucleic acid sequences designed to hybridize under stringent conditions to an RNA. In some embodiments, the oligonucleotides are chosen that are sufficiently complementary to the target, i.e., that hybridize sufficiently well and with sufficient specificity, to confer the desired effect.

In some embodiments, the ASO targets a MALAT1 RNA. In some embodiments, the ASO targets an XIST RNA. In some embodiments, the ASO targets a HSP70 RNA. In some embodiments, the ASO targets a MYC RNA. In some embodiments, the MALAT1 targetting ASO comprises the sequence CGUUAACUAGGCUUUA (SEQ ID NO: 1). In some embodiments, the XIST targeting ASO comprises the sequence GGAAGGGAATCAGCAGGTAT (SEQ ID NO: 2). In some embodiments, the HSP70 targeting ASO comprises the sequence TCTTGGGCCGAGGCTACTGA (SEQ ID NO: 3). In some embodiments, the MYC targeting ASO comprises the sequence CCTGGGGCTGGTGCATTTTC (SEQ ID NO: 4). In some embodiments, the ASO sequence is CGUUAACUAGGCUUUA (SEQ ID NO: 1). In some embodiments, the ASO sequence is GGAAGGGAATCAGCAGGTAT (SEQ ID NO: 2). In some embodiments, the ASO sequence is TCTTGGGCCGAGGCTACTGA (SEQ ID NO: 3). In some embodiments, the ASO sequence is CCTGGGGCTGGTGCATTTTC (SEQ ID NO: 4).

In some embodiments, the ASO targets MALAT1 RNA. In some embodiments, the ASO comprises a sequence having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identity to CGTTAACTAGGCTTTA (SEQ ID NO: 5). In some embodiments, the ASO comprises SEQ ID NO: 5. In some embodiments, the ASO consists of SEQ ID NO: 5.

In some embodiments, the ASO targets HSP70 RNA. In some embodiments, the ASO comprises a sequence having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identity to TCTTGGGCCGAGGCTACTGA (SEQ ID NO: 6). In some embodiments, the ASO comprises SEQ ID NO: 6. In some embodiments, the ASO consists of SEQ ID NO: 6.

In some embodiments, the ASO targets PVT1 RNA. In some embodiments, the ASO comprises a sequence having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identity to a sequence selected from the group consisting of SEQ ID NO: 7-39, 64, 67, 68, and 71 with optional one or more substitutions. In some embodiments, the ASO comprises a sequence selected from the group consisting of SEQ ID NO: 7-39, 64, 67, 68, and 71 with optional one or more substitutions. In some embodiments, the ASO is selected from the group consisting of PVT1 ASO1, PVT1 ASO2, PVT1 ASO3, PVT1 ASO4, PVT1 ASO5, PVT1 ASO6, PVT1 ASO7, PVT1 ASO8, PVT1 ASO9, PVT1 ASO10, PVT1 ASO11, PVT1 ASO12, PVT1 ASO13, PVT1 ASO14, PVT1 ASO15, PVT1 ASO16, PVT1 ASO17, PVT1 ASO18, PVT1 ASO19, PVT1 ASO20, PVT1 ASO21, PVT1 ASO22, PVT1 ASO23, PVT1 ASO24, PVT1 ASO25, PVT1 ASO26, PVT1 ASO27, PVT1 ASO28, PVT1 ASO29, PVT1 ASO30, PVT1 ASO31, PVT1 ASO32, and PVT1 ASO33 shown in Table 1A or 1B below.

In some embodiments, the ASO targets MYC RNA. In some embodiments, the ASO comprises a sequence having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identity to a sequence selected from the group consisting of SEQ ID NO: 40-45 with optional one or more substitutions. In some embodiments, the ASO comprises a sequence selected from the group consisting of SEQ ID NO: 40-45 with optional one or more substitutions. In some embodiments, the ASO is selected from the group consisting of MYC ASO1, MYC ASO2, MYC ASO3, MYC ASO4, MYC ASO5, and MYC ASO6 shown in Table 1A or 1B below.

In some embodiments, the ASO targets SCN1A RNA. In some embodiments, the ASO comprises a sequence having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identity to a sequence selected from the group consisting of SEQ ID NO: 46 with optional one or more substitutions. In some embodiments, the ASO comprises a sequence having SEQ ID NO: 46 with optional one or more substitutions. In some embodiments, the ASO is SCN1A ASO1 shown in Table 1A or 1B below.

In some embodiments, the ASO targets SYNGAP1 RNA. In some embodiments, the ASO comprises a sequence having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identity to a sequence selected from the group consisting of SEQ ID NO: 47-50 with optional one or more substitutions. In some embodiments, the ASO comprises a sequence selected from the group consisting of SEQ ID NO: 47-50 with optional one or more substitutions. In some embodiments, the ASO is selected from the group consisting of SYNGAP1 ASO1, SYNGAP1 ASO2, SYNGAP1 ASO3, and SYNGAP1 ASO4 in Table 1A or 1B below.

In some embodiments, the sequences of ASO described herein may be modified by one or more deletions, substitutions, and/or insertions at one or more of positions 1, 2, 3, 4, and 5 nucleotides from either or both ends.

TABLE 1A

ASO Sequences

| | | |
|---|---|---|
| ASO name | Sequence (5′ - 3′) | Human genome coordinate (hg38) |
| PVT1 ASO1 | GTAAGTGGAATTCCAGTTG (SEQ ID NO: 7) | chr8:127,796,050-127,796,068 |
| PVT1 ASO2 | AGCTTTAGACCACGAGGCAC (SEQ ID NO: 8) | chr8:127,796,017-127,796,036 |
| PVT1 ASO3 | AAGCTTTAGACCACGAGGCA (SEQ ID NO: 9) | chr8:127,796,018-127,796,037 |
| PVT1 ASO4 | GAAGCTTTAGACCACGAGGC (SEQ ID NO: 10) | chr8:127,796,019-127,796,038 |
| PVT1 ASO5 | CGAAGCTTTAGACCACGAGG (SEQ ID NO: 11) | chr8:127,796,020-127,796,039 |
| PVT1 ASO6 | CCGAAGCTTTAGACCACGAG (SEQ ID NO: 12) | chr8:127,796,021-127,796,040 |
| PVT1 ASO7 | GCCGAAGCTTTAGACCACGA (SEQ ID NO: 13) | chr8:127,796,022-127,796,041 |
| PVT1 ASO8 | TGCCGAAGCTTTAGACCACG (SEQ ID NO: 14) | chr8:127,796,023-127,796,042 |
| PVT1 AS09 | GTGCCGAAGCTTTAGACCAC (SEQ ID NO: 15) | chr8:127,796,024-127,796,043 |
| PVT1 ASO10 | TGTGCCGAAGCTTTAGACCA (SEQ ID NO: 16) | chr8:127,796,025-127,796,044 |
| PVT1 ASO11 | TTGTGCCGAAGCTTTAGACC (SEQ ID NO: 17) | chr8:127,796,026-127,796,045 |
| PVT1 ASO12 | CTTGTGCCGAAGCTTTAGAC (SEQ ID NO: 18) | chr8:127,796,027-127,796,046 |
| PVT1 ASO13 | CCTTGTGCCGAAGCTTTAGA (SEQ ID NO: 19) | chr8:127,796,028-127,796,047 |
| PVT1 ASO14 | CCCTTGTGCCGAAGCTTTAG (SEQ ID NO: 20) | chr8:127,796,029-127,796,048 |
| PVT1 ASO15 | GCCCTTGTGCCGAAGCTTTA (SEQ ID NO: 21) | chr8:127,796,030-127,796,049 |
| PVT1 ASO16 | GGCCCTTGTGCCGAAGCTTT (SEQ ID NO: 22) | chr8:127,796,031-127,796,050 |
| PVT1 ASO17 | GACACGGATTCTGTATTTGT (SEQ ID NO: 23) | chr8:127,795,934-127,795,953 |
| PVT1 ASO18 | AGGCCACGAGGTTTCTCCCA (SEQ ID NO: 24) | chr8:127,795,954-127,795,973 |
| PVT1 ASO19 | CATCTCAAATAATGGAGACC (SEQ ID NO: 25) | chr8:127,795,974-127,795,993 |
| PVT1 ASO20 | TTTAGACCACGAGGCACGTC (SEQ ID NO: 26) | chr8:127,796,014-127,796,033 |
| PVT1 ASO21 | AGTAAACAGAGATCTCAACC (SEQ ID NO: 27) | chr8:127,890,872-127,890,891 |
| PVT1 ASO22 | CTGGATGGAAGTATACACCA (SEQ ID NO: 28) | chr8:128,155,189-128,155,208 |
| PVT1 ASO23 | TATCACAGAACTAGGCTGTG (SEQ ID NO: 29) | chr8:128,070,278-128,070,297 |
| PVT1 ASO24 | CATTGAAGGATCATGGTCAT (SEQ ID NO: 30) | chr8:128,186,661-128,186,680 |
| PVT1 ASO25 | TTATAGACTAGATTGGCCAG (SEQ ID NO: 31) | chr8:128,186,707-128,186,726 |
| PVT1 ASO26 | TTTAATCTCCTTCTGGCCAA (SEQ ID NO: 32) | chr8:127,890,599-127,890,618 |
| PVT1 ASO27 | CAGCAGTCATCCAAATATTC (SEQ ID NO: 33) | chr8:128,155,296-128,155,315 |
| PVT1 ASO28 | AAGCTCCAGCCACAGAAACA (SEQ ID NO: 34) | chr8:127,796,324-127,796,343 |
| PVT1 ASO29 | ACTCCTCCTTTCCAGTGCAG (SEQ ID NO: 35) | chr8:127,796,346-127,796,365 |
| PVT1 ASO30 | CCACTTAACAAATCCCTCTG (SEQ ID NO: 36) | chr8:127,796,110-127,796,129 |
| PVT1 ASO31 | GCCACTCTTAACCAGGCAAA (SEQ ID NO: 37) | chr8:127,796,142-127,796,161 |
| PVT1 ASO32 | AGTCATACCCGTAAGTGGAA (SEQ ID NO: 38) | chr8:127,796,060-127,796,079 |
| PVT1 ASO33 | CACAGTCATACCCGTAAGTG (SEQ ID NO: 39) | chr8:127,796,063-127,796,082 |
| MYC ASO1 | TTTCTTCTTTCTCTCGCCGG (SEQ ID NO: 40) | chr8:127,737,628-127,737,647 |
| MYC ASO2 | AAGGTTTCAGAGGTGATGAG (SEQ ID NO: 41) | chr8:127,739,148-127,739,167 |
| MYC ASO3 | CGGAGACGCACTTAGTGAAC (SEQ ID NO: 42) | chr8:127,738,031-127,738,050 |
| MYC ASO4 | GTCCTAACACCTCTAGAGAC (SEQ ID NO: 43) | chr8:127,737,246-127,737,265 |
| MYC ASO5 | TTCATTCACTCTCAGAGATC (SEQ ID NO: 44) | chr8:127,739,383-127,739,402 |
| MYC ASO6 | GCATGAATACGTTAGAAAGG (SEQ ID NO: 45) | chr8: 127,740,291-127,740,310 |
| SCN1A ASO1¹ | AGTAAGACTGGGGTTGTT (SEQ ID NO: 46) | chr2: 166,036,141-166,036,158 |
| SYNGAP1 ASO1 | TAGGAAGTATCAAGCTGTG (SEQ ID NO: 47) | chr6:33,438,637-33,438,655 |
| SYNGAP1 ASO2 | ATCACCTCCTATAGCTCCT (SEQ ID NO: 48) | chr6:33,450,689-33,450,707 |
| SYNGAP1 ASO3 | CATCTCTCACCACGTTTGG (SEQ ID NO: 49 | chr6:33,424,530-33,424,548 |
| SYNGAP1 ASO4 | AATCTTGCCATCACCCACA (SEQ ID NO: 50) | chr6:33,429,570-33,429,588 |

¹Purchased from IDT as 5′-AzideN modified version.

In some embodiments, the ASO described herein may be chemically modified. In some embodiments, one or more nucleotides of the ASO described herein may be chemically modified with internal 2′-MethoxyEthoxy (i2MOEr) and/or 3′-Hydroxy-2′-MethoxyEthoxy (32MOEr), for example, resulting in those shown in Table 1B below.

TABLE 1B

Chemical ASO Modifications

| | |
|---|---|
| ASO name | Chemical modifications to ASO |
| PVT1 ASO1 | */i2MOErG/*/i2MOErT/*/i2MOErA/*/i2MOErA/*/i2MOErG/*/i2MOErT/*/ i2MOErG/*/i2MOErG/*/i2MOErA/*/i2MOErA/*/i2MOErT/*/i2MOErT/*/i2 MOErC/*/i2MOErC/*/i2MOErA/*/i2MOErG/*/i2MOErT/*/i2MOErT/*/32MOErG/ |
| PVT1 ASO2 | */i2MOErA/*/i2MOErG/*/i2MOErC/*/i2MOErT/*/i2MOErT/*/i2MOErT/*/ i2MOErA/*/i2MOErG/*/i2MOErA/*/i2MOErC/*/i2MOErC/*/i2MOErA/*/i 2MOErC/*/i2MOErG/*/i2MOErA/*/i2MOErG/*/i2MOErG/*/i2MOErC/*/i2 MOErA/*/32MOErC/ |
| PVT1 ASO3 | */i2MOErA/*/i2MOErA/*/i2MOErG/*/i2MOErC/*/i2MOErT/*/i2MOErT/*/ i2MOErT/*/i2MOErA/*/i2MOErG/*/i2MOErA/*/i2MOErC/*/i2MOErC/*/i2 MOErA/*/i2MOErC/*/i2MOErG/*/i2MOErA/*/i2MOErG/*/i2MOErG/*/i2 MOErC/*/32MOErA/ |
| PVT1 ASO4 | */i2MOErG/*/i2MOErA/*/i2MOErA/*/i2MOErG/*/i2MOErC/*/i2MOErT/*/ i2MOErT/*/i2MOErT/*/i2MOErA/*/i2MOErG/*/i2MOErA/*/i2MOErC/*/i2 MOErC/*/i2MOErA/*/i2MOErC/*/i2MOErG/*/i2MOErA/*/i2MOErG/*/i2 MOErG/*/32MOErC/ |
| PVT1 ASO5 | */i2MOErC/*/i2MOErG/*/i2MOErA/*/i2MOErA/*/i2MOErG/*/i2MOErC/* /i2MOErT/*/i2MOErT/*/i2MOErT/*/i2MOErA/*/i2MOErG/*/i2MOErA/*/i 2MOErC/*/i2MOErC/*/i2MOErA/*/i2MOErC/*/i2MOErG/*/i2MOErA/*/i2 MOErG/*/32MOErG/ |
| PVT1 ASO6 | */i2MOErC/*/i2MOErC/*/i2MOErG/*/i2MOErA/*/i2MOErA/*/i2MOErG/* /i2MOErC/*/i2MOErT/*/i2MOErT/*/i2MOErT/*/i2MOErA/*/i2MOErG/*/i 2MOErA/*/i2MOErC/*/i2MOErC/*/i2MOErA/*/i2MOErC/*/i2MOErG/*/i2 MOErA/*/32MOErG/ |
| PVT1 ASO7 | */i2MOErG/*/i2MOErC/*/i2MOErC/*/i2MOErG/*/i2MOErA/*/i2MOErA/* /i2MOErG/*/i2MOErC/*/i2MOErT/*/i2MOErT/*/i2MOErT/*/i2MOErA/*/i 2MOErG/*/i2MOErA/*/i2MOErC/*/i2MOErC/*/i2MOErA/*/i2MOErC/*/i2 MOErG/*/32MOErA/ |
| PVT1 ASO8 | */i2MOErT/*/i2MOErG/*/i2MOErC/*/i2MOErC/*/i2MOErG/*/i2MOErA/*/ i2MOErA/*/i2MOErG/*/i2MOErC/*/i2MOErT/*/i2MOErT/*/i2MOErT/*/i2 MOErA/*/i2MOErG/*/i2MOErA/*/i2MOErC/*/i2MOErC/*/i2MOErA/*/i2 MOErC/*/32MOErG/ |
| PVT1 ASO9 | */i2MOErG/*/i2MOErT/*/i2MOErG/*/i2MOErC/*/i2MOErC/*/i2MOErG/*/ i2MOErA/*/i2MOErA/*/i2MOErG/*/i2MOErC/*/i2MOErT/*/i2MOErT/*/i2 MOErT/*/i2MOErA/*/i2MOErG/*/i2MOErA/*/i2MOErC/*/i2MOErC/*/i2 MOErA/*/32MOErC/ |
| PVT1 ASO10 | */i2MOErT/*/i2MOErG/*/i2MOErT/*/i2MOErG/*/i2MOErC/*/i2MOErC/*/ i2MOErG/*/i2MOErA/*/i2MOErA/*/i2MOErG/*/i2MOErC/*/i2MOErT/*/i2 MOErT/*/i2MOErT/*/i2MOErA/*/i2MOErG/*/i2MOErA/*/i2MOErC/*/i2M OErC/*/32MOErA/ |
| PVT1 ASO11 | */i2MOErT/*/i2MOErT/*/i2MOErG/*/i2MOErT/*/i2MOErG/*/i2MOErC/*/ i2MOErC/*/i2MOErG/*/i2MOErA/*/i2MOErA/*/i2MOErG/*/i2MOErC/*/i 2MOErT/*/i2MOErT/*/i2MOErT/*/i2MOErA/*/i2MOErG/*/i2MOErA/*/i2 MOErC/*/32MOErC/ |
| PVT1 ASO12 | */i2MOErC/*/i2MOErT/*/i2MOErT/*/i2MOErG/*/i2MOErT/*/i2MOErG/*/ i2MOErC/*/i2MOErC/*/i2MOErG/*/i2MOErA/*/i2MOErA/*/i2MOErG/*/i 2MOErC/*/i2MOErT/*/i2MOErT/*/i2MOErT/*/i2MOErA/*/i2MOErG/*/i2 MOErA/*/32MOErC/ |
| PVT1 ASO13 | */i2MOErC/*/i2MOErC/*/i2MOErT/*/i2MOErT/*/i2MOErG/*/i2MOErT/*/i 2MOErG/*/i2MOErC/*/i2MOErC/*/i2MOErG/*/i2MOErA/*/i2MOErA/*/i2 MOErG/*/i2MOErC/*/i2MOErT/*/i2MOErT/*/i2MOErT/*/i2MOErA/*/i2M OErG/*/32MOErA/ |
| PVT1 ASO14 | */i2MOErC/*/i2MOErC/*/i2MOErC/*/i2MOErT/*/i2MOErT/*/i2MOErG/*/ i2MOErT/*/i2MOErG/*/i2MOErC/*/i2MOErC/*/i2MOErG/*/i2MOErA/*/i2 MOErA/*/i2MOErG/*/i2MOErC/*/i2MOErT/*/i2MOErT/*/i2MOErT/*/i2M OErA/*/32MOErG/ |
| PVT1 ASO15 | */i2MOErG/*/i2MOErC/*/i2MOErC/*/i2MOErC/*/i2MOErT/*/i2MOErT/*/ i2MOErG/*/i2MOErT/*/i2MOErG/*/i2MOErC/*/i2MOErC/*/i2MOErG/*/i2 MOErA/*/i2MOErA/*/i2MOErG/*/i2MOErC/*/i2MOErT/*/i2MOErT/*/i2M OErT/*/32MOErA/ |
| PVT1 ASO16 | */i2MOErG/*/i2MOErG/*/i2MOErC/*/i2MOErC/*/i2MOErC/*/i2MOErT/*/ i2MOErT/*/i2MOErG/*/i2MOErT/*/i2MOErG/*/i2MOErC/*/i2MOErC/*/i2 MOErG/*/i2MOErA/*/i2MOErA/*/i2MOErG/*/i2MOErC/*/i2MOErT/*/i2 MOErT/*/32MOErT/ |
| PVT1 ASO17 | */i2MOErG/*/i2MOErA/*/i2MOErC/*/i2MOErA/*/i2MOErC/*/i2MOErG/* /i2MOErG/*/i2MOErA/*/i2MOErT/*/i2MOErT/*/i2MOErC/*/i2MOErT/*/i 2MOErG/*/i2MOErT/*/i2MOErA/*/i2MOErT/*/i2MOErT/*/i2MOErT/*/i2 MOErG/*/32MOErT/ |
| PVT1 ASO18 | */i2MOErA/*/i2MOErG/*/i2MOErG/*/i2MOErC/*/i2MOErC/*/i2MOErA/* /i2MOErC/*/i2MOErG/*/i2MOErA/*/i2MOErG/*/i2MOErG/*/i2MOErT/*/i 2MOErT/*/i2MOErT/*/i2MOErC/*/i2MOErT/*/i2MOErC/*/i2MOErC/*/i2 MOErC/*/32MOErA/ |
| PVT1 ASO19 | */i2MOErC/*/i2MOErA/*/i2MOErT/*/i2MOErC/*/i2MOErT/*/i2MOErC/*/ i2MOErA/*/i2MOErA/*/i2MOErA/*/i2MOErT/*/i2MOErA/*/i2MOErA/*/i 2MOErT/*/i2MOErG/*/i2MOErG/*/i2MOErA/*/i2MOErG/*/i2MOErA/*/i2 MOErC/*/32MOErC/ |
| PVT1 ASO20 | */i2MOErT/*/i2MOErT/*/i2MOErT/*/i2MOErA/*/i2MOErG/*/i2MOErA/*/ i2MOErC/*/i2MOErC/*/i2MOErA/*/i2MOErC/*/i2MOErG/*/i2MOErA/*/i2 MOErG/*/i2MOErG/*/i2MOErC/*/i2MOErA/*/i2MOErC/*/i2MOErG/*/i2 MOErT/*/32MOErC/ |
| PVT1 ASO21 | */i2MOErA/*/i2MOErG/*/i2MOErT/*/i2MOErA/*/i2MOErA/*/i2MOErA/* /i2MOErC/*/i2MOErA/*/i2MOErG/*/i2MOErA/*/i2MOErG/*/i2MOErA/*/i 2MOErT/*/i2MOErC/*/i2MOErT/*/i2MOErC/*/i2MOErA/*/i2MOErA/*/i2 MOErC/*/32MOErC/ |
| PVT1 ASO22 | */i2MOErC/*/i2MOErT/*/i2MOErG/*/i2MOErG/*/i2MOErA/*/i2MOErT/*/ i2MOErG/*/i2MOErG/*/i2MOErA/*/i2MOErA/*/i2MOErG/*/i2MOErT/*/i 2MOErA/*/i2MOErT/*/i2MOErA/*/i2MOErC/*/i2MOErA/*/i2MOErC/*/i2 MOErC/*/32MOErA/ |
| PVT1 ASO23 | */i2MOErT/*/i2MOErA/*/i2MOErT/*/i2MOErC/*/i2MOErA/*/i2MOErC/*/ i2MOErA/*/i2MOErG/*/i2MOErA/*/i2MOErA/*/i2MOErC/*/i2MOErT/*/i2 MOErA/*/i2MOErG/*/i2MOErG/*/i2MOErC/*/i2MOErT/*/i2MOErG/*/i2 MOErT/*/32MOErG/ |
| PVT1 ASO24 | */i2MOErC/*/i2MOErA/*/i2MOErT/*/i2MOErT/*/i2MOErG/*/i2MOErA/*/ i2MOErA/*/i2MOErG/*/i2MOErG/*/i2MOErA/*/i2MOErT/*/i2MOErC/*/i2 MOErA/*/i2MOErT/*/i2MOErG/*/i2MOErG/*/i2MOErT/*/i2MOErC/*/i2M OErA/*/32MOErT/ |
| PVT1 ASO25 | */i2MOErT/*/i2MOErT/*/i2MOErA/*/i2MOErT/*/i2MOErA/*/i2MOErG/*/ i2MOErA/*/i2MOErC/*/i2MOErT/*/i2MOErA/*/i2MOErG/*/i2MOErA/*/i2 MOErT/*/i2MOErT/*/i2MOErG/*/i2MOErG/*/i2MOErC/*/i2MOErC/*/i2M OErA/*/32MOErG/ |
| PVT1 ASO26 | */i2MOErT/*/i2MOErT/*/i2MOErT/*/i2MOErA/*/i2MOErA/*/i2MOErT/*/i 2MOErC/*/i2MOErT/*/i2MOErC/*/i2MOErC/*/i2MOErT/*/i2MOErT/*/i2 MOErC/*/i2MOErT/*/i2MOErG/*/i2MOErG/*/i2MOErC/*/i2MOErC/*/i2M OErA/*/32MOErA/ |
| PVT1 ASO27 | */i2MOErC/*/i2MOErA/*/i2MOErG/*/i2MOErC/*/i2MOErA/*/i2MOErG/* /i2MOErT/*/i2MOErC/*/i2MOErA/*/i2MOErT/*/i2MOErC/*/i2MOErC/*/i 2MOErA/*/i2MOErA/*/i2MOErA/*/i2MOErT/*/i2MOErA/*/i2MOErT/*/i2 MOErT/*/32MOErC/ |
| PVT1 ASO28 | */i2MOErA/*/i2MOErA/*/i2MOErG/*/i2MOErC/*/i2MOErT/*/i2MOErC/*/ i2MOErC/*/i2MOErA/*/i2MOErG/*/i2MOErC/*/i2MOErC/*/i2MOErA/*/i2 MOErC/*/i2MOErA/*/i2MOErG/*/i2MOErA/*/i2MOErA/*/i2MOErA/*/i2 MOErC/*/32MOErA/ |
| PVT1 ASO29 | */i2MOErA/*/i2MOErC/*/i2MOErT/*/i2MOErC/*/i2MOErC/*/i2MOErT/*/ i2MOErC/*/i2MOErC/*/i2MOErT/*/i2MOErT/*/i2MOErT/*/i2MOErC/*/i2 MOErC/*/i2MOErA/*/i2MOErG/*/i2MOErT/*/i2MOErG/*/i2MOErC/*/i2 MOErA/*/32MOErG/ |
| PVT1 ASO30 | */i2MOErC/*/i2MOErC/*/i2MOErA/*/i2MOErC/*/i2MOErT/*/i2MOErT/*/ i2MOErA/*/i2MOErA/*/i2MOErC/*/i2MOErA/*/i2MOErA/*/i2MOErA/*/i 2MOErT/*/i2MOErC/*/i2MOErC/*/i2MOErC/*/i2MOErT/*/i2MOErC/*/i2 MOErT/*/32MOErG/ |
| PVT1 ASO31 | */i2MOErG/*/i2MOErC/*/i2MOErC/*/i2MOErA/*/i2MOErC/*/i2MOErT/*/ i2MOErC/*/i2MOErT/*/i2MOErT/*/i2MOErA/*/i2MOErA/*/i2MOErC/*/i2 MOErC/*/i2MOErA/*/i2MOErG/*/i2MOErG/*/i2MOErC/*/i2MOErA/*/i2 MOErA/*/32MOErA/ |
| PVT1 ASO32 | */i2MOErA/*/i2MOErG/*/i2MOErT/*/i2MOErC/*/i2MOErA/*/i2MOErT/*/ i2MOErA/*/i2MOErC/*/i2MOErC/*/i2MOErC/*/i2MOErG/*/i2MOErT/*/i2 MOErA/*/i2MOErA/*/i2MOErG/*/i2MOErT/*/i2MOErG/*/i2MOErG/*/i2 MOErA/*/32MOErA/ |
| PVT1 ASO33 | */i2MOErC/*/i2MOErA/*/i2MOErC/*/i2MOErA/*/i2MOErG/*/i2MOErT/*/ i2MOErC/*/i2MOErA/*/i2MOErT/*/i2MOErA/*/i2MOErC/*/i2MOErC/*/i2 MOErC/*/i2MOErG/*/i2MOErT/*/i2MOErA/*/i2MOErA/*/i2MOErG/*/i2 MOErT/*/32MOErG/ |
| MYC ASO1 | */i2MOErT/*/i2MOErT/*/i2MOErT/*/i2MOErC/*/i2MOErT/*/i2MOErT/*/i 2MOErC/*/i2MOErT/*/i2MOErT/*/i2MOErT/*/i2MOErC/*/i2MOErT/*/i2 MOErC/*/i2MOErT/*/i2MOErC/*/i2MOErG/*/i2MOErC/*/i2MOErC/*/i2M OErG/*/32MOErG/ |
| MYC ASO2 | */i2MOErA/*/i2MOErA/*/i2MOErG/*/i2MOErG/*/i2MOErT/*/i2MOErT/*/ i2MOErT/*/i2MOErC/*/i2MOErA/*/i2MOErG/*/i2MOErA/*/i2MOErG/*/i2 MOErG/*/i2MOErT/*/i2MOErG/*/i2MOErA/*/i2MOErT/*/i2MOErG/*/i2 MOErA/*/32MOErG/ |
| MYC ASO3 | */i2MOErC/*/i2MOErG/*/i2MOErG/*/i2MOErA/*/i2MOErG/*/i2MOErA/* /i2MOErC/*/i2MOErG/*/i2MOErC/*/i2MOErA/*/i2MOErC/*/i2MOErT/*/i 2MOErT/*/i2MOErA/*/i2MOErG/*/i2MOErT/*/i2MOErG/*/i2MOErA/*/i2 MOErA/*/32MOErC/ |
| MYC ASO4 | */i2MOErG/*/i2MOErT/*/i2MOErC/*/i2MOErC/*/i2MOErT/*/i2MOErA/*/ i2MOErA/*/i2MOErC/*/i2MOErA/*/i2MOErC/*/i2MOErC/*/i2MOErT/*/i2 MOErC/*/i2MOErT/*/i2MOErA/*/i2MOErG/*/i2MOErA/*/i2MOErG/*/i2 MOErA/*/32MOErC/ |
| MYC ASO5 | */i2MOErT/*/i2MOErT/*/i2MOErC/*/i2MOErA/*/i2MOErT/*/i2MOErT/*/i 2MOErC/*/i2MOErA/*/i2MOErC/*/i2MOErT/*/i2MOErC/*/i2MOErT/*/i2 MOErC/*/i2MOErA/*/i2MOErG/*/i2MOErA/*/i2MOErG/*/i2MOErA/*/i2 MOErT/*/32MOErC/ |
| MYC ASO6 | */i2MOErG/*/i2MOErC/*/i2MOErA/*/i2MOErT/*/i2MOErG/*/i2MOErA/*/ i2MOErA/*/i2MOErT/*/i2MOErA/*/i2MOErC/*/i2MOErG/*/i2MOErT/*/i2 MOErT/*/i2MOErA/*/i2MOErG/*/i2MOErA/*/i2MOErA/*/i2MOErA/*/i2 MOErG/*/32MOErG/ |
| SCN1A ASO1¹ | *A*+G*+T*A*A*G*+A*C*+T*G*G*G*G*+T*T*+G*+T*+T |
| SYNGAP1 ASO1 | */i2MOErT/*/i2MOErA/*/i2MOErG/*/i2MOErG/*/i2MOErA/*/i2MOErA/* /i2MOErG/*/i2MOErT/*/i2MOErA/*/i2MOErT/*/i2MOErC/*/i2MOErA/ */i2MOErA/*/i2MOErG/*/i2MOErC/*/i2MOErT/*/i2MOErG/*/i2MOErT/*/ 32MOErG/ |
| SYNGAP1 ASO2 | */i2MOErA/*/i2MOErT/*/i2MOErC/*/i2MOErA/*/i2MOErC/*/i2MOErC/*/ i2MOErT/*/i2MOErC/*/i2MOErC/*/i2MOErT/*/i2MOErA/*/i2MOErT/ */i2MOErA/*/i2MOErG/*/i2MOErC/*/i2MOErT/*/i2MOErC/*/i2MOErC/*/ 32MOErT/ |
| SYNGAP1 ASO3 | */i2MOErC/*/i2MOErA/*/i2MOErT/*/i2MOErC/*/i2MOErT/*/i2MOErC/*/ i2MOErT/*/i2MOErC/*/i2MOErA/*/i2MOErC/*/i2MOErC/*/i2MOErA/ */i2MOErC/*/i2MOErG/*/i2MOErT/*/i2MOErT/*/i2MOErT/*/i2MOErG/*/ 32MOErG/ |
| SYNGAP1 ASO4 | */i2MOErA/*/i2MOErA/*/i2MOErT/*/i2MOErC/*/i2MOErT/*/i2MOErT/*/ i2MOErG/*/i2MOErC/*/i2MOErC/*/i2MOErA/*/i2MOErT/*/i2MOErC/ */i2MOErA/*/i2MOErC/*/i2MOErC/*/i2MOErC/*/i2MOErA/*/i2MOErC/*/ 32MOErA/ |

¹Purchased from IDT as 5′-AzideN modified version.

Table 1A shows ASO sequences and their coordinates in the human genome. Table 1B shows exemplary chemistry modifications for each ASOs. Mod Code follows IDT Mod Code: + = LNA, * = Phosphorothioate linkage, i2MOErA = internal 2′-MethoxyEthoxy A, i2MOErC = internal 2′-MethoxyEthoxy MeC, 32MOErA = 3′-Hydroxy-2′-MethoxyEthoxy A, etc.

As used herein, the term “MALAT 1” or “metastasis associated lung adenocarcinoma transcript 1” also known as NEAT2 (noncoding nuclear-enriched abundant transcript 2) refers to a large, infrequently spliced non-coding RNA, which is highly conserved amongst mammals and highly expressed in the nucleus. In some embodiments, MALAT1 may play a role in multiple types of physiological processes, such as alternative splicing, nuclear organization, and epigenetic modulating of gene expression. In some embodiments, MALAT1 may play a role in various pathological processes, ranging from diabetes complications to cancers. In some embodiments, MALAT1 may play a role in regulation of the expression of metastasis-associated genes. In some embodiments, MALAT1 may play a role in positive regulation of cell motility via the transcriptional and/or post-transcriptional regulation of motility-related genes.

As used herein, the term “XIST” or “X-inactive specific transcript” refers to a non-coding RNA on the X chromosome of the placental mammals that acts as a major effector of the X-inactivation process. XIST is a component of the Xic (X-chromosome inactivation centre), which is involved in X-inactivation. XIST RNA is expressed exclusively from the Xic of the inactive X chromosome, but and not on the active X chromosome. The XIST transcript is processed through splicing and polyadenylation. However, the XIST RNA does not encode a protein and remains untranslated. The inactive X chromosome is coated with the XIST RNA, which is essential for the inactivation. XIST RNA has been implicated in the X-chromosome silencing by recruiting XIST silencing complex comprising a multitude of biomolecules. XIST mediated gene silencing is initiated early in the development and maintained throughout the lifetime of a cell in a female heterozygous subject.

As used herein, the terms “70 kilodalton heat shock proteins,” “Hsp70s,” or “DnaK” refers to a family of conserved ubiquitously expressed heat shock proteins. In some embodiments, the Hsp70s are an important part of the cell’s machinery for protein folding. In some embodiments, the Hsp70s help to protect cells from stress.

As used herein, the term “MYC” refers to MYC proto-oncogene, bHLH transcription factor that is a member of the myc family of transcription factors. The MYC gene is a proto-oncogene and encodes a nuclear phosphoprotein that plays a role in cell cycle progression, apoptosis and cellular transformation. The encoded protein forms a heterodimer with the related transcription factor MAX. This complex binds to the E box DNA consensus sequence and regulates the transcription of specific target genes. In some embodiments, amplification of this gene is frequently observed in numerous human cancers. In some embodiments, translocations involving this gene are associated with Burkitt lymphoma and multiple myeloma in human patients.

As used herein, the term “PVT1” or “Plasmacytoma variant translocation 1” refers to a long non-coding RNA encoded by the human PVT1 gene that is located in a cancer-related region, 8q24. PVTl’s varied activities include overexpression, modulation of miRNA expression, protein interactions, targeting of regulatory genes, formation of fusion genes, functioning as a competing endogenous RNA (ceRNA), and interactions with MYC, among many others.

As used herein, the term “SCN1A” or “Sodium Voltage-Gated Channel Alpha Subunit 1” encodes for the alpha-1 subunit of the voltage-gated sodium channel (Na(V)1.1). The transmembrane alpha subunit forms the central pore of the channel. The channel responds to the voltage difference across the cell membrane to create a pore that allows sodium ions through the membrane. In some embodiments, Diseases associated with SCN1A include Epileptic Encephalopathy, Early Infantile, 6 and Generalized Epilepsy With Febrile Seizures Plus, Type 2.

As used here, the term “SYNGAP1” or “Synaptic Ras GTPase Activating Protein 1” is located in the brain and provides instructions for making a protein, called SynGAP, that plays an important role in nerve cells in the brain. SynGAP is found at the junctions between nerve cells (synapses) where cell-to-cell communication takes place. Connected nerve cells act as the “wiring” in the circuitry of the brain. Synapses are able to change and adapt over time, rewiring brain circuits, which is critical for learning and memory. SynGAP helps regulate synapse adaptations and promotes proper brain wiring. The protein’s function is particularly important during a critical period of early brain development that affects future cognitive ability.

### First Domain Small Molecule

In some embodiments, the first domain of the bifunctional molecule as described herein, which specifically binds to a target RNA, is a small molecule. In some embodiments, the small molecule is selected from the group consisting of Table 2.

In some embodiments, the small molecule is an organic compound that is 1000 daltons or less. In some embodiments, the small molecule is an organic compound that is 900 daltons or less. In some embodiments, the small molecule is an organic compound that is 800 daltons or less. In some embodiments, the small molecule is an organic compound that is 700 daltons or less. In some embodiments, the small molecule is an organic compound that is 600 daltons or less. In some embodiments, the small molecule is an organic compound that is 500 daltons or less. In some embodiments, the small molecule is an organic compound that is 400 daltons or less.

As used herein, the term “small molecule” refers to a low molecular weight (< 900 daltons) organic compound that may regulate a biological process. In some embodiments, small molecules bind specific biological macromolecules and act as an effector recruiter, altering the activity or function of the target. In some embodiments, small molecules bind nucleotides. In some embodiments, small molecules bind RNAs. In some embodiments, small molecules bind modified nucleic acids. In some embodiments, small molecules bind endogenous nucleic acid sequences. In some embodiments, small molecules bind exogenous nucleic acid sequences. In some embodiments, small molecules bind artificial nucleic acid sequences. In some embodiments, small molecules bind proteins or polypeptides. In some embodiments, small molecules bind enzymes. In some embodiments, small molecules bind receptors. In some embodiments, small molecules bind endogenous polypeptides. In some embodiments, small molecules bind exogenous polypeptides. In some embodiments, small molecules bind artificial polypeptides. In some embodiments, small molecules bind biological macromolecules by covalent binding. In some embodiments, small molecules bind biological macromolecules by non-covalent binding. In some embodiments, small molecules bind biological macromolecules by irreversible binding. In some embodiments, small molecules bind biological macromolecules by reversible binding. In some embodiments, small molecules directly bind biological macromolecules. In some embodiments, small molecules indirectly bind biological macromolecules.

Routine methods can be used to design and identify small molecules that binds to the target sequence with sufficient specificity. In some embodiments, the methods include using bioinformatics methods known in the art to identify regions of secondary structure, e.g., one, two, or more stem-loop structures and pseudoknots, and selecting those regions to target with small molecules.

In some embodiments, the small molecule for purposes of the present methods may specifically bind the sequence to the target RNA or RNA structure and there is a sufficient degree of specificity to avoid non-specific binding of the sequence to non-target RNA sequences under conditions in which specific binding is desired, e.g., under physiological conditions in the case of in vivo assays or therapeutic treatment, and in the case of in vitro assays, under conditions in which the assays are performed under suitable conditions of stringency.

In general, the small molecule must retain specificity for their target, i.e., must not directly bind to, or directly significantly affect expression levels of, transcripts other than the intended target.

In some embodiments, the small molecules bind nucleotides. In some embodiments, the small molecules bind RNAs. In some embodiments, the small molecules bind modified nucleic acids. In some embodiments, the small molecules bind endogenous nucleic acid sequences. In some embodiments, the small molecules bind exogenous nucleic acid sequences. In some embodiments, the small molecules bind artificial nucleic acid sequences.

In some embodiments, the small molecules specifically bind to a target RNA by covalent bonds. In some embodiments, the small molecules specifically bind to a target RNA or a gene sequence by non-covalent bonds. In some embodiments, the small molecules specifically bind to a target RNA sequence by irreversible binding. In some embodiments, the small molecules specifically bind to a target RNA sequence by reversible binding. In some embodiments, the small molecules specifically bind to a target RNA or a gene sequence directly. In some embodiments, the small molecules specifically bind to a target RNA sequence indirectly.

In some embodiments, the small molecules specifically bind to a nuclear RNA or a cytoplasmic RNA. In some embodiments, the small molecules specifically bind to an RNA involved in coding, decoding, regulation and expression of genes. In some embodiments, the small molecules specifically bind to an RNA that plays roles in protein synthesis, post-transcriptional modification, or DNA replication. In some embodiments, the small molecules specifically bind to a regulatory RNA. In some embodiments, the small molecules specifically bind to a non-coding RNA.

In some embodiments, In some embodiments, the small molecules specifically bind to a specific region of the RNA sequence. For example, a specific functional region can be targeted, e.g., a region comprising a known RNA localization motif (i.e., a region complementary to the target nucleic acid on which the RNA acts). Alternatively or in addition, highly conserved regions can be targeted, e.g., regions identified by aligning sequences from disparate species such as primate (e.g., human) and rodent (e.g., mouse) and looking for regions with high degrees of identity.

TABLE 2

Exemplary First Domain Small Molecules that Bind to RNA

| | |
|---|---|
| RNA-binding drug | Target RNA |
| Branaplam | SMN2 pre-mRNA |
| SMA-C5 | SMN2 pre-mRNA |
| ribocil | ribB riboswitch, mRNA |
| 2H-K4NMeS | DM1 CUG expansion mRNA |
| linezolid | 23S rRNA |
| sars-binding | sars (pseudoknot folds) |
| rpoH-mRNA binder | rpoH mRNA |
| aminoglycosides | pre-miRNA |
| yohimbine | IRES elements |
| “134” | U1 snRNA stem-loops |
| “16, 17, 18” | HIV TAR-RNA |
| mitoxanthrone, netilmicin | HIV TAR-RNA |
| “27, 28, 29” | Hep C IRES |
| thiamine, PT | tenA TPP riboswitch |
| oxazolidinones | Tbox riboswitch |
| 2,4 diaminopurine | purine riboswitches |
| RGB1, 2a, GQC-05 | 5′ utr IRES: NRAS, KRAS, BCL-X |
| 2-aminopurine | Adenine riboswitch |
| 2,4,5,6,-tetraminopyrimidine | Mutated G riboswitch |
| 2,4,6-triamino-1,3,5-triazine | Mutated G riboswitch |
| 2,4,6-triaminopyrimidine | Adenine riboswitch |
| 2-substituted aminopyridine | Ribosomal A-site (decoding center) |
| 2,6-diamonopurine | Adenine riboswitch |
| 2,7-quinolinediamine, N2,N2,4-trimethyl- | A-site |
| 3-quinolinecarboxamide | A-site |
| 4-pyridineacetamide, N-[2(dimethylamino)-4-methyl-7-quinolinyl] | A-site |
| 5′-deoxy-5′-adenosylcobalamin (B12) | Riboswitch |
| ABT-773 | U2609 Escherichia coliribosome |
| Acetoperazine | HIV-1 TAR |
| Adenine | Adenine riboswitch |
| Amikacin | A-site |
| Anupam2b | T-box riboswitch |
| Anisomycin | Ribosome (PTC) |
| Apramycin | A-site |
| ATPA-18 | |
| Azithromycin | Ribosome (PTC) |
| B-13 and related | RNA hairpin loops |
| Benzimidazole 13ibis | HCV IRES Domain II |
| Benzimidazole3 ibis | HCV IRES domain II |
| Berenil | Poly(rA).2poly(rU) RNA triplex/TAR |
| Biotin | Biotin aptamer |
| Blasticidin S | PTC |
| Carbomycin | 50S subunit |
| Chloramphenicol | 50S subunit |
| Chlorolissoclimide | Inhibitor of translocation |
| Chlorpromazine | HIV-1 TAR |
| Chlortetracycline | Small subunit |
| Clarithromycin | PTC |
| CMC1 _dioxo-hexahydro-nitro-cyclopentaquinoxaline | HIV-1 TAR |
| CMC2 tetraaminoquinozaline | HIV-1 TAR |
| CMC3 Hoechst33258 | HIV-1 TAR |
| CMC3-1 Hoechst33258 | HIV-1 TAR/tRNA |
| CMC3-2 Hoechst33258 | HIV-1 TAR |
| CMC4 Hoechst33258 | Yeast tRNAphe |
| CMC6 diphenylfuran | HIV-1 RRE |
| CMC7 diphenylfuran | HIV-1 RRE |
| CMC8 diphenylfuran | HIV-1 RRE |
| Cycloheximide | |
| Dalfopristin | Large bacterial ribosomal subunit |
| DAPI | HIV-1 TAR |
| DB340 | HIV-1 RRE |
| Delfinidin | tRNA |
| Dichlorolissoclimide | Inhibit eukaryotic protein synthesis |
| Doxycycline | Small subunit |
| Erythromycin | PTC |
| Ethidium bromide | RNA/DNA heteroduplex, bulged RNA |
| Evernimicin | |
| FMN | Aptamer |
| Geneticin | Eubacterial A-site |
| Gentamicin C1A | Bacterial A-site |
| Glycine | Aptamer |
| Guanine | Guanine riboswitch |
| Hygromycin B | Small bacterial subunit |
| Hypoxanthine | Guanine riboswitch |
| Kanamycin A | Bacterial ribosomal A-site |
| Kanamycin B | A-site |
| Kasugamycin | Bacterial 70S ribosome |
| Linezolid | Bacterial ribosome |
| Lividomycin A | Bacterial ribosomal A-site |
| Malachite green | Aptamer |
| Methidiumpropyl | Bulged RNA |
| Micrococcin | L11 binding domain 50S subunit |
| Minocycline | Small subunit |
| Narciclasine | Eukaryotic ribosomal RNA |
| Negamycin | 50S exit tunnel |
| Neomycin | A-site, others |
| nf2 | A-site |
| nf3 | A-site |
| Nosiheptide | L11 binding domain, large subunit |
| Pactamycin | 30S subunit |
| Parkedavis1 | Group 1 intron |
| Parkdavis2 | Group 1 intron |
| Parkedavis3 | Group I Intron |
| Paromamine | Human A-site |
| Paromomycin | A-site |
| Paromomycin II | A-site |
| Pleuromutilin | PTC |
| Pristinamycin IIA | PTC |
| Promazine | HIV-1 TAR |
| Protoporphyrin IX | tRNA/M1 RNA |
| Puromycin | 50S A-site |
| Quenosine | Riboswitch |
| Quinacridone | HIV-1 TAR |
| Quinupristin | PTC |
| Ralenova (mitoxantrone) | HIV-1 psi RNA/hvg RNA |
| Rbt203 | HIV-1 TAR RNA |
| Rbt417 | HIV-1 TAR |
| Rbt418 | HIV-1 TAR |
| Rbt428 | HIV-1 TAR |
| Rbt489 | HIV-1 TAR |
| Rbt550 | HIV-1 TAR |
| Retapamulin | E. coli and Staphylococcus aureusribosomes |
| Ribostamycin | A-site/HIV dimerization site |
| S-adenosyl methionine | Riboswitch |
| Sisomicin | HCV IRES IIId |
| Spectinomycin | Small subunit |
| Spiramycin A | Exit tunnel, 50S |
| Streptogramin B | 50S subunit |
| T4-MPYP | tRNA, M1 RNA |
| Telithromycin | Large subunit |
| Tetracycline | Small subunit |
| Theophylline | Aptamer |
| Thiamine pyrophosphate | Riboswitch |
| Thiethylperazine | HIV-1 TAR |
| Thiostrepton | L11 binding domain |
| Tiamulin | PTC |
| Tigecycline | Small subunit |
| TMAP | tRNA/M1 RNA |
| Tobramicin | A-site/aptamer |
| Trifluoperazine | HIV-1 TAR |
| Tylosin | Exit tunnel, 50S |
| Usnic acid | HIV-1 TAR |
| Valnemulin | PTC |
| Viomycin | Ribosome intersubunit bridge |
| Wm5 | HIV-1 TAR |
| Xanthinol | HIV-1 TAR |
| Yohimbine | HIV-1 TAR |

Target RNA

In some embodiments, a target ribonucleotide that comprises the target ribonucleic acid sequence is a nuclear RNA or a cytoplasmic RNA. In some embodiments, the nuclear RNA or the cytoplasmic RNA is a long noncoding RNA (lncRNA), pre-mRNA, mRNA, microRNA, enhancer RNA, transcribed RNA, nascent RNA, chromosome-enriched RNA, ribosomal RNA, membrane enriched RNA, or mitochondrial RNA. In some embodiments, the target ribonucleic acid is an intron. In some embodiments, the target ribonucleic acid is an exon. In some embodiments, the target ribonucleic acid is an untranslated region. In some embodiments, the target ribonucleic acid is a region translated into proteins. In some embodiments, the target sequence is translated or untranslated region on an mRNA or pre-mRNA.

In some embodiments, the target ribonucleotide is an RNA involved in coding, noncoding, regulation and expression of genes. In some embodiments, the target ribonucleotide is an RNA that plays roles in protein synthesis, post-transcriptional modification, or DNA replication of a gene. In some embodiments, the target ribonucleotide is a regulatory RNA. In some embodiments, the target ribonucleotide is a non-coding RNA. In some embodiments, a region of the target ribonucleotide that the ASO or the small molecule specifically bind is selected from the full-length RNA sequence of the target ribonucleotide including all introns and exons.

A region that binds to the ASO or the small molecule can be a region of a target ribonucleotide. The region of the target ribonucleotide can comprise various characteristics. The ASO or the small molecule can then bind to this region of the target ribonucleotide. In some embodiments, the region of the target ribonucleotide that the ASO or the small molecule specifically binds is selected based on the following criteria: (i) a SNP frequency; (ii) a length; (iii) the absence of contiguous cytosines; (iv) the absence of contiguous identical nucleotides; (v) GC content; (vi) a sequence unique to the target ribonucleotide compared to a human transcriptome; (vii) the incapability of protein binding; and (viii) a secondary structure score. In some embodiments, the region of the target ribonucleotide comprises at least two or more of the above criteria. In some embodiments, the region of the target ribonucleotide comprises at least three or more of the above criteria. In some embodiments, the region of the target ribonucleotide comprises at least four or more of the above criteria. In some embodiments, the region of the target ribonucleotide comprises at least five or more of the above criteria. In some embodiments, the region of the target ribonucleotide comprises at least six or more of the above criteria. In some embodiments, the region of the target ribonucleotide comprises at least seven or more of the above criteria. In some embodiments, the region of the target ribonucleotide comprises eight of the above criteria. As used herein, the term “transcriptome” refers to the set of all RNA molecules (transcripts) in a specific cell or a specific population of cells. In some embodiments, it refers to all RNAs. In some embodiments, it refers to only mRNA. In some embodiments, it includes the amount or concentration of each RNA molecule in addition to the molecular identities.

In some embodiments, the region of the target ribonucleotide that the ASO or the small molecule specifically binds has a SNP frequency of less than 5%. As used herein, the term “single-nucleotide polymorphism” or “SNP” refers to a substitution of a single nucleotide that occurs at a specific position in the genome, where each variation is present at a level of more than 1% in the population. In some embodiments, the SNP falls within coding sequences of genes, non-coding regions of genes, or in the intergenic regions. In some embodiments, the SNP in the coding region is a synonymous SNP or a nonsynonymous SNP, in which the synonymous SNP does not affect the protein sequence, while the nonsynonymous SNP changes the amino acid sequence of protein. In some embodiments, the nonsynonymous SNP is missense or nonsense. In some embodiments, the SNP that is not in protein-coding regions affects gene splicing, transcription factor binding, messenger RNA degradation, or the sequence of noncoding RNA. In some embodiments, the region of the target ribonucleotide that the ASO or the small molecule specifically binds has a SNP frequency of less than 4%. In some embodiments, the region of the target ribonucleotide that the ASO or the small molecule specifically binds has a SNP frequency of less than 3%. In some embodiments, the region of the target ribonucleotide that the ASO or the small molecule specifically binds has a SNP frequency of less than 2%. In some embodiments, the region of the target ribonucleotide that the ASO or the small molecule specifically binds has a SNP frequency of less than 1%. In some embodiments, the region of the target ribonucleotide that the ASO or the small molecule specifically binds has a SNP frequency of less than 0.9%. In some embodiments, the region of the target ribonucleotide that the ASO or the small molecule specifically binds has a SNP frequency of less than 0.8%. In some embodiments, the region of the target ribonucleotide that the ASO or the small molecule specifically binds has a SNP frequency of less than 0.7%. In some embodiments, the region of the target ribonucleotide that the ASO or the small molecule specifically binds has a SNP frequency of less than 0.6%.

In some embodiments, the region of the target ribonucleotide that the ASO specifically binds has a SNP frequency of less than 0.5%. In some embodiments, the region of the target ribonucleotide that the ASO specifically binds has a SNP frequency of less than 0.4%. In some embodiments the region of the target ribonucleotide that the ASO specifically binds has a SNP frequency of less than 0.3%. the region of the target ribonucleotide that the ASO specifically binds has a SNP frequency of less than 0.2%. In some embodiments, the region of the target ribonucleotide that the ASO specifically binds has a SNP frequency of less than 0.1%.

In some embodiments, the region of the target ribonucleotide that the ASO specifically binds has a sequence comprising from 30% to 70% GC content. In some embodiments, the region of the target ribonucleotide that the ASO specifically binds has a sequence comprising from 40% to 70% GC content. In some embodiments, the region of the target ribonucleotide that the ASO specifically binds has a sequence comprising from 30% to 60% GC content. In some embodiments, the region of the target ribonucleotide that the ASO specifically binds has a sequence comprising from 40% to 60% GC content.

In some embodiments, the region of the target ribonucleotide that the ASO specifically binds has the length of from 8 to 30 nucleotides. In some embodiments, the region of the target ribonucleotide that the ASO specifically binds has the length of from 9 to 30 nucleotides. In some embodiments, the region of the target ribonucleotide that the ASO specifically binds has the length of from 10 to 30 nucleotides. In some embodiments, the region of the target ribonucleotide that the ASO specifically binds has the length of from 11 to 30 nucleotides. In some embodiments, the region of the target ribonucleotide that the ASO specifically binds has the length of from 12 to 30 nucleotides. In some embodiments, the region of the target ribonucleotide that the ASO specifically binds has the length of from 13 to 30 nucleotides. In some embodiments, the region of the target ribonucleotide that the ASO specifically binds has the length of from 14 to 30 nucleotides. In some embodiments, the region of the target ribonucleotide that the ASO specifically binds has the length of from 15 to 30 nucleotides. In some embodiments, the region of the target ribonucleotide that the ASO specifically binds has the length of from 16 to 30 nucleotides. In some embodiments, the region of the target ribonucleotide that the ASO specifically binds has the length of from 17 to 30 nucleotides. In some embodiments, the region of the target ribonucleotide that the ASO specifically binds has the length of from 18 to 30 nucleotides. In some embodiments, the region of the target ribonucleotide that the ASO specifically binds has the length of from 19 to 30 nucleotides. In some embodiments, the region of the target ribonucleotide that the ASO specifically binds has the length of from 20 to 30 nucleotides.

In some embodiments, the region of the target ribonucleotide that the ASO specifically binds has the length of from 8 to 29 nucleotides. In some embodiments, the region of the target ribonucleotide that the ASO specifically binds has the length of from 9 to 29 nucleotides. In some embodiments, the region of the target ribonucleotide that the ASO specifically binds has the length of from 10 to 29 nucleotides. In some embodiments, the region of the target ribonucleotide that the ASO specifically binds has the length of from 11 to 29 nucleotides. In some embodiments, the region of the target ribonucleotide that the ASO specifically binds has the length of from 12 to 29 nucleotides. In some embodiments, the region of the target ribonucleotide that the ASO specifically binds has the length of from 13 to 29 nucleotides. In some embodiments, the region of the target ribonucleotide that the ASO specifically binds has the length of from 14 to 29 nucleotides. In some embodiments, the region of the target ribonucleotide that the ASO specifically binds has the length of from 15 to 29 nucleotides. In some embodiments, the region of the target ribonucleotide that the ASO specifically binds has the length of from 16 to 29 nucleotides. In some embodiments, the region of the target ribonucleotide that the ASO specifically binds has the length of from 17 to 29 nucleotides. In some embodiments, the region of the target ribonucleotide that the ASO specifically binds has the length of from 18 to 29 nucleotides. In some embodiments, the region of the target ribonucleotide that the ASO specifically binds has the length of from 19 to 29 nucleotides. In some embodiments, the region of the target ribonucleotide that the ASO specifically binds has the length of from 20 to 29 nucleotides.

In some embodiments, the region of the target ribonucleotide that the ASO specifically binds has the length of from 8 to 28 nucleotides. In some embodiments, the region of the target ribonucleotide that the ASO specifically binds has the length of from 8 to 27 nucleotides. In some embodiments, the region of the target ribonucleotide that the ASO specifically binds has the length of from 8 to 26 nucleotides. In some embodiments, the region of the target ribonucleotide that the ASO specifically binds has the length of from 8 to 25 nucleotides. In some embodiments, the region of the target ribonucleotide that the ASO specifically binds has the length of from 8 to 24 nucleotides. In some embodiments, the region of the target ribonucleotide that the ASO specifically binds has the length of from 8 to 23 nucleotides. In some embodiments, the region of the target ribonucleotide that the ASO specifically binds has the length of from 8 to 22 nucleotides. In some embodiments, the region of the target ribonucleotide that the ASO specifically binds has the length of from 8 to 21 nucleotides. In some embodiments, the region of the target ribonucleotide that the ASO specifically binds has the length of from 8 to 20 nucleotides.

In some embodiments, the region of the target ribonucleotide that the ASO specifically binds has the length of from 10 to 28 nucleotides. In some embodiments, the region of the target ribonucleotide that the ASO specifically binds has the length of from 11 to 28 nucleotides. In some embodiments, the region of the target ribonucleotide that the ASO specifically binds has the length of from 12 to 28 nucleotides. In some embodiments, the region of the target ribonucleotide that the ASO specifically binds has the length of from 13 to 28 nucleotides. In some embodiments, the region of the target ribonucleotide that the ASO specifically binds has the length of from 14 to 28 nucleotides. In some embodiments, the region of the target ribonucleotide that the ASO specifically binds has the length of from 15 to 28 nucleotides.

In some embodiments, the region of the target ribonucleotide that the ASO specifically binds has the length of from 12 to 27 nucleotides. In some embodiments, the region of the target ribonucleotide that the ASO specifically binds has the length of from 12 to 26 nucleotides. In some embodiments, the region of the target ribonucleotide that the ASO specifically binds has the length of from 12 to 25 nucleotides. In some embodiments, the region of the target ribonucleotide that the ASO specifically binds has the length of from 12 to 24 nucleotides. In some embodiments, the region of the target ribonucleotide that the ASO specifically binds has the length of from 12 to 23 nucleotides. In some embodiments, the region of the target ribonucleotide that the ASO specifically binds has the length of from 12 to 22 nucleotides. In some embodiments, the region of the target ribonucleotide that the ASO specifically binds has the length of from 12 to 21 nucleotides. In some embodiments, the region of the target ribonucleotide that the ASO specifically binds has the length of from 12 to 20 nucleotides.

In some embodiments, the region of the target ribonucleotide that the ASO or the small molecule specifically binds has a sequence unique to the target ribonucleotide compared to a human transcriptome. In some embodiments, the region of the target ribonucleotide that the ASO or the small molecule specifically binds has a sequence lacking at least three contiguous cytosines. In some embodiments, the region of the target ribonucleotide that the ASO or the small molecule specifically binds has a sequence lacking at least four contiguous identical nucleotides. In some embodiments, the region of the target ribonucleotide that the ASO or the small molecule specifically binds has a sequence lacking four contiguous identical nucleotides. In some embodiments, the region of the target ribonucleotide that the ASO or the small molecule specifically binds has a sequence lacking four contiguous identical guanines. In some embodiments, the region of the target ribonucleotide that the ASO or the small molecule specifically binds has a sequence lacking four contiguous identical adenines. In some embodiments, the region of the target ribonucleotide that the ASO or the small molecule specifically binds has a sequence lacking four contiguous identical uracils.

In some embodiments, the region of the target ribonucleotide that the ASO or the small molecule specifically binds to does or does not bind a protein. In some embodiments, the region of the target ribonucleotide that the ASO or the small molecule specifically binds to does or does not comprise a sequence motif or structure motif suitable for binding to a RNA-recognition motif, double-stranded RNA-binding motif, K-homology domain, or zinc fingers of an RNA-binding protein. As a non-limiting example, the region of the target ribonucleotide that the ASO or the small molecule specifically binds does or does not have the sequence motif or structure motif listed in Pan et al., BMC Genomics, 19, 511 (2018) and Dominguez et al., Molecular Cell 70, 854-867 (2018); the contents of each of which are herein incorporated by reference in its entirety. In some embodiments, the region of the target ribonucleotide that an ASO specifically binds does or does not comprise a protein binding site. Examples of the protein binding site includes, but are not limited to, a binding site to the protein such as ACIN1, AGO, APOBEC3F, APOBEC3G, ATXN2, AUH, BCCIP, CAPRIN1, CELF2, CPSF1, CPSF2, CPSF6, CPSF7, CSTF2, CSTF2T, CTCF, DDX21, DDX3, DDX3X, DDX42, DGCR8, EIF3A, EIF4A3, EIF4G2, ELAVL1, ELAVL3, FAM120A, FBL, FIP1L1, FKBP4, FMR1, FUS, FXR1, FXR2, GNL3, GTF2F1, HNRNPA1, HNRNPA2B1, HNRNPC, HNRNPK, HNRNPL, HNRNPM, HNRNPU, HNRNPUL1, IGF2BP1, IGF2BP2, IGF2BP3, ILF3, KHDRBS1, LARP7, LIN28A, LIN28B, m6A, MBNL2, METTL3, MOV10, MSI1, MSI2, NONO, NONO-, NOP58, NPM1, NUDT21, PCBP2, POLR2A, PRPF8, PTBP1, RBFOX2, RBM10, RBM22, RBM27, RBM47, RNPS1, SAFB2, SBDS, SF3A3, SF3B4, SIRT7, SLBP, SLTM, SMNDC1, SND1, SRRM4, SRSF1, SRSF3, SRSF7, SRSF9, TAF15, TARDBP, TIA1, TNRC6A, TOP3B, TRA2A, TRA2B, U2AF1, U2AF2, UNK, UPF1, WDR33, XRN2, YBX1, YTHDC1, YTHDF1, YTHDF2, YWHAG, ZC3H7B, PDK1, AKT1, and any other protein that binds RNA.

In some embodiments, the region of the target ribonucleotide that the small molecule specifically binds has a secondary structure. In some embodiments, the region of the target ribonucleotide that the ASO specifically binds has a limited secondary structure. In some embodiments, the secondary structure of a region of the target ribonucleotide is predicted by a RNA structure prediction software, such as CentroidFold, CentroidHomfold, Context Fold, CONTRAfold, Crumple, CyloFold, GTFold, IPknot, KineFold, Mfold, pKiss, Pknots, PknotsRG, RNA123, RNAfold, RNAshapes, RNAstructure, SARNA-Predict, Sfold, Sliding Windows & Assembly, SPOT-RNA, SwiSpot, UNAFold, and vsfold/vs subopt.

In some embodiments, the region of the target ribonucleotide that the ASO or the small molecule specifically binds has at least two or more of (i) a SNP frequency of less than 5%; (ii) a length of from 8 to 30 nucleotides; (iii) a sequence lacking three contiguous cytosines; (iv) a sequence lacking four contiguous identical nucleotides; (v) a sequence comprising from 30% to 70% GC content; (vi) a sequence unique to the target ribonucleotide compared to a human transcriptome; and (vii) no protein binding. In some embodiments, the region of the target ribonucleotide that the ASO or the small molecule specifically binds has at least three or more of (i) a SNP frequency of less than 5%; (ii) a length of from 8 to 30 nucleotides; (iii) a sequence lacking three contiguous cytosines; (iv) a sequence lacking four contiguous identical nucleotides; (v) a sequence comprising from 30% to 70% GC content; (vi) a sequence unique to the target ribonucleotide compared to a human transcriptome; and (vii) no protein binding. In some embodiments, the region of the target ribonucleotide that the ASO or the small molecule specifically binds has at least four or more of (i) a SNP frequency of less than 5%; (ii) a length of from 8 to 30 nucleotides; (iii) a sequence lacking three contiguous cytosines; (iv) a sequence lacking four contiguous identical nucleotides; (v) a sequence comprising from 30% to 70% GC content; (vi) a sequence unique to the target ribonucleotide compared to a human transcriptome; and (vii) no protein binding. In some embodiments, the region of the target ribonucleotide that the ASO or the small molecule specifically binds has at least five or more of (i) a SNP frequency of less than 5%; (ii) a length of from 8 to 30 nucleotides; (iii) a sequence lacking three contiguous cytosines; (iv) a sequence lacking four contiguous identical nucleotides; (v) a sequence comprising from 30% to 70% GC content; (vi) a sequence unique to the target ribonucleotide compared to a human transcriptome; and (vii) no protein binding. In some embodiments, the region of the target ribonucleotide that the ASO or the small molecule specifically binds has at least six or more of (i) a SNP frequency of less than 5%; (ii) a length of from 8 to 30 nucleotides; (iii) a sequence lacking three contiguous cytosines; (iv) a sequence lacking four contiguous identical nucleotides; (v) a sequence comprising from 30% to 70% GC content; (vi) a sequence unique to the target ribonucleotide compared to a human transcriptome; and (vii) no protein binding. In some embodiments, the region of the target ribonucleotide that the ASO or the small molecule specifically binds has at least seven or more of (i) a SNP frequency of less than 5%; (ii) a length of from 8 to 30 nucleotides; (iii) a sequence lacking three contiguous cytosines; (iv) a sequence lacking four contiguous identical nucleotides; (v) a sequence comprising from 30% to 70% GC content; (vi) a sequence unique to the target ribonucleotide compared to a human transcriptome; and (vii) no protein binding. In some embodiments, the region of the target ribonucleotide that the ASO or the small molecule specifically binds has (i) a SNP frequency of less than 5%; (ii) a length of from 8 to 30 nucleotides; (iii) a sequence lacking three contiguous cytosines; (iv) a sequence lacking four contiguous identical nucleotides; (v) a sequence comprising from 30% to 70% GC content; (vi) a sequence unique to the target ribonucleotide compared to a human transcriptome; and (vii) no protein binding.

In some embodiments, the ASO or the small molecule can be designed to target a specific region of the RNA sequence. For example, a specific functional region can be targeted, e.g., a region comprising a known RNA localization motif (i.e., a region complementary to the target nucleic acid on which the RNA acts). Alternatively or in addition, highly conserved regions can be targeted, e.g., regions identified by aligning sequences from disparate species such as primate (e.g., human) and rodent (e.g., mouse) and looking for regions with high degrees of identity. Percent identity can be determined routinely using basic local alignment search tools (BLAST programs) (Altschul et al, J. Mol. Biol., 1990, 215, 403-410; Zhang and Madden, Genome Res., 1997, 7, 649-656), e.g., using the default parameters.

In some embodiments, the bifunctional molecules bind to the target RNA and recruit the target endogenous protein (e.g., effector) as described herein, by binding of the target endogenous protein to the second domain. Alternatively, in some embodiments, the ASOs or the small molecules may increase transcription, by binding to the target RNA or a gene sequence by way of a target endogenous protein being recruited to the target site by the interaction between the second domain (e.g., effector recruiter) of the bifunctional molecule and the target endogenous protein (e.g., effector).

In some embodiments, the target RNA or a gene is a non-coding RNA, a protein-coding RNA. In some embodiments, the target RNA or a gene comprises a MALAT1 RNA. In some embodiments, the target RNA or a gene comprises an XIST RNA. In some embodiments, the target RNA or a gene comprises a HSP70 RNA. In some embodiments, the target RNA or a gene comprises a MYC RNA. In some embodiments, the target RNA or a gene is a MALAT1 RNA. In some embodiments, the target RNA or a gene is an XIST RNA. In some embodiments, the target RNA or a gene is a HSP70 RNA. In some embodiments, the target RNA or a gene is a MYC RNA.

### Second Domain

In some embodiments, the second domain of the bifunctional molecule as described herein, which specifically binds to a target endogenous protein (e.g., an effector), comprises a small molecule or an aptamer. In some embodiments, the second domain specifically binds to an active site or an allosteric site on the target endogenous protein.

### Second Domain Small Molecule

In some embodiments, the second domain is a small molecule. In some embodiments, the small molecule is selected from Table 3.

Routine methods can be used to design small molecules that binds to the target protein with sufficient specificity. In some embodiments, the small molecule for purposes of the present methods may specifically bind the sequence to the target protein to elicit the desired effects, e.g., increasing transcription, and there is a sufficient degree of specificity to avoid non-specific binding of the sequence to non-target protein under conditions in which specific binding is desired, e.g., under physiological conditions in the case of in vivo assays or therapeutic treatment, and in the case of in vitro assays, under conditions in which the assays are performed under suitable conditions of stringency.

In some embodiments, the small molecules bind an effector. In some embodiments, the small molecules bind proteins or polypeptides. In some embodiments, the small molecules bind endogenous proteins or polypeptides. In some embodiments, the small molecules bind exogenous proteins or polypeptides. In some embodiments, the small molecules bind recombinant proteins or polypeptides. In some embodiments, the small molecules bind artificial proteins or polypeptides. In some embodiments, the small molecules bind fusion proteins or polypeptides. In some embodiments, the small molecules bind enzymes. In some embodiments, the small molecules bind enzymes a regulatory protein. In some embodiments, the small molecules bind receptors. In some embodiments, the small molecules bind signaling proteins or peptides. In some embodiments, the small molecules bind transcription factors. In some embodiments, the small molecules bind transcriptional regulators or mediators

In some embodiments, the small molecules specifically bind to a target protein by covalent bonds. In some embodiments, the small molecules specifically bind to a target protein by non-covalent bonds. In some embodiments, the small molecules specifically bind to a target protein by irreversible binding. In some embodiments, the small molecules specifically bind to a target protein by reversible binding. In some embodiments, the small molecules specifically bind to a target protein through interaction with the side chains of the target protein. In some embodiments, the small molecules specifically bind to a target protein through interaction with the N-terminus of the target protein. In some embodiments, the small molecules specifically bind to a target protein through interaction with the C-terminus of the target protein. In some embodiments, the small molecules specifically binds to an active site or an allosteric site on the target endogenous protein.

In some embodiments, the small molecules specifically bind to a specific region of the target protein sequence. For example, a specific functional region can be targeted, e.g., a region comprising a catalytic domain, a kinase domain, a protein-protein interaction domain, a protein-DNA interaction domain, a protein-RNA interaction domain, a regulatory domain, a signal domain, a nuclear localization domain, a nuclear export domain, a transmembrane domain, a glycosylation site, a modification site, or a phosphorylation site. Alternatively or in addition, highly conserved regions can be targeted, e.g., regions identified by aligning sequences from disparate species such as primate (e.g., human) and rodent (e.g., mouse) and looking for regions with high degrees of identity.

As used herein, the term “Ibrutinib” or “Imbruvica” refers to a small molecule drug that binds permanently to Bruton’s tyrosine kinase (BTK), more specifically binds to the ATP-binding pocket of BTK protein that is important in B cells. In some embodiments, Ibrutinib is used to treat B cell cancers like mantle cell lymphoma, chronic lymphocytic leukemia, and Waldenström’s macroglobulinemia.

As used herein, the term “ORY-1001” refers to a highly potent and selective Lysine-specific histone demethylase 1A (LSD1) inhibitor that induces H3K4me2 accumulation on LSD1 target genes, blast differentiation, and reduction of leukemic stem cell capacity in AML. In some embdiments, ORY-1001 exhibits potent synergy with standard-of-care drugs and selective epigenetic inhibitors. In some embodiments, ORY-1001 is currently being evaluated in patients with leukemia and solid tumors.

In some embodiments, the second domain comprises a pan-BET bromodomain inhibitor. In some embodiments, the second domain comprises a small molecule, JQ1. As used herein, “JQ1” refers to a thienotriazolodiazepine and an inhibitor of the BET family of bromodomain proteins. In some embodiments, the second domain comprises a small molecule, IBET762. As used herein, “IBET762” or “iBET762” refer to a benzodiazepine compound that selectively binds the acetyl-recognizing BET pocket with nanomolar affinity.

TABLE 3

Exemplary Second Domain Small Molecules and Aptamers

| | | |
|---|---|---|
| Group | Enzyme | Inhibitors/Aptamers |
| Lysine methyltransferase | KMT5A | SPS8I1 |
| | KMT5B/KMT5C | A196 |
| | EZH2 | UNC1999; GSK343, GSK126, EI1, CPI-169, CPI-1205, CPI-0209, EPZ-6438, DS-3201, PF-06821497 |
| | EED | A-395, MAK683 |
| | EHMT1 (KMT1C, GLP)/ EHMT2 (KMT1D, G9a) | A-366; UNC0642; UNC0638, BIX-01294, BRD9539, BRD4770 |
| | NSD2 | LEM-14 |
| | SETD7 | PFI-2 |
| | SMYD2 | PFI-5, BAY-598, LLY507, AZ505 |
| | SMYD3 | BAY-6035 |
| | | BCI-121 |
| | | EPZ030456 |
| | | EPZ031686 |
| | | EPZ028862 |
| | | GSK2807 |
| | DOT1L | SGC0946, EPZ-004777, EPZ-5676 |
| Lysine demethylase | KDM1A(LSD1) | GSK-LSD1, ORY-1001, RN-1, GSK2879552 |
| | KDM4A/KDM4B/KDM 4C/KDM4D | QC6352 |
| | KDM5A/KDM5B/KDM 5C/KDM5D | CPI-455, Compound 20, compound 1, compound 50, GSK-J1 family, KDOAM-25 |
| | KDM6A (UTX)/KDM6B (JMJD3) | GSK-J1 family |
| Arginine methyltransferase | Type I PRMTs (PRMT1,3,4,6,8) | MS023, GSK3368715 |
| | PRMT1 | TC-E 5003 |
| | PRMT3 | SGC707 and derivatives |
| | | Compound 1 |
| | CARM1 (PRMT4) | SKI-73;TP-064, EPZ-025654, EZM2302 (GSK3359088) |
| | PRMT4 (CARM1), PRMT6 | MS049 |
| | PRMT5 | GSK591, LLY-283, EPZ015666, GSK3326595 |
| | PRMT6 | SGC6870, EPZ020411 |
| SWI/SNF | BRG1 (SMARCA4)/BRM (SMARCA2) | Compounds 11-14 |
| | SNF2L (SMARCA5)/CHD4 | ED2-AD101 |
| | ARID1A (BAF250a) | A01, A11, C09 |
| | SWI/SNF | BRD-K98645985 |
| | BRD7/9 | GNE-375, BI-7273, BI-9564, i-BRD9 (GSK602), LP 99, TP472, bromosporine |
| Readers/TF | BRD2, BRD3, BRD4, BRDT (BET) | ZEN-3694, CPI-0610, CPI 203, ABBV-075, BAY1238097, BI 894999, BMS-986158, FT-1101, GS-5829, GS-626510, GSK525762, GSK2820151, INCB054329, INCB057643, OTX015, PLX51107, RO6870810, JQ1, RVX-208, AZD5153, PFI-1, RVX-208, MK-7965, CC-90010, ABBV-744 |
| | TAF1 (KAT4) | GNE-371, BAY299 |
| | ATAD2A/ATAD2B | BAY850 |
| | BAZ2A/BAZ2B | GSK2801 |
| | BPTF | rac1 (AU1), C620-0696 |
| | BRPF 1 B/TRIM24 | IACS-9571 |
| | BRPF1/2/3 | GSK6853 |
| | CECR2 | GNE-886 |
| DNA modifiers | DNMT1/DNMT3A/DN MT3B | Decitabine, Azacytidine, ATA |
| | DNMT1 | Aptamer 9, EGCG, RG108 |
| | DNMT3 | Nanomycin A |
| | TET1/2/3 | DMOG, Bobcat339 (aptamer) |
| HAT | KAT2A (GCN5) / KAT2B (PCAF) | GSK4027, BRD-IN-3 (bromo) |
| | KAT3A (CBP) / KAT3B (P300) | (HAT) A-485, C646, P300/CBP-IN-3, P300/CBP-IN-5, I-CBP112, L002, B026 |
| | | (Bromo) GNE-781, GNE-272, CPI-637, CCS1477 |
| | KAT6A (MYST3)/ KAT6B (MYST4) | WM-1119, WM-8014 |
| HDACs | HDAC1, HDAC2, HDAC3, HDAC4, | A7B4, A8B4, A12B4, A14B3, A14B4,Abexinostat, Apicidin, AR-42, Belinostat, BG45, BML-210, |
| | HDAC5, HDAC6, HDAC7, HDAC8, HDAC9, HDAC10, HDAC11, SIRT1, SIRT2, SIRT3, SIRT4, SIRT5, SIRT6, SIRT7 | BMN290, BRD0302, BRD2283, BRD3227, BRD3349, BRD3386, BRD3493, BRD4161, BRD4884, BRD6688, BRD8951, BRD9757, CBHA, Chromopeptide A, Citarinostat, CM-414, compound 25, CRA-026440, Crebinostat, CUDC-101, CUDC-907, Curcumin, Dacinostat, Depudecin, Domatinostat, Droxinostat, Entinostat, EVX001688, FR901228, FRM-0334, Givinostat, HDACi-4b, HDACi-109, HPOB, I2, KD5170, LB-205, M344, MC1742, MC2625, Merck60, Mocetinostat, OBP-801, Oxamflatin, Panobinostat, PCI-34051, PCI-48000, Pracinostat, Pyroxamide, Quisinostat, Reminostat, RG2833, RGFP963 RGFP966, RGFP968, Rocilinostat, Romidepsin, Scriptaid, sodium phenylbutyrate, Splitomicin, T247, Tacedinaline, Trapoxin, Trichostatin A (TSA), Tucidinostat, US2016031823A1, Valproic acid, vorinostat (SAHA), W2, WO2018119362A2, LBH589, PXD101, ITF2357, PCI-24781, FK228, MS-275, MGCD0103, Phenylbutyrate, AN-9, Baceca, Savicol, EX-527, Sirtinol, Cambinol, salermide, Tenovin-6, Suramin, AGK2 |
| Artificial Transcription Factors | CREB binding protein (CBP), TRRAP/Tra1 (a component of the SAGA complex), and the components of Mediator complex, Med15/Gal11 and MED23/Sur2 | Isoxazolidine, wrenchnolol |

### Aptamer

In some embodiments, the second domain of the bifunctional molecule as described herein, which specifically binds to a target endogenous protein is an aptamer. In some embodiments, the aptamer is selected from Table 3.

As used herein, the term “aptamer” refers to oligonucleotide or peptide molecules that bind to a specific target molecule. In some embodiments, the aptamers bind to a target protein.

Routine methods can be used to design and select aptamers that binds to the target protein with sufficient specificity. In some embodiments, the aptamer for purposes of the present methods bind to the target protein to recruit the protein (e.g., effector). Once recruited, the protein performs the desired effects, e.g., increasing transcription, and there is a sufficient degree of specificity to avoid non-specific binding of the sequence to non-target protein under conditions in which specific binding is desired, e.g., under physiological conditions in the case of in vivo assays or therapeutic treatment, and in the case of in vitro assays, under conditions in which the assays are performed under suitable conditions of stringency.

In some embodiments, the aptamers bind proteins or polypeptides. In some embodiments, the aptamers bind endogenous proteins or polypeptides. In some embodiments, the aptamers bind exogenous proteins or polypeptides. In some embodiments, the aptamers bind recombinant proteins or polypeptides. In some embodiments, the aptamers bind artificial proteins or polypeptides. In some embodiments, the aptamers bind fusion proteins or polypeptides. In some embodiments, the aptamers bind enzymes. In some embodiments, the aptamers bind enzymes a regulatory protein. In some embodiments, the aptamers bind receptors. In some embodiments, the aptamers bind signaling proteins or peptides. In some embodiments, the aptamers bind transcription factors. In some embodiments, the aptamers bind transcriptional regulators or mediators.

In some embodiments, the aptamers specifically bind to a target protein by covalent bonds. In some embodiments, the aptamers specifically bind to a target protein by non-covalent bonds. In some embodiments, the aptamers specifically bind to a target protein by irreversible binding. In some embodiments, the aptamers specifically bind to a target protein by reversible binding. In some embodiments, the aptamers specifically binds to an active site or an allosteric site on the target endogenous protein.

In some embodiments, In some embodiments, the aptamers specifically bind to a specific region of the target protein sequence. For example, a specific functional region can be targeted, e.g., a region comprising a catalytic domain, a kinase domain, a protein-protein interaction domain, a protein-DNA interaction domain, a protein-RNA interaction domain, a regulatory domain, a signal domain, a nuclear localization domain, a nuclear export domain, a transmembrane domain, a glycosylation site, a modification site, or a phosphorylation site. Alternatively or in addition, highly conserved regions can be targeted, e.g., regions identified by aligning sequences from disparate species such as primate (e.g., human) and rodent (e.g., mouse) and looking for regions with high degrees of identity.

In some embodiments, the aptamers reduce or interfere the activity or function of the protein, e.g., increase transcription, by binding to the target protein after recruited to the target site by the interaction between the first domain of the bifunctional molecule as described herein. Alternatively, the aptamers bind to the target protein and recruit the bifunctional molecule as described herein, thereby allowing the first domain to specifically bind to a target RNA sequence.

In some embodiments, the second domain comprises an aptamer that binds to histone deacetylases. In some embodiments, the second domain comprises an aptamer that binds to BTK. In some embodiments, the second domain comprises an aptamer that binds to LSD1.

### Plurality of Second Domains

In some embodiments, the synthetic bifunctional molecule as provided herein comprises a first domain and one or more second domains. In some embodiments, the bifunctional molecule has 1, 2, 3, 4, 5, 6, 7, 8, 9, 10 or more second domains. In some embodiments, each of the one or more second domains specifically binds to a target endogenous protein.

In one aspect, the synthetic bifunctional molecule comprises a first domain that specifically binds to a target RNA sequence, a plurality of second domains, wherein each of the plurality of second domains that specifically bind to a single target endogenous protein. In some embodiments, the bifunctional molecule further comprises a linker that conjugates the first domain to the plurality of second domains.

In some embodiments, the first domain comprises a small molecule or an ASO. In some embodiments, the bifunctional molecule comprises a plurality of second domains. Each of the plurality of second domains comprise a small molecule or an aptamer. In some embodiments, each of the plurality of second domains comprise a small molecule. In some embodiments, each of the plurality of second domains comprise an aptamer.

In some embodiments, the bifunctional molecule comprises a plurality of second domains, e.g., 2, 3, 4, 5, 6, 7, 8, 9, or 10 second domains. In one embodiment, the bifunctional molecule has 2 second domains. In one embodiment, the bifunctional molecule has 3 second domains. In one embodiment, the bifunctional molecule has 4 second domains. In one embodiment, the bifunctional molecule has 5 second domains. In one embodiment, the bifunctional molecule has 6 second domains. In one embodiment, the bifunctional molecule has 7 second domains. In one embodiment, the bifunctional molecule has 8 second domains. In one embodiment, the bifunctional molecule has 9 second domains. In one embodiment, the bifunctional molecule has 10 second domains. In one embodiment, the bifunctional molecule has more than 10 second domains.

In some embodiments, the plurality of second domains is same domains. In some embodiments, the plurality of second domains is different domains. In some embodiments, the plurality of second domains binds to a same target. In some embodiments, the plurality of second domains binds to different targets.

### Target Protein

In some embodiments, the target protein may be an effector. In other embodiments, the target proteins may be endogenous proteins or polypeptides. In some embodiments, the target proteins may be exogenous proteins or polypeptides. In some embodiments, the target proteins may be recombinant proteins or polypeptides. In some embodiments, the target proteins may be artificial proteins or polypeptides. In some embodiments, the target proteins may be fusion proteins or polypeptides. In some embodiments, the target proteins may be enzymes. In some embodiments, the target proteins may be receptors. In some embodiments, the target proteins may be signaling proteins or peptides. In some embodiments, the target proteins may be transcription factors. In some embodiments, the target proteins may be transcriptional regulators or mediators.

In some embodiments, the activity or function of the target protein, e.g., transcription, may be increased by binding to the second domain of the bifunctional molecule as provided herein. In some embodiments, the target protein recruits the bifunctional molecule as described herein by binding to the second domain of the bifunctional molecule as provided herein, thereby allowing the first domain to specifically bind to a target RNA sequence. In some embodiments, the target protein further recruits additional functional domains or proteins.

In some embodiments, the target protein comprises a transcriptional modifying enzyme. In some embodiments, the target protein comprises a histone deacetylase. In some embodiments, the target protein comprises a transcriptional activator. In some embodiments, the target protein comprises a transcriptional repressor. In some embodiments, the target protein comprises a tyrosine kinase. In some embodiments, the target protein comprises a histone demethylase.. In some embodiments, the target protein comprises an RNA modifying enzyme. In some embodiments, the target protein comprises an RNA methyltransferase.

In some embodiments, the target protein is a transcriptional modifying enzyme. In some embodiments, the target protein is a histone deacetylase. In some embodiments, the target protein is a transcriptional activator. In some embodiments, the target protein is a transcriptional repressor. In some embodiments, the target protein is a tyrosine kinase. In some embodiments, the target protein is a histone demethylase. In some embodiments, the target protein is a nuclease. In some embodiments, the target protein is an RNA modifying enzyme. In some embodiments, the target protein is an RNA methyltransferase.

In some embodiments, the target protein includes BRD4. As used herein, the term “BRD4” or “Bromodomain-containing protein 4” refers to an epigenetic reader that recognizes histone proteins and acts as a transcriptional regulator to trigger tumor growth and the inflammatory response. BRD4 is a member of the BET (bromodomain and extra terminal domain) family. The domains of mammalian BET proteins are highly conserved, including mice. The pan-BET inhibitor, (+)-JQ1, may inhibit angiogenesis that contributes to inflammation, infections, immune disorders, and carcinogenesis.

### Linkers

In some embodiments, the synthetic bifunctional molecule comprises a first domain that specifically binds to a target RNA sequence and a second domain that specifically binds to a target endogenous protein, wherein the first domain is conjugated to the second domain by a linker molecule.

In certain embodiments, the first domain and the second domain of the bifunctional molecules described herein can be chemically linked or coupled via a chemical linker (L). In certain embodiments, the linker is a group comprising one or more covalently connected structural units. In certain embodiments, the linker directly links the first domain to the second domain. In other embodiments, the linker indirectly links the first domain to the second domain. In some embodiments, one or more linkers can be used to link a first domain, one or more second domains, a third domain, or a combination thereof.

In certain embodiments, the linker is a bond, CR^{L1}R^{L2}, O, S, SO, SO₂, NR^{L3}, SO₂NR^{L3}, SONR^{L3}, CONR^{L3}, NR^{L3}CONR^{w}, NR^{L3}SO₂NR^{w}, CO, CR^{L}═CR^{L2}, C≡C, SiR^{L1}R^{L2}, P(0)R^{L1}, P(0)OR^{L1}, NR^{L3}C(═NCN)NR^{W}, NR^{L3}C(═NCN), NR^{L3}C(═CNO₂)NR^{L4}, C₃-n-cycloalkyl optionally substituted with 0-6 R^{L1} and/or R^{L2} groups, C3-n-heteocyclyl optionally substituted with 0-6 R^{LI} and/or R^{L2} groups, aryl optionally substituted with 0-6 R^{LI} and/or R^{L2} groups, heteroaryl optionally substituted with 0-6 R^{LI} and/or R^{L2} groups, where R^{LI} or R^{L2}, each independently, can be linked to other groups to form cycloalkyl and/or heterocyclyl moeity which can be further substituted with 0-4 R groups; wherein R^{L1}, R^{L2}, R^{L3}, R^{w} and R^{L5} are, each independently, H, halo, Cisalkyl, OCisalkyl, SCisalkyl, NHCisalkyl, N(Cisalkyl)₂, C₃n-cycloalkyl, aryl, heteroaryl, C₃n-heterocyclyl, OCiscycloalkyl, SCiscycloalkyl, NHCiscycloalkyl, N(Ci₈cycloalkyl)₂, N(Ciscycloalkyl)(Ci galkyl), OH, NH₂, SH, SO₂Ci₈alkyl, P(0)(OCisalkyl)(Ci_ alkyl), P(0)(OCi₈alkyl)₂, CC-Cisalkyl, CCH, CH═CH(Cisalkyl), C(Ci₈alkyl)═CH(Ci₈alkyl), C(Ci₈alkyl)═C(Ci₈alkyl)₂, Si(OH)₃, Si(Ci₈alkyl)₃, Si(OH)(Ci₈alkyl)₂, COCisalkyl, CO₂H, halogen, CN, CF₃, CHF₂, CH₂F, NO₂, SF₅, SO₂NHCi₈alkyl, SO₂N(Ci₈alkyl)₂, SONHCisalkyl, SON(Ci₈alkyl)₂, CONHCisalkyl, CON(Ci₈alkyl)₂, N(Ci₈alkyl)CONH(Ci₈alkyl), N(Ci_ alkyl)CON(Ci₈alkyl)₂, NHCONH(Cisalkyl), NHCON(Ci₈alkyl)₂, NHCONH₂, N(Ci₈alkyl)SO₂NH(Ci₈salkyl), N(Ci₈alkyl)SO₂N(Ci₈alkyl)₂, NHSO₂NH(Ci₈alkyl), NHSO₂N(Ci₈alkyl)₂, NHSO₂NH₂.

In certain embodiments, the linker (L) is selected from the group consisting of: —(CH₂)ₙ—(lower alkyl)-, —(CH₂)ₙ—(lower alkoxyl)-, —(CH₂)ₙ—(lower alkoxyl) —OCH₂—C(O)—, —(CH₂)ₙ—(lower alkoxyl)-(lower alkyl)—OCH₂—C(O)—, —(CH₂)ₙ—(cycloalkyl)-(lower alkyl)—OCH₂—C(O)—, —(CH₂)ₙ—(hetero cycloalkyl)-, —(CH₂CH₂O)ₙ—(lower alkyl)—O—CH₂—C(O)—, —(CH₂CH₂O)ₙ—(hetero cycloalkyl)—O—CH₂—C(O)—, —(CH₂CH₂O)ₙ—Aryl—O—CH₂—C(O)—, —(CH₂CH₂O)ₙ—(hetero aryl)—O—CH₂—C(O)—, —(CH₂CH₂O) —(cyclo alkyl)—O—(hetero aryl)—O—CH₂—C(O)—, —(CH₂CH₂O)ₙ—(cyclo alkyl)—O—Aryl—O—CH₂—C(O)—, —(CH₂CH₂O)ₙ—(lower alkyl)—NH—Aryl—O—CH₂—C(0)—, —(CH₂CH₂O)ₙ—(lower alkyl)—O—Aryl—C(O)—, —(CH₂CH₂O)ₙ—cycloalkyl—O—Aryl—C(O)—, —(CH₂CH₂O)ₙ—cycloalkyl—O—(hetero aryl)—C(O)—, where n can be 0 to 10;

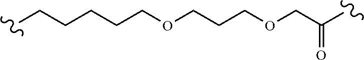

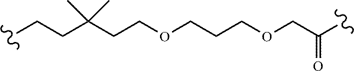

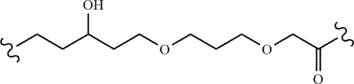

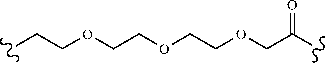

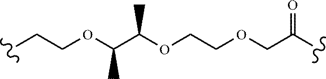

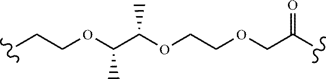

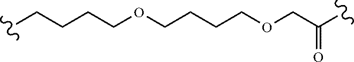

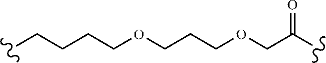

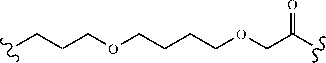

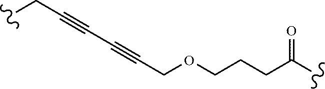

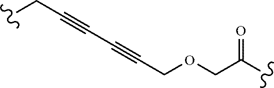

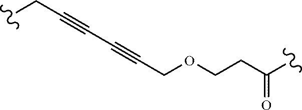

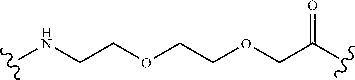

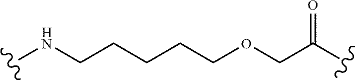

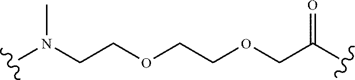

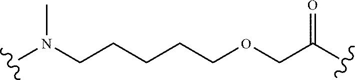

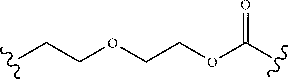

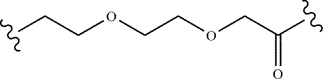

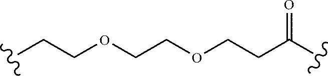

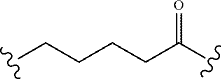

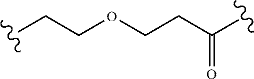

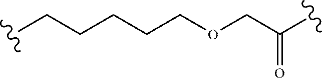

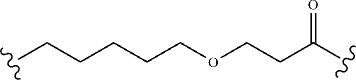

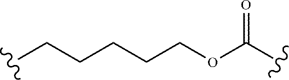

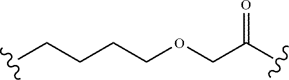

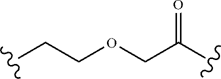

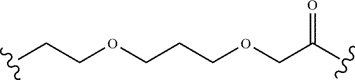

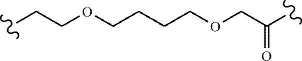

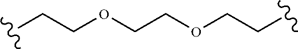

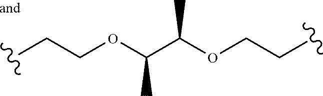

In additional embodiments, the linker group is optionally substituted (poly)ethyleneglycol having between 1 and about 100 ethylene glycol units, between about 1 and about 50 ethylene glycol units, between 1 and about 25 ethylene glycol units, between about 1 and 10 ethylene glycol units, between 1 and about 8 ethylene glycol units and 1 and 6 ethylene glycol units, between 2 and 4 ethylene glycol units, or optionally substituted alkyl groups interdispersed with optionally substituted, O, N, S, P or Si atoms. In certain embodiments, the linker is substituted with an aryl, phenyl, benzyl, alkyl, alkylene, or heterocycle group. In certain embodiments, the linker may be asymmetric or symmetrical.

In any of the embodiments described herein, the linker group may be any suitable moiety as described herein. In one embodiment, the linker is a substituted or unsubstituted polyethylene glycol group ranging in size from about 1 to about 12 ethylene glycol units, between 1 and about 10 ethylene glycol units, about 2 about 6 ethylene glycol units, between about 2 and 5 ethylene glycol units, between about 2 and 4 ethylene glycol units.

Although the first domain and the second domain may be covalently linked to the linker group through any group which is appropriate and stable to the chemistry of the linker, in some aspects, the linker is independently covalently bonded to the first domain and the second domain through an amide, ester, thioester, keto group, carbamate (urethane), carbon or ether, each of which groups may be inserted anywhere on the first domain and second domain to provide maximum binding. In certain preferred aspects, the linker may be linked to an optionally substituted alkyl, alkylene, alkene or alkyne group, an aryl group or a heterocyclic group on the first domain and/or the second domain.

In certain embodiments, the linker can be linear chains with linear atoms from 4 to 24, the carbon atom in the linear chain can be substituted with oxygen, nitrogen, amide, fluorinated carbon, etc., such as the following:

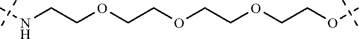

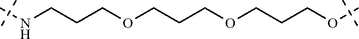

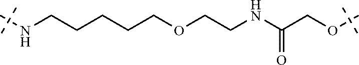

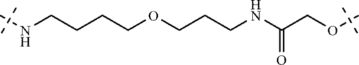

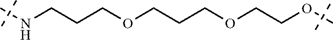

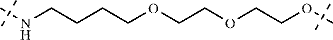

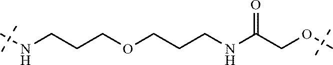

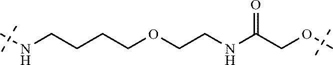

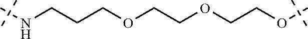

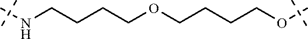

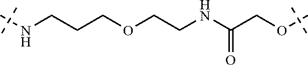

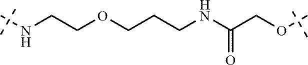

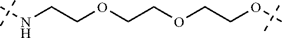

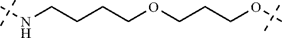

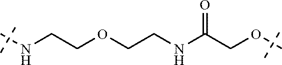

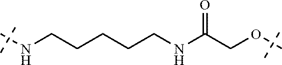

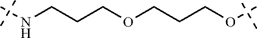

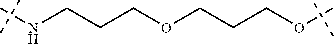

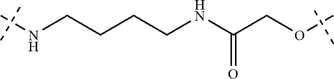

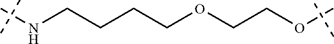

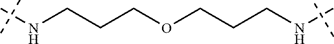

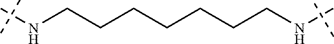

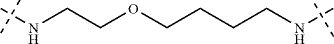

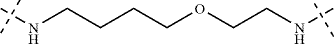

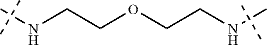

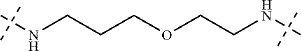

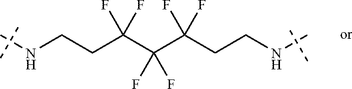

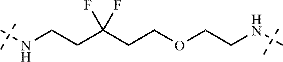

In some embodiments, the linker comprises a mixer of regioisomers. In some embodiments, the mixer of regioisomers is selected from the group consisting of Linkers 1-5:

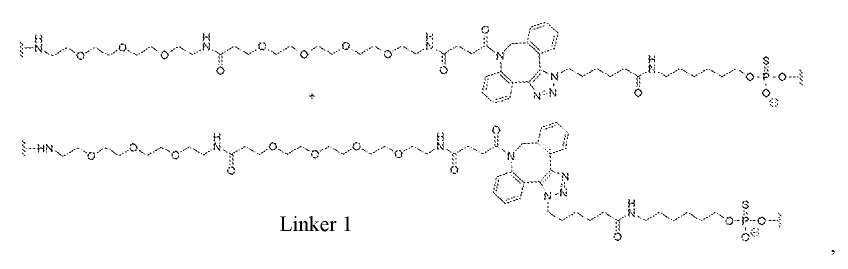

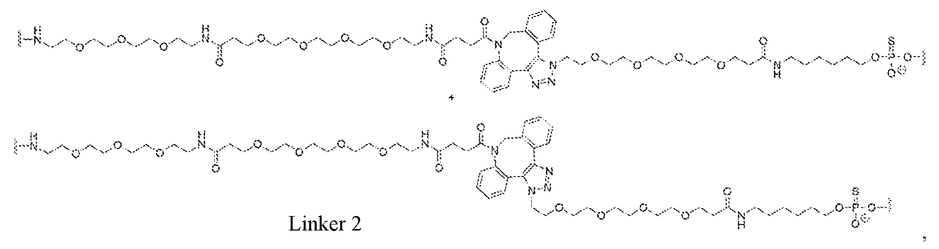

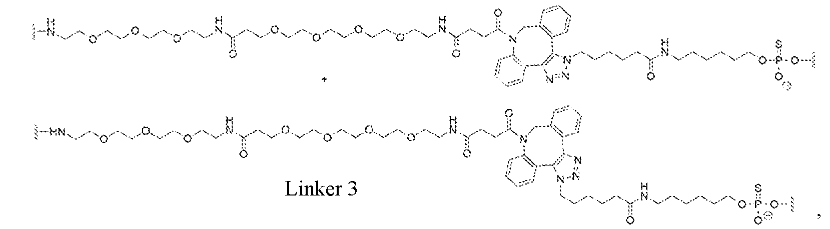

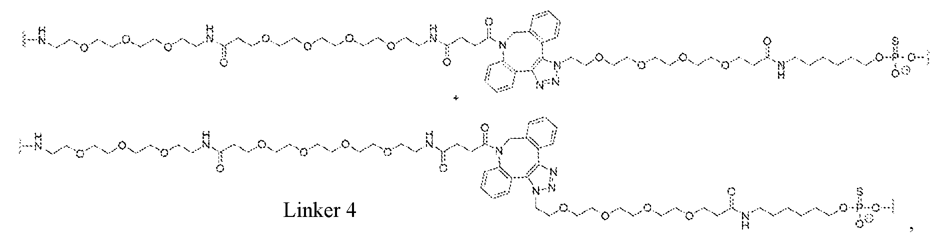

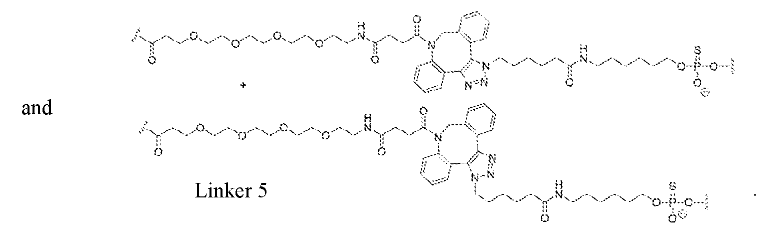

In some embodiments, the linker comprises a modular linker. In some embodiments, the modular linker comprises one or more modular regions that may be substituted with a linker module. In some embodiments, the modular linker having a modular region that can be substituted with a linker module comprises:

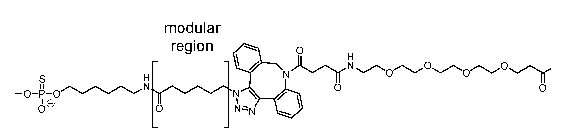

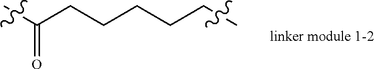

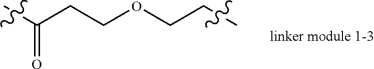

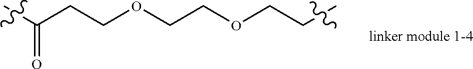

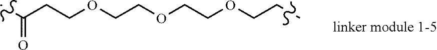

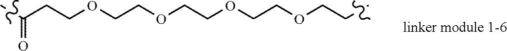

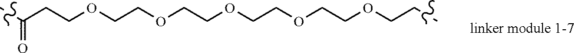

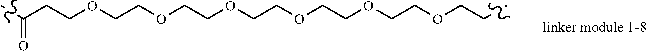

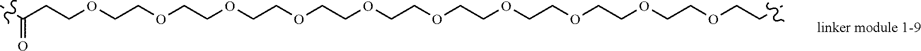

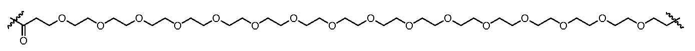

or

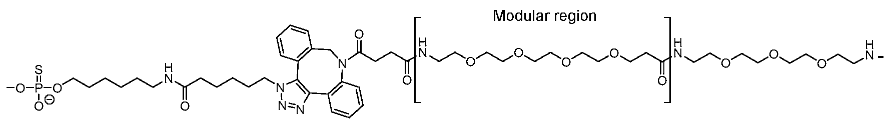

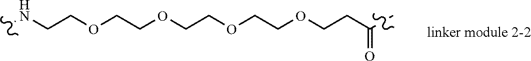

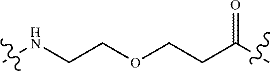

In certain embodiments, the linker can be nonlinear chains, and can be aliphatic or aromatic or heteroaromatic cyclic moieties. Some examples of linkers include but is not limited to the following:

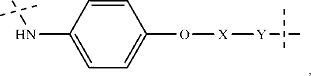

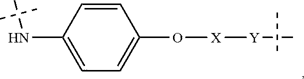

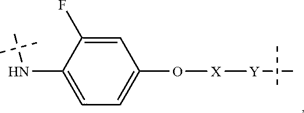

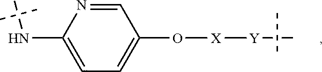

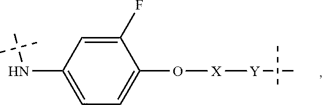

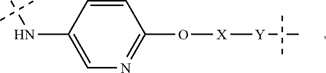

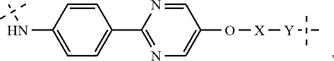

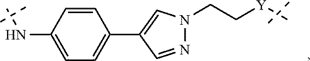

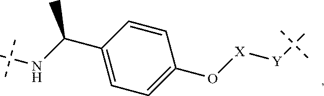

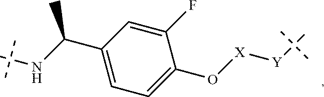

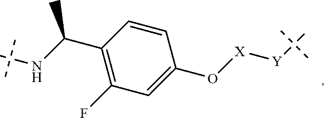

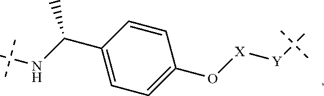

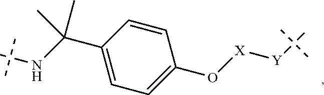

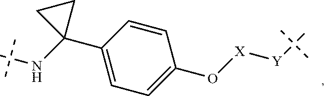

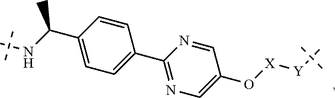

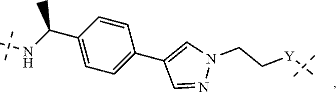

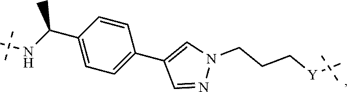

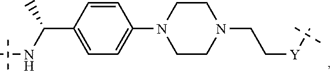

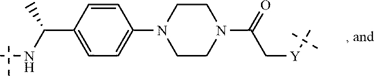

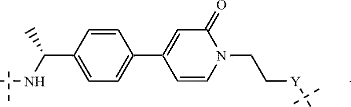

wherein “X” can be linear chain with atoms ranging from 2 to 14, and can contain heteroatoms such as oxygen and “Y” can be O, N, S(O)ₙ (n=0, 1, or 2).

Other examples of linkers include, but are not limited to: Allyl(4-methoxyphenyl)dimethylsilane, 6-(Allyloxycarbonylamino)-1-hexanol, 3-(Allyloxycarbonylamino)-1-propanol, 4-Aminobutyraldehyde diethyl acetal, (E)-N-(2-Aminoethyl)-4-{2-[4-(3-azidopropoxy)phenyl]diazenyl}benzamide hydrochloride, N-(2-Aminoethyl)maleimide trifluoroacetate salt, Amino-PEG4-alkyne, Amino-PEG4-t-butyl ester, Amino-PEG5-t-butyl ester, Amino-PEG6-t-butyl ester, 20-Azido-3,6,9,12,15,18-hexaoxaicosanoic acid, 17-Azido-3,6,9,12,15-pentaoxaheptadecanoic acid, Benzyl N-(3-hydroxypropyl)carbamate, 4-(Boc-amino)-1-butanol, 4-(Boc-amino)butyl bromide, 2-(Boc-amino)ethanethiol, 2-[2-(Boc-amino)ethoxy]ethoxyacetic acid (dicyclohexylammonium) salt, 2-(Boc-amino)ethyl bromide, 6-(Boc-amino)-1-hexanol, 21-(Boc-amino)-4,7,10,13,16,19-hexaoxaheneicosanoic acid purum, 6-(Boc-amino)hexyl bromide, 3-(Boc-amino)-1-propanol, 3-(Boc-amino)propyl bromide, 15-(Boc-amino)-4,7,10,13-tetraoxapentadecanoic acid purum, N-Boc-1,4-butanediamine, N-Boc-cadaverine, N-Boc-ethanolamine, N-Boc-ethylenediamine, N-Boc- 2,2′-(ethylenedioxy)diethylamine, N-Boc-1,6-hexanediamine, N-Boc-1,6-hexanediamine hydrochloride, N-Boc-4-isothiocyanatoaniline, N-Boc-3-isothiocyanatopropylamine, N-Boc-N-methylethylenediamine, BocNH-PEG4-acid, BocNH-PEG5-acid, N-Boc-m-phenylenediamine, N-Boc-p-phenylenediamine, N-Boc-1,3-propanediamine, N-Boc-1,3-propanediamine, N-Boc-N′-succinyl-4,7,10-trioxa-1,13-tridecanediamine, N-Boc-4,7,10-trioxa-1, 13-tridecanediamine, N-(4-Bromobutyl)phthalimide, 4-Bromobutyric acid, 4-Bromobutyryl chloride, N-(2-Bromoethyl)phthalimide, 6-Bromo-1-hexanol, 8-Bromooctanoic acid, 8-Bromo-1-octanol, 3-(4-Bromophenyl)-3-(trifluoromethyl)-3H-diazirine, N-(3-Bromopropyl)phthalimide, 4-(tert-Butoxymethyl)benzoic acid, tert-Butyl 2-(4-{[4-(3-azidopropoxy)phenyl]azo}benzamido)ethylcarbamate, 2-[2-(tert-Butyldimethylsilyloxy)ethoxy]ethanamine, tert-Butyl 4-hydroxybutyrate, Chloral hydrate, 4-(2-Chloropropionyl)phenylacetic acid, 1,11-Diamino-3,6,9-trioxaundecane, di-Boc-cystamine, Diethylene glycol monoallyl ether, 3,4-Dihydro-2H-pyran-2-methanol, 4-[(2,4-Dimethoxyphenyl)(Fmoc-amino)methyl]phenoxyacetic acid, 4-(Diphenylhydroxymethyl)benzoic acid, 4-(Fmoc-amino)-1-butanol, 2-(Fmoc-amino)ethanol, 2-(Fmoc-amino)ethyl bromide, 6-(Fmoc-amino)-1-hexanol, 5-(Fmoc-amino)-1-pentanol, 3-(Fmoc-amino)-1-propanol, 3-(Fmoc-amino)propyl bromide, N-Fmoc-2-bromoethylamine, N-Fmoc-1,4-butanediamine hydrobromide, N-Fmoc-cadaverine hydrobromide, N-Fmoc-ethylenediamine hydrobromide, N-Fmoc-1,6-hexanediamine hydrobromide, N-Fmoc-1,3-propanediamine hydrobromide, N-Fmoc-N″-succinyl-4,7,10-trioxa-1,13-tridecanediamine, (3-Formyl-1-indolyl)acetic acid, 4-Hydroxybenzyl alcohol, N-(4-Hydroxybutyl)trifluoroacetamide, 4′-Hydroxy-2,4-dimethoxybenzophenone, N-(2-Hydroxyethyl)maleimide, 4-[4-(1-Hydroxyethyl)-2-methoxy-5-nitrophenoxy]butyric acid, N-(2-Hydroxyethyl)trifluoroacetamide, N-(6-Hydroxyhexyl)trifluoroacetamide, 4-Hydroxy-2-methoxybenzaldehyde, 4-Hydroxy-3-methoxybenzyl alcohol, 4-(Hydroxymethyl)benzoic acid, 4-(Hydroxymethyl)phenoxyacetic acid, Hydroxy-PEG4-t-butyl ester, Hydroxy-PEG5-t-butyl ester, Hydroxy-PEG6-t-butyl ester, N-(5-Hydroxypentyl)trifluoroacetamide, 4-(4′-Hydroxyphenylazo)benzoic acid, 2-Maleimidoethyl mesylate, 6-Mercapto-1-hexanol, Phenacyl 4-(bromomethyl)phenylacetate, Propargyl-PEG6-acid, 4-Sulfamoylbenzoic acid, 4-Sulfamoylbutyric acid, 4-(Z-Amino)-1-butanol, 6-(Z-Amino)-1-hexanol, 5-(Z-Amino)-1-pentanol, N-Z-1,4-Butanediamine hydrochloride, N-Z-Ethanolamine, N-Z-Ethylenediamine hydrochloride, N-Z-1,6-hexanediamine hydrochloride, N-Z-1,5-pentanediamine hydrochloride, and N-Z-1,3-Propanediamine hydrochloride.

In some embodiments, the linker is conjugated at a 5′ end or a 3′ end of the ASO. In some embodiments, the linker is conjugated at a position on the ASO that is not at the 5′ end or at the 3′ end.

In some embodiments, the synthetic bifunctional molecule comprises a first domain that specifically binds to a target RNA sequence, a plurality of second domains, wherein each of the plurality of second domains that specifically bind to a single target endogenous protein, and a linker that conjugates the first domain to the plurality of second domains.

In some embodiments, linkers comprise 1-10 linker-nucleosides. In some embodiments, such linker-nucleosides are modified nucleosides. In certain embodiments such linker-nucleosides comprise a modified sugar moiety. In some embodiments, linker-nucleosides are unmodified. In some embodiments, linker-nucleosides comprise an optionally protected heterocyclic base selected from a purine, substituted purine, pyrimidine or substituted pyrimidine. In some embodiments, a cleavable moiety is a nucleoside selected from uracil, thymine, cytosine, 4-N-benzoylcytosine, 5-methylcytosine, 4-N -benzoyl-5 -methylcytosine, adenine, 6-N-benzoyladenine, guanine and 2-N-isobutyrylguanine. It is typically desirable for linker-nucleosides to be cleaved from the oligomeric compound after it reaches a target tissue.

In some embodiments, linker-nucleosides are linked to one another and to the remainder of the oligomeric compound through cleavable bonds. In some embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid.

In some embodiments, the linker may be a non-nucleic acid linker. The non-nucleic acid linker may be a chemical bond, e.g., one or more covalent bonds or non-covalent bonds. In some embodiments, the non-nucleic acid linker is a peptide or protein linker. Such a linker may be between 2-30 amino acids, or longer. The linker includes flexible, rigid or cleavable linkers described herein.

In some embodiments, the linker is a single chemical bond (i.e., conjugate moiety is attached to an oligonucleotide via a conjugate linker through a single bond). In some embodiments, the linker comprises a chain structure, such as a hydrocarbyl chain, or an oligomer of repeating units such as ethylene glycol, nucleosides, or amino acid units.

Examples of linkers include but are not limited to pyrrolidine, 8-amino-3,6-dioxaoctanoic acid (ADO), succinimidyl 4-(N-maleimidomethyl) cyclohexane- 1-carboxylate (SMCC) and 6-aminohexanoic acid (AHEX or AHA). Other linkers include but are not limited to substituted or unsubstituted Ci-Cio alkyl, substituted or unsubstituted C2-C10 alkenyl or substituted or unsubstituted C2-C10 alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

The most commonly used flexible linkers have sequences consisting primarily of stretches of Gly and Ser residues (“GS” linker). Flexible linkers may be useful for joining domains that require a certain degree of movement or interaction and may include small, nonpolar (e.g., Gly) or polar (e.g., Ser or Thr) amino acids. Incorporation of Ser or Thr can also maintain the stability of the linker in aqueous solutions by forming hydrogen bonds with the water molecules, and therefore reduce unfavorable interactions between the linker and the protein moieties.

Rigid linkers are useful to keep a fixed distance between domains and to maintain their independent functions. Rigid linkers may also be useful when a spatial separation of the domains is critical to preserve the stability or bioactivity of one or more components in the fusion. Rigid linkers may have an alpha helix-structure or Pro-rich sequence, (XP)ₙ, with X designating any amino acid, preferably Ala, Lys, or Glu.

Cleavable linkers may release free functional domains in vivo. In some embodiments, linkers may be cleaved under specific conditions, such as the presence of reducing reagents or proteases. In vivo cleavable linkers may utilize the reversible nature of a disulfide bond. One example includes a thrombin-sensitive sequence (e.g., PRS) between the two Cys residues. In vitro thrombin treatment of CPRSC results in the cleavage of the thrombin-sensitive sequence, while the reversible disulfide linkage remains intact. Such linkers are known and described, e.g., in Chen et al. 2013. Fusion Protein Linkers: Property, Design and Functionality. Adv Drug Deliv Rev. 65(10): 1357-1369. In vivo cleavage of linkers in fusions may also be carried out by proteases that are expressed in vivo under pathological conditions (e.g. cancer or inflammation), in specific cells or tissues, or constrained within certain cellular compartments. The specificity of many proteases offers slower cleavage of the linker in constrained compartments.

Examples of linking molecules include a hydrophobic linker, such as a negatively charged sulfonate group; lipids, such as a poly (—CH₂—) hydrocarbon chains, such as polyethylene glycol (PEG) group, unsaturated variants thereof, hydroxylated variants thereof, amidated or otherwise N-containing variants thereof, noncarbon linkers; carbohydrate linkers; phosphodiester linkers, or other molecule capable of covalently linking two or more polypeptides. Non-covalent linkers are also included, such as hydrophobic lipid globules to which the polypeptide is linked, for example through a hydrophobic region of the polypeptide or a hydrophobic extension of the polypeptide, such as a series of residues rich in leucine, isoleucine, valine, or perhaps also alanine, phenylalanine, or even tyrosine, methionine, glycine or other hydrophobic residue. The polypeptide may be linked using charge-based chemistry, such that a positively charged moiety of the polypeptide is linked to a negative charge of another polypeptide or nucleic acid.

In some embodiments, a linker comprises one or more groups selected from alkyl, amino, oxo, amide, disulfide, polyethylene glycol, ether, thioether, and hydroxylamino. In certain such embodiments, the linker comprises groups selected from alkyl, amino, oxo, amide and ether groups. In some embodiments, the linker comprises groups selected from alkyl and amide groups. In some embodiments, the linker comprises groups selected from alkyl and ether groups. In some embodiments, the linker comprises at least one phosphorus moiety. In some embodiments, the linker comprises at least one phosphate group. In some embodiments, the linker includes at least one neutral linking group.

In some embodiments, the linkers are bifunctional linking moieties, e.g., those known in the art to be useful for attaching conjugate groups to oligomeric compounds, such as the ASOs provided herein. In general, a bifunctional linking moiety comprises at least two functional groups. One of the functional groups is selected to bind to a particular site on an oligomeric compound and the other is selected to bind to a conjugate group. Examples of functional groups used in a bifunctional linking moiety include but are not limited to electrophiles for reacting with nucleophilic groups and nucleophiles for reacting with electrophilic groups. In some embodiments, bifunctional linking moieties comprise one or more groups selected from amino, hydroxyl, carboxylic acid, thiol, alkyl, alkenyl, and alkynyl.

### Third Binding Domain

In some embodiments, the bifunctional molecule as provided herein further comprises a third domain. The third domain is conjugated to the first domain, the linker, the second domain, or a combination thereof. In some embodiments, the third domain comprises a small molecule or a peptide. In some embodiments, the third domain enhances uptake of the synthetic bifunctional molecule by a cell. In other embodiments, the third domain targets delivery of the synthetic molecule to a particular site (e.g., a cell).

### Third Domain Small Molecule

In some embodiments, the third domain is a small molecule.

Routine methods can be used to design small molecules that binds to the target endogenous protein with sufficient specificity. In some embodiments, the small molecule for purposes of the present methods may specifically bind the sequence to the target protein to elicit the desired effects, e.g., enhancing uptake of the bifunctional molecule by a cell, and there is a sufficient degree of specificity to avoid non-specific binding of the sequence to non-target protein under conditions in which specific binding is desired, e.g., under physiological conditions in the case of in vivo assays or therapeutic treatment, and in the case of in vitro assays, under conditions in which the assays are performed under suitable conditions of stringency.

In some embodiments, the third domain small molecules bind an effector. In some embodiments, the small molecules bind proteins or polypeptides. In some embodiments, the small molecules bind endogenous proteins or polypeptides. In some embodiments, the small molecules bind exogenous proteins or polypeptides. In some embodiments, the small molecules bind recombinant proteins or polypeptides. In some embodiments, the small molecules bind artificial proteins or polypeptides. In some embodiments, the small molecules bind fusion proteins or polypeptides. In some embodiments, the small molecules bind cell receptors. In some embodiments, the small molecules bind to cell receptors involved in endocytosis or pinocytosis. In some embodiments, the small molecules bind to cell membranes for endocytosis or pinocytosis. In some embodiments, the small molecules bind enzymes. In some embodiments, the small molecules bind enzymes a regulatory protein. In some embodiments, the small molecules bind receptors. In some embodiments, the small molecules bind signaling proteins or peptides. In some embodiments, the small molecules bind transcription factors. In some embodiments, the small molecules bind transcriptional regulators or mediators.

In some embodiments, the small molecules specifically bind to a target protein by covalent bonds. In some embodiments, the small molecules specifically bind to a target protein by non-covalent bonds. In some embodiments, the small molecules specifically bind to a target protein by irreversible binding. In some embodiments, the small molecules specifically bind to a target protein by reversible binding. In some embodiments, the small molecules specifically bind to a target protein through interaction with the side chains of the target protein. In some embodiments, the small molecules specifically bind to a target protein through interaction with the N-terminus of the target protein. In some embodiments, the small molecules specifically bind to a target protein through interaction with the C-terminus of the target protein. In some embodiments, the small molecules specifically binds to an active site or an allosteric site on the target endogenous protein.

In some embodiments, the third domain small molecules specifically bind to a specific region of the target protein sequence. For example, a specific functional region can be targeted, e.g., a region comprising a catalytic domain, a kinase domain, a protein-protein interaction domain, a protein-DNA interaction domain, a protein-RNA interaction domain, a regulatory domain, a signal domain, a nuclear localization domain, a nuclear export domain, a transmembrane domain, a glycosylation site, a modification site, or a phosphorylation site. Alternatively or in addition, highly conserved regions can be targeted, e.g., regions identified by aligning sequences from disparate species such as primate (e.g., human) and rodent (e.g., mouse) and looking for regions with high degrees of identity.

### Certain Conjugated Compounds

In certain embodiments, the third domain may comprise one or more small molecules or oligomeric compounds comprising or consisting of an oligonucleotide (modified or unmodified), optionally comprising one or more conjugate groups and/or terminal groups. Conjugate groups consist of one or more conjugate moiety and a conjugate linker that links the conjugate moiety to the small molecule or oligonucleotide. Conjugate groups may be attached to either or both ends of an small molecule or oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (orterminal groups) are attached at the 3′-end of oligonucleotides. In certain embodiments, conjugate groups are attached near the 3′-end of oligonucleotides. In certain embodiments, conjugate groups (orterminal groups) are attached at the 5′-end of oligonucleotides. In certain embodiments, conjugate groups are attached near the 5′-end of oligonucleotides. Examples of terminal groups include but are not limited to conjugate groups, capping groups, phosphate moieties, protecting groups, modified or unmodified nucleosides, and two or more nucleosides that are independently modified or unmodified.

### A. Certain Conjugate Groups

In certain embodiments, the small molecules or oligonucleotides are covalently attached to one or more conjugate groups. In certain embodiments, conjugate groups modify one or more properties of the attached small molecule or oligonucleotide, including but not limited to pharmacodynamics, pharmacokinetics, stability, binding, absorption, tissue distribution, cellular distribution, cellular uptake, charge and clearance. In certain embodiments, conjugate groups impart a new property on the attached small molecule or oligonucleotide, e.g., fluorophores or reporter groups that enable detection of the small molecule or oligonucleotide.

Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. NY. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic, a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, i, 923-937), a tocopherol group (Nishina et al., Molecular Therapy Nucleic Acids, 2015, 4, e220; doi: 10.1038/mtna.2014.72 and Nishina et al., Molecular Therapy, 2008, 16, 734-740), or a GalNAc cluster (e.g., WO2014/179620).

### 1. Conjugate Moieties

Conjugate moieties include, without limitation, intercalators, reporter molecules, polyamines, polyamides, peptides, carbohydrates (e.g., GalNAc), vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

### 2. Conjugate Linkers

Conjugate moieties are attached to small molecules or oligonucleotides through conjugate linkers. In certain small molecules or oligomeric compounds, a conjugate linker is a single chemical bond (i.e. conjugate moiety is attached to an small molecule or oligonucleotide via a conjugate linker through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to small molecules or oligomeric compounds, such as the oligonucleotides provided herein. In general, a bifunctional linking moiety comprises at least two functional groups. One of the functional groups is selected to bind to a particular site on an oligomeric compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C2-C10 alkenyl or substituted or unsubstituted C2-C10 alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N -benzoyl-5-methylcytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such a compound is more than 30. Alternatively, an oligomeric compound may comprise a modified oligonucleotide consisting of 8-30 nucleosides and no conjugate group. The total number of contiguous linked nucleosides in such a compound is no more than 30. Unless otherwise indicated conjugate linkers comprise no more than 10 linker-nucleosides. In certain embodiments, conjugate linkers comprise no more than 5 linker-nucleosides.

In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the small molecule or oligonucleotide. For example, in certain circumstances small molecule or oligomeric compounds comprising a particular conjugate moiety are better taken up by a particular cell type, but once the compound has been taken up, it is desirable that the conjugate group be cleaved to release the unconjugated small molecule or oligonucleotide. Thus, certain conjugate may comprise one or more cleavable moieties, typically within the conjugate linker. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate or phosphodiester linkage between an oligonucleotide and a conjugate moiety or conjugate group.

In certain embodiments, a cleavable moiety comprises or consists of one or more linker-nucleosides. In certain such embodiments, one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is a nucleoside comprising a 2′-deoxyfuranosyl that is attached to either the 3′ or 5 ′-terminal nucleoside of an oligonucleotide by a phosphodiester intemucleoside linkage and covalently attached to the remainder of the conjugate linker or conjugate moiety by a phosphodiester or phosphorothioate linkage. In certain such embodiments, the cleavable moiety is a nucleoside comprising a 2′-β-D-deoxyribosyl sugar moiety. In certain such embodiments, the cleavable moiety is 2′-deoxyadenosine.

### 3. Certain Cell-Targeting Conjugate Moieties

In certain embodiments, a conjugate group comprises a cell-targeting conjugate moiety. In certain embodiments, a conjugate group has the general formula:

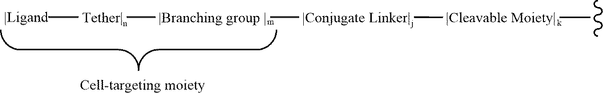

wherein n is from 1 to about 3, m is 0 when n is 1, m is 1 when n is 2 or greater, j is 1 or 0, and k is 1 or 0.

.In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell -targeting moieties that have at least one tethered ligand. In certain embodiments, cell-targeting moieties comprise two tethered ligands covalently attached to a branching group. In certain embodiments, cell -targeting moieties comprise three tethered ligands covalently attached to a branching group.

In certain embodiments, the cell-targeting moiety comprises a branching group comprising one or more groups selected from alkyl, amino, oxo, amide, disulfide, polyethylene glycol, ether, thioether and hydroxylamino groups. In certain embodiments, the branching group comprises a branched aliphatic group comprising groups selected from alkyl, amino, oxo, amide, disulfide, polyethylene glycol, ether, thioether and hydroxylamino groups. In certain such embodiments, the branched aliphatic group comprises groups selected from alkyl, amino, oxo, amide and ether groups. In certain such embodiments, the branched aliphatic group comprises groups selected from alkyl, amino and ether groups. In certain such embodiments, the branched aliphatic group comprises groups selected from alkyl and ether groups. In certain embodiments, the branching group comprises a mono or polycyclic ring system.

In certain embodiments, each tether of a cell-targeting moiety comprises one or more groups selected from alkyl, substituted alkyl, ether, thioether, disulfide, amino, oxo, amide, phosphodiester, and polyethylene glycol, in any combination. In certain embodiments, each tether is a linear aliphatic group comprising one or more groups selected from alkyl, ether, thioether, disulfide, amino, oxo, amide, and polyethylene glycol, in any combination. In certain embodiments, each tether is a linear aliphatic group comprising one or more groups selected from alkyl, phosphodiester, ether, amino, oxo, and amide, in any combination. In certain embodiments, each tether is a linear aliphatic group comprising one or more groups selected from alkyl, ether, amino, oxo, and amid, in any combination. In certain embodiments, each tether is a linear aliphatic group comprising one or more groups selected from alkyl, amino, and oxo, in any combination. In certain embodiments, each tether is a linear aliphatic group comprising one or more groups selected from alkyl and oxo, in any combination. In certain embodiments, each tether is a linear aliphatic group comprising one or more groups selected from alkyl and phosphodiester, in any combination. In certain embodiments, each tether comprises at least one phosphorus linking group or neutral linking group. In certain embodiments, each tether comprises a chain from about 6 to about 20 atoms in length. In certain embodiments, each tether comprises a chain from about 10 to about 18 atoms in length. In certain embodiments, each tether comprises about 10 atoms in chain length.

In certain embodiments, each ligand of a cell-targeting moiety has an affinity for at least one type of receptor on a target cell. In certain embodiments, each ligand has an affinity for at least one type of receptor on the surface of a mammalian lung cell.

In certain embodiments, each ligand of a cell-targeting moiety is a carbohydrate, carbohydrate derivative, modified carbohydrate, polysaccharide, modified polysaccharide, or polysaccharide derivative. In certain such embodiments, the conjugate group comprises a carbohydrate cluster (see, e.g., Maier et al., “Synthesis of Antisense Oligonucleotides Conjugated to a Multivalent Carbohydrate Cluster for Cellular Targeting,” Bioconjugate Chemistry, 2003, 14, 18-29, or Rensen et al., “Design and Synthesis of Novel N-Acetylgalactosamine-Terminated Glycolipids for Targeting of Lipoproteins to the Hepatic Asiaglycoprotein Receptor,” J. Med. Chem. 2004, 47, 5798-5808, which are incorporated herein by reference in their entirety). In certain such embodiments, each ligand is an amino sugar or athio sugar. For example, amino sugars may be selected from any number of compounds known in the art, such as sialic acid, α-D-galactosamine, β-muramic acid, 2-deoxy-2-methylamino-L-glucopyranose, 4,6-dideoxy-4-formamido-2,3-di-(O-methyl-D-mannopyranose, 2-deoxy-2-sulfoamino-D-glucopyranose and N-sulfo-D-glucosamine, and N-glycoloyl-α-neuraminic acid. For example, thio sugars may be selected from 5-Thio-β-D-glucopyranose, methyl 2,3,4-tri-O-acetyl-1-thio-6-O-trityl-α-D-glucopyranoside, 4-thio-β-D-galactopyranose, and ethyl 3,4,6,7-tetra-O-acetyl-2-deoxy-1,5-dithio-α-D-gluco-heptopyranoside.

In certain embodiments, oligomeric compounds or oligonucleotides described herein comprise a conjugate group found in any of the following references: Lee, Carbohydr Res, 1978, 67, 509-514; Connolly et al., J Biol Chem, 1982, 257, 939-945; Pavia et al., Int JPep Protein Res, 1983, 22, 539-548; Lee et al., Biochem, 1984, 23, 4255-4261; Lee et al., Glycoconjugate J, 1987, 4, 317-328; Toyokuni et al., Tetrahedron Lett, 1990, 31, 2673-2676; Biessen et al., J Med Chem, 1995, 38, 1538-1546; Valentijn et al., Tetrahedron, 1997, 53, 759-770; Kim et al., Tetrahedron Lett, 1997, 38, 3487-3490; Lee et al., Bioconjug Chem, 1997, 8, 762-765; Kato et al., Glycobiol, 2001, 11, 821-829; Rensen et al., J Biol Chem, 2001, 276, 37577-37584; Lee et al., Methods Enzymol, 2003, 362, 38-43; Westerlind et al., Glycoconj J, 2004, 21, 227-241; Lee et al., Bioorg Med Chem Lett, 2006, 16(19), 5132-5135; Maierhofer et al., Bioorg Med Chem, 2007, 15, 7661-7676; Khorev et al., Bioorg Med Chem, 2008, 16, 5216-5231; Lee et al., Bioorg Med Chem, 2011, 19, 2494-2500; Kornilova et al., Analyt Biochem, 2012, 425, 43-46; Pujol et al., Angew Chemie Int EdEngl, 2012, 51, 7445-7448; Biessen et al., J Med Chem, 1995, 38, 1846-1852; Sliedregt et al., J Med Chem, 1999, 42, 609-618; Rensen et al., J Med Chem, 2004, 47, 5798-5808; Rensen et al., Arterioscler Thromb Vase Biol, 2006, 26, 169-175; van Rossenberg et al., Gene Ther, 2004, 11, 457-464; Sato et al., J Am Chem Soc, 2004, 126, 14013-14022; Lee et al., J Org Chem, 2012, 77, 7564-7571; Biessen et al., FASEB J, 2000, 14, 1784-1792; Rajur et al., Bioconjug Chem, 1997, 8, 935-940; Duff et al., Methods Enzymol, 2000, 313, 297-321; Maier et al., Bioconjug Chem, 2003, 14, 18-29; Jayaprakash et al., Org Lett, 2010, 12, 5410-5413; Manoharan, Antisense Nucleic Acid Drug Dev, 2002, 12, 103-128; Merwin et al., Bioconjug Chem, 1994, 5, 612-620; Tomiya et al., BioorgMed Chem, 2013, 21, 5275-5281; International applications WO1998/013381; WO2011/038356; WO 1997/046098; WO2008/098788; WO2004/101619; WO2012/037254; WO2011/120053; WO2011/100131; WO2011/163121; WO2012/177947; WO2013/033230; WO2013/075035; WO2012/083185; WO2012/083046; WO2009/082607; WO2009/134487; WO2010/144740; WO2010/148013; WO1997/020563; WO2010/088537; WO2002/043771; WO2010/129709; WO2012/068187; WO2009/126933; WO2004/024757; WO2010/054406; WO2012/089352; WO2012/089602; WO2013/166121; WO2013/165816; U.S. Pat. 4,751,219; 8,552,163; 6,908,903; 7,262,177; 5,994,517; 6,300,319; 8,106,022; 7,491,805; 7,491,805; 7,582,744; 8,137,695; 6,383,812; 6,525,031; 6,660,720; 7,723,509; 8,541,548; 8,344,125; 8,313,772; 8,349,308; 8,450,467; 8,501,930; 8,158,601: 7,262,177: 6,906,182: 6,620,916: 8,435,491: 8,404,862: 7,851,615: Published U.S. Pat. Application Publications US2011/0097264; US2011/0097265; US2013/0004427; US2005/0164235; US2006/0148740; US2008/0281044; US2010/0240730; US2003/0119724; US2006/0183886; US2008/0206869; US2011/0269814; US2009/0286973; US2011/0207799; US2012/0136042; US2012/0165393; US2008/0281041; US2009/0203135; US2012/0035115; US2012/0095075; US2012/0101148; US2012/0128760; US2012/0157509; US2012/0230938; US2013/0109817; US2013/0121954; US2013/0178512; US2013/0236968; US2011/0123520; US2003/0077829; US2008/0108801 ; and US2009/0203132.

### Aptamer

In some embodiments, the third domain of the bifunctional molecule as described herein, which specifically binds to a target endogenous protein is an aptamer.

Routine methods can be used to design and select aptamers that binds to the target protein with sufficient specificity. In some embodiments, the aptamer for purposes of the present methods bind to the target protein (e.g., receptor). The protein performs the desired effects, e.g., enhancing uptake of the bifunctional molecule by a cell, and there is a sufficient degree of specificity to avoid non-specific binding of the sequence to non-target protein under conditions in which specific binding is desired, e.g., under physiological conditions in the case of in vivo assays or therapeutic treatment, and in the case of in vitro assays, under conditions in which the assays are performed under suitable conditions of stringency.

In some embodiments, the aptamers bind proteins or polypeptides. In some embodiments, the aptamers bind endogenous proteins or polypeptides. In some embodiments, the aptamers bind exogenous proteins or polypeptides. In some embodiments, the aptamers bind recombinant proteins or polypeptides. In some embodiments, the aptamers bind artificial proteins or polypeptides. In some embodiments, the aptamers bind fusion proteins or polypeptides. In some embodiments, the aptmers bind cell receptors. In some embodiments, the aptamers bind to cell receptors involved in endocytosis or pinocytosis. In some embodiments, the aptamers bind to cell membranes for endocytosis or pinocytosis. In some embodiments, the aptamers bind enzymes. In some embodiments, the aptamers bind enzymes a regulatory protein. In some embodiments, the aptamers bind receptors. In some embodiments, the aptamers bind signaling proteins or peptides. In some embodiments, the aptamers bind transcription factors. In some embodiments, the aptamers bind transcriptional regulators or mediators.

In some embodiments, the aptamers specifically bind to a target protein by covalent bonds. In some embodiments, the aptamers specifically bind to a target protein by non-covalent bonds. In some embodiments, the aptamers specifically bind to a target protein by irreversible binding. In some embodiments, the aptamers specifically bind to a target protein by reversible binding. In some embodiments, the aptamers specifically binds to an active site or an allosteric site on the target endogenous protein.

In some embodiments, In some embodiments, the aptamers specifically bind to a specific region of the target protein sequence. For example, a specific functional region can be targeted, e.g., a region comprising a catalytic domain, a kinase domain, a protein-protein interaction domain, a protein-DNA interaction domain, a protein-RNA interaction domain, a regulatory domain, a signal domain, a nuclear localization domain, a nuclear export domain, a transmembrane domain, a glycosylation site, a modification site, or a phosphorylation site. Alternatively or in addition, highly conserved regions can be targeted, e.g., regions identified by aligning sequences from disparate species such as primate (e.g., human) and rodent (e.g., mouse) and looking for regions with high degrees of identity.

### Plurality of Third Domains

In some embodiments, the synthetic bifunctional molecule as provided herein comprises a first domain, one or more second domains, and one or more third domains. In some embodiments, the bifunctional molecule has 1, 2, 3, 4, 5, 6, 7, 8, 9, 10 or more third domains. In some embodiments, each of the one or more third domains specifically binds to a target endogenous protein.

In one aspect, the synthetic bifunctional molecule comprises a first domain that specifically binds to a target RNA sequence, a plurality of second domains, wherein each of the plurality of second domains specifically bind to a target endogenous protein, and a plurality of third domains, wherein each of the plurality of third domains specifically bind to a target endogenous protein to enhance uptake of the synthetic bifunctional molecule by a cell. In some embodiments, the bifunctional molecule further comprises a linker that conjugates the first domain to the plurality of second domains. In some embodiments, the bifunctional molecule further comprises a linker that conjugates the first domain to the plurality of third domains, a linker that conjugates the second domain domain to the plurality of third domains, or a combination thereof.

In some embodiments, the first domain comprises a small molecule or an ASO. In some embodiments, the bifunctional molecule comprises a plurality of second domains. Each of the plurality of second domains comprise a small molecule or an aptamer. In some embodiments, the bifunctional molecule comprises a plurality of third domains. Each of the plurality of third domains comprise a small molecule or an aptamer. In some embodiments, each of the plurality of third domains comprise a small molecule. In some embodiments, each of the plurality of third domains comprise an aptamer.

In some embodiments, the bifunctional molecule comprises a plurality of third domains, e.g., 2, 3, 4, 5, 6, 7, 8, 9, or 10 second domains. In one embodiment, the bifunctional molecule has 2 third domains. In one embodiment, the bifunctional molecule has 3 third domains. In one embodiment, the bifunctional molecule has 4 third domains. In one embodiment, the bifunctional molecule has 5 third domains. In one embodiment, the bifunctional molecule has 6 third domains. In one embodiment, the bifunctional molecule has 7 third domains. In one embodiment, the bifunctional molecule has 8 third domains. In one embodiment, the bifunctional molecule has 9 third domains. In one embodiment, the bifunctional molecule has 10 third domains. In one embodiment, the bifunctional molecule has more than 10 third domains.

In some embodiments, the plurality of third domains is same domains. In some embodiments, the plurality of third domains is different domains. In some embodiments, the plurality of third domains binds to a same target. In some embodiments, the plurality of third domains binds to different targets.

### Target Protein of Third Domain

In some embodiments, the target proteins may be endogenous proteins or polypeptides. In some embodiments, the target proteins may be exogenous proteins or polypeptides. In some embodiments, the target proteins may be recombinant proteins or polypeptides. In some embodiments, the target proteins may be artificial proteins or polypeptides. In some embodiments, the target proteins may be fusion proteins or polypeptides. In some embodiments, the target proteins may be enzymes. In some embodiments, the target proteins may be receptors. In some embodiments, the target proteins may be signaling proteins or peptides. In some embodiments, the target proteins may be transcription factors. In some embodiments, the target proteins may be transcriptional regulators or mediators.

In some embodiments, the activity or function of the target protein, e.g., enhancing cellular uptake of the bifunctional molecule, may be modulated by binding to the third domain of the bifunctional molecule as provided herein. In some embodiments, the target protein is involved in endocytosis or pinocytosis.

### Target Protein (Effector) Function

In some embodiments, the bifunctional molecule comprises a second domain that specifically binds to a target protein. In some embodiments, the target protein is an effector. In some embodiments, the target protein is an endogenous protein. In other embodiments, the target protein is an intracellular protein. In another embodiment, the target protein is an endogenous and intracellular protein. In some embodiments, the target endogenous protein is an enzyme or a regulatory protein. In some embodiments, the second domain specifically binds to an active site or an allosteric site on the target endogenous protein.

### Transcription: Upregulation

In some embodiments, the second domain of the bifunctional molecules as provided herein targets a protein that increases transcription of a gene from Table 4. In some embodiments, the first domain of the bifunctional molecules as provided herein targets a ribonucleic acid sequence that increases transcription of a gene from Table 4. In some embodiments, the first domain of the bifunctional molecules as provided herein targets a ribonucleic acid sequence that is proximal or near to a sequences that increases transcription of a gene from Table 4.

TABLE 4

Exemplary Genes whose transcription is increased by a Bifunctional Molecule
Neoplasia PTEN; ATM; ATR; EGFR; ERBB2; ERBB3; ERBB4; Notch1; Notch2; Notch3; Notch4; AKT; AKT2; AKT3; HIF; HIF1a; HIF3a; Met; HRG; Bcl2; PPAR alpha; PPAR gamma; WT1 (Wilms Tumor); FGF Receptor Family members (5 members: 1, 2, 3, 4, 5); CDKN2a; APC; RB (retinoblastoma); MEN1; VHL; BRCA1; BRCA2; AR (Androgen Receptor); TSG101; IGF; IGF Receptor; Igf1 (4 variants); Igf2 (3 variants); Igf 1 Receptor; Igf 2 Receptor; Bax; Bcl2; caspases family (9 members: 1, 2, 3, 4, 6, 7, 8, 9, 12); Kras; Apc Age-related Macular Abcr; Ccl2; Cc2; cp (ceruloplasmin); Timp3; cathepsinD; Degeneration Vldlr; Ccr2 Schizophrenia Neuregulin1 (Nrg1); Erb4 (receptor for Neuregulin); Complexin1 (Cplx1); Tph1 Tryptophan hydroxylase; Tph2 Tryptophan hydroxylase 2; Neurexin 1; GSK3; GSK3a; GSK3b Disorders 5-HTT (Slc6a4); COMT; DRD (Drd1a); SLC6A3; DAOA
DTNBP1; Dao (Dao1) Trinucleotide Repeat HTT (Huntington’s Dx); SBMA/SMAX1/AR (Kennedy’s Disorders Dx); FXN/X25 (Friedrich’s Ataxia); ATX3 (Machado- Joseph’s Dx); ATXN1 and ATXN2 (spinocerebellar ataxias); DMPK (myotonic dystrophy); Atrophin-1 and Atn1 (DRPLA Dx); CBP (Creb-BP—global instability); VLDLR (Alzheimer’s); Atxn7; Atxn10 Fragile X Syndrome FMR2; FXR1; FXR2; mGLUR5 Secretase Related APH-1 (alpha and beta); Presenilin (Psen1); nicastrin Disorders (Ncstn); PEN-2 Others Nos1; Parp1; Nat1; Nat2 Prion - related disorders Prp ALS SOD1; ALS2; STEX; FUS; TARDBP; VEGF (VEGF-a; VEGF-b; VEGF-c) Drug addiction Prkce (alcohol); Drd2; Drd4; ABAT (alcohol); GRIA2; Grm5; Grin1; Htr1b; Grin2a; Drd3; Pdyn; Gria1 (alcohol) Autism Mecp2; BZRAP1; MDGA2; Sema5A; Neurexin 1; Fragile X (FMR2 (AFF2); FXR1; FXR2; Mglur5) Alzheimer’s Disease E1; CHIP; UCH; UBB; Tau; LRP; PICALM; Clusterin; PS1; SORL1; CR1; Vldlr; Uba1; Uba3; CHIP28 (Aqp1, Aquaporin 1); Uchl1; Uchl3; APP Inflammation IL-10; IL-1 (IL-1a; IL-1b); IL-13; IL-17 (IL-17a (CTLA8); IL- 17b; IL-17c; IL-17d; IL-17f); II-23; Cx3cr1; ptpn22; TNFa; NOD2/CARD15 for IBD; IL-6; IL-12 (IL-12a; IL-12b); CTLA4; Cx3cl1 Parkinson’s Disease x-Synuclein; DJ-1; LRRK2; Parkin; PINK1; SCN1A; SYNGAP; OP1A

In some embodiments, transcription of the gene is upregulated/increased. In some embodiments, transcription of the gene is upregulated. In some embodiments, transcription of the gene is increased.

In some embodiments, RNA is artificially localized to a defined gene locus in cells, and the localized RNA is targeted by an ASO that is conjugated to a small molecule inhibitor. The bifunctional molecule as provided herein recruits a protein to the genomic site and effects a change in the underlying gene expression. In some embodiments, specific RNAs may demarcate every gene in the genome. By targeting these RNAs to recruit transcriptional modifying enzymes, the local concentration of the transcriptional modifying enzyme near the gene is increased, thereby increasing transcription of the underlying gene (either repressing or activating transcription). In some embodiments, recruiting a histone deacetylase by the bifunctional molecule as provided herein to a gene may result in local histone deacetylation and repression of gene expression.

In some embodiments, the target proteins may be enzymes. In some embodiments, the target proteins may be receptors. In some embodiments, the target proteins may be signaling proteins or peptides. In some embodiments, the target proteins may be transcription factors. In some embodiments, the target proteins may be transcriptional regulators or mediators. In some embodiments, the target proteins may be proteins or peptides involved in or regulate post-transcriptional modifications. In some embodiments, the target proteins may be proteins or peptides involved in or regulate post-translational modifications. In some embodiments, the target proteins may be proteins or peptides that bind RNAs.

In some embodiments, the target protein comprises a transcriptional modifying enzyme. In some embodiments, the target protein comprises a histone deacetylase. In some embodiments, the target protein comprises a histone demethylase. In some embodiment, the target protein comprises a transcriptional activator. In some embodiments, the target protein comprises a transcriptional repressor. In some embodiments, the target protein is a transcriptional modifying enzyme. In some embodiments, the target protein is a histone deacetylase. In some embodiments, the target protein is a histone demethylase. In some embodiments, the target protein is a transcriptional activator. In some embodiments, the target protein is a transcriptional repressor.

In some embodiments, the first domain recruits the bifunctional molecule as described herein to the target site by binding to the target RNA or gene sequence, in which the second domain interacts with the target protein and increase transcription of the gene. In some embodiments, the target protein recruits the bifunctional molecule as described herein by binding to the second domain of the bifunctional molecule as provided herein, in which the first domain specifically binds to a target RNA sequence and increase transcription of the gene. In some embodiments, the target protein after interacting with the second domain of the bifunctional molecule as provided herein further recruits proteins or peptides involved in mediating transcription or increasing transcription through interaction with the proteins or peptides.

### Pharmaceutical Compositions

In some aspects, the bifunction molecules described herein comprises pharmaceutical compositions, or the composition comprising the bifunctional molecule as described herein.

In some embodiments, the pharmaceutical composition further comprises a pharmaceutically acceptable excipient. Pharmaceutical compositions may be sterile and/or pyrogen-free. General considerations in the formulation and/or manufacture of pharmaceutical agents may be found, for example, in Remington: The Science and Practice of Pharmacy 21^{st} ed., Lippincott Williams & Wilkins, 2005 (incorporated herein by reference).

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g., non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals, e.g., pet and live-stock animals, such as cattle, pigs, horses, sheep, cats, dogs, mice, and/or rats; and/or birds, including commercially relevant birds such as poultry, chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product.

The term “pharmaceutical composition” is intended to also disclose that the bifunctional molecules as described herein comprised within a pharmaceutical composition can be used for the treatment of the human or animal body by therapy. It is thus meant to be equivalent to the “bifunctional molecule as described herein for use in therapy.”

### Delivery

Pharmaceutical compositions as described herein can be formulated for example to include a pharmaceutical excipient. A pharmaceutical carrier may be a membrane, lipid bilayer, and/or a polymeric carrier, e.g., a liposome or particle such as a nanoparticle, e.g., a lipid nanoparticle, and delivered by known methods to a subject in need thereof (e.g., a human or non-human agricultural or domestic animal, e.g., cattle, dog, cat, horse, poultry). Such methods include, but not limited to, transfection (e.g., lipid-mediated, cationic polymers, calcium phosphate); electroporation or other methods of membrane disruption (e.g., nucleofection), fusion, and viral delivery (e.g., lentivirus, retrovirus, adenovirus, AAV).

In some aspects, the methods comprise delivering the bifunctional molecule as described herein, the composition comprising the bifunctional molecule as described herein, or the pharmaceutical compositions comprising the bifunctional molecule as described herein to a subject in need thereof.

### Methods of Delivery

A method of delivering the bifunctional molecule as described herein, the composition comprising the bifunctional molecule as described herein, or the pharmaceutical compositions comprising the bifunctional molecule as described herein to a cell, tissue, or subject, comprises administering the bifunctional molecule as described herein, the composition comprising the bifunctional molecule as described herein, or the pharmaceutical compositions comprising the bifunctional molecule as described herein to the cell, tissue, or subject.

In some embodiments the bifunctional molecule as described herein, the composition comprising the bifunctional molecule as described herein, or the pharmaceutical compositions comprising the bifunctional molecule as described herein is administered parenterally. In some embodiments the bifunctional molecule as described herein, the composition comprising the bifunctional molecule as described herein, or the pharmaceutical compositions comprising the bifunctional molecule as described herein is administered by injection. The administration can be systemic administration or local administration. In some embodiments, the bifunctional molecule as described herein, the composition comprising the bifunctional molecule as described herein, or the pharmaceutical compositions comprising the bifunctional molecule as described herein is administered intravenously, intraarterially, intraperitoneally, intradermally, intracranially, intrathecally, intralymphaticly, subcutaneously, or intramuscularly.

In some embodiments, the cell is a eukaryotic cell. In some embodiments, the cell is a mammalian cell. In some embodiments, the cell is a human cell. In some embodiments, the cell is an animal cell.

### Methods Using Bifunctional Molecules

### Methods of Increasing Transcription

In some embodiments, the second domain of the bifunctional molecules as provided herein targets a protein that increases transcription of a gene from Table 4.

In some embodiments, the first domain of the bifunctional molecules as provided herein targets the ribonucleic acid sequence that increases transcription of a gene from Table 4.

In some embodiments, transcription of the gene is upregulated/increased. In some embodiments, transcription of the gene is upregulated. In some embodiments, transcription of the gene is increased.

In one aspect, a method of increasing transcription of a gene in a cell comprises administering to a cell a synthetic bifunctional molecule comprising a first domain comprising an antisense oligonucleotide (ASO) that specifically binds to a target ribonucleic acid sequence, a second domain that specifically binds to a target endogenous protein and a linker that conjugates the first domain to the second domain, wherein the target endogenous protein increases transcription of a gene in the cell.

In some embodiments, the second domain comprising a small molecule or an aptamer.

In some embodiments, the cell is a human cell. In some embodiments, the human cell is infected with a virus. In some embodiments, the cell is a cancer cell. In some embodiments, the cell is a bacterial cell.

In some embodiments, the first domain is conjugated to the second domain by a linker molecule.

In some embodiments, the first domain is an antisense oligonucleotide.

In some embodiments, the first domain is a small molecule. In some embodiments, the small molecule is selected from the group consisting of Table 2. In some embodiments, the second domain is a small molecule. In some embodiments, the small molecule is selected from Table 3.

In some embodiments, the second domain is an aptamer. In some embodiments, the aptamer is selected from Table 3.

In some embodiments, the synthetic bifunctional molecule further comprises a third domain conjugated to the first domain, linker, the second domain, or a combination thereof. In some embodiments, the third domain comprises a small molecule. In some embodiments, the third domain enhances uptake of the synthetic bifunctional molecule by a cell.

In some embodiments, the synthetic bifunctional molecule further comprises one or more second domains. In some embodiments, each of the one or more second domains specifically binds to a single target endogenous protein.

In one aspect, the method of increasing transcription of a gene in a cell comprises administering to a cell a synthetic bifunctional molecule comprising a first domain that specifically binds to a target RNA sequence, a plurality of second domains that specifically bind to a single target endogenous protein, and a linker that conjugates the first domain to the plurality of second domains, wherein the target endogenous protein increases transcription of a gene in the cell.

In some embodiments, the first domain comprises a small molecule or an antisense oligonucleotide (ASO). In some embodiments, the plurality of second domains, each comprising a small molecule or an aptamer. In some embodiments, each of plurality of second domains comprises a small molecule. In some embodiments, the plurality of second domains is 2, 3, 4, or 5 second domains.

In some embodiments, the synthetic bifunctional molecule as provided herein further comprising a third domain conjugated to the first domain, linker, the second domain, or a combination thereof. In some embodiments, the third domain comprises a small molecule. In some embodiments, the third domain enhances uptake of the synthetic bifunctional molecule by a cell.

In some embodiments, the target endogenous protein is an intracellular protein. In some embodiments, the target endogenous protein is an enzyme or a regulatory protein. In some embodiments, the second domain specifically binds to an active site or an allosteric site on the target endogenous protein.

The term “transcription,” as used herein, refers to the first of several steps of DNA based gene expression, in which a particular segment of DNA is copied into RNA (especially mRNA) by the enzyme RNA polymerase. In some embodiments, for example, during transcription, a DNA sequence is read by an RNA polymerase, which produces a complementary, antiparallel RNA strand called a primary transcript. The method as provided herein may increase transcription at the initiation step, promoter escape step, elongation step or termination step.

Increase of molecules may be measured by conventional assays known to a person of skill in the art, including, but not limited to, measuring RNA levels by, e.g., quantitative real-time RT- PCR (qRT- PCR), RNA FISH, measuring protein levels by, e.g., immunoblot.

In some embodiments, transcription of the gene is upregulated/increased. In some embodiments, transcription of the gene is upregulated. In some embodiments, transcription of the gene is increased.

In some embodiments, RNA is artificially localized to a defined gene locus in cells, and the localized RNA is targeted by an ASO that is conjugated to a small molecule inhibitor. The inhibitor recruits a protein to the genomic site and effects a change in the underlying gene expression. In some embodiments, specific RNAs may demarcate every gene in the genome. By targeting these RNAs to recruit transcriptional modifying enzymes, the local concentration of the transcriptional modifying enzyme near the gene is increased, thereby increasing transcription of the underlying gene (either repressing or activating transcription). In some embodiments, recruiting a histone deacetylase to a gene may result in local histone deacetylation and repression of gene expression. ). In some embodiments, recruiting a histone acetylase to a gene may result in local histone acetylation and activation of gene expression. In some embodiments, recruiting a transcriptional activator or repressor by the bifunctional molecule as provided herein to a gene may result in activation or repression of gene expression

In some embodiments, transcription of the gene is upregulated or increased by at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, at least 1000%, at least 2000%, at least 3000%, at least 4000%, at least 5000%, at least 6000%, at least 7000%, at least 8000%, at least 9000%, at least 10000%, at least 20000%, at least 30000%, at least 40000%, at least 50000%, at least 60000%, at least 70000%, at least 80000%, at least 90000%, or at least 100000% as compared to an untreated control cell, tissue or subject, or compared to the corresponding activity in the same type of cell, tissue or subject before treatment with synthetic bifunctional molecule described herein as measured by any standard technique. In some embodiments, transcription of the gene is upregulated or increased by at least 2 fold, at least 3 fold, at least 4 fold, at least 5 fold, at least 10 fold, at least 20 fold, at least 25 fold, at least 30 fold, at least 40 fold, at least 50 fold, at least 60 fold, at least 70 fold, at least 80 fold, at least 90 fold, at least 100 fold, at least 200 fold, at least 300 fold, at least 400 fold, at least 500 fold, at least 600 fold, at least 700 fold, at least 800 fold, at least 900 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold as compared to an untreated control cell, tissue or subject, or compared to the corresponding activity in the same type of cell, tissue or subject before treatment with synthetic bifunctional molecule described herein as measured by any standard technique.

### Methods of Treatment

The bifunctional molecules as described herein can be used in a method of treatment for a subject in need thereof. A subject in need thereof, for example, has a disease or condition. In some embodiments, the disease is a cancer, a metabolic disease, an inflammatory disease, a cardiovascular disease, an infectious disease, a genetic disease, or a neurological disease. In some embodiments, the disease is a cancer and wherein the target gene is an oncogene. In some embodiments, the gene of which transcription is increased by the bifunctional molecule as provided herein or the composition comprising the bifunctional molecule as provided herein is associated with a disease from Table 5.

TABLE 5

Exemplary Diseases (and associated genes) for treatment with a Bifunctional Molecule
Blood and Anemia (CDAN1, CDA1, RPS19, DBA, PKLR, PK1, NT5C3, UMPH1, coagulation diseases PSN1, RHAG, RH50A, NRAMP2, SPTB, ALAS2, ANH1, ASB, and disorders ABCB7, ABC7, ASAT); Bare lymphocyte syndrome (TAPBP, TPSN, TAP2, ABCB3, PSF2, RING11, MHC2TA, C2TA, RFX5, RFXAP, RFX5), Bleeding disorders (TBXA2R, P2RX1, P2X1); Factor H and factor H-like 1 (HF1, CFH, HUS); Factor V and factor VIII (MCFD2); Factor VII deficiency (F7); Factor X deficiency (F10); Factor XI deficiency (F11); Factor XII deficiency (F12, HAF); Factor XIIIA deficiency (F13A1, F13A); Factor XIIIB deficiency (F13B); Fanconi anemia (FANCA, FACA, FA1, FA, FAA, FAAP95
FAAP90, FLJ34064, FANCB, FANCC, FACC, BRCA2, FANCD1, FANCD2, FANCD, FACD, FAD, FANCE, FACE, FANCF, XRCC9, FANCG, BRIP1, BACH1, FANCJ, PHF9, FANCL, FANCM, KIAA1596); Hemophagocytic lymphohistiocytosis disorders (PRF1, HPLH2, UNC13D, MUNC13-4, HPLH3, HLH3, FHL3); Hemophilia A (F8, F8C, HEMA); Hemophilia B (F9, HEMB), Hemorrhagic disorders (PI, ATT, F5); Leukocyde deficiencies and disorders (ITGB2, CD18, LCAMB, LAD, EIF2B1, EIF2BA, EIF2B2, EIF2B3, EIF2B5, LVWM, CACH, CLE, EIF2B4); Sickle cell anemia (HBB); Thalassemia (HBA2, HBB, HBD, LCRB, HBA1). Cell dysregulation B-cell non-Hodgkin lymphoma (BCL7A, BCL7); Leukemia (TAL1, and oncology TCL5, SCL, TAL2, FLT3, NBS1, NBS, ZNFN1A1, IK1, LYF1, diseases and disorders HOXD4, HOX4B, BCR, CML, PHL, ALL, ARNT, KRAS2, RASK2, GMPS, AF10, ARHGEF12, LARG, KIAA0382, CALM, CLTH, CEBPA, CEBP, CHIC2, BTL, FLT3, KIT, PBT, LPP, NPM1, NUP214, D9S46E, CAN, CAIN, RUNX1, CBFA2, AML1, WHSC1L1, NSD3, FLT3, AF1Q, NPM1, NUMA1, ZNF145, PLZF, PML, MYL, STAT5B, AF10, CALM, CLTH, ARL11, ARLTS1, P2RX7, P2X7, BCR, CML, PHL, ALL, GRAF, NF1, VRNF, WSS, NFNS, PTPN11, PTP2C, SHP2, NS1, BCL2, CCND1, PRAD1, BCL1, TCRA, GATA1, GF1, ERYF1, NFE1, ABL1, NQO1, DIA4, NMOR1, NUP214, D9S46E, CAN, CAIN). Inflammation and AIDS (KIR3DL1, NKAT3, NKB1, AMB11, KIR3DS1, IFNG, CXCL12, immune related SDF1); Autoimmune lymphoproliferative syndrome (TNFRSF6, APT1, diseases and disorders FAS, CD95, ALPS1A); Combined immunodeficiency, (IL2RG, SCIDX1, SCIDX, IMD4); HIV-1 (CCL5, SCYA5, D17S136E, TCP228), HIV susceptibility or infection (IL10, CSIF, CMKBR2, CCR2, CMKBR5, CCCKR5 (CCR5)); Immunodeficiencies (CD3E, CD3G, AICDA, AID, HIGM2, TNFRSF5, CD40, UNG, DGU, HIGM4, TNFSF5, CD40LG, HIGM1, IGM, FOXP3, IPEX, AIID, XPID, PIDX, TNFRSF14B, TACI); Inflammation (IL-10, IL-1 (IL-1a, IL-1b), IL-13, IL-17 (IL-17a (CTLA8), IL-17b, IL-17c, IL-17d, IL-17f), II-23, Cx3cr1, ptpn22, TNFa, NOD2/CARD15 for IBD, IL-6, IL-12 (IL-12a, IL-12b), CTLA4, Cx3cl1); Severe combined immunodeficiencies (SCIDs)(JAK3, JAKL, DCLRE1C, ARTEMIS, SCIDA, RAG1, RAG2, ADA, PTPRC, CD45, LCA, IL7R, CD3D, T3D, IL2RG, SCIDX1, SCIDX, IMD4). Metabolic, liver, Amyloid neuropathy (TTR, PALB); Amyloidosis (APOA1, APP, AAA, kidney and protein CVAP, AD1, GSN, FGA, LYZ, TTR, PALB); Cirrhosis (KRT18, KRT8, diseases and disorders CIRH1A, NAIC, TEX292, KIAA1988); Cystic fibrosis (CFTR, ABCC7, CF, MRP7); Glycogen storage diseases (SLC2A2, GLUT2, G6PC, G6PT, G6PT1, GAA, LAMP2, LAMPB, AGL, GDE, GBE1, GYS2, PYGL, PFKM); Hepatic adenoma, 142330 (TCF1, HNF1A, MODY3), Hepatic failure, early onset, and neurologic disorder
(SCOD1, SCO1), Hepatic lipase deficiency (LIPC), Hepatoblastoma, cancer and carcinomas (CTNNB1, PDGFRL, PDGRL, PRLTS, AXIN1, AXIN, CTNNB1, TP53, P53, LFS1, IGF2R, MPRI, MET, CASP8, MCH5; Medullary cystic kidney disease (UMOD, HNFJ, FJHN, MCKD2, ADMCKD2); Phenylketonuria (PAH, PKU1, QDPR, DHPR, PTS); Polycystic kidney and hepatic disease (FCYT, PKHD1, ARPKD, PKD1, PKD2, PKD4, PKDTS, PRKCSH, G19P1, PCLD, SEC63). Muscular/Skeletal Becker muscular dystrophy (DMD, BMD, MYF6), Duchenne Muscular diseases and disorders Dystrophy (DMD, BMD); Emery-Dreifuss muscular dystrophy (LMNA, LMN1, EMD2, FPLD, CMD1A, HGPS, LGMD1B, LMNA, LMN1, EMD2, FPLD, CMD1A); Facioscapulohumeral muscular dystrophy (FSHMD1A, FSHD1A); Muscular dystrophy (FKRP, MDC1C, LGMD2I, LAMA2, LAMM, LARGE, KIAA0609, MDC1D, FCMD, TTID, MYOT, CAPN3, CANP3, DYSF, LGMD2B, SGCG, LGMD2C, DMDA1, SCG3, SGCA, ADL, DAG2, LGMD2D, DMDA2, SGCB, LGMD2E, SGCD, SGD, LGMD2F, CMD1L, TCAP, LGMD2G, CMD1N, TRIM32, HT2A, LGMD2H, FKRP, MDC1C, LGMD2I, TTN, CMD1G, TMD, LGMD2J, POMT1, CAV3, LGMD1C, SEPN1, SELN, RSMD1, PLEC1, PLTN, EBS1); Osteopetrosis (LRP5, BMND1, LRP7, LR3, OPPG, VBCH2, CLCN7, CLC7, OPTA2, OSTM1, GL, TCIRG1, TIRC7, OC116, OPTB1); Muscular atrophy (VAPB, VAPC, ALS8, SMN1, SMA1, SMA2, SMA3, SMA4, BSCL2, SPG17, GARS, SMAD1, CMT2D, HEXB, IGHMBP2, SMUBP2, CATF1, SMARD1). Neurological and ALS (SOD1, ALS2, STEX, FUS, TARDBP, VEGF (VEGF-a, VEGF-b, neuronal diseases and VEGF-c); Alzheimer disease (APP, AAA, CVAP, AD1, APOE, AD2, disorders PSEN2, AD4, STM2, APBB2, FE65L1, NOS3, PLAU, URK, ACE, DCP1, ACE1, MPO, PACIP1, PAXIP1L, PTIP, A2M, BLMH, BMH, PSEN1, AD3); Autism (Mecp2, BZRAP1, MDGA2, Sema5A, Neurexin1, GLO1, MECP2, RTT, PPMX, MRX16, MRX79, NLGN3, NLGN4, KIAA1260, AUTSX2); Fragile X Syndrome (FMR2, FXR1, FXR2, mGLUR5); Huntington’s disease and disease like disorders (HD, IT15, PRNP, PRIP, JPH3, JP3, HDL2, TBP, SCA17); Parkinson disease (NR4A2, NURR1, NOT, TINUR, SNCAIP, TBP, SCA17, SNCA, NACP, PARK1, PARK4, DJ1, PARK7, LRRK2, PARK8, PINK1, PARK6, UCHL1, PARK5, SNCA, NACP, PARK1, PARK4, PRKN, PARK2, PDJ, DBH, NDUFV2); Rett syndrome (MECP2, RTT, PPMX, MRX16, MRX79, CDKL5, STK9, MECP2, RTT, PPMX, MRX16, MRX79, x-Synuclein, DJ-1); Schizophrenia (Neuregulin1 (Nrg1), Erb4 (receptor for Neuregulin), Complexin1 (Cplx1), Tph1 Tryptophan hydroxylase, Tph2, Tryptophan hydroxylase 2, Neurexin 1, GSK3, GSK3a, GSK3b, 5-HTT (Slc6a4), COMT, DRD (Drd1a), SLC6A3, DAOA, DTNBP1, Dao (Dao1)); Secretase Related Disorders (APH-1 (alpha and beta), Presenilin (Psen1), nicastrin, (Ncstn), PEN-2, Nos1, Parp1, Nat1,
Nat2); Trinucleotide Repeat Disorders (HTT (Huntington’s Dx), SBMA/SMAX1/AR (Kennedy’s Dx), FXN/X25 (Friedrich’s Ataxia), ATX3 (Machado-Joseph’s Dx), ATXN1 and ATXN2 (spinocerebellar ataxias), DMPK (myotonic dystrophy), Atrophin-1 and Atn1 (DRPLA Dx), CBP (Creb-BP-global instability), VLDLR (Alzheimer’s), Atxn7, Atxn10). Occular diseases and Age-related macular degeneration (Abcr, Ccl2, Cc2, cp (ceruloplasmin), disorders Timp3, cathepsinD, Vldlr, Ccr2); Cataract (CRYAA, CRYA1, CRYBB2, CRYB2, PITX3, BFSP2, CP49, CP47, CRYAA, CRYA1, PAX6, AN2, MGDA, CRYBA1, CRYB1, CRYGC, CRYG3, CCL, LIM2, MP19, CRYGD, CRYG4, BFSP2, CP49, CP47, HSF4, CTM, HSF4, CTM, MIP, AQP0, CRYAB, CRYA2, CTPP2, CRYBB1, CRYGD, CRYG4, CRYBB2, CRYB2, CRYGC, CRYG3, CCL, CRYAA, CRYA1, GJA8, CX50, CAE1, GJA3, CX46, CZP3, CAE3, CCM1, CAM, KRIT1); Corneal clouding and dystrophy (APOA1, TGFBI, CSD2, CDGG1, CSD, BIGH3, CDG2, TACSTD2, TROP2, M1S1, VSX1, RINX, PPCD, PPD, KTCN, COL8A2, FECD, PPCD2, PIP5K3, CFD); Cornea plana congenital (KERA, CNA2); Glaucoma (MYOC, TIGR, GLC1A, JOAG, GPOA, OPTN, GLC1E, FIP2, HYPL, NRP, CYP1B1, GLC3A, OPA1, NTG, NPG, CYP1B1, GLC3A); Leber congenital amaurosis (CRB1, RP12, CRX, CORD2, CRD, RPGRIP1, LCA6, CORD9, RPE65, RP20, AIPL1, LCA4, GUCY2D, GUC2D, LCA1, CORD6, RDH12, LCA3); Macular dystrophy (ELOVL4, ADMD, STGD2, STGD3, RDS, RP7, PRPH2, PRPH, AVMD, AOFMD, VMD2)

In some aspects, the methods of treating a subject in need thereof comprises administering the bifunctional molecule as provided herein or the composition comprising the bifunctional molecule as provided herein or the pharmaceutical compositions comprising the bifunctional molecule as provided herein to the subject, wherein the administering is effective to treat the subject.

In some embodiments, the subject is a mammal. In some embodiments, the subject is a human.

In some embodiments, the method further comprises administering a second therapeutic agent or a second therapy in combination with the bifunctional molecule as provided herein. In some embodiments, the method comprises administering a first composition comprising the bifunctional molecule as provided herein and a second composition comprising a second therapeutic agent or a second therapy. In some embodiments, the method comprises administering a first pharmaceutical composition comprising the bifunctional molecule as provided herein and a second pharmaceutical composition comprising a second therapeutic agent or a second therapy. In some embodiments, the first composition or the first pharmaceutical composition comprising the bifunctional molecule as provided herein and the second composition or the second pharmaceutical comprising a second therapeutic agent or a second therapy are administered to a subject in need thereof simultaneously, separately, or consecutively.

The terms “treat,” “treating,” and “treatment,” and the like are used herein to generally mean obtaining a desired pharmacological and/or physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly, a human, and includes: (a) preventing the disease from occurring in a subject which may be predisposed to the disease but has not yet been diagnosed as having it; (b) inhibiting the disease, i.e., arresting its development; or (c) relieving the disease, i.e., mitigating or ameliorating the disease and/or its symptoms or conditions. The term “prophylaxis” is used herein to refer to a measure or measures taken for the prevention or partial prevention of a disease or condition.

By “treating or preventing a disease or a condition” is meant ameliorating any of the conditions or signs or symptoms associated with the disorder before or after it has occurred. As compared with an equivalent untreated control, such reduction or degree of prevention is at least 3%, 5%, 10%, 20%, 40%, 50%, 60%, 80%, 90%, 95%, or 100% as measured by any standard technique. A patient who is being treated for a disease or a condition is one who a medical practitioner has diagnosed as having such a disease or a condition. Diagnosis may be by any suitable means. A patient in whom the development of a disease or a condition is being prevented may or may not have received such a diagnosis. One in the art will understand that these patients may have been subjected to the same standard tests as described above or may have been identified, without examination, as one at high risk due to the presence of one or more risk factors (e.g., family history or genetic predisposition).

### Diseases and Disorders

In some embodiments, exemplary diseases in a subject to be treated by the bifunctional molecules as provided herein the composition or the pharmaceutical composition comprising the bifunctional molecule as provided herein include, but are not limited to, a cancer, a metabolic disease, an inflammatory disease, a cardiovascular disease, an infectious disease, a genetic disease, or a neurological disease.

For instance, examples of cancer, includes, but are not limited to, a malignant, pre-malignant or benign cancer. Cancers to be treated using the disclosed methods include, for example, a solid tumor, a lymphoma or a leukemia. In one embodiment, a cancer can be, for example, a brain tumor (e.g., a malignant, pre-malignant or benign brain tumor such as, for example, a glioblastoma, an astrocytoma, a meningioma, a medulloblastoma or a peripheral neuroectodermal tumor), a carcinoma (e.g., gall bladder carcinoma, bronchial carcinoma, basal cell carcinoma, adenocarcinoma, squamous cell carcinoma, small cell carcinoma, large cell undifferentiated carcinoma, adenomas, cystadenoma, etc.), a basalioma, a teratoma, a retinoblastoma, a choroidea melanoma, a seminoma, a sarcoma (e.g., Ewing sarcoma, rhabdomyosarcoma, craniopharyngeoma, osteosarcoma, chondrosarcoma, myosarcoma, liposarcoma, fibrosarcoma, leimyosarcoma, Askin’s tumor, lymphosarcoma, neurosarcoma, Kaposi’s sarcoma, dermatofibrosarcoma, angiosarcoma, etc.), a plasmocytoma, a head and neck tumor (e.g., oral, laryngeal, nasopharyngeal, esophageal, etc.), a liver tumor, a kidney tumor, a renal cell tumor, a squamous cell carcinoma, a uterine tumor, a bone tumor, a prostate tumor, a breast tumor including, but not limited to, a breast tumor that is Her2- and/or ER- and/or PR-, a bladder tumor, a pancreatic tumor, an endometrium tumor, a squamous cell carcinoma, a stomach tumor, gliomas, a colorectal tumor, a testicular tumor, a colon tumor, a rectal tumor, an ovarian tumor, a cervical tumor, an eye tumor, a central nervous system tumor (e.g., primary CNS lymphomas, spinal axis tumors, brain stem gliomas, pituitary adenomas, etc.), a thyroid tumor, a lung tumor (e.g., non-small cell lung cancer (NSCLC) or small cell lung cancer), a leukemia or a lymphoma (e.g., cutaneous T-cell lymphomas (CTCL), non-cutaneous peripheral T-cell lymphomas, lymphomas associated with human T-cell lymphotrophic virus (HTLV) such as adult T-cell leukemia/lymphoma (ATLL), B-cell lymphoma, acute non-lymphocytic leukemias, chronic lymphocytic leukemia, chronic myelogenous leukemia, acute myelogenous leukemia, lymphomas, and multiple myeloma, non-Hodgkin lymphoma, acute lymphatic leukemia (ALL), chronic lymphatic leukemia (CLL), Hodgkin’s lymphoma, Burkitt lymphoma, adult T-cell leukemia lymphoma, acute-myeloid leukemia (AML), chronic myeloid leukemia (CML), or hepatocellular carcinoma, etc.), a multiple myeloma, a skin tumor (e.g., basal cell carcinomas, squamous cell carcinomas, melanomas such as malignant melanomas, cutaneous melanomas or intraocular melanomas, Dermatofibrosarcoma protuberans, Merkel cell carcinoma or Kaposi’s sarcoma), a gynecologic tumor (e.g., uterine sarcomas, carcinoma of the fallopian tubes, carcinoma of the endometrium, carcinoma of the cervix, carcinoma of the vagina, carcinoma of the vulva, etc.), Hodgkin’s disease, a cancer of the small intestine, a cancer of the endocrine system (e.g., a cancer of the thyroid, parathyroid or adrenal glands, etc.), a mesothelioma, a cancer of the urethra, a cancer of the penis, tumors related to Gorlin’s syndrome (e.g., medulloblastomas, meningioma, etc.), a tumor of unknown origin; or metastases of any thereto. In some embodiments, the cancer is a lung tumor, a breast tumor, a colon tumor, a colorectal tumor, a head and neck tumor, a liver tumor, a prostate tumor, a glioma, glioblastoma multiforme, a ovarian tumor or a thyroid tumor; or metastases of any thereto. In some other embodiments, the cancer is an endometrial tumor, bladder tumor, multiple myeloma, melanoma, renal tumor, sarcoma, cervical tumor, leukemia, and neuroblastoma.

For another instance, examples of the metabolic disease include, but are not limited to diabetes, metabolic syndrome, obesity, hyperlipidemia, high cholesterol, arteriosclerosis, hypertension, non-alcoholic steatohepatitis, non-alcoholic fatty liver, non-alcoholic fatty liver disease, hepatic steatosis, and any combination thereof.

For example, the inflammatory disorder partially or fully results from obesity, metabolic syndrome, an immune disorder, an Neoplasm, an infectious disorder, a chemical agent, an inflammatory bowel disorder, reperfusion injury, necrosis, or combinations thereof. In some embodiments, the inflammatory disorder is an autoimmune disorder, an allergy, a leukocyte defect, graft versus host disease, tissue transplant rejection, or combinations thereof. In some embodiments, the inflammatory disorder is a bacterial infection, a protozoal infection, a protozoal infection, a viral infection, a fungal infection, or combinations thereof. In some embodiments, the inflammatory disorder is Acute disseminated encephalomyelitis; Addison’s disease; Ankylosing spondylitis; Antiphospholipid antibody syndrome; Autoimmune hemolytic anemia; Autoimmune hepatitis; Autoimmune inner ear disease; Bullous pemphigoid; Chagas disease; Chronic obstructive pulmonary disease; Coeliac disease; Dermatomyositis; Diabetes mellitus type 1; Diabetes mellitus type 2; Endometriosis; Goodpasture’s syndrome; Graves’ disease; Guillain-Barre syndrome; Hashimoto’s disease; Idiopathic thrombocytopenic purpura; Interstitial cystitis; Systemic lupus erythematosus (SLE); Metabolic syndrome, Multiple sclerosis; Myasthenia gravis; Myocarditis, Narcolepsy; Obesity; Pemphigus Vulgaris; Pernicious anaemia; Polymyositis; Primary biliary cirrhosis; Rheumatoid arthritis; Schizophrenia; Scleroderma; Sjëgren’s syndrome; Vasculitis; Vitiligo; Wegener’s granulomatosis; Allergic rhinitis; Prostate cancer; Non-small cell lung carcinoma; Ovarian cancer; Breast cancer; Melanoma; Gastric cancer; Colorectal cancer; Brain cancer; Metastatic bone disorder; Pancreatic cancer; a Lymphoma; Nasal polyps; Gastrointestinal cancer; Ulcerative colitis; Crohn’s disorder; Collagenous colitis; Lymphocytic colitis; Ischaemic colitis; Diversion colitis; Behçet’s syndrome; Infective colitis; Indeterminate colitis; Inflammatory liver disorder, Endotoxin shock, Rheumatoid spondylitis, Ankylosing spondylitis, Gouty arthritis, Polymyalgia rheumatica, Alzheimer’s disorder, Parkinson’s disorder, Epilepsy, AIDS dementia, Asthma, Adult respiratory distress syndrome, Bronchitis, Cystic fibrosis, Acute leukocyte-mediated lung injury, Distal proctitis, Wegener’s granulomatosis, Fibromyalgia, Bronchitis, Cystic fibrosis, Uveitis, Conjunctivitis, Psoriasis, Eczema, Dermatitis, Smooth muscle proliferation disorders, Meningitis, Shingles, Encephalitis, Nephritis, Tuberculosis, Retinitis, Atopic dermatitis, Pancreatitis, Periodontal gingivitis, Coagulative Necrosis, Liquefactive Necrosis, Fibrinoid Necrosis, Hyperacute transplant rejection, Acute transplant rejection, Chronic transplant rejection, Acute graft-versus-host disease, Chronic graft-versus-host disease, abdominal aortic aneurysm (AAA); or combinations thereof.

For another instance, examples of the neurological disease include, but are not limited to, Aarskog syndrome, Alzheimer’s disease, amyotrophic lateral sclerosis (Lou Gehrig’s disease), aphasia, Bell’s Palsy, Creutzfeldt-Jakob disease, cerebrovascular disease, Cornelia de Lange syndrome, epilepsy and other severe seizure disorders, dentatorubral-pallidoluysian atrophy, fragile X syndrome, hypomelanosis of Ito, Joubert syndrome, Kennedy’s disease, Machado-Joseph’s diseases, migraines, Moebius syndrome, myotonic dystrophy, neuromuscular disorders, Guillain-Barre, muscular dystrophy, neuro-oncology disorders, neurofibromatosis, neuro-immunological disorders, multiple sclerosis, pain, pediatric neurology, autism, dyslexia, neuro-otology disorders, Meniere’s disease, Parkinson’s disease and movement disorders, Phenylketonuria, Rubinstein-Taybi syndrome, sleep disorders, spinocerebellar ataxia I, Smith-Lemli-Opitz syndrome, Sotos syndrome, spinal bulbar atrophy, type 1 dominant cerebellar ataxia, Tourette syndrome, tuberous sclerosis complex and William’s syndrome.

The term “cardiovascular disease,” as used herein, refers to a disorder of the heart and blood vessels, and includes disorders of the arteries, veins, arterioles, venules, and capillaries. Non-limiting examples of cardiovascular diseases include coronary artery diseases, cerebral strokes (cerebrovascular disorders), peripheral vascular diseases, myocardial infarction and angina, cerebral infarction, cerebral hemorrhage, cardiac hypertrophy, arteriosclerosis, and heart failure.

The term “infectious disease,” as used herein, refer to any disorder caused by organisms, such as prions, bacteria, viruses, fungi and parasites. Examples of an infectious disease include, but are not limited to, strep throat, urinary tract infections or tuberculosis caused by bacteria, the common cold, measles, chickenpox, or AIDS caused by viruses, skin diseases, such as ringworm and athlete’s foot, lung infection or nervous system infection caused by fungi, and malaria caused by a parasite. Examples of viruses that can cause an infectious disease include, but are not limited to, Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Coronavirus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, Epstein-Barr virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, 70, Human herpesvirus 1, Human herpesvirus 2, Human herpesvirus 6, Human herpesvirus 7, Human herpesvirus 8, Human immunodeficiency virus, Human papillomavirus 1, Human papillomavirus 2, Human papillomavirus 16,18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria Marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, Norovirus, O’nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, Semliki forest virus, Seoul virus, Severe acute respiratory syndrome coronavirus 2, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, and Zika virus. Examples of infectious diseases caused by parasites include, but are not limited to, Acanthamoeba Infection, Acanthamoeba Keratitis Infection, African Sleeping Sickness (African trypanosomiasis), Alveolar Echinococcosis (Echinococcosis, Hydatid Disease), Amebiasis (Entamoeba histolytica Infection), American Trypanosomiasis (Chagas Disease), Ancylostomiasis (Hookworm), Angiostrongyliasis (Angiostrongylus Infection), Anisakiasis (Anisakis Infection, Pseudoterranova Infection), Ascariasis (Ascaris Infection, Intestinal Roundworms), Babesiosis (Babesia Infection), Balantidiasis (Balantidium Infection), Balamuthia, Baylisascariasis (Baylisascaris Infection, Raccoon Roundworm), Bed Bugs, Bilharzia (Schistosomiasis), Blastocystis hominis Infection, Body Lice Infestation (Pediculosis), Capillariasis (Capillaria Infection), Cercarial Dermatitis (Swimmer’s Itch), Chagas Disease (American Trypanosomiasis), Chilomastix mesnili Infection (Nonpathogenic [Harmless] Intestinal Protozoa), Clonorchiasis (Clonorchis Infection), CLM (Cutaneous Larva Migrans, Ancylostomiasis, Hookworm), “Crabs” (Pubic Lice), Cryptosporidiosis (Cryptosporidium Infection), Cutaneous Larva Migrans (CLM, Ancylostomiasis, Hookworm), Cyclosporiasis (Cyclospora Infection), Cysticercosis (Neurocysticercosis), Cystoisospora Infection (Cystoisosporiasis) formerly Isospora Infection, Dientamoeba fragilis Infection, Diphyllobothriasis (Diphyllobothrium Infection), Dipylidium caninum Infection (dog or cat tapeworm infection), Dirofilariasis (Dirofilaria Infection), DPDx, Dracunculiasis (Guinea Worm Disease), Dog tapeworm (Dipylidium caninum Infection), Echinococcosis (Cystic, Alveolar Hydatid Disease), Elephantiasis (Filariasis, Lymphatic Filariasis), Endolimax nana Infection (Nonpathogenic [Harmless] Intestinal Protozoa), Entamoeba coli Infection (Nonpathogenic [Harmless] Intestinal Protozoa), Entamoeba dispar Infection (Nonpathogenic [Harmless] Intestinal Protozoa), Entamoeba hartmanni Infection (Nonpathogenic [Harmless] Intestinal Protozoa), Entamoeba histolytica Infection (Amebiasis), Entamoeba polecki, Enterobiasis (Pinworm Infection), Fascioliasis (Fasciola Infection), Fasciolopsiasis (Fasciolopsis Infection), Filariasis (Lymphatic Filariasis, Elephantiasis), Giardiasis (Giardia Infection), Gnathostomiasis (Gnathostoma Infection), Guinea Worm Disease (Dracunculiasis), Head Lice Infestation (Pediculosis), Heterophyiasis (Heterophyes Infection), Hookworm Infection, Human, Hookworm Infection, Zoonotic (Ancylostomiasis, Cutaneous Larva Migrans [CLM]), Hydatid Disease (Cystic, Alveolar Echinococcosis), Hymenolepiasis (Hymenolepis Infection), Intestinal Roundworms (Ascariasis, Ascaris Infection), Iodamoeba buetschlii Infection (Nonpathogenic [Harmless] Intestinal Protozoa), Isospora Infection (see Cystoisospora Infection ), Kala-azar (Leishmaniasis, Leishmania Infection), Keratitis (Acanthamoeba Infection), Leishmaniasis (Kala-azar, Leishmania Infection), Lice Infestation (Body, Head, or Pubic Lice, Pediculosis, Pthiriasis), Liver Flukes (Clonorchiasis, Opisthorchiasis, Fascioliasis), Loiasis (Loa loa Infection), Lymphatic filariasis (Filariasis, Elephantiasis), Malaria (Plasmodium Infection), Microsporidiosis (Microsporidia Infection), Mite Infestation (Scabies), Myiasis, Naegleria Infection, Neurocysticercosis (Cysticercosis), Ocular Larva Migrans (Toxocariasis, Toxocara Infection, Visceral Larva Migrans), Onchocerciasis (River Blindness), Opisthorchiasis (Opisthorchis Infection), Paragonimiasis (Paragonimus Infection), Pediculosis (Head or Body Lice Infestation), Pthiriasis (Pubic Lice Infestation), Pinworm Infection (Enterobiasis), Plasmodium Infection (Malaria), Pneumocystis jirovecii Pneumonia, Pseudoterranova Infection (Anisakiasis, Anisakis Infection), Pubic Lice Infestation (“Crabs,” Pthiriasis), Raccoon Roundworm Infection (Baylisascariasis, Baylisascaris Infection), River Blindness (Onchocerciasis), Sappinia, Sarcocystosis (Sarcocystosis Infection), Scabies, Schistosomiasis (Bilharzia), Sleeping Sickness (Trypanosomiasis, African; African Sleeping Sickness), Soil-transmitted Helminths, Strongyloidiasis (Strongyloides Infection), Swimmer’s Itch (Cercarial Dermatitis), Taeniasis (Taenia Infection, Tapeworm Infection), Tapeworm Infection (Taeniasis, Taenia Infection), Toxocariasis (Toxocara Infection, Ocular Larva Migrans, Visceral Larva Migrans), Toxoplasmosis (Toxoplasma Infection), Trichinellosis (Trichinosis),Trichinosis (Trichinellosis), Trichomoniasis (Trichomonas Infection), Trichuriasis (Whipworm Infection, Trichuris Infection), Trypanosomiasis, African (African Sleeping Sickness, Sleeping Sickness), Trypanosomiasis, American (Chagas Disease), Visceral Larva Migrans (Toxocariasis, Toxocara Infection, Ocular Larva Migrans), Whipworm Infection (Trichuriasis, Trichuris Infection), Zoonotic Diseases (Diseases spread from animals to people), and Zoonotic Hookworm Infection (Ancylostomiasis, Cutaneous Larva Migrans [CLM]). Examples of infectious diseases caused by fungi include, but are not limited to, Apergillosis, Balsomycosis, Candidiasis, Cadidia auris, Coccidioidomycosis, C. neoformans infection, C gattii infection, fungal eye infections, fungal nail infections, histoplasmosis, mucormycosis, mycetoma, Pneuomcystis pneumonia, ringworm, sporotrichosis, cyrpococcosis, and Talaromycosis. Examples of bacteria that can cause an infectious disease include, but are not limited to, Acinetobacter baumanii, Actinobacillus sp., Actinomycetes, Actinomyces sp. (such as Actinomyces israelii and Actinomyces naeslundii), Aeromonas sp. (such as Aeromonas hydrophila, Aeromonas veronii biovar sobria (Aeromonas sobria), and Aeromonas caviae), Anaplasma phagocytophilum, Anaplasma marginale Alcaligenes xylosoxidans, Acinetobacter baumanii, Actinobacillus actinomycetemcomitans, Bacillus sp. (such as Bacillus anthracis, Bacillus cereus, Bacillus subtilis, Bacillus thuringiensis, andBacillus stearothermophilus), Bacteroides sp. (such as Bacteroides fragilis), Bartonella sp. (such as Bartonella bacilliformis and Bartonella henselae, Bifidobacterium sp., Bordetella sp. (such as Bordetella pertussis, Bordetella parapertussis, and Bordetella bronchiseptica), Borrelia sp. (such as Borrelia recurrentis, and Borrelia burgdorferi), Brucella sp. (such as Brucella abortus, Brucella canis, Brucella melintensis and Brucella suis), Burkholderia sp. (such as Burkholderia pseudomallei and Burkholderia cepacia), Campylobacter sp. (such as Campylobacter jejuni, Campylobacter coli, Campylobacter lari and Campylobacter fetus), Capnocytophaga sp., Cardiobacterium hominis, Chlamydia trachomatis, Chlamydophila pneumoniae, Chlamydophila psittaci, Citrobacter sp. Coxiella burnetii, Corynebacterium sp. (such as, Corynebacterium diphtheriae, Corynebacterium jeikeum and Corynebacterium), Clostridium sp. (such as Clostridium perfringens, Clostridium dificile, Clostridium botulinum and Clostridium tetani), Eikenella corrodens, Enterobacter sp. (such as Enterobacter aerogenes, Enterobacter agglomerans, Enterobacter cloacae and Escherichia coli, including opportunistic Escherichia coli, such as enterotoxigenic E.coli, enteroinvasive E.coli, enteropathogenic E.coli, enterohemorrhagic E.coli, enteroaggregative E.coli and uropathogenic E. coli) Enterococcus sp. (such as Enterococcus faecalis and Enterococcus faecium) Ehrlichia sp. (such as Ehrlichia chafeensia and Ehrlichia canis), Epidermophyton floccosum, Erysipelothrix rhusiopathiae, Eubacterium sp., Francisella tularensis, Fusobacterium nucleatum, Gardnerella vaginalis, Gemella morbillorum, Haemophilus sp. (such as Haemophilus influenzae, Haemophilus ducreyi, Haemophilus aegyptius, Haemophilus parainfluenzae, Haemophilus haemolyticus and Haemophilus parahaemolyticus, Helicobacter sp. (such as Helicobacter pylori, Helicobacter cinaedi and Helicobacter fennelliae), Kingella kingii, Klebsiella sp. (such as Klebsiella pneumoniae, Klebsiella granulomatis and Klebsiella oxytoca), Lactobacillus sp., Listeria monocytogenes, Leptospira interrogans, Legionella pneumophila, Leptospira interrogans, Peptostreptococcus sp., Mannheimia hemolytica, Microsporum canis, Moraxella catarrhalis, Morganella sp., Mobiluncus sp., Micrococcus sp., Mycobacterium sp. (such as Mycobacterium leprae, Mycobacterium tuberculosis, Mycobacterium paratuberculosis, Mycobacterium intracellulare, Mycobacterium avium, Mycobacterium bovis, and Mycobacterium marinum), Mycoplasm sp. (such as Mycoplasma pneumoniae, Mycoplasma hominis, and Mycoplasma genitalium), Nocardia sp. (such as Nocardia asteroides, Nocardia cyriacigeorgica and Nocardia brasiliensis), Neisseria sp. (such as Neisseria gonorrhoeae and Neisseria meningitidis), Pasteurella multocida, Pityrosporum orbiculare (Malassezia furfur), Plesiomonas shigelloides. Prevotella sp., Porphyromonas sp., Prevotella melaninogenica, Proteus sp. (such as Proteus vulgaris and Proteus mirabilis), Providencia sp. (such as Providencia alcalifaciens, Providencia rettgeri and Providencia stuartii), Pseudomonas aeruginosa, Propionibacterium acnes, Rhodococcus equi, Rickettsia sp. (such as Rickettsia rickettsii, Rickettsia akari and Rickettsia prowazekii, Orientia tsutsugamushi (formerly: Rickettsia tsutsugamushi) and Rickettsia typhi), Rhodococcus sp., Serratia marcescens, Stenotrophomonas maltophilia, Salmonella sp. (such as Salmonella enterica, Salmonella typhi, Salmonella paratyphi, Salmonella enteritidis, Salmonella cholerasuis and Salmonella typhimurium), Serratia sp. (such as Serratia marcesans and Serratia liquifaciens), Shigella sp. (such as Shigella dysenteriae, Shigella flexneri, Shigella boydii and Shigella sonnei), Staphylococcus sp. (such as Staphylococcus aureus, Staphylococcus epidermidis, Staphylococcus hemolyticus, Staphylococcus saprophyticus), Streptococcus sp. (such as Streptococcus pneumoniae (for example chloramphenicol-resistant serotype 4 Streptococcus pneumoniae, spectinomycin-resistant serotype 6B Streptococcus pneumoniae, streptomycin-resistant serotype 9V Streptococcus pneumoniae, erythromycin-resistant serotype 14 Streptococcus pneumoniae, optochin-resistant serotype 14 Streptococcus pneumoniae, rifampicin-resistant serotype 18C Streptococcus pneumoniae, tetracycline-resistant serotype 19F Streptococcus pneumoniae, penicillin-resistant serotype 19F Streptococcus pneumoniae, and trimethoprim-resistant serotype 23F Streptococcus pneumoniae, chloramphenicol-resistant serotype 4 Streptococcus pneumoniae, spectinomycin-resistant serotype 6B Streptococcus pneumoniae, streptomycin-resistant serotype 9V Streptococcus pneumoniae, optochin-resistant serotype 14 Streptococcus pneumoniae, rifampicin-resistant serotype 18C Streptococcus pneumoniae, penicillin-resistant serotype 19F Streptococcus pneumoniae, or trimethoprim-resistant serotype 23F Streptococcus pneumoniae), Streptococcus agalactiae, Streptococcus mutans, Streptococcus pyogenes, Group A streptococci, Streptococcus pyogenes, Group B streptococci, Streptococcus agalactiae, Group C streptococci, Streptococcus anginosus, Streptococcus equismilis, Group D streptococci, Streptococcus bovis, Group F streptococci, and Streptococcus anginosus Group G streptococci), Spirillum minus, Streptobacillus moniliformi, Treponema sp. (such as Treponema carateum, Treponema petenue, Treponema pallidum and Treponema endemicum, Trichophyton rubrum, T. mentagrophytes, Tropheryma whippelii, Ureaplasma urealyticum, Veillonella sp., Vibrio sp. (such as Vibrio cholerae, Vibrio parahemolyticus, Vibrio vulnificus, Vibrio parahaemolyticus, Vibrio vulnificus, Vibrio alginolyticus, Vibrio mimicus, Vibrio hollisae, Vibrio fluvialis, Vibrio metchnikovii, Vibrio damsela and Vibrio furnisii), Yersinia sp. (such as Yersinia enterocolitica, Yersinia pestis, and Yersinia pseudotuberculosis) and Xanthomonas maltophilia

The term “genetic disease,” as used herein, refers to a health problem caused by one or more abnormalities in the genome. It can be caused by a mutation in a single gene (monogenic) or multiple genes (polygenic) or by a chromosomal abnormality. The single gene disease may be related to an autosomal dominant, autosomal recessive, X-linked dominant, X-linked recessive, Y-linked, or mitochondrial mutation. Examples of genetic diseases include, but are not limited to, 1p36 deletion syndrome, 18p deletion syndrome, 21-hydroxylase deficiency, 47,XXX (triple X syndrome), AAA syndrome (achalasia-addisonianism-alacrima syndrome), Aarskog-Scott syndrome, ABCD syndrome, Aceruloplasminemia, Acheiropodia, Achondrogenesis type II, achondroplasia, Acute intermittent porphyria, adenylosuccinate lyase deficiency, Adrenoleukodystrophy, ADULT syndrome, Aicardi-Goutieres syndrome, Alagille syndrome, Albinism, Alexander disease, alkaptonuria, Alpha 1-antitrypsin deficiency, Alport syndrome, Alström syndrome, Alternating hemiplegia of childhood, Alzheimer’s disease, Amelogenesis imperfecta, Aminolevulinic acid dehydratase deficiency porphyria, Amyotrophic lateral sclerosis - Frontotemporal dementia, Androgen insensitivity syndrome, Angelman syndrome, Apert syndrome, Arthrogryposis-renal dysfunction-cholestasis syndrome, Ataxia telangiectasia, Axenfeld syndrome, Beare-Stevenson cutis gyrata syndrome, Beckwith-Wiedemann syndrome, Benjamin syndrome, biotinidase deficiency, Birt-Hogg-Dube syndrome, Björnstad syndrome, Bloom syndrome, Brody myopathy, Brunner syndrome, CADASIL syndrome, Campomelic dysplasia, Canavan disease, CARASIL syndrome, Carpenter Syndrome, Cerebral dysgenesis-neuropathy-ichthyosis-keratoderma syndrome (SEDNIK), Charcot-Marie-Tooth disease, CHARGE syndrome, Chediak-Higashi syndrome, Chronic granulomatous disorder, Cleidocranial dysostosis, Cockayne syndrome, Coffin-Lowry syndrome, Cohen syndrome, collagenopathy, types II and XI, Congenital insensitivity to pain with anhidrosis (CIPA), Congenital Muscular Dystrophy, Cornelia de Lange syndrome (CDLS), Cowden syndrome, CPO deficiency (coproporphyria), Cranio-lenticulo-sutural dysplasia, Cri du chat, Crohn’s disease, Crouzon syndrome, Crouzonodermoskeletal syndrome (Crouzon syndrome with acanthosis nigricans), Cystic fibrosis, Darier’s disease, De Grouchy syndrome, Dent’s disease (Genetic hypercalciuria), Denys-Drash syndrome, Di George’s syndrome, Distal hereditary motor neuropathies, multiple types, Distal muscular dystrophy, Down Syndrome, Dravet syndrome, Duchenne muscular dystrophy, Edwards Syndrome, Ehlers-Danlos syndrome, Emery-Dreifuss syndrome, Epidermolysis bullosa, Erythropoietic protoporphyria, Fabry disease, Factor V Leiden thrombophilia, Familial adenomatous polyposis, Familial Creutzfeld-Jakob Disease, Familial dysautonomia, Fanconi anemia (FA), Fatal familial insomnia, Feingold syndrome, FG syndrome, Fragile X syndrome, Friedreich’s ataxia, G6PD deficiency, Galactosemia, Gaucher disease, Gerstmann-Straussler-Scheinker syndrome, Gillespie syndrome, Glutaric aciduria, type I and type 2, GRACILE syndrome, Griscelli syndrome, Hailey-Hailey disease, Harlequin type ichthyosis, Hemochromatosis, hereditary, Hemophilia, Hepatoerythropoietic porphyria, Hereditary coproporphyria, Hereditary hemorrhagic telangiectasia (Osler-Weber-Rendu syndrome), Hereditary inclusion body myopathy, Hereditary multiple exostoses, Hereditary neuropathy with liability to pressure palsies (HNPP), Hereditary spastic paraplegia (infantile-onset ascending hereditary spastic paralysis), Hermansky-Pudlak syndrome, Heterotaxy, Homocystinuria, Hunter syndrome, Huntington’s disease, Hurler syndrome, Hutchinson-Gilford progeria syndrome, Hyperlysinemia, Hyperoxaluria, Hyperphenylalaninemia, Hypoalphalipoproteinemia (Tangier disease), Hypochondrogenesis, Hypochondroplasia, Immunodeficiency-centromeric instability-facial anomalies syndrome (ICF syndrome), Incontinentia pigmenti, Ischiopatellar dysplasia, Isodicentric 15, Jackson-Weiss syndrome, Joubert syndrome, Juvenile primary lateral sclerosis (JPLS), Keloid disorder, Kniest dysplasia, Kosaki overgrowth syndrome, Krabbe disease, Kufor-Rakeb syndrome, LCAT deficiency, Lesch-Nyhan syndrome, Li-Fraumeni syndrome, Limb-Girdle Muscular Dystrophy, lipoprotein lipase deficiency, Lynch syndrome, Malignant hyperthermia, Maple syrup urine disease, Marfan syndrome, Maroteaux-Lamy syndrome, McCune-Albright syndrome, McLeod syndrome, Mediterranean fever, familial, MEDNIK syndrome, Menkes disease, Methemoglobinemia, Methylmalonic acidemia, Micro syndrome, Microcephaly, Morquio syndrome, Mowat-Wilson syndrome, Muenke syndrome, Multiple endocrine neoplasia type 1 (Wermer’s syndrome), Multiple endocrine neoplasia type 2, Muscular dystrophy, Muscular dystrophy, Duchenne and Becker type, Myostatin-related muscle hypertrophy, myotonic dystrophy, Natowicz syndrome, Neurofibromatosis type I, Neurofibromatosis type II, Niemann-Pick disease, Nonketotic hyperglycinemia, Nonsyndromic deafness, Noonan syndrome, Norman-Roberts syndrome, Ogden syndrome, Omenn syndrome, Osteogenesis imperfecta, Pantothenate kinase-associated neurodegeneration, Patau syndrome (Trisomy 13), PCC deficiency (propionic acidemia), Pendred syndrome, Peutz-Jeghers syndrome, Pfeiffer syndrome, Phenylketonuria, Pipecolic acidemia, Pitt-Hopkins syndrome, Polycystic kidney disease, Polycystic ovary syndrome (PCOS), Porphyria, Porphyria cutanea tarda (PCT), Prader-Willi syndrome, Primary ciliary dyskinesia (PCD), Primary pulmonary hypertension, Protein C deficiency, Protein S deficiency, Pseudo-Gaucher disease, Pseudoxanthoma elasticum, Retinitis pigmentosa, Rett syndrome, Roberts syndrome, Rubinstein-Taybi syndrome (RSTS), Sandhoff disease, Sanfilippo syndrome, Schwartz-Jampel syndrome, Shprintzen-Goldberg syndrome, Sickle cell anemia, Siderius X-linked mental retardation syndrome, Sideroblastic anemia, Sjogren-Larsson syndrome, Sly syndrome, Smith-Lemli-Opitz syndrome, Smith-Magenis syndrome, Snyder-Robinson syndrome, Spinal muscular atrophy, Spinocerebellar ataxia (types 1-29), Spondyloepiphyseal dysplasia congenita (SED), SSB syndrome (SADDAN), Stargardt disease (macular degeneration), Stickler syndrome (multiple forms), Strudwick syndrome (spondyloepimetaphyseal dysplasia, Strudwick type), Tay-Sachs disease, Tetrahydrobiopterin deficiency, Thanatophoric dysplasia, Treacher Collins syndrome, Tuberous sclerosis complex (TSC), Turner syndrome, Usher syndrome, Variegate porphyria, von Hippel-Lindau disease, Waardenburg syndrome, Weissenbacher-Zweymuller syndrome, Williams syndrome, Wilson disease, Wolf-Hirschhorn syndrome, Woodhouse-Sakati syndrome, X-linked intellectual disability and macroorchidism (fragile X syndrome), X-linked severe combined immunodeficiency (X-SCID), X-linked sideroblastic anemia (XLSA), X-linked spinal-bulbar muscle atrophy (spinal and bulbar muscular atrophy), Xeroderma pigmentosum, Xp11.2 duplication syndrome, XXXX syndrome (48, XXXX), XXXXX syndrome (49, XXXXX), XYY syndrome (47,XYY), Zellweger syndrome.

All references, publications, patents, and patent applications mentioned in this specification are herein incorporated by reference to the same extent as if each individual publication, patent, or patent application was specifically and individually indicated to be incorporated by reference.

The above described embodiments can be combined to achieve the afore-mentioned functional characteristics. This is also illustrated by the below examples which set forth exemplary combinations and functional characteristics achieved.

### EXAMPLES

The following examples are provided to further illustrate some embodiments of the present disclosure, but are not intended to limit the scope of the present disclosure; it will be understood by their exemplary nature that other procedures, methodologies, or techniques known to those skilled in the art may alternatively be used.

### Example 1: Generating Binding ASOs to RNA Targets

Methods to design antisense oligonucleotides to PVT1, MYC and SCN1A were developed.

The sequences of PVT1, MYC and SCN1A were run into a publicly-available program (sfold, sfold.wadsworth.org) to identify regions suitable for high binding energy ASOs, typically lower than -8 kcal, using 20 nucleotides as sequence length. ASOs with more than 3 consecutive G nucleotides were excluded. The ASOs with the highest binding energy were then processed through BLAST to check their potential binding selectivity based on nucleotide sequence, and those with at least 2 mismatches to other sequences were retained. The selected ASOs were then synthesized as follows:

### 5′-Amino ASO Synthesis

5′-Amino ASO was synthesized with a typical step-wise solid phase oligonucleotide synthesis method on a Dr. Oligo 48 (Biolytic Lab Performance Inc.) synthesizer, according to manufacturer’s protocol. A 1000 nmol scale universal CPG column (Biolytic Lab Performance Inc. part number 168-108442-500) was utilized as the solid support. The monomers were modified RNA phosphoramidites with protecting groups (5′-O-(4,4′-Dimethoxytrityl)-2′-O-methoxyethyl-N6-benzoyl-adenosine -3′-O-[(2-cyanoethyl)-(N,N-diisopropyl)]-phosphoramidite, 5′-O-(4,4′-Dimethoxytrityl)-2′-O-methoxyethyl-5-methyl-N4-benzoyl-cytidine-3′-O-[(2-cyanoethyl)-(N,N-diisopropyl)]-phosphoramidite, 5′-O-(4,4′-Dimethoxytrityl)-2′-O-methoxyethyl-N2-isobutyryl- guanosine-3′-O-[(2-cyanoethyl)-(N,N-diisopropyl)]-phosphoramidite, 5′-O-(4,4′-Dimethoxytrityl)-2′-O-methoxyethyl-5-methyl-uridine-3′-O-[(2-cyanoethyl)-(N,N-diisopropyl)]-phosphoramidite) purchased from Chemgenes Corporation. The 5′-amino modification required the use of the TFA-amino C6-CED phosphoramidite (6-(Trifluoroacetylamino)-hexyl-(2-cyanoethyl)-(N,N-diisopropyl)-phosphoramidite) in the last step of synthesis. All monomers were diluted to 0.1 M with anhydrous acetonitrile (Fisher Scientific BP1170) prior to being used on the synthesizer.

The commercial reagents used for synthesis on the oligonucleotide synthesizer, including 3% trichloroacetic acid in dichloromethane (DMT removal reagent, RN-1462), 0.3 M benzylthiotetrazole in acetonitrile (activation reagent, RN-1452), 0.1 M ((Dimethylamino-methylidene)amino)-3H-1,2,4-dithiazoline-3-thione in 9:1 pyridine/acetonitrile (sulfurizing reagent, RN-1689), 0.2 M iodine/pyridine/water/tetrahydrofuran (oxidation solution, RN-1455), acetic anhydride/pyridine/tetrahydrofuran (CAP A solution, RN-1458), 10% N-methylimidazole in tetrahydrofuran (CAP B solution, RN-1481), were purchased from ChemGenes Corporation. Anhydrous acetonitrile (wash reagent, BP1170) was purchased from Fisher Scientific for use on the synthesizer. All solutions and reagents were kept anhydrous with the use of drying traps (DMT-1975, DMT-1974, DMT-1973, DMT-1972) purchased from ChemGenes Corporation.

### Cyanoethyl Protecting Group Removal

In order to prevent acrylonitrile adduct formation on the primary amine, the 2′-cyanoethyl protecting groups were removed prior to deprotection of the amine. A solution of 10% diethylamine in acetonitrile was added to column as needed to maintain contact with the column for 5 minutes. The column was then washed 5 times with 500 uL of acetonitrile.

### Deprotection and Cleavage

The oligonucleotide was cleaved from the support with simultaneous deprotection of other protecting groups. The column was transferred to a screw cap vial with a pressure relief cap (ChemGlass Life Sciences CG-4912-01). 1 mL of ammonium hydroxide was added to the vial and the vial was heated to 55° C. for 16 hours. The vial was cooled to room temperature and the ammonia solution was transferred to a 1.5 mL microfuge tube. The CPG support was washed with 200 uL of RNAse free molecular biology grade water and the water was added to the ammonia solution. The resulting solution was concentrated in a centrifugal evaporator (SpeedVac SPD1030).

### Precipitation

The residue was dissolved in 360 uL of RNAse free molecular biology grade water and 40 uL of a 3 M sodium acetate buffer solution was added. To remove impurities, the microfuge tube was centrifuged at a high speed (14000 g) for 10 minutes. The supernatant was transferred to a tared 2 mL microfuge tube. 1.5 mL of ethanol was added to the clear solution and tube was vortexed and then stored at -20° C. for 1 hour. The microfuge tube was then centrifuged at a high speed (14000 g) at 5° C. for 15 minutes. The supernatant was carefully removed, without disrupting the pellet, and the pellet was dried in the SpeedVac. The oligonucleotide yield was estimated by mass calculation and the pellet was resuspended in RNAse free molecular biology grade water to give an 8 mM solution which was used in subsequent steps.

ASOs targeting specific RNA targets were designed and synthesized successfully according to this example.

### Example 2: Conjugating ASO to Small Molecules

Methods to conjugate PVT1, MYC, and SCN1A ASOs to a small molecule were developed.

To target PVT1, MYC, and SCN1A, a bi-functional modality was used. The modality includes two domains, a first domain that targets the RNA that demarcates the gene (this can be a RNA binding protein, an ASO, a small molecule) and a second domain that binds/recruits a transcriptional modifying enzyme (this can be a protein, aptamer, small molecule/inhibitor etc), with the two domains connected by a linker.

The modality used in this example was a PVT1, MYC, or SCN1A specific ASO linked to a small molecule JQ1 or iBET762 that binds/recruits Bromodomain-containing protein 4 (BRD4).

The synthesized 5′-amino ASOs from Example 1 were used to make ASO-small molecule conjugates following the scheme (linker2 as representative) below.

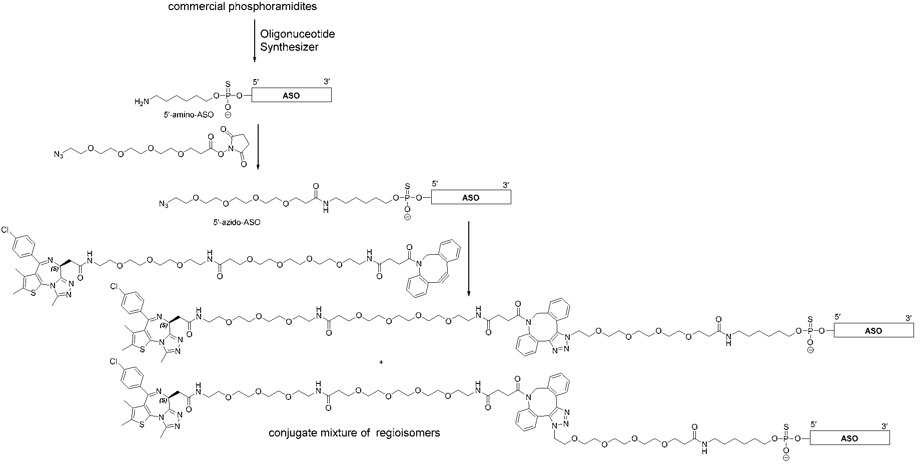

The following protocol was used to make 5′-azido-ASO from 5′-amino-ASO.

A solution of 5′-amino ASO (2 mM, 15 µL, 30 nmole) was mixed with a sodium borate buffer (pH 8.5, 75 µL). A solution of N₃-PEG₄-NHS ester (10 mM in DMSO, 30 µL, 300 nmol) was then added, and the mixture was orbitally shaken at room temperature for 16 hours. The solution was dried overnight with SpeedVac. The resulting residue was redissolved in water (20 µL) and purified by reverse phase HPLC to provide 5′-azido ASO (12-21 nmol by nanodrop UV-VIS quantitation). This 5′-azido ASO solution in water (2 mM in water, 7 µL) was mixed with DBCO-PEG₄-JQ1 (synthesized from DBCO-PEG₄-NHS and amino-PEG3-JQ1 and purified by reverse phase HPLC, 2 mM in DMSO, 28 µL) in a PCR tube and was orbitally shaken at room temperature for 16 hours. The reaction mixture was dried over night with SpeedVac. The resulting residue was redissolved in water (20 µL), centrifuged to provide clear supernatant, which was purified by reverse phase HPLC to provide ASO-Linker-JQ1 conjugate as a mixture of regioisomers (4.2-9.8 nmol by nanodrop UV-VIS quantitation). The conjugate was characterized by matrix-assisted laser desorption/ionization time of flight mass spectrometry (MALDI-TOF MS).

ASOs that were conjugated to small molecule JQ1 or iBET762 were successfully synthesized using the above methods.

### Example 3: Formation of RNA-Bifunctional-Protein Ternary Complex In Vitro

Methods to form the RNA-bifunctional-protein ternary complex were developed.

### Bifunctional Design

A ternary complex is a complex containing three different molecules bound together. A bifunctional molecule was shown to interact with target RNA (by ASO) and target protein (by small molecule). As shown in FIG. 1, an inhibitor-conjugated antisense oligonucleotide (hereafter referred to as Ibrutinib-ASOi) was mixed together with the protein target of the inhibitor and the RNA target of the ASO, and allowed to react with the protein and hybridize with the RNA target to form a ternary complex including all 3 molecules. Binding of the Ibrutinib-ASOi to the target protein caused the protein to migrate higher (shift up) on a polyacrylamide gel because of its increased molecular weight. Additional hybridization of the target RNA to the ASOi-protein complex was determined by observing a “supershifted” protein band even higher on the gel, indicating that all 3 components were stably associated in the complex. Furthermore, labeling the target RNA with a fluorescent dye was used to enable direct visualization of the target RNA in the supershifted protein complex.

### Example 3a: Formation of Ibrutinib-ASO

The inhibitor Ibrutinib covalently binds the ATP-binding pocket of Bruton’s Tyrosine Kinase (BTK) protein (doi.org/10.1124/mol.116.107037) and so was conjugated to ASOs.

To generate the conjugate, 10 uL of a 50 mM Dibenzocyclooctyne-PEG4-N-hydroxysuccinimidyl ester (Sigma-Aldrich) solution in DMSO was added to a mixture of 15 uL of 50 mM solution of Ibrutinib-MPEA (Chemscene) in DMSO and 15 uL of a 50 mM diisopropylethylamine in DMSO. The mixture was orbitally shaken for 4 h at room temperature, and the product was used without further analysis or purification in the next step. 10 ul of the previous solution was added to 10 nmol of azido-ASO (2 mM solution in water), and 30 uL of DMSO was added to the mixture. The mixture was orbitally shaken overnight at room temperature. The mixture was then transferred onto a 0.5 mL amicon column (3 kDa) and spun at 10 g. The residue is then diluted with water and spun. This process was repeated three times to afford the expected ASO-Ibrutinib conjugate which was characterized by matrix-assisted laser desorption/ionization time of flight mass spectrometry (MALDI-TOF MS).

### Example 3b: In Vitro Ternary Complex Formation Assay

In one reaction (#5), 5 pmol antisense RNA oligo of the sequence 5′CGUUAACUAGGCUUUA3′ (hereafter called N33-ASOi) conjugated at the 5′ end with Ibrutinib was mixed in PBS with 1 pmol purified BTK protein (Active Motif #81083), 200 pmol yeast rRNA (as non-specific blocker) and 10 pmol Cy5-labeled IVT RNA of the sequence 5′CCUUGAAAUCCAUGACGCAGGGAGAAUUGCGUCAUUUAAAGCCUAGUUAACGC AUUUACUAAACGCAGACGAAAAUGGAAAGAUUAAUUGGGAGUGGUAGGAUGAAA CAAUUUGGAGAAGAUAGAAGUUUGAAGUGGAAAACUGGAAGACAGAAGUACGGG AAGGCGAA3′ (SEQ ID NO: 51).

As controls, the following reactions were mixed in PBS with 200 pmol yeast tRNA and the following components:
(#1) 10 pmol Cy5-IVT RNA only (to identify band size on gel of RNA transcript. FIG. 1, arrow D);
(#2) 1 pmol purified BTK protein only (to identify band size on gel of non-complexed protein FIG. 1, arrow C);
(#3) 1 pmol purified BTK protein and 10 pmol Cy5-IVT RNA (to test whether the target RNA interacts directly with BTK protein);
(#4) 1 pmol purified BTK protein and 10 pmol N33-ASOi (to identify size of 2-component shifted band, FIG. 1, arrow B);
(#6) 5 pmol non-complementary RNA oligo of the sequence 5′AGAGGUGGCGUGGUAG3′ (hereafter called SCR-ASOi) conjugated at the 5′ end with Ibrutinib, 10 pmol Cy5-IVT RNA and 1 pmol purified BTK protein (to test whether formation of the ternary complex requires a complementary ASO sequence); and
(#7) 1 pmol purified BTK protein and 5 pmol SCR-ASOi (to show that the Ibrutinib-modified scrambled ASO is capable of size-shifting the BTK protein band).
(#8) 5 pmol N33-ASOi and 10 pmol Cy5-IVT RNA (to show binding between target RNA and ASO)
(#9) 5 pmol SCR-ASOi and 10 pmol Cy5-IVT RNA (to show ASO - RNA interaction requires complementary sequences)

All reactions were incubated at room temperature for 90 minutes protected from light, then mixed with a loading buffer containing final 0.5% SDS and 10% glycerol, and complexes separated by PAGE on a Bis-Tris 4-12% gel including an IRDye700 pre-stained protein molecular weight marker (LiCor). Immediately following electrophoresis, the gel was imaged using a LiCor Odyssey system with the 700 nm channel to identify the position of Cy5-IVT-RNA bands and MW marker. Subsequently, proteins in the gel were stained using InstantBlue colloidal coomassie stain (Expedeon) and re-imaged using transmitted light. The two images were lined up using size markers and lane positions to identify the relative positions of BTK protein bands and Cy5-IVT target RNA. (FIG. 1)

An increase in MW of the BTK protein band when reacted with N33-ASOi (sample 2 and 3 vs. 4, arrows C and B) was observed to indicate binary complex formation, and a further supershift in the presence of Cy5-IVT RNA (Sample 5, arrow A) observed with N33-ASOi but not with SCR-ASOi (Sample 6, complex stayed at arrow B level) demonstrated that all 3 components were present in the complex and that formation was specific to hybridizing a complementary sequence. This complex was further confirmed by Cy5-IVT-RNA fluorescence signal overlapping the super-shifted BTK protein band.

The bifunctional molecule was shown to interact with the target RNA via the ASO and the target protein by the small molecule.

### Example 4: Increasing Gene Expression With Endogenous Factors (RNA and Effector)

Gene expression was increased with endogenous factors (RNA and effector).

Methods to increase gene expression by targeting endogenous RNAs and effector proteins with bifunctional molecules were developed.

Specific RNAs may demarcate every gene in the genome. By targeting these RNAs to recruit transcriptional modifying enzymes, the local concentration of the transcriptional modifying enzyme near the gene is increased, thereby increasing transcription of the underlying gene (either repressing or activating transcription).

### Example 4a: Design of Bifunctional Molecule

The ASO and ASO-Linker2-JQ1 syntheses are described in Examples 1 and 2. ASO-Linker1-JQ1 is synthesized according to Examples 1 and 2, using 6-azidohexanoic acid NHS ester in the place of N3-PEG4-NHS ester.

ASO-JQ1 conjugates were generated as the following general chemical structure. Herein the ASO-Linker2-JQ1 conjugates were made from all ASOs in Table 1B, except for the SCN1A-ASO1 which is made as SCN1A-ASO1-Linker1-JQ1. Besides PVT1-ASO1-Linker2-JQ1, PVT1-ASO1-Linker1-JQ1 was also made as the chemical structures below. Simplified General Chemical Structure of ASO-Linker1-JQ1 (mixture of isomers) 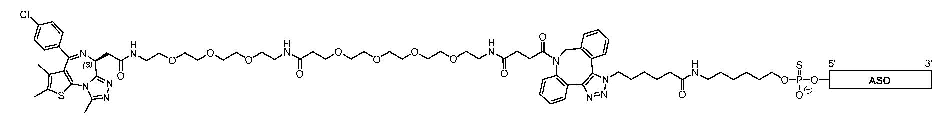 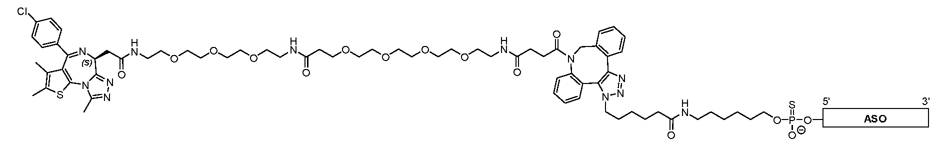 Simplified General Chemical Structure of ASO-Linker2-JQ1 (mixture of isomers) 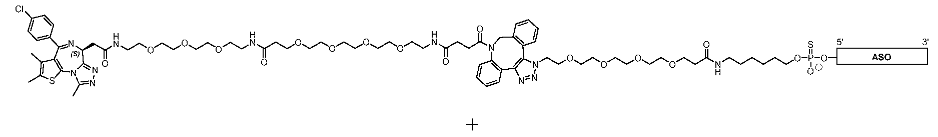 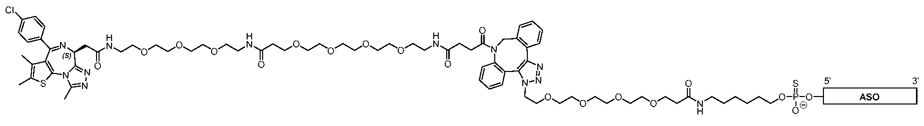 Chemical Structure of PVT1-ASO1-Linker1-JQ1 (isomer 1) 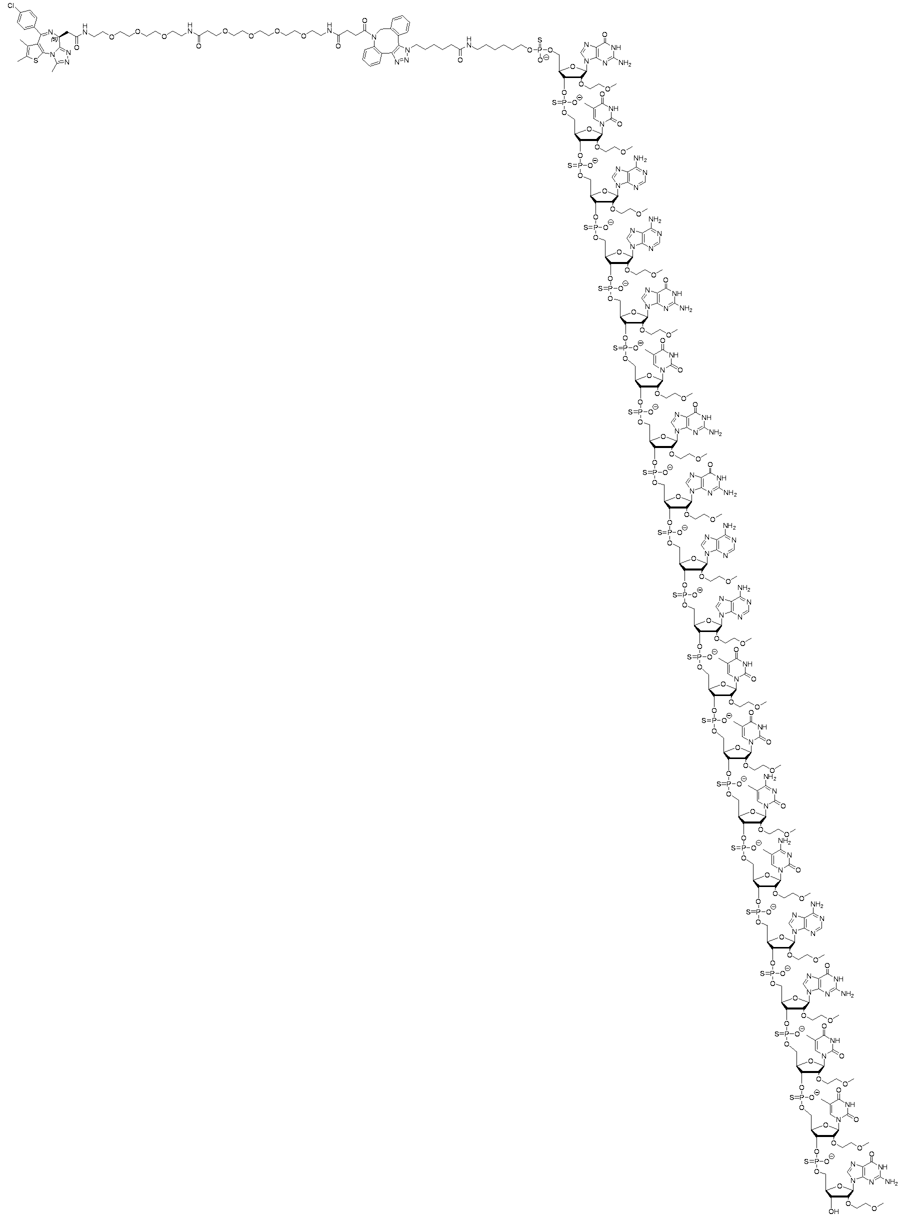 Chemical Structure of PVT1-ASO1-Linkerl-JQ1 (isomer 2) 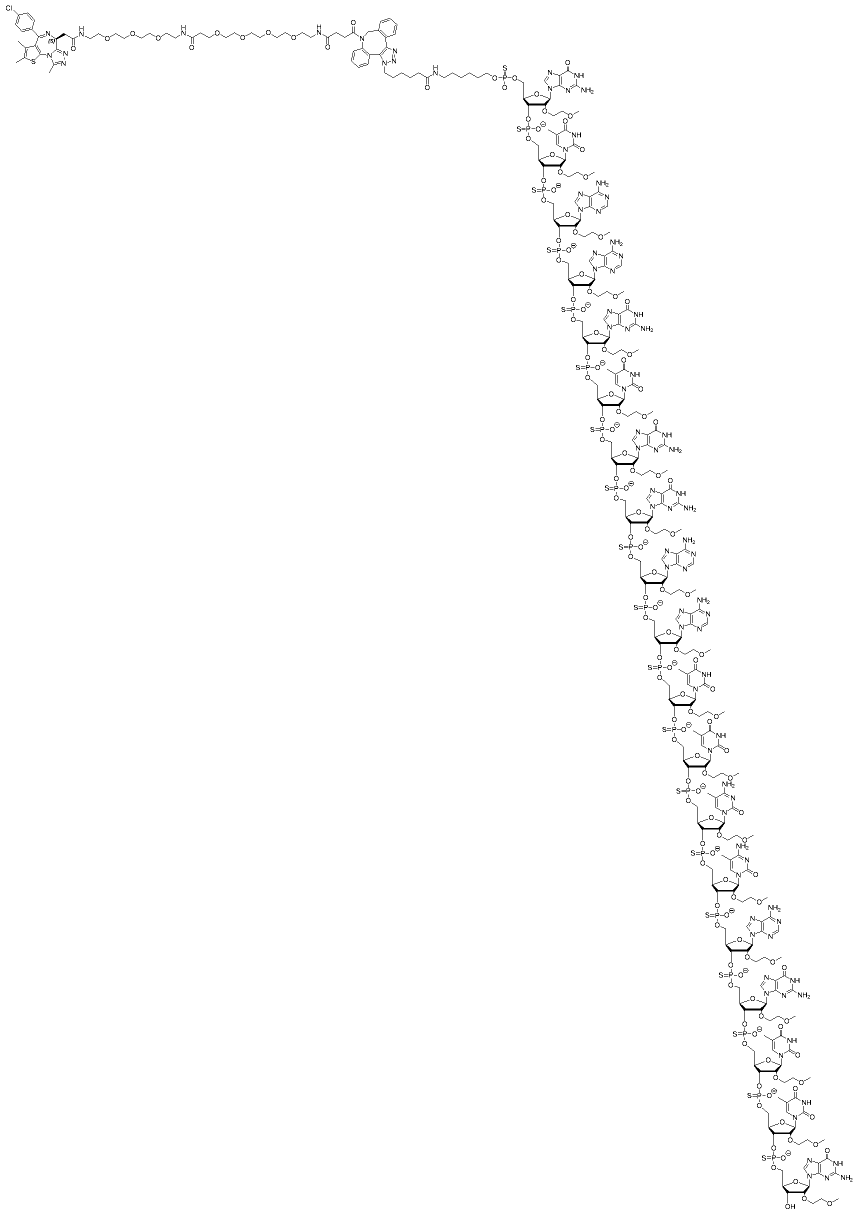 Chemical Structure of PVT1-ASO1-Linker2-JQ1 (isomer 1) 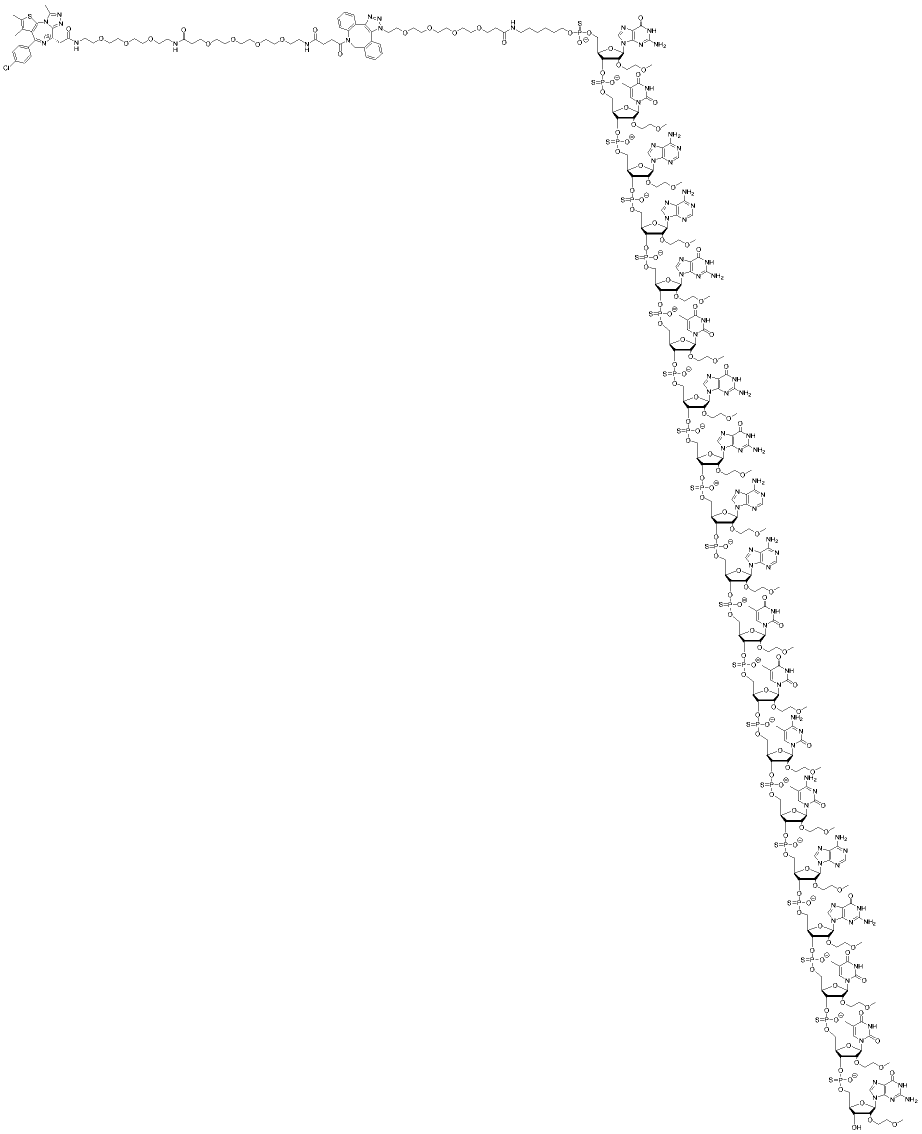 Chemical Structure of PVT1-ASO1-Linker2-JQ1 (isomer 2)

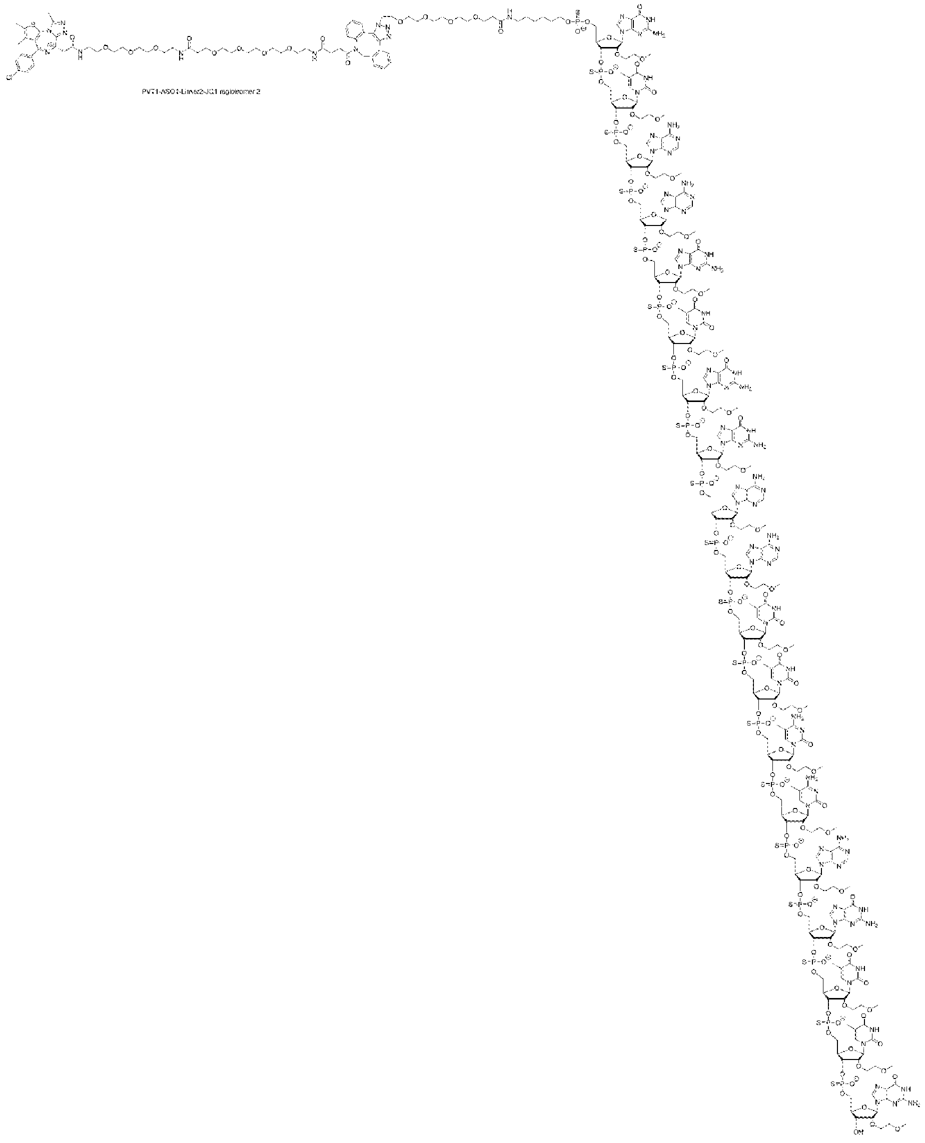

### Example 4b: Transfection of Bi-Functional Molecule

Methods to transfect cells with a bi-functional ASO small molecule were developed.

HEK293T cells were seeded at 30k cells/well in a 96 well tissue culture vessel day before transfection. The next day cells were transfected with 400, 200, 100, 50 nM of PVT1 ASO1-JQ1 with Lipofectamine RNAiMax (ThermoFisher Cat# 13778150). PVT1 ASO1-JQ1:RNAiMax ratios in transfection were: 400 nM:1.2 ul, 200 nM:0.6 ul, 100 nM:0.3 ul, 50 nM:0.15 ul. Transfected cells were allowed to recover and were harvested after 24 hours.

### Example 4c: Measuring MYC Gene Expression

Methods to measure MYC expression levels were developed. It was expected that delivering JQ1 to the vicinity of a gene promoter would recruit BRD4 protein, resulting in the increase of gene expression.

MYC expression was measured by RNA level using qPCR analysis after transfection with each of the bi-functional molecule or control molecules.

Cell samples for qPCR analysis were prepared by Cells to Ct 1 Step TaqMan Kit (ThermoFisher A25602) following manufractuer’s recommendations. qPCR assays were performed using Cells to Ct qPCR master mix, gene specific TaqMan probe (ThermoFisher), and Cells to Ct cell lysate. Relative levels of MYC were normalized to β-actin as a stably expressed control. MYC TaqMan probe: ThermoFisher Assay ID Hs00153408_ml; ACTB TaqMan probe: ThermoFisher Assay ID Hs01060665_g1. During qPCR amplification, FAM fluorescence intensity for each target gene was recorded by QuantStudio7 qPCR instrument (ThermoFisher Scientific) as a measurement of the amount of double-stranded DNA produced during each PCR cycle. Ct values for each gene in each sample were computed by the instrument software based on the amplification curves, and used to determine relative expression values for target and β-actin in each sample.

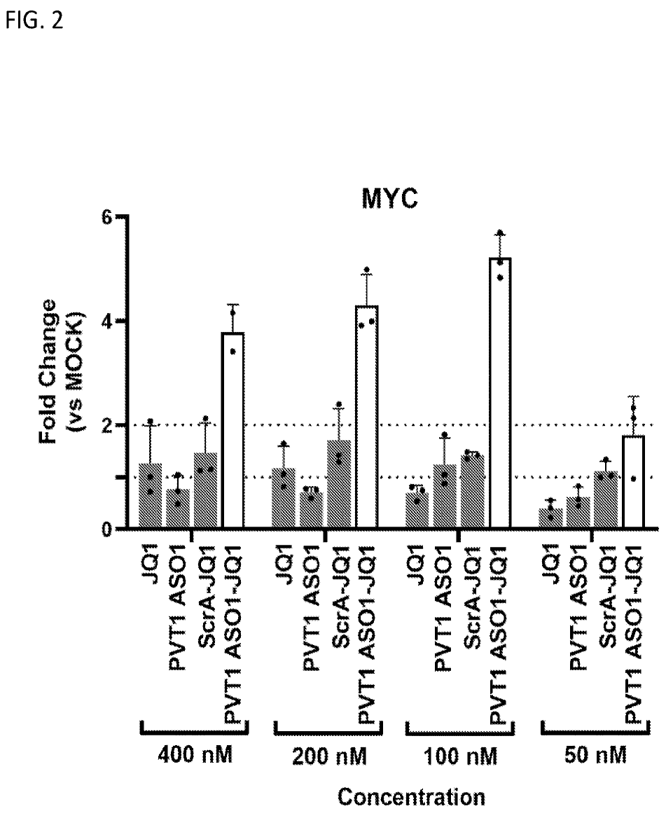

As a result of PVT1 ASO1-JQ1 treatment, an about 4-fold increase in MYC expression was observed while the control molecules were observed not to increase MYC expression (FIG. 2). The results demonstrated that an ASO-small molecule modality can target a lncRNA (long non-coding RNA) and manipulate the expression of another gene.

### Example 5: Specificity of PVT1 ASO1-JQ1 to Increase MYC Expression

Example 5a: ASOs which do not target PVT1 did not increase MYC expression when conjugated to JQ1.

The non-PVT1 targeting ASOs and chemically modified ASOs thereof were synthesized as controls (Tables 6A and 6B) according to Example 1 or purchased from IDT as noted.

TABLE 6A

non-PVT1 targeting ASO (NPT ASO) and scramble ASO Sequences

| | | |
|---|---|---|
| ASO name | Sequence (5′ - 3′) | Human genome coordinate (hg38) |
| Non PVT1 targeting ASO 1¹ | GTCGAATAAACCAGTATC (SEQ ID NO: 52) | chr15:92,884,585-92,884,602 |
| Non PVT1 targeting ASO2 | GATCCAAGTAAATCAGCACGACC (SEQ ID NO: 53) | chr11:118,768,497-118,768,519 |
| Non PVT1 targeting ASO3 | ATAGGTGGTCTCTGATGGTC (SEQ ID NO: 54) | chr11:118,771,186-118,771,205 |
| Non PVT1 targeting ASO4¹ | AGTAAGACTGGGGTTGTT (SEQ ID NO: 55) | chr2:166,036,141-166,036,158 |
| Non PVT1 targeting ASO5 | GTATGTGTACCGCATTGTTT (SEQ ID NO: 56) | chr2:166,069,924-166,069,943 |
| Non PVT1 targeting ASO6¹ | GAGCCAGTCACAAATTCAGATCACC C (SEQ ID NO: 57) | chr2:166,036,680-166,036,705 |
| Non PVT1 targeting ASO7 | TTGTCGTAAGTGTTGCAAAC (SEQ ID NO: 58) | chr2:178,797,518-178,797,537 |
| Non PVT1 targeting ASO8¹ | ACTGAATTCTGACAAATGAC (SEQ ID NO: 59) | chr6:144,292,026-144,292,045 |
| Scramble A (ScrA) | AGAGGTGGCGTGGTAG (SEQ ID NO: 60) | None |
| Scramble B (ScrB) | AACACGTCTATACGCC(SEQ ID NO: 61) | None |

1 Purchased from IDT as 5′-AzideN version

TABLE 6B

Chemical Modifications of non-PVT1 targeting ASO and Scramble ASO

| | |
|---|---|
| ASO name | Chemical modifications to ASO |
| NPT ASO1¹ | */i2MOErG/*/i2MOErT/*/i2MOErC/*/i2MOErG/*/i2MOErA/*/i2MOErA/*/i2MOErT/*/i2MOErA/*/i2MOErA/*/i2MOErA/*/i2MOErC/*/i2MOErC/*/i2MOErA/*/i2MOErG/*/i2MOErT/*/i2MOErA/^{∗}/i2MOErT/^{∗}/32MOErC/ |
| NPT ASO2 | */i2MOErG/*/i2MOErA/*/i2MOErT/*/i2MOErC/*/i2MOErC/*/i2MOErA/*/i2MOErA/*/i2MOErG/*/i2MOErT/*/i2MOErA/*/i2MOErA/*/i2MOErA/*/i2MOErT/*/i2MOErC/*/i2MOErA/*/i2MOErG/^{∗}/i2MOErC/^{∗}/i2MOErA/^{∗}/i2MOErC/^{∗}/i2MOErG/^{∗}/i2MOErA/^{∗}/i2MOErC/^{∗}/32MOErC/ |
| NPT ASO3 | */i2MOErA/*/i2MOErT/*/i2MOErA/*/i2MOErG/*/i2MOErG/*/i2MOErT/*/i2MOErG/*/i2MOErG/*/i2MOErT/*/i2MOErC/*/i2MOErT/*/i2MOErC/*/i2MOErT/*/i2MOErG/*/i2MOErA/*/i2MOErT/^{∗}/i2MOErG/^{∗}/i2MOErG/^{∗}/i2MOErT/^{∗}/32MOErC/ |
| NPT ASO4¹ | *A*+G*+T*A*A*G*+A*C*+T*G*G*G*G*+T*T*+G*+T*+T |
| NPT ASO5 | */i2MOErG/*/i2MOErT/*/i2MOErA/*/i2MOErT/*/i2MOErG/*/i2MOErT/*/i2MOErG/*/i2MOE rT/*/i2MOErA/*/i2MOErC/*/i2MOErC/*/i2MOErG/*/i2MOErC/*/i2MOErA/*/i2MOErT/*/i2MOErT/^{∗}/i2MOErG/^{∗}/i2MOErT/^{∗}/i2MOErT/^{∗}/32MOErT/ |
| NPT ASO6¹ | */i2MOErG/*/i2MOErA/*/i2MOErG/*C*C*A*G*/i2MOErT/*C*A*/i2MOErC/*A*A*A*/i2MOErT/^{∗}T^{∗}C^{∗}A^{∗}G^{∗}/i2MOErA/^{∗}T^{∗}C^{∗}A^{∗}/i2MOErC/^{∗}/i2MOErC/^{∗}/32MOErC/ |
| NPT ASO7 | */i2MOErT/*/i2MOErT/*/i2MOErG/*/i2MOErT/*/i2MOErC/*/i2MOErG/*/i2MOErT/*/i2MOErA/*/i2MOErA/*/i2MOErG/*/i2MOErT/*/i2MOErG/*/i2MOErT/*/i2MOErT/*/i2MOErG/*/i2MOErC/^{∗}/i2MOErA/^{∗}/i2MOErA/^{∗}/i2MOErA/^{∗}/32MOErC/ |
| NPT ASO8¹ | */i2MOErA/*/i2MOErC/*/i2MOErT/*/i2MOErG/*/i2MOErA/*/i2MOErA/*/i2MOErT/*/i2MOErT/*/i2MOErC/*/i2MOErT/*/i2MOErG/*/i2MOErA/*/i2MOErC/*/i2MOErA/*/i2MOErA/*/i2MOErA/^{∗}/i2MOErT/^{∗}/i2MOErG/^{∗}/i2MOErA/^{∗}/32MOErC/ |
| ScrA | */i2MOErA/*/i2MOErG/*/i2MOErA/*/i2MOErG/*/i2MOErG/*/i2MOErT/*/i2MOErG/*/i2MOErG/*/i2MOErC/*/i2MOErG/*/i2MOErT/*/i2MOErG/*/i2MOErG/*/i2MOErT/*/i2MOErA/*/32MOErG/ |
| ScrB | /i2MOErA/*/i2MOErA/*/i2MOErC/*/i2MOErA/*/i2MOErC/*/i2MOErG/*/i2MOErT/*/i2MOErC/*/i2MOErT/*/i2MOErA/*/i2MOErT/*/i2MOErA/*/i2MOErC/*/i2MOErG/*/i2MOErC/*/32MOErC/ |

1 Purchased from IDT as 5′-AzideN version

Table 6A shows non-PVT1 targeting control ASO and scramble ASO sequences and their coordinates in the human genome. Table 6B shows chemical modifications for each ASO. Mod Code follows IDT Mod Code: + = LNA, * = Phosphorothioate linkage, “r” signifies ribonucleotide, i2MOErA = internal 2′-MethoxyEthoxy A, i2MOErC = internal 2′-MethoxyEthoxy MeC, 32MOErA = 3′-Hydroxy-2′-MethoxyEthoxy A etc.

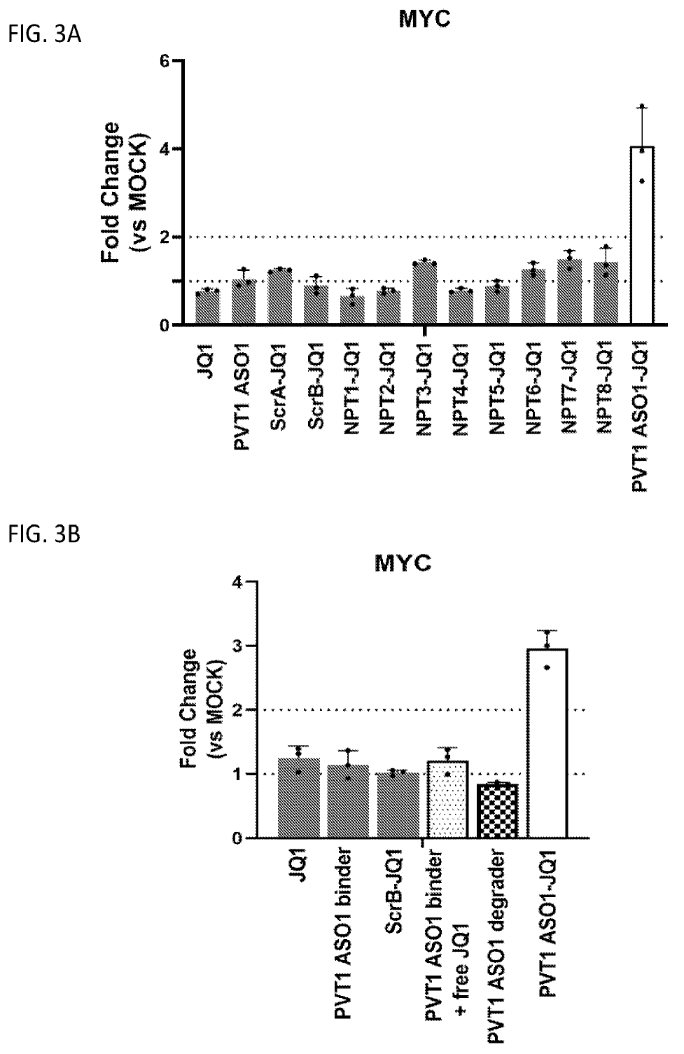

JQ1 conjugated to two scrambled sequences and eight non-PVT1 targeting sequences above, synthesized according to Example 2, were transfected to HEK293T cells at 100 nM with 0.3 ul of RNAiMax. Cells were harvested 24 hours after transfection and MYC expression changes was monitored by qPCR. Results from the test showed that none of the 10 JQ1 conjugates induced MYC expression above background levels (FIG. 3A).

Example 5b: It was demonstrated that covalent linkage of PVT1 ASO1 and JQ1 is essential to increase MYC expression, and treating cells with PVT1 ASO1 degrader does not increase MYC expression

(PVT1 ASO1+ free JQ1) and PVT1 ASO1 degrader (an LNA/DNA gapmer with a 3-13-3 motif and a phosphorothioate backbone modification, purchased from Qiagen with the following sequence: +G*+T*+A*A*G*T*G*G*A*A*T*T*C*C*A*G*+T*+T*+G) were transfected to HEK293T cells at 100 nM with RNAiMax. 0.3 ul of RNAiMax was used for each well for transfection. Cells were harvested 24 hours after transfection and MYC expression changes was monitored by qPCR. Results from the test showed that (PVT1 ASO1 + JQ1) and PVT1 ASO1 degrader were both inactive to increase MYC expression (FIG. 3B).

Example 5c: The critical role of small molecule inhibitor JQ1 in increasing MYC expression was demonstrated.

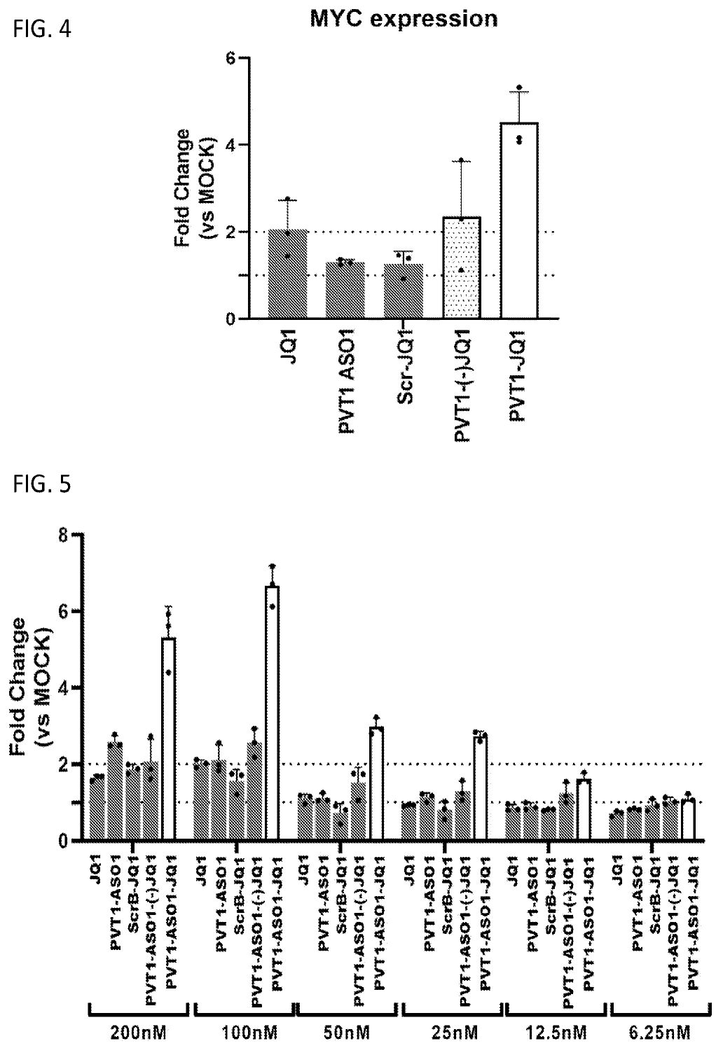

(-)JQ1 is an enantiomer of JQ1 and has >100x weaker biochemical activity (thesgc.org/chemical-probes/JQ1) as compared to JQ1. PVT1 ASO1-(-)JQ1 was transfected to HEK293T cells at 100 nM with RNAiMax. 0.3 ul of RNAiMax was used for each well for transfection. Cells were harvested 24 hours after transfection and MYC expression changes was monitored by qPCR. Results from the test showed that PVT1 ASO1-(-)JQ1 was inactive to increase MYC expression above background (FIG. 4).

Example 5d: The dose dependent response of MYC expression upon the titration of PVT1 ASO1-JQ1 was demonstrated.

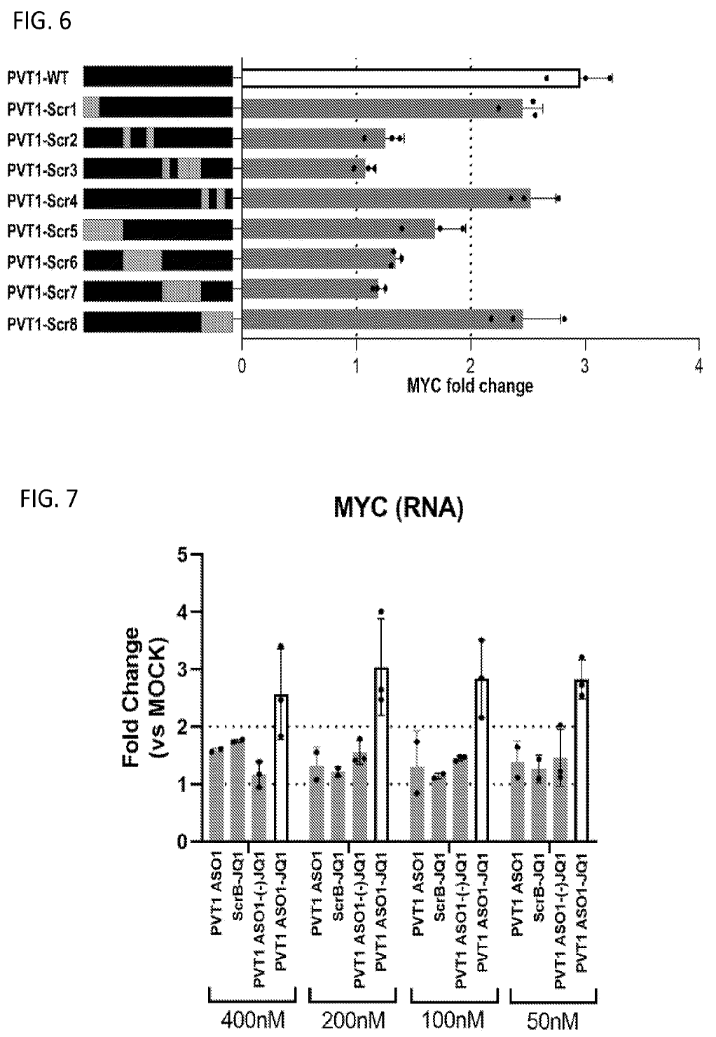

PVT1 ASO1-JQ1 and control molecules were transfected to HEK293T cells at 200, 100, 50, 25, 12.5, 6.25, and 3.125 nM with RNAiMax. PVT1 ASO1-JQ1:RNAiMax ratios in transfection were: 200 nM:0.6 ul, 100 nM:0.3 ul, 50 nM and below:0.15 ul. Cells were harvested 24 hours after transfection and MYC expression changes were monitored by qPCR. Results from the test showed a dose dependent response of MYC expression changes (FIG. 5). The slight decrease of MYC response at 200 nM could be the result of a hook effect (EBioMedicine. 2018 Oct; 36: 553-562) observed in bifunctional compound treatments.

Example 5e: The requirement of PVT1 ASO1 sequence in inducing MYC expression was demonstrated.

Table 7 below lists nucleotide sequences and chemical modifications of PVT1 ASO1 and eight PVT1 scrambled ASO synthesized in this example, synthesized according to Example 1. Mod Code follows IDT Mod Code: + = LNA, * = Phosphorothioate linkage, “r” signifies ribonucleotide, i2MOErA = internal 2′-MethoxyEthoxy A, i2MOErC = internal 2′-MethoxyEthoxy MeC, 32MOErA = 3′-Hydroxy-2′-MethoxyEthoxy A, etc.

TABLE 7

PVT1-ASO1 and PVT1-scrambled ASO sequences and nucleotide modifications

| | | |
|---|---|---|
| ASO name | Sequence (5′ - 3′) | Nucleotide Modification |
| PVT1- ASO1 | GTAAGTGGAATTCCA GTTG (SEQ ID NO: 62) | ^{∗}/i2MOErG/^{∗}/i2MOErT/^{∗}/i2MOErA/^{∗}/i2MOErA/^{∗}/i2MOErG/ ^{∗}/i2MOErT/^{∗}/i2MOErG/^{∗}/i2MOErG/^{∗}/i2MOErA/^{∗}/i2MOErA/ */i2MOErT/*/i2MOErT/*/i2MOErC/*/i2MOErC/*/i2MOErA/* /i2MOErG/^{∗}/i2MOErT/^{∗}/i2MOErT/^{∗}/32MOErG/ |
| PVT1-ASO1-scr1 | TGAAGTGGAATTCCA GTTG (SEQ ID NO: 63) | ^{∗}/i2MOErT/^{∗}/i2MOErG/^{∗}/i2MOErA/^{∗}/i2MOErA/^{∗}/i2MOErG/ ^{∗}/i2MOErT/^{∗}/i2MOErG/^{∗}/i2MOErG/^{∗}/i2MOErA/^{∗}/i2MOErA/ */i2MOErT/*/i2MOErT/*/i2MOErC/*/i2MOErC/*/i2MOErA/* /i2MOErG/^{∗}/i2MOErT/^{∗}/i2MOErT/^{∗}/32MOErG/ |
| PVT1-ASO1-scr2 | GTAAGAGGTATTCCA GTTG (SEQ ID NO: 64) | ∗/i2MOErG/^{∗}/i2MOErT/^{∗}/i2MOErA/^{∗}/i2MOErA/^{∗}/i2MOErG/ ^{∗}/i2MOErA/^{∗}/i2MOErG/^{∗}/i2MOErG/^{∗}/i2MOErT/^{∗}/i2MOErA/ */i2MOErT/*/i2MOErT/*/i2MOErC/*/i2MOErC/*/i2MOErA/* /i2MOErG/^{∗}/i2MOErT/^{∗}/i2MOErT/^{∗}/32MOErG/ |
| PVT1-ASO1-scr3 | GTAAGTGGAACTATC GTTG (SEQ ID NO: 65) | ^{∗}/i2MOErG/^{∗}/i2MOErT/^{∗}/i2MOErA/^{∗}/i2MOErA/^{∗}/i2MOErG/ ^{∗}/i2MOErT/^{∗}/i2MOErG/^{∗}/i2MOErG/^{∗}/i2MOErA/^{∗}/i2MOErA/ */i2MOErC/*/i2MOErT/*/i2MOErA/*/i2MOErT/*/i2MOErC/* /i2MOErG/^{∗}/i2MOErT/^{∗}/i2MOErT/^{∗}/32MOErG/ |
| PVT1-ASO1-scr4 | GTAAGTGGAATTCCA TTGG (SEQ ID NO: 66) | ^{∗}/i2MOErG/^{∗}/i2MOErT/^{∗}/i2MOErA/^{∗}/i2MOErA/^{∗}/i2MOErG/ ^{∗}/i2MOErT/^{∗}/i2MOErG/^{∗}/i2MOErG/^{∗}/i2MOErA/^{∗}/i2MOErA/ */i2MOErT/*/i2MOErT/*/i2MOErC/*/i2MOErC/*/i2MOErA/* /i2MOErT/^{∗}/i2MOErT/^{∗}/i2MOErG/^{∗}/32MOErG/ |
| PVT1-ASO1-scr5 | TAGGATGGAATTCCA GTTG (SEQ ID NO: 67) | ∗/i2MOErT/^{∗}/i2MOErA/^{∗}/i2MOErG/^{∗}/i2MOErG/^{∗}/i2MOErA/ ^{∗}/i2MOErT/^{∗}/i2MOErG/^{∗}/i2MOErG/^{∗}/i2MOErA/^{∗}/i2MOErA/ */i2MOErT/*/i2MOErT/*/i2MOErC/*/i2MOErC/*/i2MOErA/* /i2MOErG/^{∗}/i2MOErT/^{∗}/i2MOErT/^{∗}/32MOErG/ |
| PVT1-ASO1-scr6 | GTAAGATAGGTTCCA GTTG (SEQ ID NO: 68) | ^{∗}/i2MOErG/^{∗}/i2MOErT/^{∗}/i2MOErA/^{∗}/i2MOErA/^{∗}/i2MOErG/ ^{∗}/i2MOErA/^{∗}/i2MOErT/^{∗}/i2MOErA/^{∗}/i2MOErG/^{∗}/i2MOErG/ */i2MOErT/*/i2MOErT/*/i2MOErC/*/i2MOErC/*/i2MOErA/* /i2MOErG/^{∗}/i2MOErT/^{∗}/i2MOErT/^{∗}/32MOErG/ |
| PVT1-ASO1-scr7 | GTAAGTGGAACATTC GTTG (SEQ ID NO: 69) | ^{∗}i2MOErG/^{∗}/i2MOErT/^{∗}/i2MOErA/^{∗}/i2MOErA/^{∗}/i2MOErG/ ^{∗}/i2MOErT/^{∗}/i2MOErG/^{∗}/i2MOErG/^{∗}/i2MOErA/^{∗}/i2MOErA/ */i2MOErC/*/i2MOErA/*/i2MOErT/*/i2MOErT/*/i2MOErC/* /i2MOErG/^{∗}/i2MOErT/^{∗}/i2MOErT/^{∗}/32MOErG/ |
| PVT1-ASO1-scr8 | GTAAGTGGAATTCCA TGGT (SEQ ID NO: 70) | ^{∗}/i2MOErG/^{∗}/i2MOErT/^{∗}/i2MOErA/^{∗}/i2MOErA/^{∗}/i2MOErG/ ^{∗}/i2MOErT/^{∗}/i2MOErG/^{∗}/i2MOErG/^{∗}/i2MOErA/^{∗}/i2MOErA/ */i2MOErT/*/i2MOErT/*/i2MOErC/*/i2MOErC/*/i2MOErA/* /i2MOErT/^{∗}/i2MOErG/^{∗}/i2MOErG/^{∗}/32MOErT/ |

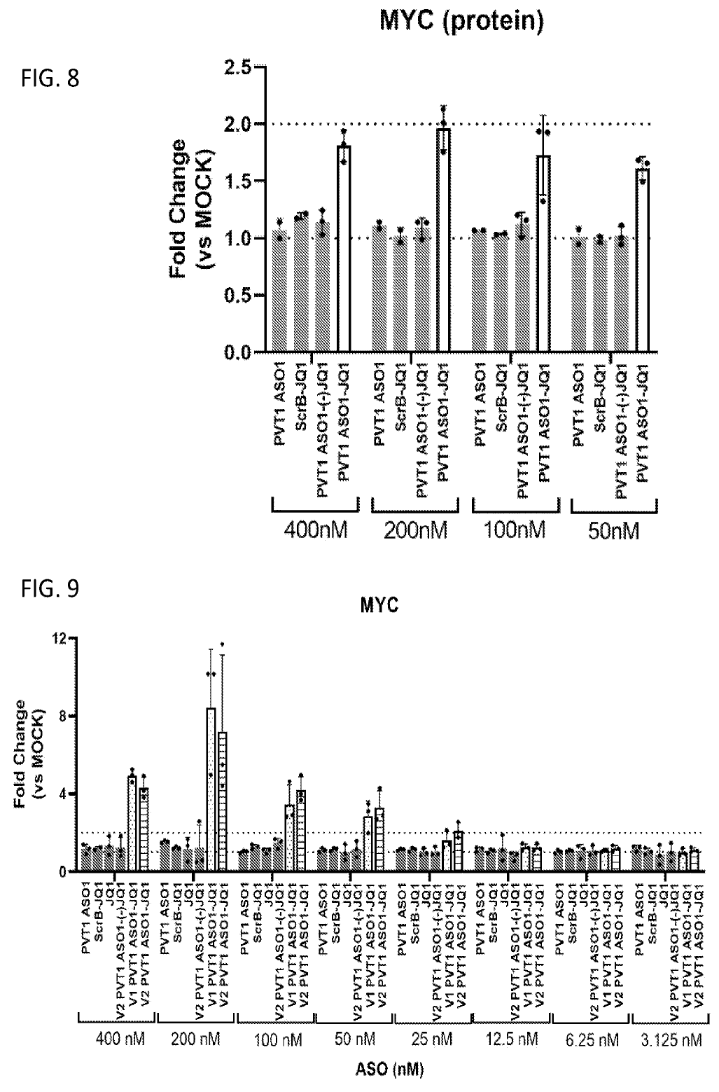

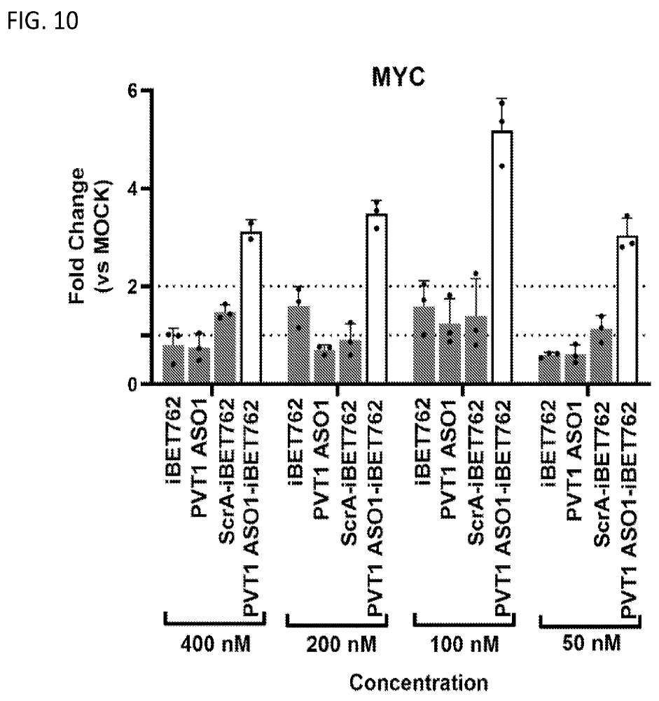

Between 2 to 5 nucleotides within PVT1 ASO1 sequence were swapped to generate 8 partially scrambled PVT1 ASO1 sequences (Table 7). Scrambled PVT1 ASO1-JQ1 molecules were transfected to HEK293T cells at 100 nM with 0.3 ul RNAiMax per 96 well. Cells were harvested 24 hours after transfection and MYC expression changes were monitored by qPCR. Results from the test showed that swapping nucleotides at both ends of PVT1-ASO1 have less impact on the activities of PVT1 ASO1-JQ1, while swapping as little as two nucleotides within the middle 10 nucleotides significantly reduced the activites (FIGS. 6 and 7).

### Example 6: This Example Demonstrates That PVT1 ASO1-JQ1 Treatment Increases MYC Gene Transcript (FIG. 7) and also MYC Protein (FIG. 8) in Cells

PVT1 ASO1-JQ1 and control molecules were transfected to HEK293T cells at 400, 200, 100, and 50 nM with RNAiMax. PVT1 ASO1-JQ1:RNAiMax ratios in transfection are: 400 nM: 1.2 ul, 200 nM:0.6 ul, 100 nM:0.3 ul, 50 nM:0.15 ul. Cells were harvested 24 hours after transfection and MYC expression changes was monitored by qPCR and by enzyme-linked immunosorbent assay (ELISA). Results from the qPCR test showed an increase of MYC RNA transcripts (FIG. 7). For a fluorescence resonance energy transfer (FRET) based ELISA assays, cell samples were prepared by Human c-Myc Cell-based kit (Cisbio # 63ADK053PEH) following manufractuer’s recommendation. MYC protein is detected in a sandwich assay using two specific antibodies, labeled with Europium Cryptate (donor) and with d2 (acceptor). FRET signal was read with Varioskan LUX Multimode Microplate Reader (ThermoFisher) with a 6 hr kinetic read. Results from the ELISA assay showed that at 200 nM PVT1 ASO1-JQ1, MYC protein level increased by about 2 fold at 24 hours (FIG. 8).

### Example 7: Use of Different Chemical Linkers to Covalently Conjugate JQ1 and PVT1 ASO1 While Maintaining the Acitivites of the Compounds

PVT1 ASO1-Linker1-JQ1 was synthesized according to Example 1 and Example 2, using 6-azidohexanoic acid NHS ester in place of N3-PEG4-NHS ester. PVT1-ASO1-Linker2-JQ1 was synthesized according to Example 1 and Example 2.

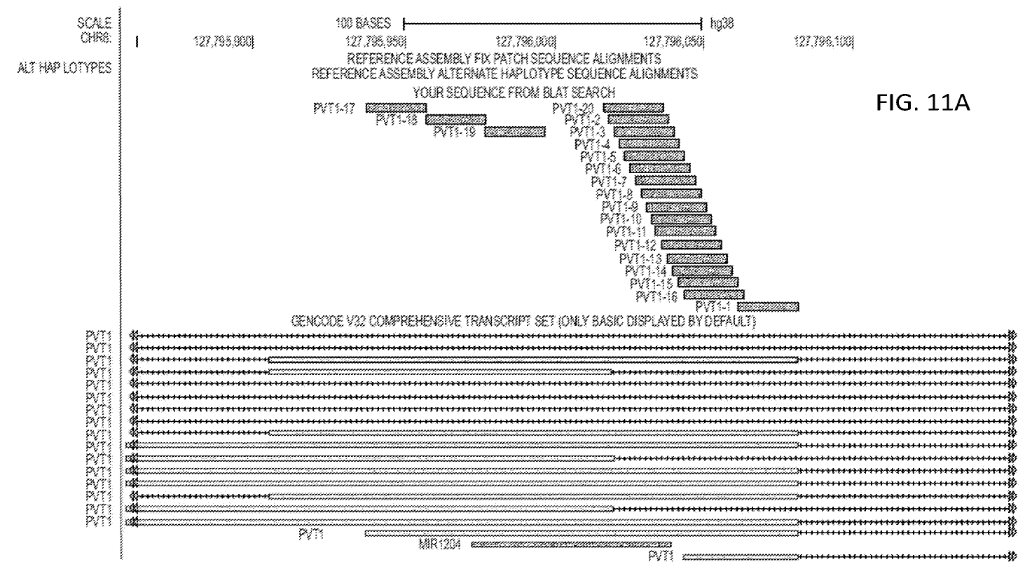

PVT1-ASO1-Linker1-JQ1 (V1-PVT1 ASO1-JQ1) and PVT1-ASO1-Linker2-JQ1 (V2-PVT1 ASO1-JQ1) were transfected to HEK293T cells at 400, 200, 100, 50, 25, 12.5, 6.25, and 3.125 nM with RNAiMax. PVT1 ASO1-JQ1:RNAiMax ratios in transfection were: 400 nM:1.2 ul, 200 nM:0.6 ul, 100 nM:0.3 ul, 50 nM and below:0.15 ul. Cells were harvested 24 hours after transfection and MYC expression changes were monitored by qPCR. Results from the test showed that molecules using V1 and V2 linkers were both active and increased MYC expression to similar levels (FIG. 9).

### Example 8: An Additional BET Inhibitor to Substitute JQ1 in PVT1 ASO-JQ1 Molecule

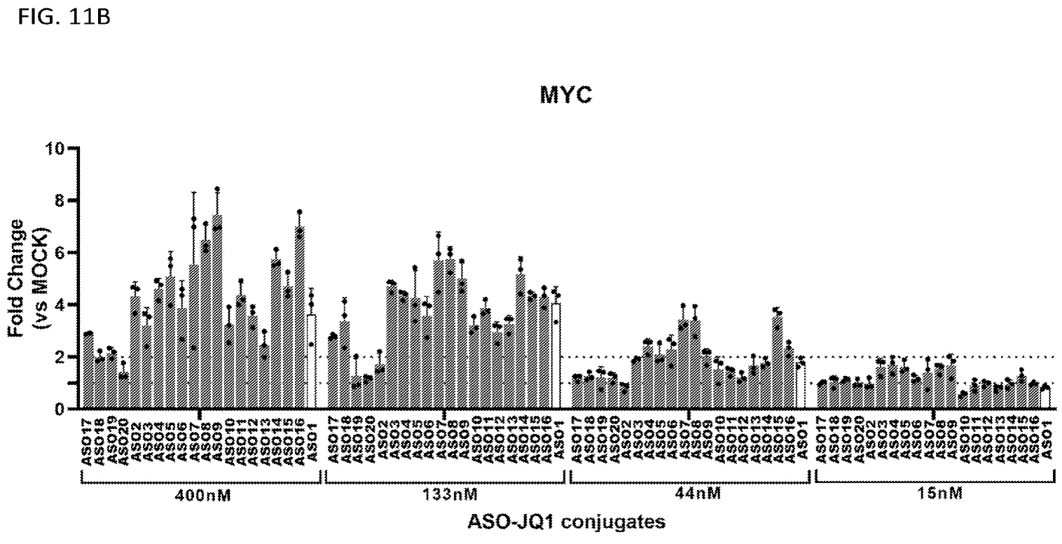

PVT1 ASO1-Linker1-iBET762, synthesized according to Example 1 and Example 2 using DBCO-PEG4-iBET762 (synthesized from DBCO-PEG4-NHS and amino-PEG3-iBET762), was transfected to HEK293T cells at 400, 200, 100, and 50 nM with RNAiMax.PVT1 ASO1-iBET762:RNAiMax ratios in transfection were: 400 nM:1.2 ul, 200 nM:0.6 ul, 100 nM:0.3 ul, 50 nM: 0.15 ul. Cells were harvested 24 hours after transfection and MYC expression changes were monitored by qPCR. Results from the test showed that treatment of PVT1 ASO1-iBET762 also increases MYC expression (FIG. 10).

The chemical structure of PVT1-ASO1-Linker1-iBET762 (regioisomer 1) is

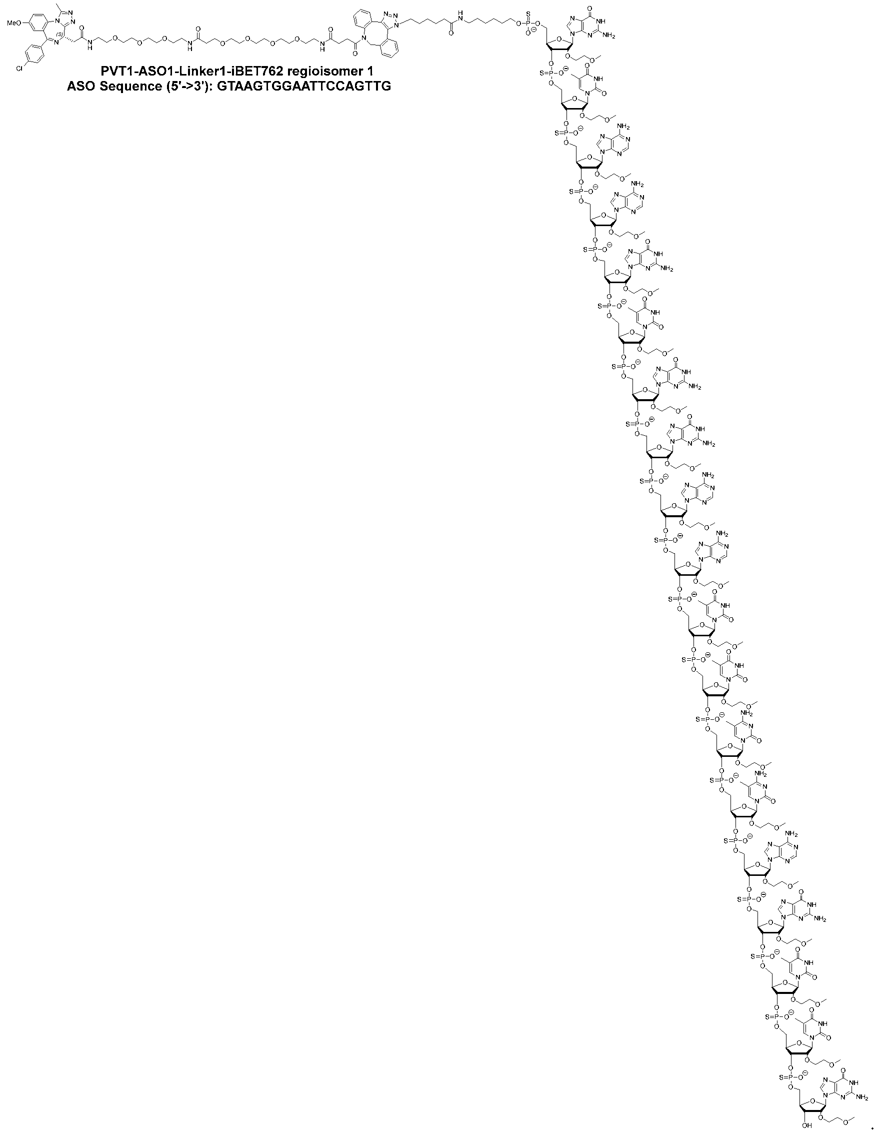

The chemical structure of PVT1-ASO1-Linker1-iBET762 (regioisomer 2) is

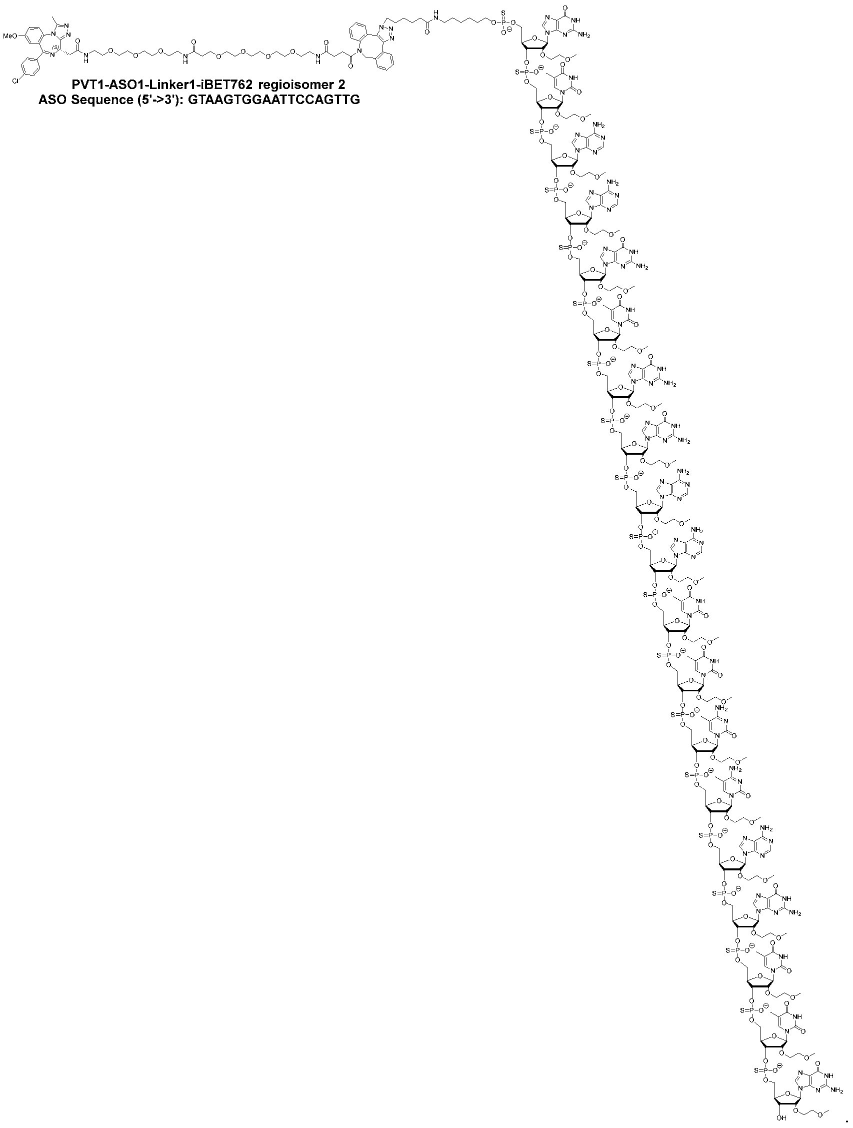

### Example 9: Increase in MYC Expression Using Additional PVT1 ASOs 3′ to ASO1, When Conjugated to JQ1

The synthesis of PVT1 ASO2-ASO20 conjugated to JQ1 with linker 2 is carried out according the the procedure described in Example 1 and Example 2

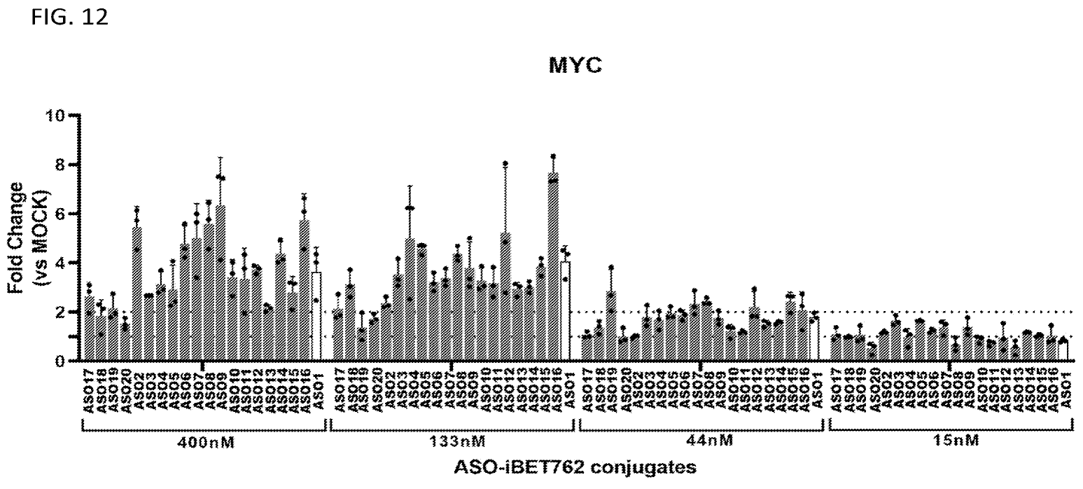

PVT1 ASO2 to ASO20 were designed 3′ to PVT1 ASO1, or more upstream from PVT1 ASO1 annealing site on PVT1 transcript (FIG. 11A). PVT1 ASO2-Linker2-JQ1 to PVT1 ASO20-Linker2-JQ1 were transfected to HEK293T cells at 400, 133, 44, and 15 nM with RNAiMax. PVT1 ASO-JQ1:RNAiMax ratios in transfection are: 400 nM:1.2 ul, 133 nM:0.4 ul, 44 nM:0.13 ul, 15 nM:0.13 ul. Cells were harvested 24 hours after transfection and MYC expression changes was monitored by qPCR. Results from the test demonstrated that at 133 nM, PVT1 ASO3-JQ1 - PVT1 ASO16-JQ1 showed similar levels of activities as PVT1 ASO1-JQ1. (FIG. 11B).

### Example 10: Increase in MYC Expression Using Additional PVT1 ASOs, When Conjugated to Ibet762

The synthesis of PVT1 ASO2-ASO20 conjugated to iBET762 with linker 2 is carried out according the the procedure described in Example 1 and Example 2, using DBCO-PEG4-iBET762 (synthesized from DBCO-PEG4-NHS and amino-PEG3-iBET762).

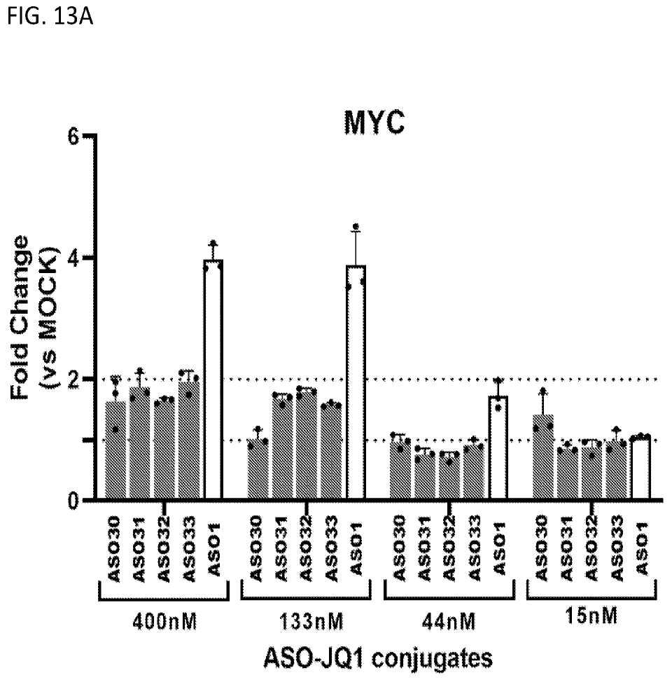

PVT1 ASO2-Linker2-iBET762 to PVT1 ASO20-Linker2-iBET762 were transfected to HEK293T cells at 400, 133, 44, and 15 nM with RNAiMax. PVT1 ASO-iBET762:RNAiMax ratios in transfection are: 400 nM:1.2 ul, 133 nM:0.4 ul, 44 nM:0.13 ul, 15 nM:0.13 ul. Cells were harvested 24 hours after transfection and MYC expression changes was monitored by qPCR. Results from the test demonstrated that PVT1 ASO3―Linker2-iBET762 - PVT1 ASO16-Linker2-iBET762 showed similar levels of activities as PVT1 ASO1-Linker2-JQ1. (FIG. 12).

### Example 11: An Active Pocket Defined on PVT1 When ASOs Designed From Within the Boundary Are Active to Increase MYC Expression When Conjugated to JQ1

The synthesis of PVT1 ASO30-ASO33 conjugated to JQ1 with linker 2 is carried out according the the procedure described in Example 1 and Example 2.

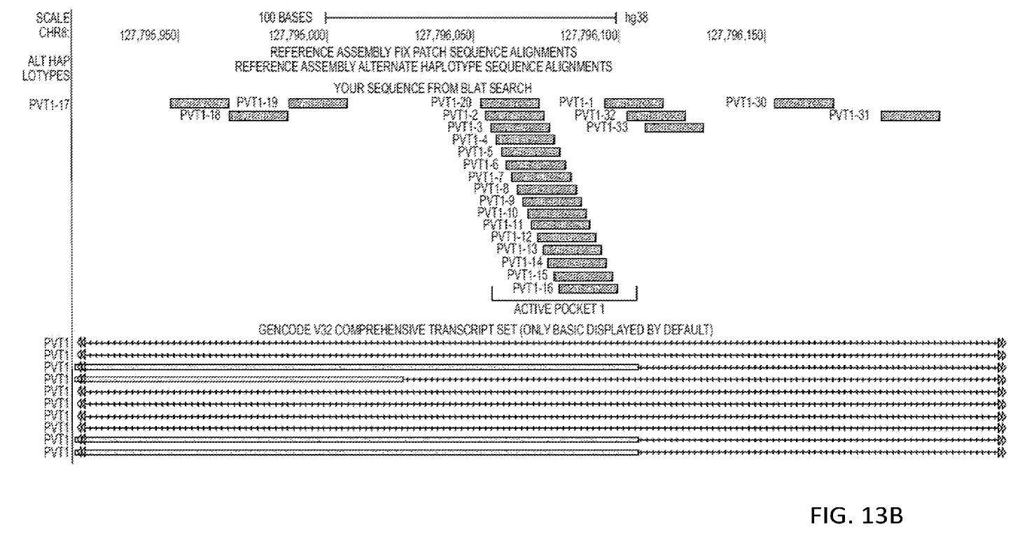

PVT1 ASO30-Linker2-JQ1 to PVT1 ASO33-Linker2-JQ1 were transfected to HEK293T cells at 400, 133, 44, and 15 nM with RNAiMax. PVT1 ASO-JQ1:RNAiMax ratios in transfection were: 400 nM:1.2 ul, 133 nM:0.4 ul, 44 nM:0.13 ul, 15 nM:0.13 ul. Cells were harvested 24 hours after transfection and MYC expression changes were monitored by qPCR. Results from the test demonstrated that PVT1 ASO30-JQ1 to PVT1 ASO33-JQ1 were inert to increase MYC expression. (FIG. 13A). Combining the results from Examples 9 and 11, an active pocket of about 51 nucleotides (Chr8: 127796018-127796068) was identified along an exonic region of PVT1 gene where all ASOs targeting this region increased MYC expression by more than 2-fold at 133 nM (FIG. 13A, FIG. 13B, and FIG. 11B).

### Example 12: Increase in MYC Expression Using Additional PVT1 ASOs 5′ to ASO1, When Conjugated To JQ1

The synthesis of PVT1 ASO21-ASO29 conjugated to JQ1 with linker 2 is carried out according the the procedure described in Example 1 and Example 2.

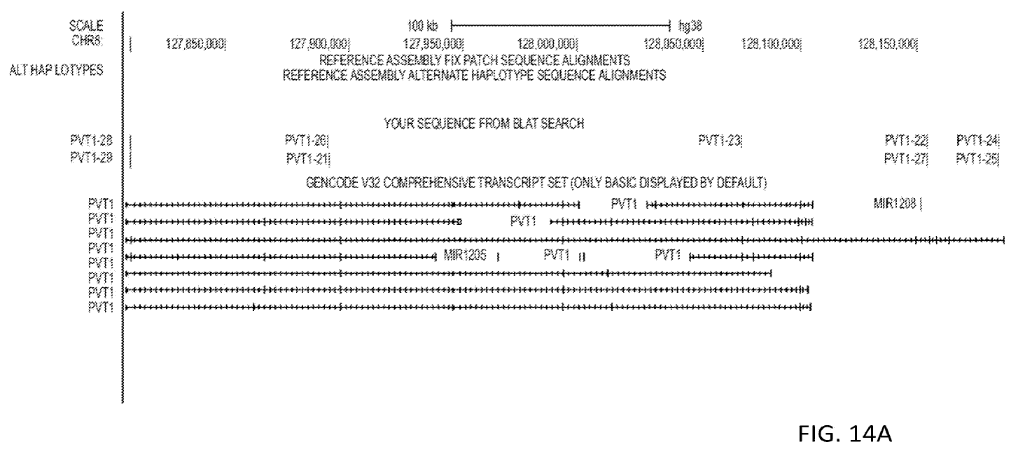

Genomic localization of PVT1 ASO21 to ASO29 was shown (FIG. 14A). PVT1 ASO21-Linker2-JQ1 to PVT1 ASO29-Linker2-JQ1 were transfected to HEK293T cells at 400, 133, 44, and 15 nM with RNAiMax. PVT1 ASO-JQ1:RNAiMax ratios in transfection were: 400 nM:1.2 ul, 133 nM:0.4 ul, 44 nM:0.13 ul, 15 nM:0.13 ul. Cells were harvested 24 hours after transfection and MYC expression changes were monitored by qPCR. Results from the test demonstrated that PVT1 ASO24-JQ1 and PVT1 ASO25-JQ1 increased MYC expression level similar to PVT1 ASO1-JQ1 and defined a second active pocket about 65 nucleotides in size (Chr8:128186661-128186726) within the last exon of PVT1 gene that supported the manipulation of MYC expression when ASOs were designed against this region (FIG. 14B). The identified active pocket (active pocket 2) is indicated in FIG. 14C.

### Example 13: Manipulation of MYC Expression by Targeting MYC Pre-mRNA With MYC ASO-JQ1

The synthesis of MYC-ASO1-ASO6 conjugated to JQ1 with linker 2 is carried out according the procedure described in Example 1 and Example 2.

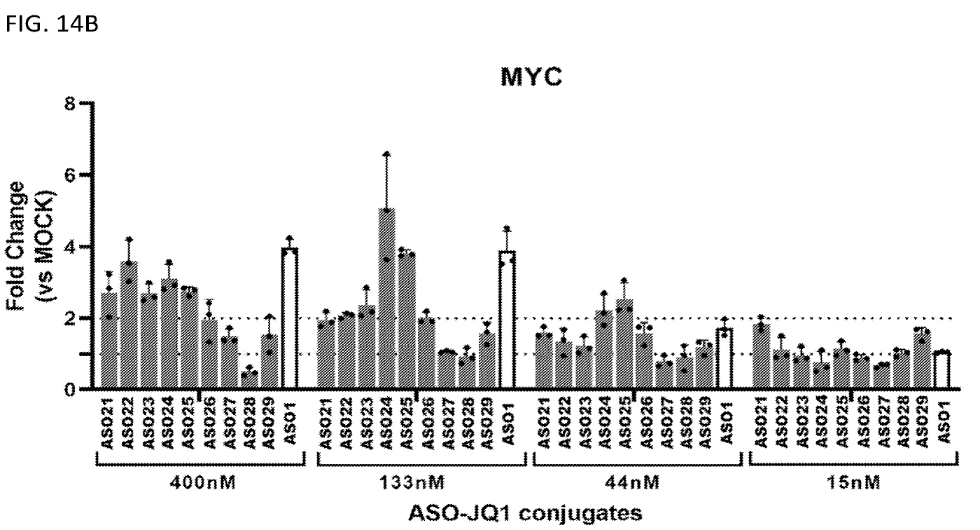

MYC-ASOs 1 to 6 shown in Table 1A were designed against the intronic region of MYC pre-mRNA. MYC-ASO1-Linker2-JQ1 to MYC-ASO6-Linker2-JQ1 were transfected to HEK293T cells at 400, 133, 44, and 15 nM with RNAiMax. PVT1 ASO-JQ1:RNAiMax ratios in transfection are: 400 nM:1.2 ul, 133 nM:0.4 ul, 44 nM:0.13 ul, 15 nM:0.13 ul. Cells were harvested 24 hours after transfection and MYC expression changes were monitored by qPCR. Results from the test demonstrated that MYC ASO3-JQ1, MYC ASO4-JQ1, and MYC ASO6-JQ1 molecules increased MYC expression by more than 2 fold at 133 nM. (FIG. 15). The results demonstrated that an ASO-SM modality can target an intronic region of a pre-mRNA to manipulate the expression of the self gene.

### Example 14: Manipulation of MYC Expression by Targeting MYC Pre-mRNA With MYC ASO-iBET762

MYC ASO1-ASO6 conjugated to iBET762 with linker 2 is synthesized according to Example 1 and Example 2 using DBCO-PEG4-iBET762 (synthesized from DBCO-PEG4-NHS and amino-PEG3-iBET762) in place of DBCO-PEG4-JQ1.

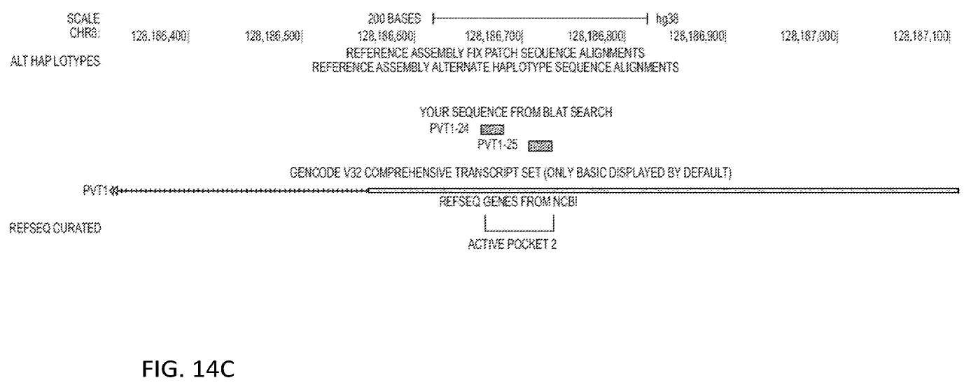

MYC ASO1-Linker2-iBET762 to MYC ASO6-Linker2-iBET762 were transfected to HEK293T cells at 400, 133, 44, and 15 nM with RNAiMax. PVT1 ASO-iBET762:RNAiMax ratios in transfection were: 400 nM:1.2 ul, 133 nM:0.4 ul, 44 nM:0.13 ul, 15 nM:0.13 ul. Cells were harvested 24 hours after transfection and MYC expression changes were monitored by qPCR. Results from the test demonstrated that MYC ASO3-iBET762, MYC ASO4-iBET762, and MYC ASO6-iBET762 molecules increased MYC expression by more than 2-fold at 133 nM (FIG. 16).

### Example 15: Manipulation of SCN1A Expression by Targeting SCN1A mRNA With SCN1A ASO-JQ1

SCN1A ASO1 was purchased from IDT as the 5′ Azide-N modified LNA mixmer (A*+G*+T*A*A*G*+A*C*+T*G*G*G*G*+T*T*+G*+T*+T). It is conjugated to JQ1 according the the procedure described in Example 2.

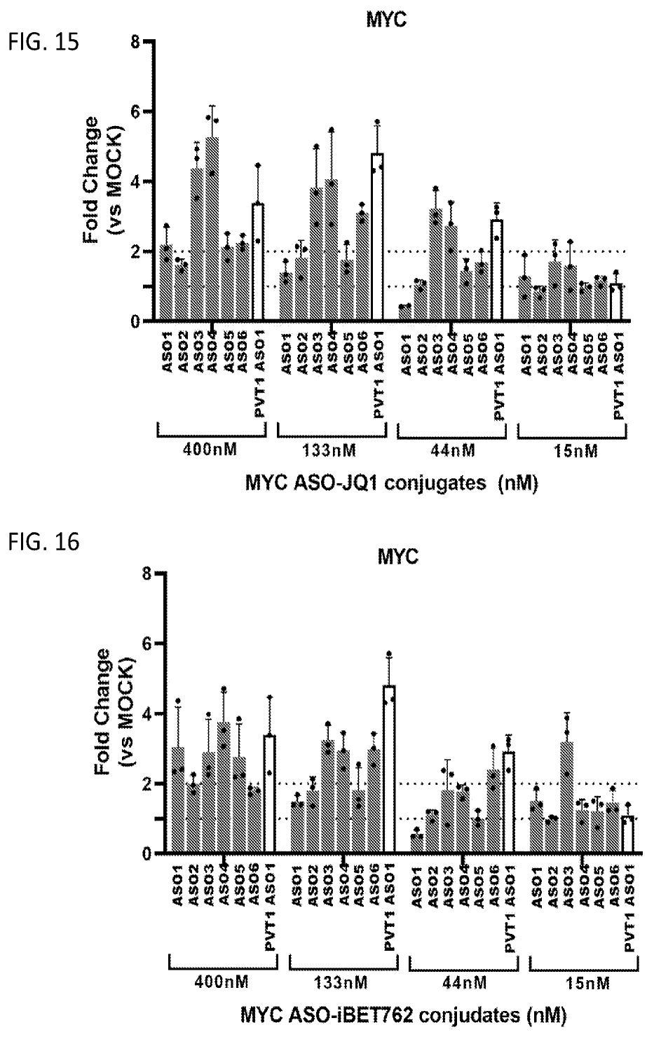

SCN1A-ASO1 shown in Table 5A was designed against the exonic region of SCN1A mRNA. SCN1A ASO1-Linker1-JQ1 was transfected to SK-N-AS cells at 100, 50, 25, 12.5, 6.25, and 3.125 nM with RNAiMax. SCN1A ASO1-JQ1:RNAiMax ratios in transfection were: 100 nM:0.3 ul, 50 nM and below:0.15 ul. Cells were harvested 48 hours after transfection and SCN1A expression changes were monitored by qPCR. TaqMan probe used in the assay for quantitation: SCN1A Hs00374696_ml (ThermoFisher), GAPDH Hs02786624_g1 (ThermoFisher). Results from the test showed that SCN1A ASO1-JQ1 increased SCN1A expression by about 2-fold (FIG. 17). The results demonstrated that an ASO-SM modality could target an exonic region of an mRNA to manipulate the expression of the self gene.

### Example 16: Manipulation of SCN1A Expression by Targeting SCN1A mRNA With SCN1A ASO-iBET762

SCN1A-ASO1 was purchased from IDT as the 5′ Azide-N modified LNA/DNA mixmer with a phosphorothioate backbone (A*+G*+T*A*A*G*+A*C*+T*G*G*G*G*+T*T*+G*+T*+T). It is conjugated to iBET762 according the the procedure described in Example 2 using DBCO-PEG4-iBET762 (synthesized from DBCO-PEG4-NHS and amino-PEG3-iBET762) in place of DBCO-PEG4-JQ1.

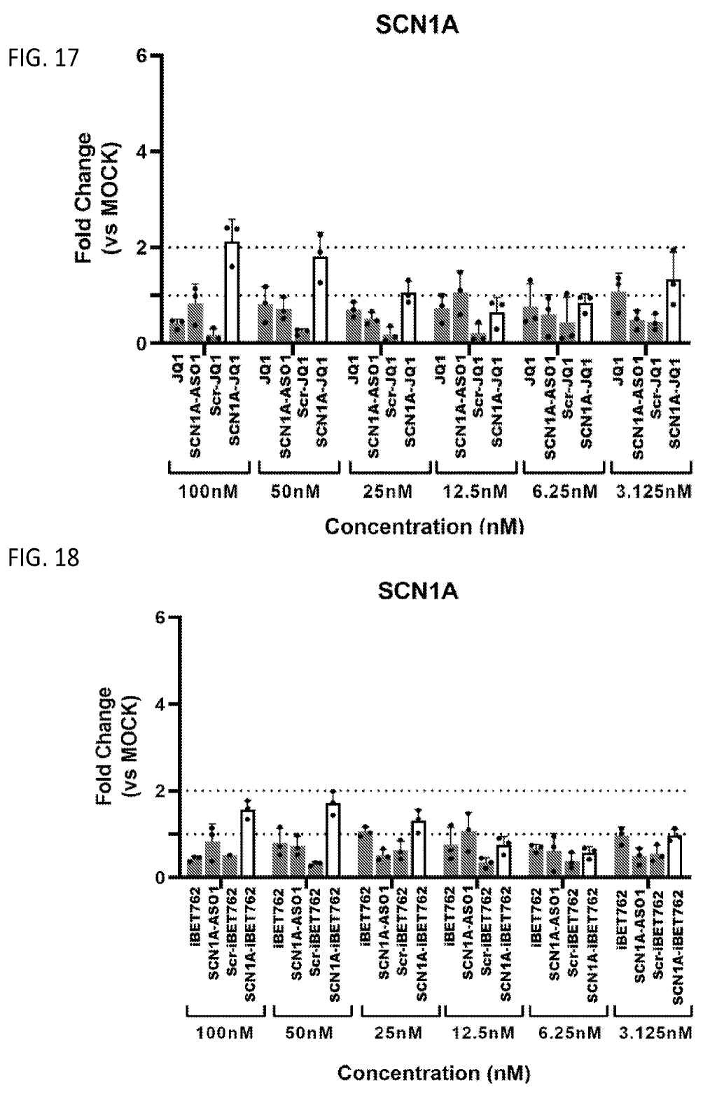

SCN1A ASO1-Linker1-iBET762 was transfected to SK-N-AS cells at 100, 50, 25, 12.5, 6.25, and 3.125 nM with RNAiMax. SCN1A ASO1-iBET762:RNAiMax ratios in transfection are: 100 nM:0.3 ul, 50 nM and below:0.15 ul. Cells were harvested 48 hours after transfection and SCN1A expression changes were monitored by qPCR. TaqMan probe used in the assay for quantitation: SCN1A Hs00374696_m1 (ThermoFisher), GAPDH Hs02786624_g1 (ThermoFisher). Results from the test showed that SCN1A ASO1-Linker1-iBET762 increased SCN1A expression by nearly 2-fold (FIG. 18). SCN1A encodes for the alpha-1 subunit of the voltage-gated sodium channel (Na(V)1.1), and patients with SCN1A loss of function mutations suffers from Dravet syndrome, a neurological disorder.

### Example 17: Rip Assay for BTK

### Methods

For expression of BTK, an expression plasmid was generated by cloning a DNA fragment (synthesized by Integrated DNA Technologies) encoding BTK with the following amino acid sequence:

KNAPSTAGLGYGSWEIDPKDLTFLKELGTGQFGVVKYGKWRGQYDVAIKM
IKEGSMSEDEFIEEAKVMMNLSHEKLVQLYGVCTKQRPIFIITEYMANGC
LLNYLREMRHRFQTQQLLEMCKDVCEAMEYLESKQFLHRDLAARNCLVND
QGVVKVSDFGLSRYVLDDEYTSSVGSKFPVRWSPPEVLMYSKFSSKSDIW
AFGVLMWEIYSLGKMPYERFTNSETAEHIAQGLRLYRPHLASEKVYTIMY
SCWHEKADERPTFKILLSNILDVMDEES (SEQ ID NO: 71)

The gene encoding BTK was directly fused to a sequence encoding three FLAG affinity tags with the following amino acid sequence:

DYKDHDGDYKDHDIDYKDDDDK (SEQ ID NO: 72)

For RNA immunoprecipitation assay (RIP), three million HEK293 cells were seeded onto 6-well cell culture plate on day 0. On day 1 (24 hours after cell seeding), 20 micrograms of the FLAG-BTK expression plasmid (described above) were transfected into the cells by Lipofectamine 2000 (Thermo Fisher Scientific) according to manufacturer’s instruction (45 microliters of lipofectamine mixed with 20 micrograms of DNA for 6 wells of a 6-well plate). On day 2 (24 hours after transfection of DNA), ibrutinib-conjugated anti-sense oligo (ASO-Linker1-Ib) targeting MALAT1 and HSP70 RNA transcripts were transfected into the cells at the final concentration of 150 nM using Lipofectamine RNAiMAX (Thermo Fisher Scientific) according to manufacturer’s recommendation (45 microliter of lipofectamine RNAiMAX for one 6-well culture plate).

Sequence of ASOs were as follows: MALAT1 ASO sequence:

CGTTAACTAGGCTTTA (SEQ ID NO: 5)

MALAT1 ASO Modifications (i2MOEr: “i” signifies internal base, “2MOE” indicate the 2′-O-methoxyethyl (2′-MOE) modification, “r” signifies ribonucleotide. The * indicates a phosphorothioate bond):

/i2MOErC/*/i2MOErG/*/i2MOErT/*/i2MOErT/*/i2MOErA/*
/i2MOErA/*/i2MOErC/*/i2MOErT/*/i2MOErA/*/i2MOErG/*
/i2MOErG/*/i2MOErC/*/i2MOErT/*/i2MOErT/*/i2MOErT/*
/32MOErA/HSP70 ASO: TCTTGGGCCGAGGCTACTGA (SEQ ID N
O: 6)

HSP70 ASO Modifications (i2MOEr: “i” signifies internal base, “2MOE” indicate the 2′-O-methoxyethyl (2′-MOE) modification, “r” signifies ribonucleotide. The * indicates a phosphorothioate bond):

*/i2MOErT/*/i2MOErC/*/i2MOErT/*/i2MOErT/*/i2MOErG/
*/i2MOErG/*/i2MOErG/*/i2MOErC/*/i2MOErC/*/i2MOErG/
*/i2MOErA/*/i2MOErG/*/i2MOErG/*/i2MOErC/*/i2MOErT/
*/i2MOErA/*/i2MOErC/*/i2MOErT/*/i2MOErG/*/32MOErA/

On day 3 (24 hours after the transfection of Ibrutinib ASOs), nuclei were extracted by suspending 6 million transfected cells in a hypotonic buffer (20 mM Tris-HCl, pH 7.4, 10 mM NaCl, 3 mM MgCl2) followed by centrifugation (500 g for 5 minutes at 4° C.). The nuclear lysate was prepared by resuspending the precipitated nuclei in the RIP buffer (150 mM KCl, 25 mM Tris pH 7.4, 5 mM EDTA, 0.5 mM DTT, 0.5% NP40, 100 U/ml RNAase inhibitor, and protease inhibitor). The lysate was divided into two portions and each portion was incubated with 1 microgram of either an anti-FLAG antibody (Sigma) or a control non-specific IgG (Cell Signaling Technology) for 4 hours at 4° C. on a rotator. Forty microliters of protein-G magnetic beads (Thermo Fisher Scientific) were subsequently added to the lysates and incubated for an additional one hour at 4° C. on a rotator. Beads were washed three times with RIP buffer. RNA was extracted by resuspending the washed beads in 1 milliliter of the Trizol reagent (Thermo Fisher Scientific) followed by addition of 200 ul of chloroform, centrifugation (10,000 g), and precipitation by isopropanol, according to the manufacturer’s instruction. Complementary DNA (cDNA) was produced from RNA by the iScript cDNA synthesis kit (BioRad). cDNA levels corresponding to RNA levels were quantified by quantitative PCR (qPCR) (Thermo Fisher Scientific). MALAT1 TaqMan probe: ThermoFisher Assay ID Hs00273907_s1; HSPA4/HSP70 TaqMan probe: ThermoFisher Assay ID Hs00382884_m1; ACTB TaqMan probe: ThermoFisher Assay ID Hs01060665_g1.

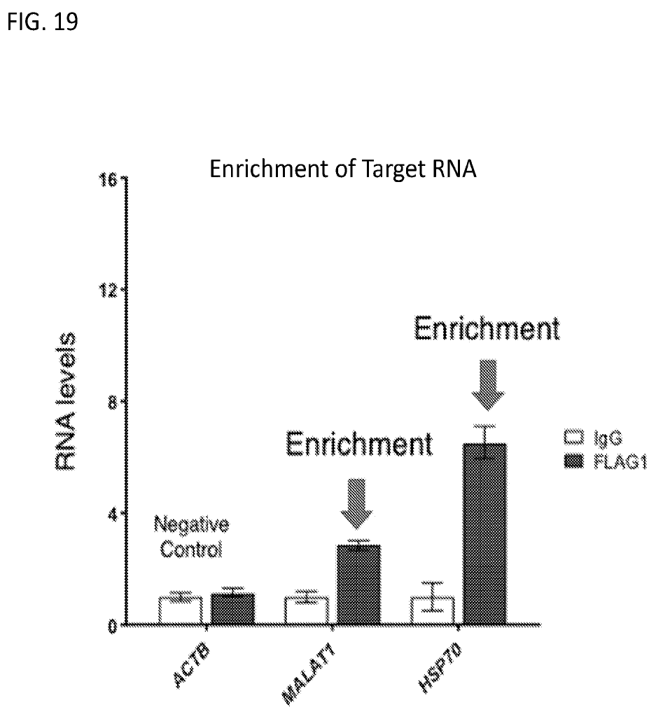

qRT-PCR shows the RNA levels of HSP70, MALAT1, and ACTB after RNA immunoprecipitation (RIP) of BTK protein in cells that were transfected with BTK and ibrutinib-conjugated ASOs targeting HSP70 and MALAT1 (FIG. 19). Enrichment of HSP70 and MALAT1 transcripts is observed in samples in which BTK is specifically pulled-down by an anti-FLAG antibody, but not with the non-specific IgG, which indicates the engagement of BTK with targets (MALAT1 and HSP70) through its interaction with the ibrutinib-conjugated ASOs.

### Example 18. Increase of SYNGAP1 Expression by Targeting SYNGAP1 mRNA With SYNGAP1 ASO-JQ1

5′amino modified SYNGAP1 ASOs were synthesized according to Example 2 and SYNGAP1 ASO1-JQ1 to SYNGAP1 ASO4-JQ1 were synthesized using Linker 2 according to the procedure described in Examples 2. SYNGAP1 ASO sequences and their modified versions are shown in Tables 1A and 1B.

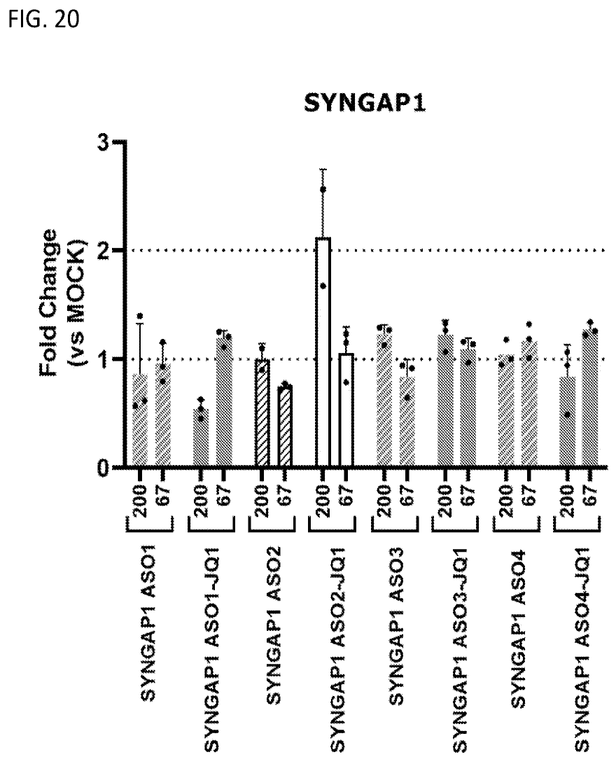

SYNGAP1 ASO1-JQ1 to SYNGAP1 ASO4-JQ1 were transfected to HEK293T cells at 200, and 67 nM with RNAiMax. SYNGAP1 ASO-JQ1:RNAiMax ratios in transfection are: 200 nM:0.6 ul, 67 nM:0.2 ul. Cells were harvested 48 hours after transfection and SYNGAP1 expression changes was monitored by qPCR. TaqMan probe used in the assay for quantitation: SYNGAP1: Assay ID Hs00405348_m1 (ThermoFisher), ACTB Assay ID Hs01060665_g1(ThermoFisher). Results from the test showed that at 200 nM, SYNGAP1 ASO2-JQ1 increased SYNGAP1 expression by about 2 fold (FIG. 20).

What is claimed is:

1. A method of increasing transcription of a gene and/or an RNA level of the gene in a cell comprising:
administering to a cell a synthetic bifunctional molecule comprising:
a first domain comprising a first small molecule or an antisense oligonucleotide (ASO), wherein the first domain specifically binds to a target ribonucleic acid (RNA) sequence;
a second domain comprising a second small molecule or an aptamer, wherein the second domain specifically binds to a target endogenous protein; and
a linker that conjugates the first domain to the second domain;
wherein the target endogenous protein increases transcription of a gene and/or an RNA level of the gene in the cell.

2. The method of claim 1, wherein the method increases transcription of the gene, and the target endogenous protein increases transcription of the gene in the cell.

3. The method of claim 1, wherein the method increases the RNA level of the gene, and the target endogenous protein increases the RNA level of the gene in the cell.

4. The method of claim 3, wherein increasing the RNA level increases a protein level in the cell.

5. The method of any one of the preceding claims, wherein the cell is a human cell.

6. The method of any one of the preceding claims, wherein the target endogenous protein is an intracellular endogenous protein.

7. The method of any one of the preceding claims, wherein the target endogenous protein is BRD4.

8. The method of any one of the preceding claims, wherein the first domain comprises the ASO.

9. The method of any one of the preceding claims, wherein the first domain comprises the ASO, and the ASO comprises one or more locked nucleic acids (LNA), one or more modified nucleobases, or a combination thereof.

10. The method of any one of the preceding claims, wherein the first domain comprises the ASO, and the ASO comprises a 5′ locked terminal nucleotide, a 3′ locked terminal nucleotide, or a 5′ and a 3′ locked terminal nucleotide.

11. The method of any one of the preceding claims, wherein the first domain comprises the ASO, and the ASO comprises a locked nucleotide at an internal position in the ASO.

12. The method of any one of the preceding claims, wherein the first domain comprises the ASO, and the ASO comprises a sequence comprising 30% to 60% GC content.

13. The method of any one of the preceding claims, wherein the first domain comprises the ASO, and the ASO comprises a length from 8 to 30 nucleotides.

14. The method of any one of the preceding claims, wherein the first domain comprises the first small molecule.

15. The method of any one of the preceding claims, wherein the second domain comprises the second small molecule.

16. The method of claim 15, wherein the second small molecule is an organic compound having a molecular weight of 900 daltons or less.

17. The method of claim 15, wherein the second small molecule comprises JQ1.

18. The method of claim 15, wherein the second small molecule comprises iBET762.

19. The method of claim 15, wherein the second small molecule comprises ibrutinib.

20. The method of any one of the preceding claims, wherein the second domain comprises the aptamer.

21. The method of any one of the preceding claims, wherein the linker is conjugated at a 5′ end or a 3′ end of the ASO.

22. The method of any one of the preceding claims, wherein the linker comprises at least one molecule selected from the group consisting of: 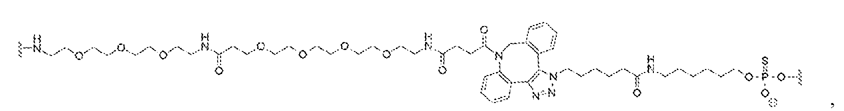 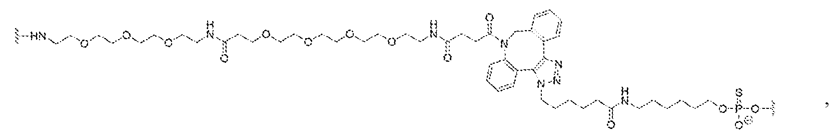 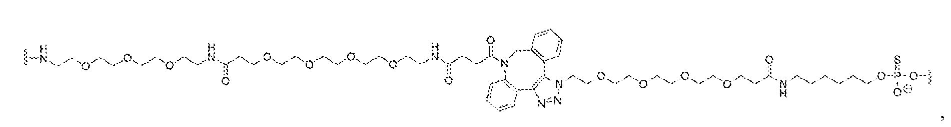 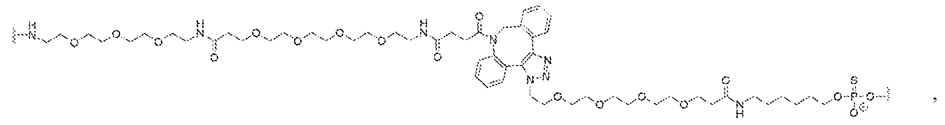 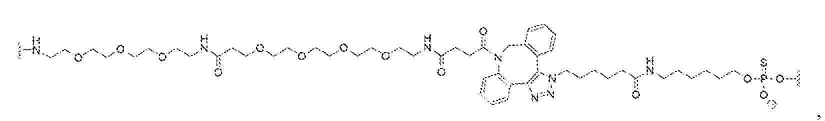 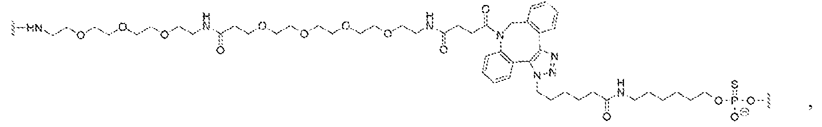 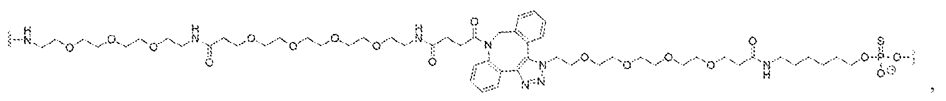 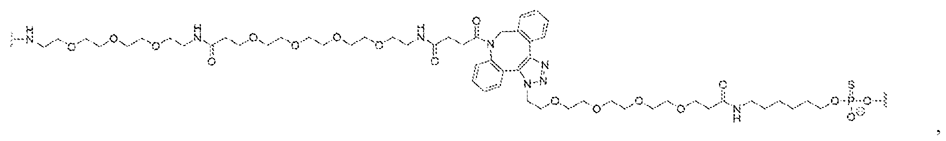 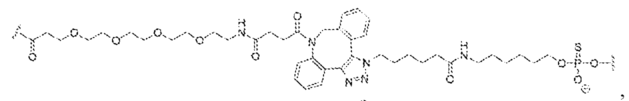 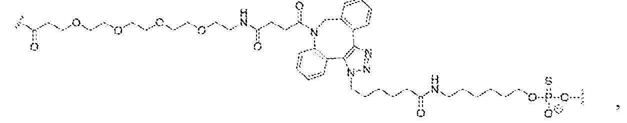 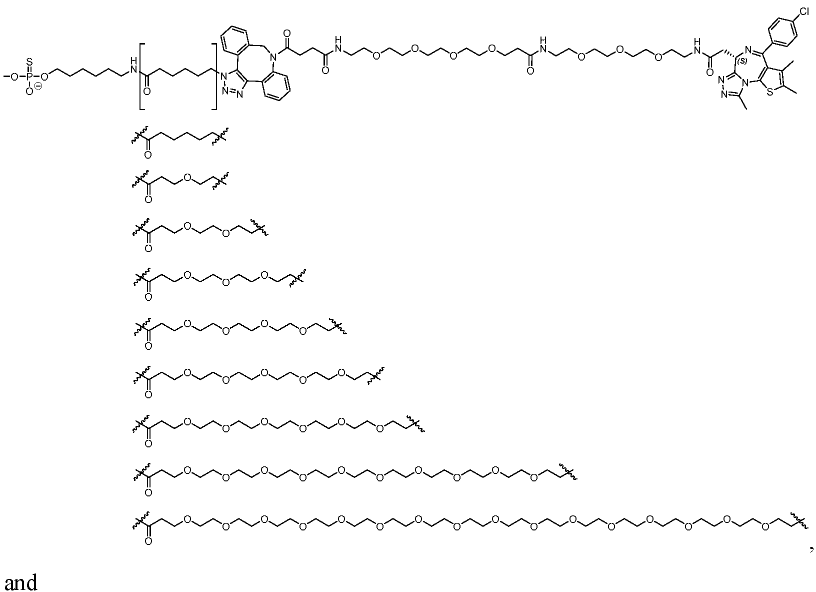 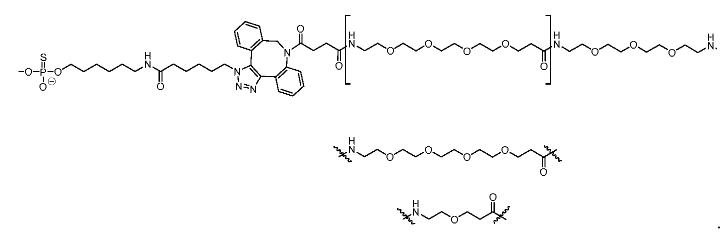 .

23. The method of any one of the preceding claims, wherein the target ribonucleic acid sequence is a nuclear RNA or a cytoplasmic RNA.

24. The method of claim 23, wherein the nuclear RNA or the cytoplasmic RNA is a long noncoding RNA (lncRNA), pre-mRNA, mRNA, microRNA, enhancer RNA, transcribed RNA, nascent RNA, chromosome-enriched RNA, ribosomal RNA, membrane enriched RNA, or mitochondrial RNA.

25. The method of any one of the preceding claims, wherein gene is associated with a disease or disorder.

26. A synthetic bifunctional molecule comprising:
a first domain comprising a first small molecule or an antisense oligonucleotide (ASO), wherein the first domain specifically binds to a target ribonucleic acid (RNA) sequence; and
a second domain comprising a second small molecule or an aptamer, wherein the second domain specifically binds to a target endogenous protein; and
wherein the first domain is conjugated to the second domain.

27. The method of claim 26, wherein the target endogenous protein is an intracellular endogenous protein.

28. The method of claim 26 or 27, wherein the target endogenous protein is BRD4.

29. The synthetic bifunctional molecule of any one of claims 26-28, wherein the first domain is conjugated to the second domain by a linker molecule.

30. The synthetic bifunctional molecule of claim 29, wherein the linker molecule is conjugated at a 5′ end or a 3′ end of the ASO.

31. The synthetic bifunctional molecule of claim 29 or 30, wherein the linker molecule comprises at least one molecule selected from the group consisting of: 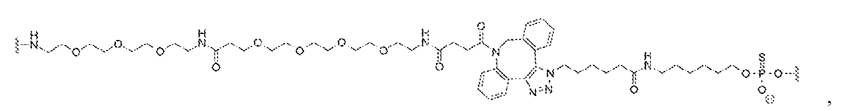 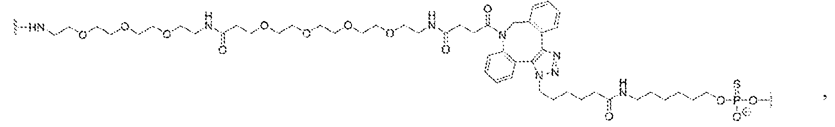 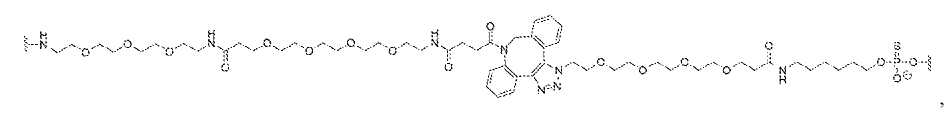 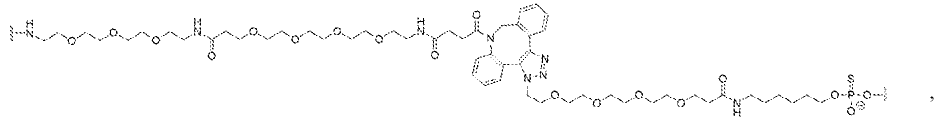 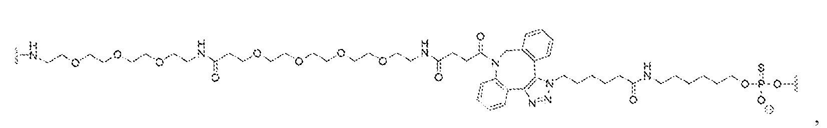 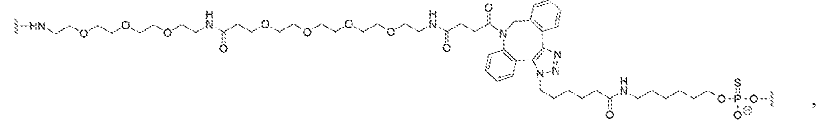 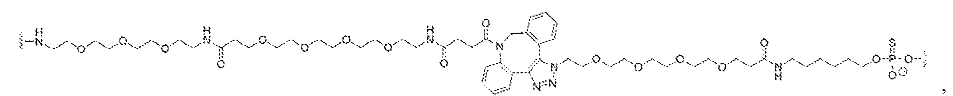 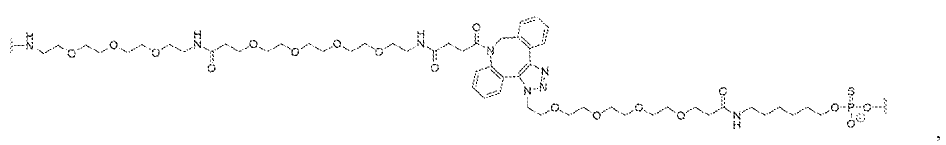 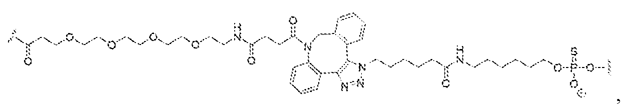 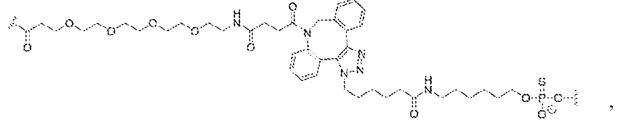 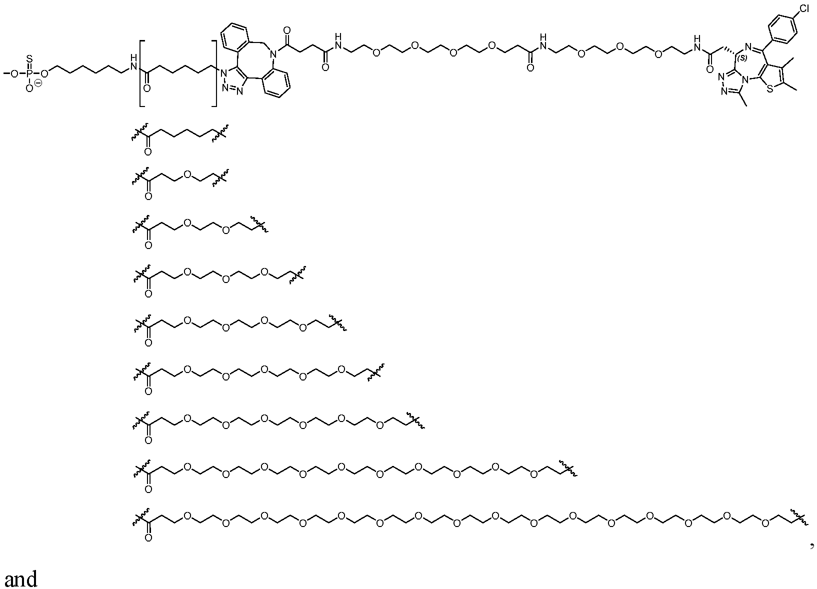 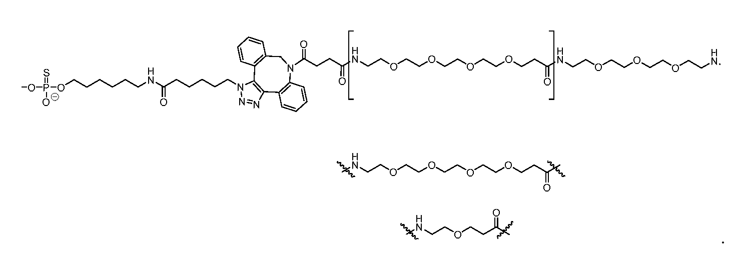 .

32. The synthetic bifunctional molecule of any one of claims 26-31, wherein the first domain comprises the ASO.

33. The synthetic bifunctional molecule of any one of claims 26-32, wherein the first domain comprises the ASO, and the ASO comprises one or more locked nucleic acids (LNA), one or more modified nucleobases, or a combination thereof.

34. The synthetic bifunctional molecule of any one of claims 26-33, wherein the first domain comprises the ASO, and the ASO comprises a 5′ locked terminal nucleotide, a 3′ locked terminal nucleotide, or a 5′ and a 3′ locked terminal nucleotide.

35. The synthetic bifunctional molecule of any one of claims 26-34, wherein the first domain comprises the ASO, and the ASO comprises a locked nucleotide at an internal position in the ASO.

36. The synthetic bifunctional molecule of any one of claims 26-35, wherein the first domain comprises the ASO, and the ASO comprises a sequence comprising 30% to 60% GC content.

37. The synthetic bifunctional molecule of any one of claims 26-36, wherein the first domain comprises the ASO, and the ASO comprises a length from 8 to 30 nucleotides.

38. The synthetic bifunctional molecule of any one of claims 26-37, wherein the first domain comprises the first small molecule.

39. The synthetic bifunctional molecule of any one of claims 26-38, wherein the second domain comprises the second small molecule.

40. The synthetic bifunctional molecule of claim 39, wherein the second small molecule comprises JQ1.

41. The synthetic bifunctional molecule of claim 39, wherein the second small molecule comprises iBET762.

42. The synthetic bifunctional molecule of claim 39, wherein the second small molecule comprises ibrutinib.

43. The synthetic bifunctional molecule of any one of claims 26-42, wherein the second domain comprises the aptamer.

44. The synthetic bifunctional molecule of any one of claims 26-43, wherein the target ribonucleic acid sequence is a nuclear RNA or a cytoplasmic RNA.

45. The synthetic bifunctional molecule of claim 44, wherein the nuclear RNA or the cytoplasmic RNA is a long noncoding RNA (lncRNA), pre-mRNA, mRNA, microRNA, enhancer RNA, transcribed RNA, nascent RNA, chromosome-enriched RNA, ribosomal RNA, membrane enriched RNA, or mitochondrial RNA.


SEQUENCE LISTING (72 sequences)

SEQ ID NO: 1 - RNA, 16 nt, Artificial Sequence
  MALAT1 targetting ASO
cguuaacuaggcuuua

SEQ ID NO: 2 - DNA, 20 bp, Artificial Sequence
  XIST targeting ASO
ggaagggaatcagcaggtat

SEQ ID NO: 3 - DNA, 20 bp, Artificial Sequence
  HSP70 targeting ASO
tcttgggccgaggctactga

SEQ ID NO: 4 - DNA, 20 bp, Artificial Sequence
  MYC targeting ASO
cctggggctggtgcattttc

SEQ ID NO: 5 - DNA, 16 bp, Artificial Sequence
  MALAT1 targetting ASO
cgttaactaggcttta

SEQ ID NO: 6 - DNA, 20 bp, Artificial Sequence
  HSP70 targetting ASO
tcttgggccgaggctactga

SEQ ID NO: 7 - DNA, 19 bp, Artificial Sequence
  PVT1 ASO1
gtaagtggaattccagttg

SEQ ID NO: 8 - DNA, 20 bp, Artificial Sequence
  PVT1 ASO2
agctttagaccacgaggcac

SEQ ID NO: 9 - DNA, 20 bp, Artificial Sequence
  PVT1 ASO3
aagctttagaccacgaggca

SEQ ID NO: 10 - DNA, 20 bp, Artificial Sequence
  PVT1 ASO4
gaagctttagaccacgaggc

SEQ ID NO: 11 - DNA, 20 bp, Artificial Sequence
  PVT1 ASO5
cgaagctttagaccacgagg

SEQ ID NO: 12 - DNA, 20 bp, Artificial Sequence
  PVT1 ASO6
ccgaagctttagaccacgag

SEQ ID NO: 13 - DNA, 20 bp, Artificial Sequence
  PVT1 ASO7
gccgaagctttagaccacga

SEQ ID NO: 14 - DNA, 20 bp, Artificial Sequence
  PVT1 ASO8
tgccgaagctttagaccacg

SEQ ID NO: 15 - DNA, 20 bp, Artificial Sequence
  PVT1 ASO9
gtgccgaagctttagaccac

SEQ ID NO: 16 - DNA, 20 bp, Artificial Sequence
  PVT1 ASO10
tgtgccgaagctttagacca

SEQ ID NO: 17 - DNA, 20 bp, Artificial Sequence
  PVT1 ASO11
ttgtgccgaagctttagacc

SEQ ID NO: 18 - DNA, 20 bp, Artificial Sequence
  PVT1 ASO12
cttgtgccgaagctttagac

SEQ ID NO: 19 - DNA, 20 bp, Artificial Sequence
  PVT1 ASO13
ccttgtgccgaagctttaga

SEQ ID NO: 20 - DNA, 20 bp, Artificial Sequence
  PVT1 ASO14
cccttgtgccgaagctttag

SEQ ID NO: 21 - DNA, 20 bp, Artificial Sequence
  PVT1 ASO15
gcccttgtgccgaagcttta

SEQ ID NO: 22 - DNA, 20 bp, Artificial Sequence
  PVT1 ASO16
ggcccttgtgccgaagcttt

SEQ ID NO: 23 - DNA, 20 bp, Artificial Sequence
  PVT1 ASO17
gacacggattctgtatttgt

SEQ ID NO: 24 - DNA, 20 bp, Artificial Sequence
  PVT1 ASO18
aggccacgaggtttctccca

SEQ ID NO: 25 - DNA, 20 bp, Artificial Sequence
  PVT1 ASO19
catctcaaataatggagacc

SEQ ID NO: 26 - DNA, 20 bp, Artificial Sequence
  PVT1 ASO20
tttagaccacgaggcacgtc

SEQ ID NO: 27 - DNA, 20 bp, Artificial Sequence
  PVT1 ASO21
agtaaacagagatctcaacc

SEQ ID NO: 28 - DNA, 20 bp, Artificial Sequence
  PVT1 ASO22
ctggatggaagtatacacca

SEQ ID NO: 29 - DNA, 20 bp, Artificial Sequence
  PVT1 ASO23
tatcacagaactaggctgtg

SEQ ID NO: 30 - DNA, 20 bp, Artificial Sequence
  PVT1 ASO24
cattgaaggatcatggtcat

SEQ ID NO: 31 - DNA, 20 bp, Artificial Sequence
  PVT1 ASO25
ttatagactagattggccag

SEQ ID NO: 32 - DNA, 20 bp, Artificial Sequence
  PVT1 ASO26
tttaatctccttctggccaa

SEQ ID NO: 33 - DNA, 20 bp, Artificial Sequence
  PVT1 ASO27
cagcagtcatccaaatattc

SEQ ID NO: 34 - DNA, 20 bp, Artificial Sequence
  PVT1 ASO28
aagctccagccacagaaaca

SEQ ID NO: 35 - DNA, 20 bp, Artificial Sequence
  PVT1 ASO29
actcctcctttccagtgcag

SEQ ID NO: 36 - DNA, 20 bp, Artificial Sequence
  PVT1 ASO30
ccacttaacaaatccctctg

SEQ ID NO: 37 - DNA, 20 bp, Artificial Sequence
  PVT1 ASO31
gccactcttaaccaggcaaa

SEQ ID NO: 38 - DNA, 20 bp, Artificial Sequence
  PVT1 ASO32
agtcatacccgtaagtggaa

SEQ ID NO: 39 - DNA, 20 bp, Artificial Sequence
  PVT1 ASO33
cacagtcatacccgtaagtg

SEQ ID NO: 40 - DNA, 20 bp, Artificial Sequence
  MYC ASO1
tttcttctttctctcgccgg

SEQ ID NO: 41 - DNA, 20 bp, Artificial Sequence
  MYC ASO2
aaggtttcagaggtgatgag

SEQ ID NO: 42 - DNA, 20 bp, Artificial Sequence
  MYC ASO3
cggagacgcacttagtgaac

SEQ ID NO: 43 - DNA, 20 bp, Artificial Sequence
  MYC ASO4
gtcctaacacctctagagac

SEQ ID NO: 44 - DNA, 20 bp, Artificial Sequence
  MYC ASO5
ttcattcactctcagagatc

SEQ ID NO: 45 - DNA, 20 bp, Artificial Sequence
  MYC ASO6
gcatgaatacgttagaaagg

SEQ ID NO: 46 - DNA, 18 bp, Artificial Sequence
  SCN1A ASO1
agtaagactggggttgtt

SEQ ID NO: 47 - DNA, 19 bp, Artificial Sequence
  SYNGAP1 ASO1
taggaagtatcaagctgtg

SEQ ID NO: 48 - DNA, 19 bp, Artificial Sequence
  SYNGAP1 ASO2
atcacctcctatagctcct

SEQ ID NO: 49 - DNA, 19 bp, Artificial Sequence
  SYNGAP1 ASO3
catctctcaccacgtttgg

SEQ ID NO: 50 - DNA, 19 bp, Artificial Sequence
  SYNGAP1 ASO4
aatcttgccatcacccaca

SEQ ID NO: 51 - RNA, 169 nt, Artificial Sequence
  Cy5-labeled IVT RNA
ccuugaaauccaugacgcagggagaauugcgucauuuaaagccuaguuaacgcauuuacuaaacgcagacgaaaauggaaagauuaauugggagugguaggaugaaacaauuuggagaagauagaaguuugaaguggaaaacuggaagacagaaguacgggaaggcgaa

SEQ ID NO: 52 - DNA, 18 bp, Artificial Sequence
  Non PVT1 targeting ASO1
gtcgaataaaccagtatc

SEQ ID NO: 53 - DNA, 23 bp, Artificial Sequence
  Non PVT1 targeting ASO2
gatccaagtaaatcagcacgacc

SEQ ID NO: 54 - DNA, 20 bp, Artificial Sequence
  Non PVT1 targeting ASO3
ataggtggtctctgatggtc

SEQ ID NO: 55 - DNA, 18 bp, Artificial Sequence
  Non PVT1 targeting ASO4
agtaagactggggttgtt

SEQ ID NO: 56 - DNA, 20 bp, Artificial Sequence
  Non PVT1 targeting ASO5
gtatgtgtaccgcattgttt

SEQ ID NO: 57 - DNA, 26 bp, Artificial Sequence
  Non PVT1 targeting ASO6
gagccagtcacaaattcagatcaccc

SEQ ID NO: 58 - DNA, 20 bp, Artificial Sequence
  Non PVT1 targeting ASO7
ttgtcgtaagtgttgcaaac

SEQ ID NO: 59 - DNA, 20 bp, Artificial Sequence
  Non PVT1 targeting ASO8
actgaattctgacaaatgac

SEQ ID NO: 60 - DNA, 16 bp, Artificial Sequence
  Scramble A (ScrA)
agaggtggcgtggtag

SEQ ID NO: 61 - DNA, 16 bp, Artificial Sequence
  Scramble B (ScrB)
aacacgtctatacgcc

SEQ ID NO: 62 - DNA, 19 bp, Artificial Sequence
  PVT1- ASO1
gtaagtggaattccagttg

SEQ ID NO: 63 - DNA, 19 bp, Artificial Sequence
  PVT1-ASO1-scr1
tgaagtggaattccagttg

SEQ ID NO: 64 - DNA, 19 bp, Artificial Sequence
  PVT1-ASO1-scr2
gtaagaggtattccagttg

SEQ ID NO: 65 - DNA, 19 bp, Artificial Sequence
  PVT1-ASO1-scr3
gtaagtggaactatcgttg

SEQ ID NO: 66 - DNA, 19 bp, Artificial Sequence
  PVT1-ASO1-scr4
gtaagtggaattccattgg

SEQ ID NO: 67 - DNA, 19 bp, Artificial Sequence
  PVT1-ASO1-scr5
taggatggaattccagttg

SEQ ID NO: 68 - DNA, 19 bp, Artificial Sequence
  PVT1-ASO1-scr6
gtaagataggttccagttg

SEQ ID NO: 69 - DNA, 19 bp, Artificial Sequence
  PVT1-ASO1-scr7
gtaagtggaacattcgttg

SEQ ID NO: 70 - DNA, 19 bp, Artificial Sequence
  PVT1-ASO1-scr8
gtaagtggaattccatggt

SEQ ID NO: 71 - protein, 278 aa, Artificial Sequence
  BTK
KNAPSTAGLGYGSWEIDPKDLTFLKELGTGQFGVVKYGKWRGQYDVAIKMIKEGSMSEDEFIEEAKVMMNLSHEKLVQLYGVCTKQRPIFIITEYMANGCLLNYLREMRHRFQTQQLLEMCKDVCEAMEYLESKQFLHRDLAARNCLVNDQGVVKVSDFGLSRYVLDDEYTSSVGSKFPVRWSPPEVLMYSKFSSKSDIWAFGVLMWEIYSLGKMPYERFTNSETAEHIAQGLRLYRPHLASEKVYTIMYSCWHEKADERPTFKILLSNILDVMDEES

SEQ ID NO: 72 - protein, 22 aa, Artificial Sequence
  Synthetic Sequence
DYKDHDGDYKDHDIDYKDDDDK